(19) United States
(12) Patent Application Publication
(10) Pub. No.: US 2016/0319307 A1
(43) Pub. Date: Nov. 3, 2016

(54) Fermentive Production of Isobutanol Using Highly Effective Ketol-Acid Reductoisomerase Enzymes

(71) Applicant: BUTAMAX ADVANCED BIOFUELS LLC, Wilmington, DE (US)

(72) Inventors: Vasantha Nagarajan, Wilmington, DE (US); Brian James Paul, Wilmington, DE (US); Wonchul Suh, Hockessin, DE (US); Jean-Francois Tomb, Wilmington, DE (US); Rick W. Ye, Hockessin, DE (US)

(21) Appl. No.: 15/209,841

(22) Filed: Jul. 14, 2016

Related U.S. Application Data
(63) Continuation of application No. 12/893,077, filed on Sep. 29, 2010, now abandoned.
(60) Provisional application No. 61/246,844, filed on Sep. 29, 2009.

(51) Int. Cl. C12P 7/16; C12N 9/88; C12N 15/52; C12N 9/04

(52) U.S. Cl. CPC C12P 7/16; C12N 9/0006; C12N 9/88; C12Y 101/01086; C12Y 401/01001; C12Y 101/01008; C12Y 401/02009; C12Y 401/01072; C12Y 402/01009; C12N 15/52

(57) ABSTRACT

Ketol-acid reductoisomerase enzymes have been identified that provide high effectiveness in vivo as a step in an isobutanol biosynthetic pathway in bacteria and in yeast. These KARIs are members of a clade identified through molecular phylogenetic analysis called the SLSL Clade.

### CROSS-REFERENCE TO RELATED APPLICATIONS

This application claims priority to U.S. Provisional Patent Application No. 61/246,844, filed on Sep. 29, 2009, the entirety of which is herein incorporated by reference.

### FIELD OF THE INVENTION

The invention relates to the field of industrial microbiology and fermentation, specifically for production of isobutanol. More specifically, ketol-acid reductoisomerase (KARI) enzymes were found that are highly effective in an engineered isobutanol biosynthetic pathway for production of isobutanol in lactic acid bacteria and yeast.

### BACKGROUND OF THE INVENTION

Butanol is an important industrial chemical, useful as a fuel additive, as a feedstock chemical in the plastics industry, and as a foodgrade extractant in the food and flavor industry. Each year 10 to 12 billion pounds of butanol are produced by petrochemical means and the need for this commodity chemical will likely increase.

Microorganisms have been engineered to produce butanols by expressing butanol biosynthetic pathways. Pathways for biosynthesis of isobutanol are disclosed in US Patent Publication No. US 20070092957. To obtain commercially viable production of isobutanol, a very efficient isobutanol pathway is needed. The second step of the pathway is catalyzed by ketol-acid reductoisomerase (KARI), which converts acetolactate to dihydroxy-isovalerate. KARI enzymes with high activity and use of these enzymes in an isobutanol biosynthetic pathway have been disclosed in US Patent Publication No. US20080261230A1.

There remains a need to further improve the step of converting acetolactate to dihydroxy-isovalerate in a microorganism that is engineered with an isobutanol biosynthetic pathway, to maximize production of isobutanol.

### SUMMARY OF THE INVENTION

The invention provides microbial host cells that express a ketol-acid reductoisomerase (KARI) enzyme that provides highly effective conversion of acetolactate to dihydroxy-isovalerate in vivo such that more isobutanol is produced in a host cell having an engineered isobutanol biosynthetic pathway. The highly effective KARIs are in a molecular phylogenetic grouping that includes the Lactococcus lactis and Streptococcus mutans KARIs.

In one aspect of the invention, a yeast cell comprising at least one nucleic acid molecule encoding a polypeptide having ketol-acid reductoisomerase activity wherein said polypeptide is a member of the SLSL Clade of KARIs, is provided. In one another aspect the yeast cell is a member of a genus of yeast selected from the group consisting of Saccharomyces, Schizosaccharomyces, Hansenula, Candida, Kluyveromyces, Yarrowia and Pichia.

In another aspect said SLSL Clade consists of ketol-acid reductoisomerases that are endogenous to bacteria selected from the group consisting of Staphylococcus, Listeria, Enterococcus, Macrococcus, Streptococcus, Lactococcus, Leuconostoc, Lactobacillus.

In another aspect the polypeptide having ketol-acid reductoisomerase activity has an amino acid sequence that is at least about 80% identical to a sequence selected from the group consisting of SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, and 245.

Another aspect of the invention is an isobutanol producing microbial cell comprising at least one nucleic acid molecule encoding a polypeptide having ketol-acid reductoisomerase activity wherein said polypeptide is a member of the SLSL Clade of KARIs.

In another aspect said microbial cell is a bacteria cell of a genus selected from the group consisting of Escherichia, Rhodococcus, Pseudomonas, Bacillus, Enterococcus, Lactococcus, Lactobacillus, Leuconostoc, Oenococcus, Pediococcus, Streptococcus, Clostridium, Zymomonas, Salmonella, Pediococcus, Alcaligenes, Klebsiella, Paenibacillus, Arthrobacter, Corynebacterium, and Brevibacterium.

In another aspect, a method for converting acetolactate to dihydroxy-isovalerate is provided, said method comprising:
a) providing a yeast cell comprising at least one nucleic acid molecule encoding a polypeptide having ketol-acid reductoisomerase activity wherein said polypeptide is a member of the SLSL Clade of KARIs, and
b) contacting the yeast cell of (a) with acetolactate wherein 2,3-dihydroxy-isovalerate is produced.

In another aspect, a method for the production of isobutanol is provided, said method comprising:
a) providing a microbial cell comprising an isobutanol biosynthetic pathway comprising at least one nucleic acid molecule encoding a polypeptide having ketol-acid reductoisomerase activity wherein said polypeptide is a member of the SLSL Clade of KARIs,
b) growing the microbial cell of step (a) under conditions wherein isobutanol is produced.

Also provided herein are yeast cells engineered to have at least one pyruvate decarboxylase gene inactivated and comprising a plasmid having the coding regions of a plasmid selected from the group consisting of SEQ ID NO: 198, 203, 204, 208, or 211 and those with coding regions having at least about 80%, at least about 90%, at least about 95%, or at least about 99% identity to the coding regions of a plasmid selected from the group consisting of SEQ ID NO: 198, 203, 204, 208, or 211. Also provided are yeast cells engineered to have at least one pyruvate decarboxylase gene inactivated and comprising a plasmid having the chimeric genes of a plasmid selected from the group consisting of SEQ ID NO: 198, 203, 204, 208, or 211 and those with chimeric genes with at least about 80% at least about 85%, at least about 90%, at least about 95%, or at least about 99% identity to the chimeric genes of a plasmid selected from the group consisting of SEQ ID NO: 198, 203, 204, 208, or 211. Also provided are plasmids having the sequence of SEQ ID NO: 198, 203, 204, 208, or 211 and those with at least about 80%, at least about 90%, at least about 95%, or at least about 99% identity.

### BRIEF DESCRIPTION OF THE DRAWINGS

The various embodiments of the invention can be more fully understood from the following detailed description, the figures, and the accompanying sequence descriptions, which form a part of this application.

FIG. 1 shows three different isobutanol biosynthetic pathways.

FIG. 2 shows a molecular phylogenetic tree of KARIs including 667 sequences with a 95% sequence identity cut-off. The symbols outside of the circle mark the SLSL Clade.

FIG. 3 shows a portion of the phylogenetic tree of FIG. 2 that includes the SLSL Clade of KARI sequences. Diamonds mark members of the order Lactobacillales and circles mark members of the order Bacillales. A species representing the 95% identity group for each sub-branch is listed in the key.

FIG. 4 shows a graph of the growth curves of isobutanol producing yeast with different KARI enzymes. SYK: single yeast ILV5; SLK: single L. lactis IlvC, SPK: single Pf-5 ilvC.

FIG. 5 shows a graph of isobutanol titers for isobutanol producing yeast with different KARI enzymes. SYK: single yeast ILV5; SLK: single L. Lactis IlvC, SPK: single Pf-5 ilvC.

The invention can be more fully understood from the following detailed description and the accompanying sequence descriptions which form a part of this application.

The following sequences conform with 37 C.F.R. 1.821-1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (1998) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

TABLE 1
SEQ ID numbers of Coding Regions and
Proteins for highly effective KARIs

| | SEQ ID NO: | SEQ ID NO: |
|---|---|---|
| Description | Nucleic acid | Amino acid |
| Staphylococcus capitis SK14 | 1 | 2 |
| Staphylococcus epidermidis M23864-W1 | 3 | 4 |
| Staphylococcus hominis SK119 | 244 | 245 |
| Staphylococcus aureus subsp. aureus | 5 | 6 |
| TCH130 | | |
| Staphylococcus warneri L37603 | 7 | 8 |
| Staphylococcus epidermidis W23144 | 9 | 10 |
| Staphylococcus saprophyticus subsp. | 11 | 12 |
| Saprophyticus ATCC15305 | | |
| Staphylococcus carnosus subsp. Carnosus | 13 | 14 |
| TM300 | | |
| Listeria monocytogenes EGD-e | 15 | 16 |
| Listeria grayi DSM 20601 | 17 | 18 |
| Enterococcus casseliflavus EC30 | 19 | 20 |
| Enterococcus gallinarum EG2 | 21 | 22 |
| Macrococcus caseolyticus JCSC5402 | 23 | 24 |
| Streptococcus vestibularis | 25 | 26 |
| Streptococcus mutans UA159 | 27 | 28 |
| Streptococcus gordonii str, cgakkus | 29 | 30 |
| sybstr. CH1 | | |
| Streptococcus suis 89/1591 | 31 | 32 |
| Streptococcus infantarius subsp. | 33 | 34 |
| infantarius ATCC BAA-102 | | |
| Lactococcus lactis subsp cremoris MG1363 | 35 | 36 |
| Lactococcus lactis | 37 | 38 |
| Leuconostoc mesenteroides subsp | 39 | 40 |
| mesenteroides ATCC8293 | | |
| Lactobacillus buchneri ATCC 11577 | 41 | 42 |
| Staphylococcus haemolyticus JCSC1435 | 43 | 44 |
| Staphylococcus epidermidis ATCC12228 | 45 | 46 |
| Streptococcus pneumoniae CGSP14 | 47 | 48 |
| Streptococcus pneumoniae TIGR4 | 49 | 50 |
| Streptococcus sanguinis SK36 | 51 | 52 |
| Streptococcus salivarius SK126 | 53 | 54 |
| Streptococcus thermophilus LMD-9 | 55 | 56 |
| Streptococcus pneumoniae CCRI 1974M2 | 57 | 58 |
| Lactococcus lactis subsp. lactis II1403 | 59 | 60 |
| Leuconostoc mesenteroides subsp cremoris | 61 | 62 |
| ATCC19254 | | |
| Leuconostoc mesenteroides subsp cremoris | 63 | 64 |
| Lactobacillus brevis subsp. gravesensis | 65 | 66 |
| ATCC27305 | | |
| Lactococcus lactis subsp lactis NCDO2118 | 67 | 68 |

TABLE 2
SEQ ID NOs of expression coding regions and proteins

| | SEQ ID NO: | SEQ ID NO: |
|---|---|---|
| Description | nucleic acid | amino acid |
| ALS from Bacillus subtilis | 69 | 70 |
| ALS from Bacillus subtilis coding region | 71 | 70* |
| optimized for Lactobacillus plantarum | | |
| ALS from Klebsiella pneumoniae (budB) | 72 | 73 |
| ALS from Lactococcus lactis | 74 | 75 |
| ALS from Staphylococcus aureus | 76 | 77 |
| ALS from Listeria monocytogenes | 78 | 79 |
| ALS from Streptococcus mutans | 80 | 81 |
| ALS from Streptococcus thermophilus | 82 | 83 |
| ALS from Vibrio angustum | 84 | 85 |
| ALS from Bacillus cereus | 86 | 87 |
| KARI from Pseudomonas fluorescens ilvC | 88 | 89 |
| PF5 | | |
| KARI from Pseudomonas fluorescens ilvC | 90 | 89* |
| PF5 codon optimized for L. plantarum | | |
| KARI from Pseudomonas fluorescens ilvC | 91 | 89* |
| PF5 codon optimized for S. cerevisiae | | |
| KARI from Saccharomyces cerevisiae ILV3 | 92 | 93 |
| DHAD from Lactococcus lactis ilvD | 94 | 95 |
| DHAD from Streptococcus mutans ilvD | 96 | 97 |
| DHAD from Saccharomyces cerevisiae | 98 | 99 |
| ILV3 | | |
| branched chain keto acid decarboxylase | 100 | 101 |
| from Lactococcus lactis kivD | | |
| Lactococcus lactis kivD opt for L. plantarum | 102 | 101* |
| secondary alcohol dehydrogenase from | 103 | 104 |
| Achromobacter xylosoxidans sadB | | |
| A. xylosoxidans sadB opt for L. plantarum | 105 | 104* |
| Horse liver alcohol dehydrogenase ADH | 106 | 107 |
| codon optimized for S. cerevisiae | | |
| Tn5 transposase | 108 | 109 |

*same protein sequence encoded by native and optimized sequence

SEQ ID NO:110 is the sequence of plasmid pFP996.

SEQ ID NOs:111-121. 123-126, 130, 131, 133, 134, 136-141, 143-148, 151-154, 156-159, 161-163, 165, 166, 168, 170-173, 177-181, 186-197, 199-202, 205, 206, 209, 210, 213-222, 224-243 are PCR and sequencing primers.

SEQ ID NO:122 is the sequence of pyrF.

SEQ ID NO:127 is a ribosome binding site (RBS).

SEQ ID NO:128 is the sequence of plasmid pDM20-ilvD (L. lactis).

SEQ ID NO:129 is the sequence of plasmid pDM1.

SEQ ID NO:132 is the sequence of a PCR fragment including a RBS and ilvD coding region from Lactococcus lactis.

SEQ ID NO:135 is a right homologous arm DNA fragment containing the 5′ portion of the suf operon (sufC and part of sufD).

SEQ ID NO:142 is a left homologous arm DNA fragment containing the native suf promoter and sequences upstream into the feoBA operon.

SEQ ID NO:149 is the sequence of plasmid pTN6.

SEQ ID NO:150 is the sequence of a Tn5IE-loxP-cm-Pspac-loxP cassette.

SEQ ID NO:155 is the Pnpr promoter.

SEQ ID NO:160 is a Pnpr-tnp fusion DNA fragment.

SEQ ID NO:164 is a PgroE promoter sequence.

SEQ ID NO:167 is a PCR fragment containing the kivD(o) coding region together with a RBS.

SEQ ID NO:169 a DNA fragment containing an RBS and sadB(o) coding region.

SEQ ID NO:174 is the sequence of plasmid pFP352.

SEQ ID NO:175 is the sequence of plasmid pDM5.

SEQ ID NO:176 is a lacI-PgroE/lacO fragment.

SEQ ID NO:182 is the sequence of plasmid pDM5-PldhL1-ilvC (L. lactis).

SEQ ID NO:183 is a DNA fragment including a RBS and coding region for PF5-ilvC codon optimized for L. plantarum expression.

SEQ ID NO:184 is the sequence of plasmid pFP996-PldhL1.

SEQ ID NO:185 is a PldhL1-ilvC(P. fluorescens PF5) DNA fragment.

SEQ ID NO:198 is the sequence of plasmid pYZ090.

SEQ ID NO:203 is the sequence of plasmid pLH475-IlvC (L. lactis).

SEQ ID NO:204 is the sequence of plasmid pYZ091.

SEQ ID NO:207 is the sequence of plasmid pLH532.

SEQ ID NO:208 is the sequence of plasmid pYZ058.

SEQ ID NO:211 is the sequence of plasmid pYZ067.

SEQ ID NO:212 is the sequence of the pUC19-URA3r vector.

SEQ ID NO:223 is the sequence of the ilvD-FBA1t fragment.

### DETAILED DESCRIPTION

The present invention relates to recombinant microbial host cells engineered for improved production of isobutanol. Isobutanol is an important compound for use in replacing fossil fuels.

The following abbreviations and definitions will be used for the interpretation of the specification and the claims.

As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

The term “invention” or “present invention” as used herein is a non-limiting term and is not intended to refer to any single embodiment of the particular invention but encompasses all possible embodiments as described in the specification and the claims.

As used herein, the term “about” modifying the quantity of an ingredient or reactant of the invention employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities. In one embodiment, the term “about” means within 10% of the reported numerical value, preferably within 5% of the reported numerical value.

The term “isobutanol biosynthetic pathway” refers to an enzyme pathway to produce isobutanol from pyruvate.

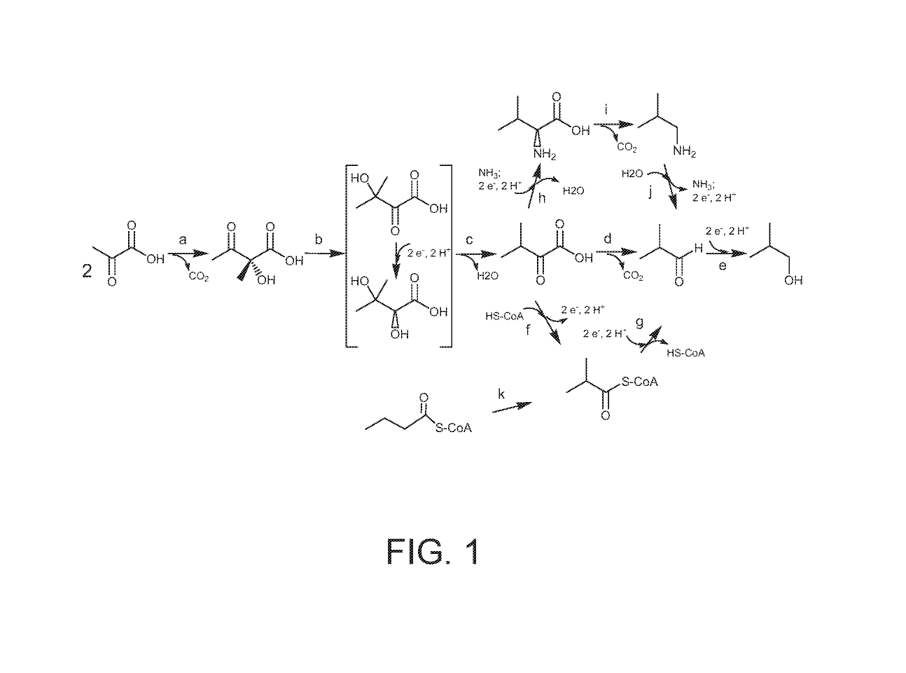

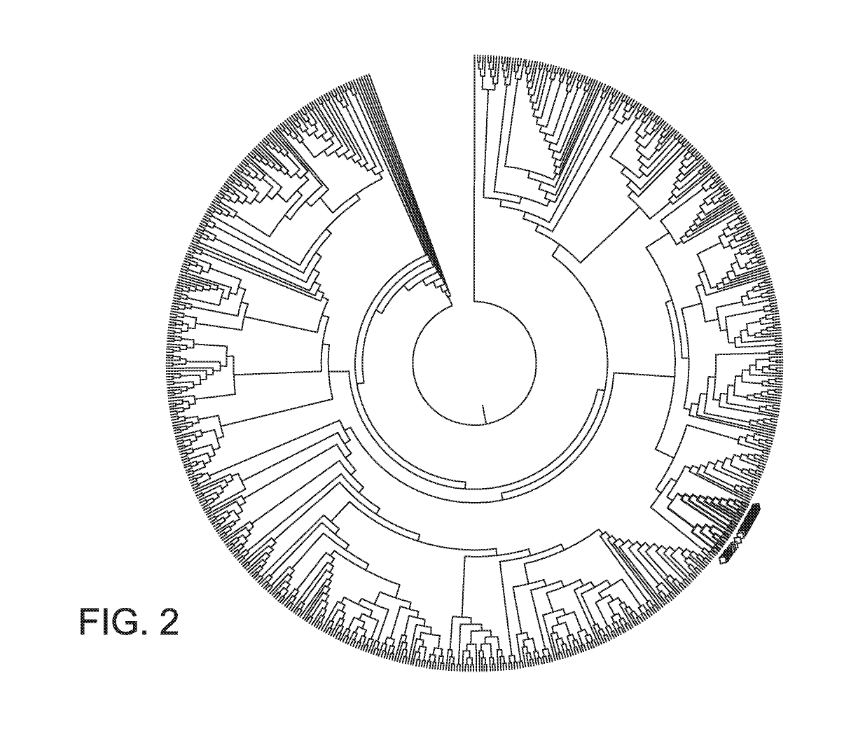

The term “SLSL Clade” refers to a branch of KARI sequences that was identified through molecular phylogenetic analysis that includes KARIs from Staphylococcus, Listeria, Streptococcus, Lactococcus, Leuconostoc, Enterococcus, Macrococcus, and Lactobacillus. FIGS. 1 and 2 show the relationship of the SLSL Clade to other KARIs and the SLSL Clade itself, respectively.

The term “carbon substrate” or “fermentable carbon substrate” refers to a carbon source capable of being metabolized by host organisms of the present invention and particularly carbon sources selected from the group consisting of monosaccharides, oligosaccharides, and polysaccharides.

The term “gene” refers to a nucleic acid fragment that is capable of being expressed as a specific protein, optionally including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign gene” or “heterologous gene” refers to a gene not normally found in the host organism, but that is introduced into the host organism by gene transfer or is modified in some way from its native state such as to alter its expression. Foreign genes can comprise native genes inserted into a non-native organism, or chimeric genes. A “transgene” is a gene that has been introduced into the genome by a transformation procedure.

As used herein the term “coding region” refers to a DNA sequence that codes for a specific amino acid sequence. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing sites, effector bindings site and stem-loop structures.

The term “promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

The term “operably linked” refers to the association of nucleic acid sequences on a single nucleic acid fragment so that the function of one is affected by the other. For example, a promoter is operably linked with a coding sequence when it is capable of effecting the expression of that coding sequence (i.e., that the coding sequence is under the transcriptional control of the promoter). Coding sequences can be operably linked to regulatory sequences in sense or antisense orientation.

The term “expression”, as used herein, refers to the transcription and stable accumulation of sense RNA (mRNA). Expression may also refer to translation of mRNA into a polypeptide.

As used herein the term “transformation” refers to the transfer of a nucleic acid molecule into a host cell, which may be maintained as a plasmid or integrated into the genome. Host cells containing the transformed nucleic acid molecules are referred to as “transgenic” or “recombinant” or “transformed” cells.

The terms “plasmid” and “vector” as used herein, refer to an extra chromosomal element often carrying genes which are not part of the central metabolism of the cell, and usually in the form of circular double-stranded DNA molecules. Such elements may be autonomously replicating sequences, genome integrating sequences, phage or other nucleotide sequences that may be linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

As used herein the term “codon degeneracy” refers to the nature in the genetic code permitting variation of the nucleotide sequence without affecting the amino acid sequence of an encoded polypeptide. The skilled artisan is well aware of the “codon-bias” exhibited by a specific host cell in usage of nucleotide codons to specify a given amino acid. Therefore, when synthesizing a coding region for improved expression in a host cell, it is desirable to design the coding region such that its frequency of codon usage approaches the frequency of preferred codon usage of the host cell.

The term “codon-optimized” as it refers to coding regions of nucleic acid molecules for transformation of various hosts, refers to the alteration of codons in the coding regions of the nucleic acid molecules to reflect the typical codon usage of the host organism without altering the polypeptide encoded by the DNA.

As used herein, an “isolated nucleic acid fragment” or “isolated nucleic acid molecule” will be used interchangeably and will mean a polymer of RNA or DNA that is single- or double-stranded, optionally containing synthetic, non-natural or altered nucleotide bases. An isolated nucleic acid fragment in the form of a polymer of DNA may be comprised of one or more segments of cDNA, genomic DNA or synthetic DNA.

A nucleic acid fragment is “hybridizable” to another nucleic acid fragment, such as a cDNA, genomic DNA, or RNA molecule, when a single-stranded form of the nucleic acid fragment can anneal to the other nucleic acid fragment under the appropriate conditions of temperature and solution ionic strength. Hybridization and washing conditions are well known and exemplified in Sambrook, J., Fritsch, E. F. and Maniatis, T. Molecular Cloning: A Laboratory Manual, 2^{nd} ed., Cold Spring Harbor Laboratory: Cold Spring Harbor, N.Y. (1989), particularly Chapter 11 and Table 11.1 therein (entirely incorporated herein by reference). The conditions of temperature and ionic strength determine the “stringency” of the hybridization. Stringency conditions can be adjusted to screen for moderately similar fragments (such as homologous sequences from distantly related organisms), to highly similar fragments (such as genes that duplicate functional enzymes from closely related organisms). Post-hybridization washes determine stringency conditions. One set of preferred conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. A more preferred set of stringent conditions uses higher temperatures in which the washes are identical to those above except for the temperature of the final two 30 min washes in 0.2×SSC, 0.5% SDS was increased to 60° C. Another preferred set of highly stringent conditions uses two final washes in 0.1×SSC, 0.1% SDS at 65° C. An additional set of stringent conditions include hybridization at 0.1×SSC, 0.1% SDS, 65° C. and washes with 2×SSC, 0.1% SDS followed by 0.1×SSC, 0.1% SDS, for example.

Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementarity, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (see Sambrook et al., supra, 9.50-9.51). For hybridizations with shorter nucleic acids, i.e., oligonucleotides, the position of mismatches becomes more important, and the length of the oligonucleotide determines its specificity (see Sambrook et al., supra, 11.7-11.8). In one embodiment the length for a hybridizable nucleic acid is at least about 10 nucleotides. Preferably a minimum length for a hybridizable nucleic acid is at least about 15 nucleotides; more preferably at least about 20 nucleotides; and most preferably the length is at least about 30 nucleotides. Furthermore, the skilled artisan will recognize that the temperature and wash solution salt concentration may be adjusted as necessary according to factors such as length of the probe.

A “substantial portion” of an amino acid or nucleotide sequence is that portion comprising enough of the amino acid sequence of a polypeptide or the nucleotide sequence of a gene to putatively identify that polypeptide or gene, either by manual evaluation of the sequence by one skilled in the art, or by computer-automated sequence comparison and identification using algorithms such as BLAST (Altschul, S. F., et al., J. Mol. Biol., 215:403-410 (1993)). In general, a sequence of ten or more contiguous amino acids or thirty or more nucleotides is necessary in order to putatively identify a polypeptide or nucleic acid sequence as homologous to a known protein or gene. Moreover, with respect to nucleotide sequences, gene specific oligonucleotide probes comprising 20-30 contiguous nucleotides may be used in sequence-dependent methods of gene identification (e.g., Southern hybridization) and isolation (e.g., in situ hybridization of bacterial colonies or bacteriophage plaques). In addition, short oligonucleotides of about 17 bases may be used as amplification primers in PCR in order to obtain a particular nucleic acid fragment comprising the primers. Accordingly, a “substantial portion” of a nucleotide sequence comprises enough of the sequence to specifically identify and/or isolate a nucleic acid fragment comprising the sequence. The instant specification teaches the complete amino acid and nucleotide sequence encoding particular proteins. The skilled artisan, having the benefit of the sequences as reported herein, may now use all or a substantial portion of the disclosed sequences for purposes known to those skilled in this art. Accordingly, the instant invention comprises the complete sequences as reported in the accompanying Sequence Listing, as well as substantial portions of those sequences as defined above.

The term “complementary” is used to describe the relationship between nucleotide bases that are capable of hybridizing to one another. For example, with respect to DNA, adenosine is complementary to thymine and cytosine is complementary to guanine.

The term “percent identity”, as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in: 1.) Computational Molecular Biology (Lesk, A. M., Ed.) Oxford University: NY (1988); 2.) Biocomputing: Informatics and Genome Projects (Smith, D. W., Ed.) Academic: NY (1993); 3.) Computer Analysis of Sequence Data, Part I (Griffin, A. M., and Griffin, H. G., Eds.) Humania: NJ (1994); 4.) Sequence Analysis in Molecular Biology (von Heinje, G., Ed.) Academic (1987); and 5.) Sequence Analysis Primer (Gribskov, M. and Devereux, J., Eds.) Stockton: NY (1991).

Preferred methods to determine identity are designed to give the best match between the sequences tested. Methods to determine identity and similarity are codified in publicly available computer programs. Sequence alignments and percent identity calculations may be performed using the MegAlign™ program of the LASERGENE bioinformatics computing suite (DNASTAR Inc., Madison, Wis.). Multiple alignment of the sequences is performed using the “Clustal method of alignment” which encompasses several varieties of the algorithm including the “Clustal V method of alignment” corresponding to the alignment method labeled Clustal V (described by Higgins and Sharp, CABIOS. 5:151-153 (1989); Higgins, D. G. et al., Comput. Appl. Biosci., 8:189-191 (1992)) and found in the MegAlign™ program of the LASERGENE bioinformatics computing suite (DNASTAR Inc.). For multiple alignments, the default values correspond to GAP PENALTY=10 and GAP LENGTH PENALTY=10. Default parameters for pairwise alignments and calculation of percent identity of protein sequences using the Clustal method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences using the Clustal V program, it is possible to obtain a “percent identity” by viewing the “sequence distances” table in the same program. Additionally the “Clustal W method of alignment” is available and corresponds to the alignment method labeled Clustal W (described by Higgins and Sharp, CABIOS. 5:151-153 (1989); Higgins, D. G. et al., Comput. Appl. Biosci. 8:189-191(1992)) and found in the MegAlign™ v6.1 program of the LASERGENE bioinformatics computing suite (DNASTAR Inc.). Default parameters for multiple alignment (GAP PENALTY=10, GAP LENGTH PENALTY=0.2, Delay Divergen Seqs (%)=30, DNA Transition Weight=0.5, Protein Weight Matrix=Gonnet Series, DNA Weight Matrix=IUB). After alignment of the sequences using the Clustal W program, it is possible to obtain a “percent identity” by viewing the “sequence distances” table in the same program.

It is well understood by one skilled in the art that many levels of sequence identity are useful in identifying polypeptides, such as from other species, wherein such polypeptides have the same or similar function or activity. Useful examples of percent identities include, but are not limited to: 24%, 30%, 35%, 40%, 45%, 50%, 55%, 60%, 65%, 70%, 75%, 80%, 85%, 90%, or 95%, or any integer percentage from 24% to 100% may be useful in describing the present invention, such as 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%. Suitable nucleic acid fragments not only have the above homologies but typically encode a polypeptide having at least 50 amino acids, preferably at least 100 amino acids, more preferably at least 150 amino acids, still more preferably at least 200 amino acids, and most preferably at least 250 amino acids.

The term “sequence analysis software” refers to any computer algorithm or software program that is useful for the analysis of nucleotide or amino acid sequences. “Sequence analysis software” may be commercially available or independently developed. Typical sequence analysis software will include, but is not limited to: 1.) the GCG suite of programs (Wisconsin Package Version 9.0, Genetics Computer Group (GCG), Madison, Wis.); 2.) BLASTP, BLASTN, BLASTX (Altschul et al., J. Mol. Biol., 215:403-410 (1990)); 3.) DNASTAR (DNASTAR, Inc. Madison, Wis.); 4.) Sequencher (Gene Codes Corporation, Ann Arbor, Mich.); and 5.) the FASTA program incorporating the Smith-Waterman algorithm (W. R. Pearson, Comput. Methods Genome Res., [Proc. Int. Symp.] (1994), Meeting Date 1992, 111-20. Editor(s): Suhai, Sandor. Plenum: New York, N.Y.). Within the context of this application it will be understood that where sequence analysis software is used for analysis, the results of the analysis will be based on the “default values” of the program referenced, unless otherwise specified. As used herein “default values” will mean any set of values or parameters that originally load with the software when first initialized.

Standard recombinant DNA and molecular cloning techniques used here are well known in the art and are described by Sambrook, J., Fritsch, E. F. and Maniatis, T., Molecular Cloning: A Laboratory Manual, Second Edition, Cold Spring Harbor Laboratory Press, Cold Spring Harbor, N.Y. (1989) (hereinafter “Maniatis”); and by Silhavy, T. J., Bennan, M. L. and Enquist, L. W., Experiments with Gene Fusions, Cold Spring Harbor Laboratory Press, Cold Spring Harbor, N.Y. (1984); and by Ausubel, F. M. et al., Current Protocols in Molecular Biology, published by Greene Publishing Assoc. and Wiley-Interscience (1987). Additional methods used here are in Methods in Enzymology, Volume 194, Guide to Yeast Genetics and Molecular and Cell Biology (Part A, 2004, Christine Guthrie and Gerald R. Fink (Eds.), Elsevier Academic Press, San Diego, Calif.).

Discovery of KARIs with High In Vivo Efficiency

Biosynthetic pathways for production of isobutanol that were disclosed in US Patent Publication No. US 20070092957 are shown in FIG. 1. Maximizing the steps of a biosynthetic pathway is desirable to maximize isobutanol production. The second step of all pathways in FIG. 1 is conversion of acetolactate to dihydroxy-isovalerate by ketol-acid reductoisomerase (KARI). Applicants have identified KARIs that, when used in an isobutanol biosynthetic pathway, provide for increased isobutanol production in yeast and bacteria over levels previously obtained using other KARIs.

In yeast expressing the Lactococcus lactis KARI (coding sequence of SEQ ID NO:67; protein of SEQ ID NO:68) as the KARI of the expressed isobutanol pathway, production of isobutanol was found to be greater than isobutanol production using either the Pseudomonas fluorescens KARI (coding sequence of SEQ ID NO:91; protein of SEQ ID NO:89) or Saccharomyces cerevisiae KARI (coding sequence of SEQ ID NO:92; protein of SEQ ID NO:93). Isobutanol production in the conditions tested was at least about doubled. In contrast, in vitro activity of the L. lactis KARI was less than that of the P. fluorescens KARI. In Lactobacillus plantarum expressing the KARI from Lactococcus lactis (coding sequence of SEQ ID NO:67; protein of SEQ ID NO:68), Streptococcus mutans (coding sequence of SEQ ID NO:27; protein of SEQ ID NO:28), Streptococcus thermophilis (coding sequence of SEQ ID NO:55; protein of SEQ ID NO:56), or Leuconostoc mesenteroides (coding sequence of SEQ ID NO:39; protein of SEQ ID NO:40) as the KARI of the expressed isobutanol pathway, production of isobutanol was found to be greater than isobutanol production using the Pseudomonas fluorescens KARI (coding sequence of SEQ ID NO:90; protein of SEQ ID NO:89). Isobutanol production in the conditions tested was increased by at least three-fold. The amount of increase in isobutanol production may vary depending on factors such as the host strain, the other isobutanol pathway enzymes present, culture media, and culture conditions. Isobutanol production is at least about doubled in both bacteria and yeast when using a highly effective KARI as compared to when using the Pseudomonas fluorescens KARI. Isobutanol production may be increased 2-fold, 3-fold, 4-fold, or more.

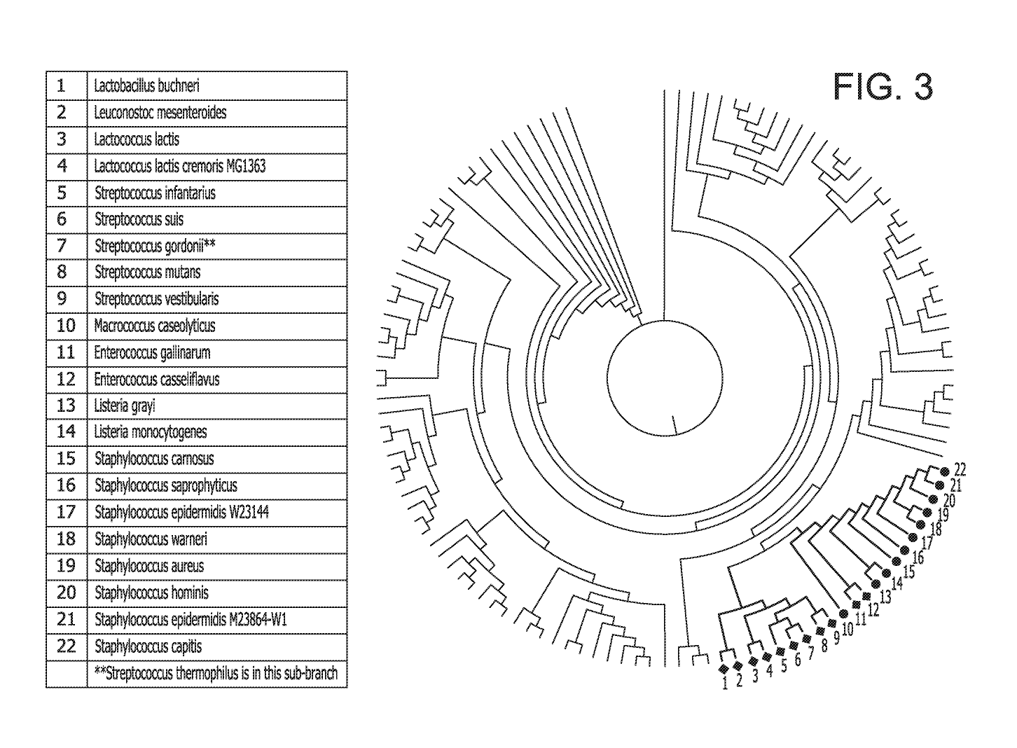

KARIs that are highly effective for isobutanol production, that may be used in the present cells and methods, are those that are members of a group identified through molecular phylogenetic analysis of KARI amino acid sequences. The molecular phylogenetic analysis was performed on KARI sequences collected from public databases by BLAST analysis of the Pseudomonas fluorescens KARI Pf-5 (SEQ ID NO:89). A multiple sequence alignment (MSA) was generated from the KARI sequences and a phylogenetic tree of the sequences was generated from the MSA using the neighbor-joining method of the Jalview program (Waterhouse et al. (2009) Bioinformatics doi: 10.1093/bioinformatics/btp033), which is publicly available. The resulting phylogenetic tree, in which KARI sequences with 95% or higher identities are represented by a single sequence, is shown in FIG. 2. Through this analysis of KARI sequences it was found that the KARIs tested as described above from Lactococcus lactis (SEQ ID NO: 68), Streptococcus mutans (SEQ ID NO:28), Streptococcus thermophilis (SEQ ID NO:56) and Leuconostoc mesenteroides (SEQ ID NO:40) are all members of a well-defined phylogenetic branch, or clade, of KARIs that in addition includes KARIs from other strains of Lactococcus, Streptococcus, and Leuconostoc, as well as KARIs from Staphylococcus, Listeria, Enterococcus, Macrococcus, and Lactobacillus species. This clade of KARIs is thus identified as the SLSL Clade and is marked in FIG. 1. In addition, the portion of the phylogenetic tree containing the SLSL Clade is shown in FIG. 3.

Any KARI that is a member of the SLSL Clade may be used in the present cells and methods. Members of this phylogenetic branch identified herein include KARIs from different species of Staphylococcus, Listeria, Streptococcus, Lactococcus, Leuconostoc, Enterococcus, Macrococcus, and Lactobacillus including those that are listed in Table 1 as SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, and 245. Coding region sequences for these KARIs have SEQ ID NOs:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, and 244, respectively. Most sequences with 99% or greater identities to any of the sequences in Table 1 are not listed but may also be used in the present cells and methods and are readily identified by one skilled in the art using bioinformatics analysis as described above. Therefore, sequences having at least about 99% identity to the sequences in Table 1 may be used in the present cells.

Additional KARIs that belong to the SLSL CLade of KARIs may be readily identified in the literature and in bioinformatics databases as is well known to the skilled person. Identification of coding and/or protein sequences using bioinformatics is typically through BLAST (described above) searching of publicly available databases with KARI encoding sequences or encoded amino acid sequences, such as those provided herein. Molecular phylogenetic analysis as described above may be used to determine whether a KARI is a member of the SLSL Clade. Additional KARIs include those that are members of the SLSL Clade having amino acid sequence identity of at least about 80-85%, 85%-90%, 90%-95%, or at least about 96%, 97%, 98%, or 99% sequence identity to any of the KARI amino acid sequences of SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, and 245. Identities are based on the Clustal W method of alignment using the default parameters of GAP PENALTY=10, GAP LENGTH PENALTY=0.1, and Gonnet 250 series of protein weight matrix.

Additionally, the sequences described herein or those recited in the art may be used to identify other homologs in nature. For example each of the KARI encoding nucleic acid fragments described herein may be used to isolate genes encoding homologous proteins. Isolation of homologous genes using sequence-dependent protocols is well known in the art. Examples of sequence-dependent protocols include, but are not limited to: 1) methods of nucleic acid hybridization; 2) methods of DNA and RNA amplification, as exemplified by various uses of nucleic acid amplification technologies [e.g., polymerase chain reaction (PCR), Mullis et al., U.S. Pat. No. 4,683,202; ligase chain reaction (LCR), Tabor, S. et al., Proc. Acad. Sci. USA 82:1074 (1985); or strand displacement amplification (SDA), Walker, et al., Proc. Natl. Acad. Sci. U.S.A., 89:392 (1992)]; and 3) methods of library construction and screening by complementation.

### KARI Expression in Yeast and Bacteria Cells

Any of the KARIs described above may be expressed in a yeast or bacterial cell to convert acetolactate to dihydroxy-isovalerate providing a step in an isobutanol biosynthetic pathway. Yeast cells that may be host cells include, but are not limited to, those belonging to genera of Saccharomyces, Schizosaccharomyces, Hansenula, Candida, Kluyveromyces, Yarrowia, Issatchenkia, and Pichia. Bacterial cells that may be host cells include, but are not limited to, those belonging to genera of Escherichia, Rhodococcus, Pseudomonas, Bacillus, Enterococcus, Lactococcus, Lactobacillus, Leuconostoc, Oenococcus, Pediococcus, Streptococcus, Clostridium, Zymomonas, Salmonella, Pediococcus, Alcaligenes, Klebsiella, Paenibacillus, Arthrobacter, Corynebacterium, and Brevibacterium. Particularly useful are host cells that are lactic acid bacteria (LAB) such as Lactococcus, Lactobacillus, Leuconostoc, Oenococcus, Pediococcus, and Streptococcus.

Methods for expressing the KARI coding regions described above are well known to one skilled in the art. For example, methods for gene expression in yeasts are known and described, such as in Methods in Enzymology, Volume 194, Guide to Yeast Genetics and Molecular and Cell Biology (Part A, 2004, Christine Guthrie and Gerald R. Fink (Eds.), Elsevier Academic Press, San Diego, Calif.).

Typically the coding region for the desired KARI is constructed as part of a chimeric gene by operably linking the coding region to a promoter for expression in the target cell. A termination control region may be included in the chimeric expression gene, and for bacterial cell expression a ribosome binding site may be included. The coding region may be codon optimized for expression in the particular host cell being engineered as known to one skilled in the art.

Promoters that may be used for expression in yeast are, for example, constitutive promoters FBA1, TDH3, ADH1, and GPM1, and the inducible promoters GAL1, GAL10, and CUP1. Suitable transcriptional terminators that may be used in a chimeric gene construct for expression in yeast include, but are not limited to FBA1t, TDH3t, GPM1t, ERG10t, GAL1t, CYC1t, and ADH1t.

Suitable promoters, transcriptional terminators, and coding regions may be cloned into E. coli-yeast shuttle vectors, and transformed into yeast cells. These vectors allow for propagation in both E. coli and yeast strains. Typically the vector contains a selectable marker and sequences allowing autonomous replication or chromosomal integration in the desired host. Typically used plasmids in yeast are shuttle vectors pRS423, pRS424, pRS425, and pRS426 (American Type Culture Collection, Rockville, Md.), which contain an E. coli replication origin (e.g., pMB1), a yeast 2μ origin of replication, and a marker for nutritional selection. The selection markers for these four vectors are HIS3 (vector pRS423), TRP1 (vector pRS424), LEU2 (vector pRS425) and URA3 (vector pRS426). Construction of expression vectors with a chimeric gene may be performed by either standard molecular cloning techniques in E. coli or by the gap repair recombination method in yeast. Chimeric genes may be expressed from a plasmid or integrated into the cell genome.

Promoters for expression of chimeric genes in bacterial cells are numerous and familiar to those skilled in the art, including, but not limited to, lac, ara, tet, trp, IPL, IPR, T7, tac, and trc promoters (useful for expression in Escherichia coli, Alcaligenes, and Pseudomonas); the amy, apr, and npr promoters, and various phage promoters useful for expression in Bacillus subtilis, Bacillus licheniformis, and Paenibacillus macerans; nisA (useful for expression Gram-positive bacteria, Eichenbaum et al. Appl. Environ. Microbiol. 64(8):2763-2769 (1998)); and the synthetic P11 promoter (useful for expression in Lactobacillus plantarum, Rud et al., Microbiology 152:1011-1019 (2006)). In addition, the ldhL1, and fabZ1 promoters of L plantarum are useful for expression of chimeric genes in LAB. The fabZ1 promoter directs transcription of an operon with the first gene, fabZ1, encoding (3R)-hydroxymyristoyl-[acyl carrier protein] dehydratase.

Termination control regions may also be derived from various bacterial genes native to the preferred hosts.

Vectors useful for the transformation of a variety of bacterial cells are common and commercially available from companies such as EPICENTRE® (Madison, Wis.), Invitrogen Corp. (Carlsbad, Calif.), Stratagene (La Jolla, Calif.), and New England Biolabs, Inc. (Beverly, Mass.). Certain vectors are capable of replicating in a broad range of host bacteria and can be transferred by conjugation. The complete and annotated sequence of pRK404 and three related vectors: pRK437, pRK442, and pRK442(H), are available. These derivatives have proven to be valuable tools for genetic manipulation in Gram-negative bacteria (Scott et al., Plasmid 50(1):74-79 (2003)). Several plasmid derivatives of broad-host-range Inc P4 plasmid RSF1010 are also available with promoters that can function in a range of Gram-negative bacteria. Plasmid pAYC36 and pAYC37, have active promoters along with multiple cloning sites to allow for heterologous gene expression in Gram-negative bacteria. Some vectors that are useful for transformation of Bacillus subtilis and Lactobacillus include pAMβ1 and derivatives thereof (Renault et al., Gene 183:175-182 (1996); and O'Sullivan et al., Gene 137:227-231 (1993)); pMBB1 and pHW800, a derivative of pMBB1 (Wyckoff et al. Appl. Environ. Microbiol. 62:1481-1486 (1996)); pMG1, a conjugative plasmid (Tanimoto et al., J. Bacteriol. 184:5800-5804 (2002)); pNZ9520 (Kleerebezem et al., Appl. Environ. Microbiol. 63:4581-4584 (1997)); pAM401 (Fujimoto et al., Appl. Environ. Microbiol. 67:1262-1267 (2001)); and pAT392 (Arthur et al., Antimicrob. Agents Chemother. 38:1899-1903 (1994)). Several plasmids from Lactobacillus plantarum have also been reported (van Kranenburg et al., Appl. Environ. Microbiol. 71(3):1223-1230 (2005)).

Vectors or plasmids may be introduced into a host cell using methods known in the art, such as electroporation and conjugation.

### Host Cells for Isobutanol Production

Yeast and bacteria cells that are engineered with an isobutanol biosynthesis pathway, including with a KARI described herein, may have additional modifications. Any modifications may be made that improve the host cell, such as modifications that increase flux to isobutanol synthesis.

For example, yeast cells that may be used may be engineered to have at least one pyruvate decarboxylase (PDC) gene inactivated creating a pdc-cell, so that pyruvate conversion to acetaldehyde is reduced and more pyruvate can flow to the isobutanol pathway. Yeasts may have one or more genes encoding pyruvate decarboxylase. For example, there is one gene encoding pyruvate decarboxylase in Candida glabrata and Schizosaccharomyces pombe, while there are three isozymes of pyruvate decarboxylase encoded by the PDC1, PCD5, and PDC6 genes in Saccharomyces. If the yeast cell used has more than one expressed (active) PDC gene, then each of the active PDC genes is inactivated thereby producing a pdc-cell. For example, in S. cerevisiae the PDC1, PDC5, and PDC6 genes may be inactivated. Though if a PDC gene is not active under the fermentation conditions to be used, such as PDC6, then this gene does not need to be inactivated.

Saccharomyces strains having no pyruvate decarboxylase activity are available from the ATCC with Accession #200027 and #200028. In addition, yeast may be engineered to inactivate the endogenous active PDC genes as described in US Patent Application Publication No. 20090305363, which is herein incorporated by reference, and in Example 1 herein.

In addition, yeast cells that may be used may be engineered to have reduced glucose repression. Glucose repression, which occurs in crabtree-positive yeasts, is a phenomenon whereby in the presence of high glucose, repression of expression of genes involved in respiratory metabolism and utilization of non-glucose carbon sources occurs (Gancedo (1998) Microbiol. Mol. Bio. Rev. 62:334-361). Disclosed in U.S. Provisional Patent Application No. 61/246,709, filed Sep. 29, 2009, is the finding that yeast cells with reduced glucose repression in combination with a pdc-phenotype and an isobutanol pathway had improved isobutanol production and growth in high gludose. Glucose repression may be reduced, as described in U.S. Provisional Patent Application No. 61/246,709, which is herein incorporated by reference, by methods such as: (1) altering expression of transcription factors involved in glucose repression effects. For example, increased expression of the Hap1 transcription activator or reduced expression of the Mig1 or Mig2 transcription repressor; (2) reducing expression of GRR1 (Glucose Repression Resistant) which is a component of the SCF ubiquitin-ligase complex and appears to be a primary factor in the glucose repression pathway, (3) attenuating glucose transport capacity by inactivation of hexose transporter genes including HXT1, HXT2, HX3, HXT4, HXT5, HXT6, and/or HXT7, or (4) deletion of the endogenous hexokinase2 gene (HXK2). Altering expression of the above-mentioned genes may be engineered by methods well known in the art, including as exemplified in Example 1 herein.

For example, lactic acid bacteria (LAB) cells that may be used may be engineered to have reduced lactate dehydrogenase activity so that production of lactate from pyruvate is reduced to enhance pyruvate flux to another pathway as described in US Patent Application Publication No. 20100112655, which is herein incorporated by reference. LAB may have one or more genes, typically one, two or three genes, encoding lactate dehydrogenase. For example, Lactobacillus plantarum has three genes encoding lactate dehydrogenase which are named ldhL2, ldhD, and ldhL1. Lactococcus lactis has one gene encoding lactate dehydrogenase which is named ldhL, and Pediococcus pentosaceus has two genes named ldhD and ldhL. When more than one lactate dehydrogenase gene is active under the growth conditions to be used, each of these active genes may be modified to reduce expression as in Example 1 herein.

In addition, an LAB host cell may be engineered for increased expression of Fe—S cluster forming proteins to improve the activity of the Fe—S cluster requiring dihydroxy-acid dehydratase enzyme of the isobutanol pathway as disclosed in US Patent Application No. 20100081182, which is herein incorporated by reference. For example, expression of the endogenous suf operon encoding Fe—S cluster forming proteins may be increased as described in Example 2 herein.

Additional modifications that may be useful in cells provided herein include modifications to reduce glycerol-3-phosphate dehydrogenase activity as described in US Patent Application Publication No. 20090305363 (incorporated herein by reference), modifications to a host cell that provide for increased carbon flux through an Entner-Doudoroff Pathway or reducing equivalents balance as described in US Patent Application Publication No. 20100120105 (incorporated herein by reference). Yeast cells with reduced activity of certain enzymes involved in branched chain amino acid biosynthesis in yeast mitochondria are described in US Application Publication No. 20100129887 (incorporated herein by reference) and yeast strains with increased activity of heterologous proteins that require binding of an Fe—S cluster as a cofactor for their activity are described in US Application Publication No. 20100081179 (incorporated herein by reference). Other modifications include modifications in an endogenous polynucleotide encoding a polypeptide having dual-role hexokinase activity, described in U.S. Provisional Application No. 61/290,639, integration of at least one polynucleotide encoding a polypeptide that catalyzes a step in a pyruvate-utilizing biosynthetic pathway described in U.S. Provisional Application No. 61/380,563 (both referenced provisional applications are incorporated herein by reference in their entirety).

Additionally, host cells comprising at least one deletion, mutation, and/or substitution in an endogenous gene encoding a polypeptide affecting Fe—S cluster biosynthesis are described in U.S. Provisional Patent Application No. 61/305,333 (incorporated herein by reference), and host cells comprising a heterologous polynucleotide encoding a polypeptide with phosphoketolase activity and host cells comprising a heterologous polynucleotide encoding a polypeptide with phosphotransacetylase activity are described in U.S. Provisional Patent Application No. 61/356,379.

Methods for engineering host cells with the above modifications are well known in the art. Methods for gene expression include those described above for expression of KARIs. Methods for gene inactivation include, but are not limited to, deletion of the entire or a portion of the encoding gene, inserting a DNA fragment into the encoding gene (in either the promoter or coding region) so that the encoded protein cannot be expressed, introducing a mutation into the coding region which adds a stop codon or frame shift such that a functional protein is not expressed, and introducing one or more mutations into the coding region to alter amino acids so that a non-functional protein is expressed. In addition expression may be blocked by expression of an antisense RNA or an interfering RNA, and constructs may be introduced that result in cosuppression.

### Isobutanol Production

The present cells having a KARI that is highly effective in vivo as described herein produce isobutanol using a biosynthetic pathway such as one disclosed in US Patent Application Publication US 20070092957 A1, which is herein incorporated by reference, and shown in FIG. 1.

As described in US 20070092957 A1, steps in an example isobutanol biosynthetic pathway include conversion of:
pyruvate to acetolactate (FIG. 1 pathway step a) as catalyzed for example by acetolactate synthase (ALS) known by the EC number 2.2.1.69;
acetolactate to 2,3-dihydroxyisovalerate (FIG. 1 pathway step b) as catalyzed for example by acetohydroxy acid isomeroreductase, also called ketol-acid reductoisomerase (KARI) known by the EC number 1.1.1.86;
2,3-dihydroxyisovalerate to α-ketoisovalerate (FIG. 1 pathway step c) as catalyzed for example by acetohydroxy acid dehydratase, also called dihydroxy-acid dehydratase (DHAD) known by the EC number 4.2.1.9;
α-ketoisovalerate to isobutyraldehyde (FIG. 1 pathway step d) as catalyzed for example by branched-chain α-keto acid decarboxylase known by the EC number 4.1.1.72 or 4.1.1.1; and
isobutyraldehyde to isobutanol (FIG. 1 pathway step e) as catalyzed for example by branched-chain alcohol dehydrogenase known by the EC number 1.1.1.265, but may also be classified under other alcohol dehydrogenases (specifically, EC 1.1.1.1 or 1.1.1.2).

The substrate to product conversions, and enzymes involved in these reactions, for steps f, g, h, l, j, and k of alternative pathways are described in US 20070092957 A1.

Genes that may be used for expression of these enzymes, as well as those for two additional isobutanol pathways, are described in US 20070092957 A1, and additional genes that may be used can be identified in the literature and using bioinformatics approaches, as is well known to the skilled person as described above. Additionally, sequences provided therein may be used to isolate genes encoding homologous proteins using sequence-dependent protocols is well known in the art, as described above.

For example, some representative ALS enzymes that may be used include those encoded by alsS of Bacillus and budB of Klebsiella (Gollob et al., J. Bacteriol. 172(6):3444-3449 (1990); Holtzclaw et al., J. Bacteriol. 121(3):917-922 (1975)). ALS from Bacillus subtilis (DNA: SEQ ID NO:69; protein: SEQ ID NO:70), from Klebsiella pneumoniae (DNA: SEQ ID NO:72; protein: SEQ ID NO:73), and from Lactococcus lactis (DNA: SEQ ID NO:74; protein: SEQ ID NO:75) are provided herein, as well as a Bacillus subtilis als coding region optimized for expression in Lactobacillus plantarum (SEQ ID NO:71). Additional als coding regions and encoded proteins that may be used include those from Staphylococcus aureus (DNA: SEQ ID NO:76; protein: SEQ ID NO:77), Listeria monocytogenes (DNA: SEQ ID NO:78; protein: SEQ ID NO:79), Streptococcus mutans (DNA: SEQ ID NO:80; protein: SEQ ID NO:81), Streptococcus thermophilus (DNA: SEQ ID NO:82; protein: SEQ ID NO:83), Vibrio angustum (DNA: SEQ ID NO:84; protein: SEQ ID NO:85), and Bacillus cereus (DNA: SEQ ID NO:86; protein: SEQ ID NO:87). Any als gene that encodes an acetolactate synthase having at least about 80-85%, 85%-90%, 90%-95%, or at least about 96%, 97%, or 98% sequence identity to any one of those with SEQ ID NOs:70, 73, 75, 77, 79, 81, 83, 85, or 87 that converts pyruvate to acetolactate may be used. Identities are based on the Clustal W method of alignment using the default parameters of GAP PENALTY=10, GAP LENGTH PENALTY=0.1, and Gonnet 250 series of protein weight matrix.

Additionally, US Patent Application Publication No. 20090305363, incorporated herein by reference, provides a phylogenetic tree depicting acetolactate synthases that are the 100 closest neighbors of the B. subtilis AlsS sequence, any of which may be used. Additional Als sequences that may be used in the present strains may be identified in the literature and in bioinformatics databases as is well known to the skilled person. Identification of coding and/or protein sequences using bioinformatics is typically through BLAST (described above) searching of publicly available databases with known Als encoding sequences or encoded amino acid sequences, such as those provided herein. Identities are based on the Clustal W method of alignment as specified above. Additionally, the sequences listed herein or those recited in the art may be used to identify other homologs in nature as described above.

Cytosolic expression of acetolactate synthase in yeast is achieved by transforming with a gene comprising a sequence encoding an acetolactate synthase protein, with no mitochondrial targeting signal sequence. Methods for gene expression in yeasts are known in the art (see for example Methods in Enzymology, Volume 194, Guide to Yeast Genetics and Molecular and Cell Biology (Part A, 2004, Christine Guthrie and Gerald R. Fink (Eds.), Elsevier Academic Press, San Diego, Calif.). Expression using chimeric genes (including promoters and terminators), vectors, cloning methods, and integration methods are as described above.

KARI enzymes that may be used are described above. It is also contemplated that additional KARI enzymes could be used in conjunction with the KARI enzymes described herein. Suitable KARI enzymes include those described in US Application Publication Nos. 20080261230, 20090163376, and 20100197519, all of which are herein incorporated by reference.

For example, DHAD enzymes that may be used may be from the ilvD gene of Lactococcus lactis (DNA: SEQ ID NO:94; protein SEQ ID NO:95) or Streptococcus mutans (DNA: SEQ ID NO:96; protein SEQ ID NO:97), or from the ILV3 gene of Saccharoomyces cerevisiae (DNA: SEQ ID NO:98; protein SEQ ID NO:99). Additional DHAD sequences that may be used to obtain additional DHAD sequences that may be used are disclosed in US Patent Application Publication No. 20100081154, which is herein incorporated by reference. This reference also includes descriptions for obtaining additional DHAD sequences that may be used.

For example, branched chain keto acid decarboxylase enzymes that may be used include one from the kivD gene of Lactococcus lactis (DNA: SEQ ID NO:100; protein SEQ ID NO:101), as well as an L. lactis kivD coding region that is codon optimized for expression in Lactobacillus plantarum (SEQ ID NO:102), and others that may be identified by one skilled in the art using bioinformatics as described above.

For example, branched-chain alcohol dehydrogenases that may be used are known by the EC number 1.1.1.265, but may also be classified under other alcohol dehydrogenases (specifically, EC 1.1.1.1 or 1.1.1.2). These enzymes utilize NADH (reduced nicotinamide adenine dinucleotide) and/or NADPH as electron donors and sequences of branched-chain alcohol dehydrogenase enzymes and their coding regions that may be used are provided in US20070092957 A1.

In addition, useful for the last step of converting isobutyraldehyde to isobutanol is a new butanol dehydrogenase, sadB, isolated from an environmental isolate of a bacterium identified as Achromobacter xylosoxidans (DNA: SEQ ID NO:103, protein SEQ ID NO:104) that is disclosed in US Patent Application Publication No. 20090269823, which is herein incorporated by reference. A sadB coding region that is optimized for expression in L. plantarum (SEQ ID NO:105) may be used. In addition, an alcohol dehydrogenase from horse liver (NADH; codon optimized for expression in S. cerevisiae; DNA: SEQ ID NO:106; protein SEQ ID NO:107) as well as others readily identified by one skilled in the art using bioinformatics as described above. Additional alcohol dehydrogenases are described in U.S. Provisional Patent Application No. 61/290,636, incorporated by reference herein.

Improved activity of DHAD in LAB cells that are substantially free of lactate dehydrogenase activity was disclosed in US Patent Application Publication No. 20100081183, which is herein incorporated by reference. Additionally, increased expression of iron-sulfur cluster forming proteins to improve activity of DHAD is disclosed in US Patent Application Publication No. 2010-0081182, which is herein incorporated by reference.

Described in US 20070092957 A1 is construction of chimeric genes and genetic engineering of LAB, exemplified by Lactobacillus plantarum, for isobutanol production using disclosed biosynthetic pathways. Chimeric genes for pathway enzyme expression may be present in a cell on a replicating plasmid or integrated into the cell genome, as well known to one skilled in the art. Additionally described in US 20070092957 A1 are construction of chimeric genes and genetic engineering of yeast, exemplified by Saccharomyces cerevisiae, for isobutanol production using the disclosed biosynthetic pathways. Futher description for gene construction and expression is above and in the Examples herein.

### Growth for Production

Bacteria and yeast cells disclosed herein may be grown in fermentation media for production of isobutanol. For maximal production the strains used as production hosts preferably have enhanced tolerance to isobutanol, and have a high rate of carbohydrate utilization. These characteristics may be conferred by mutagenesis and selection, genetic engineering, or may be natural.

The cells are grown in fermentation media which contains suitable carbon substrates. Suitable substrates may include but are not limited to monosaccharides such as glucose and fructose, oligosaccharides such as lactose or sucrose, polysaccharides such as starch or cellulose or mixtures thereof and unpurified mixtures from renewable feedstocks such as cheese whey permeate, cornsteep liquor, sugar beet molasses, and barley malt. Other carbon substrates may include ethanol, lactate, succinate, or glycerol. In addition, fermentable sugars may be derived from renewable cellulosic or lignocellulosic biomass through processes of pretreatment and saccharification, as described, for example, U.S. Patent Application Publication No. 2007/0031918A1, which is herein incorporated by reference. Hence it is contemplated that the source of carbon utilized in the present invention may encompass a wide variety of carbon containing substrates and will only be limited by the choice of organism.

In addition to an appropriate carbon source, fermentation media must contain suitable minerals, salts, cofactors, buffers and other components, known to those skilled in the art, suitable for the growth of the cultures and promotion of the enzymatic pathway necessary for production of isobutanol.

### Culture Conditions

Typically bacteria cells are grown at a temperature in the range of about 25° C. to about 40° C. while yeast cells are grown at a temperature in the range of about 20° C. to about 37° C., in an appropriate medium. Suitable growth media are common commercially prepared media and the appropriate medium for growth of the particular cells used will be known by one skilled in the art of microbiology or fermentation science

For bacteria, suitable pH ranges for the fermentation are between pH 5.0 to pH 9.0, where pH 6.0 to pH 8.0 is preferred as the initial condition. For yeast, suitable pH ranges for the fermentation are between pH 3.0 to pH 7.5, where pH 4.5 to pH 6.5 is preferred as the initial condition.

Fermentations may be performed under aerobic or anaerobic conditions, where anaerobic or microaerobic conditions are preferred.

It is contemplated that the production of isobutanol may be practiced using either batch, fed-batch or continuous processes and that any known mode of fermentation would be suitable. Additionally, it is contemplated that cells may be immobilized on a substrate as whole cell catalysts and subjected to fermentation conditions for isobutanol production.

Methods for Isobutanol Isolation from the Fermentation Medium

Bioproduced isobutanol may be isolated from the fermentation medium using methods known in the art for ABE fermentations (see for example, Durre, Appl. Microbiol. Biotechnol. 49:639-648 (1998), Groot et al., Process. Biochem. 27:61-75 (1992), and references therein). For example, solids may be removed from the fermentation medium by centrifugation, filtration, decantation, or the like. Then, the isobutanol may be isolated from the fermentation medium using methods such as distillation, azeotropic distillation, liquid-liquid extraction, adsorption, gas stripping, membrane evaporation, or pervaporation.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

The meaning of abbreviations is as follows: “s” means second(s), “min” means minute(s), “h” means hour(s), “psi” means pounds per square inch, “nm” means nanometers, “d” means day(s), “μl” means microliter(s), “ml” means milliliter(s), “L” means liter(s), “mm” means millimeter(s), “nm” means nanometers, “mM” means millimolar, “M” means molar, “mmol” means millimole(s), “μmol” means micromole(s)”, “g” means gram(s), “μg” means microgram(s) and “ng” means nanogram(s), “PCR” means polymerase chain reaction, “OD” means optical density, “OD₆₀₀” means the optical density measured at a wavelength of 600 nm, “kDa” means kilodaltons, “g” means the gravitation constant, “bp” means base pair(s), “kbp” means kilobase pair(s), “% w/v” means weight/volume percent, % v/v” means volume/volume percent, “wt %” means percent by weight, “HPLC” means high performance liquid chromatography, and “GC” means gas chromatography.

Standard recombinant DNA and molecular cloning techniques used in the Examples are well known in the art and are described by Sambrook, J., Fritsch, E. F. and Maniatis, T. Molecular Cloning: A Laboratory Manual; Cold Spring Harbor Laboratory Press: Cold Spring Harbor, N.Y. (1989) (Maniatis) and by T. J. Silhavy, M. L. Bennan, and L. W. Enquist, Experiments with Gene Fusions, Cold Spring Harbor Laboratory Press, Cold Spring Harbor, N.Y. (1984) and by Ausubel, F. M. et al., Current Protocols in Molecular Biology, pub. by Greene Publishing Assoc. and Wiley-Interscience (1987), and by Methods in Yeast Genetics, 2005, Cold Spring Harbor Laboratory Press, Cold Spring Harbor, N.Y.

Materials and methods suitable for the maintenance and growth of bacterial cultures are well known in the art. Techniques suitable for use in the following Examples may be found as set out in Manual of Methods for General Bacteriology (Phillipp Gerhardt, R. G. E. Murray, Ralph N. Costilow, Eugene W. Nester, Willis A. Wood, Noel R. Krieg and G. Briggs Phillips, eds), American Society for Microbiology, Washington, D.C. (1994)) or by Thomas D. Brock in Biotechnology: A Textbook of Industrial Microbiology, Second Edition, Sinauer Associates, Inc., Sunderland, Mass. (1989). All reagents, restriction enzymes and materials used for the growth and maintenance of microbial cells were obtained from Aldrich Chemicals (Milwaukee, Wis.), BD Diagnostic Systems (Sparks, Md.), Life Technologies (Rockville, Md.), or Sigma Chemical Company (St. Louis, Mo.) unless otherwise specified. Microbial strains were obtained from The American Type Culture Collection (ATCC), Manassas, Va., unless otherwise noted. The oligonucleotide primers used in the following Examples are given in Table 3. All the oligonucleotide primers were synthesized by Sigma-Genosys (Woodlands, Tex.) Integrated DNA Technologies (Coralsville, Iowa) or or Invitrogen Corp (Carlsbad, Calif.).

DNA fragments were purified with Qiaquick PCR Purification Kit (Qiagen Inc., Valencia, Calif.). Plasmid DNA was prepared with QIAprep Spin Miniprep Kit (Qiagen Inc., Valencia, Calif.). L. plantarum PN0512 genomic DNA was prepared with MasterPure DNA Purification Kit (Epicentre, Madison, Wis.).

Synthetic complete medium is described in Amberg, Burke and Strathern, 2005, Methods in Yeast Genetics, Cold Spring Harbor Laboratory Press, Cold Spring Harbor, N.Y.

### Transformation

Lactobacillus plantarum PN0512 was transformed by the following procedure: 5 ml of Lactobacilli MRS medium (Accumedia, Neogen Corporation, Lansing, Mich.) containing 1% glycine (Sigma-Aldrich, St. Louis, Mo.) was inoculated with PN0512 cells and grown overnight at 30° C. 100 ml MRS medium with 1% glycine was inoculated with overnight culture to an OD600 of 0.1 and grown to an OD600 of 0.7 at 30° C. Cells were harvested at 3700×g for 8 min at 4° C., washed with 100 ml cold 1 mM MgCl₂ (Sigma-Aldrich, St. Louis, Mo.), centrifuged at 3700×g for 8 min at 4° C., washed with 100 ml cold 30% PEG-1000 (Sigma-Aldrich, St. Louis, Mo.), recentrifuged at 3700×g for 20 min at 4° C., then resuspended in 1 ml cold 30% PEG-1000. 60 μl cells were mixed with ˜100 ng plasmid DNA in a cold 1 mm gap electroporation cuvette and electroporated in a BioRad Gene Pulser (Hercules, Calif.) at 1.7 kV, 25 μF, and 400Ω. Cells were resuspended in 1 ml MRS medium containing 500 mM sucrose (Sigma-Aldrich, St. Louis, Mo.) and 100 mM MgCl₂, incubated at 30° C. for 2 hrs, plated on MRS medium plates containing 1 or 2 μg/ml of erythromycin (Sigma-Aldrich, St. Louis, Mo.), then placed in an anaerobic box containing a Pack-Anaero sachet (Mitsubishi Gas Chemical Co., Tokyo, Japan) and incubated at 30° C.

### HPLC Method

Analysis for fermentation by-product composition is well known to those skilled in the art. For example, one high performance liquid chromatography (HPLC) method utilizes a Shodex SH-1011 column with a Shodex SH-G guard column (both available from Waters Corporation, Milford, Mass.), with refractive index (RI) detection. Chromatographic separation is achieved using 0.01 M H₂SO₄ as the mobile phase with a flow rate of 0.5 mL/min and a column temperature of 50° C. Isobutanol retention time is about 47.6 minutes.

### Example 1

### Construction of the ilvD Integration Vector and PN0512ΔldhDΔldhL1::ilvDLI⁺ Integration Strain

This example describes integration of the Lactococcus lactis ilvD gene into the chromosome of L. plantarum strain PN0512 ΔldhDΔldhL1 for expression of DHAD. The construction of L. plantarum PN0512 ΔldhDΔldhL1 was described in Example 1 of U.S. Patent Application No. 61/100,786. This strain is deleted for the two genes that encode the major lactate dehydrogenases: ldhD and ldhL1. The double deletion was made in Lactobacillus plantarum PN0512 (ATCC strain # PTA-7727).

Gene knockouts were constructed using a process based on a two-step homologous recombination procedure to yield unmarked gene deletions (Ferain et al., 1994, J. Bact. 176:596). The procedure utilized a shuttle vector, pFP996 (SEQ ID NO110). pFP996 is a shuttle vector for gram-positive bacteria. It can replicate in both E. coli and gram-positive bacteria. It contains the origins of replication from pBR322 (nucleotides #2628 to 5323) and pE194 (nucleotides #43 to 2627). pE194 is a small plasmid isolated originally from a gram positive bacterium, Staphylococcus aureus (Horinouchi and Weisblum J. Bacteriol. (1982) 150(2):804-814). In pFP996, the multiple cloning sites (nucleotides #1 to 50) contain restriction sites for EcoRI, BglII, XhoI, SmaI, ClaI, KpnI, and HindIII. There are two antibiotic resistance markers; one is for resistance to ampicillin and the other for resistance to erythromycin. For selection purposes, ampicillin was used for transformation in E. coli and erythromycin was used for selection in L. plantarum.

Two segments of DNA, each containing 900 to 1200 bp of sequence either upstream or downstream of the intended deletion, were cloned into the plasmid to provide the regions of homology for the two genetic cross-overs. Cells were grown for an extended number of generations (30-50) to allow for the cross-over events to occur. The initial cross-over (single cross-over) integrated the plasmid into the chromosome by homologous recombination through one of the two homology regions on the plasmid. The second cross-over (double cross-over) event yielded either the wild type sequence or the intended gene deletion. A cross-over between the sequences that led to the initial integration event would yield the wild type sequence, while a cross-over between the other regions of homology would yield the desired deletion. The second cross-over event was screened for by antibiotic sensitivity. Single and double cross-over events were analyzed by PCR and DNA sequencing.

ΔldhD

The knockout cassette to delete the ldhD gene was created by amplifying from PN0512 genomic DNA an upstream flanking region with primers Top D F1 (SEQ ID NO:111) containing an EcoRI site and Top D R1 (SEQ ID NO:112). The downstream homology region including part of the coding sequence of ldhD was amplified with primers Bot D F2 (SEQ ID NO:113) and Bot D R2 (SEQ ID NO:114) containing an XhoI site. The two homology regions were joined by PCR SOE as follows. The 0.9 kbp upstream and downstream PCR products were gel-purified. The PCR products were mixed in equal amounts in a PCR reaction and re-amplified with primers Top D F1 and Bot D R2. The final 1.8 kbp PCR product was gel-purified and TOPO cloned into pCR4BluntII-TOPO (Invitrogen) to create vector pCRBluntII::ldhD. To create the integration vector carrying the internal deletion of the ldhD gene, pFP996 was digested with EcoRI and XhoI and the 5311-bp fragment gel-purified. Vector pCRBluntII::ldhD was digested with EcoRI and XhoI and the 1.8 kbp fragment gel-purified. The ldhD knockout cassette and vector were ligated using T4 DNA ligase, resulting in vector pFP996::ldhD ko.

Electrocompetent Lactobacillus plantarum PN0512 cells were prepared, transformed with pFP996::ldhD ko, and plated on MRS containing 1 μg/ml of erythromycin. To obtain the single-crossover event (sco), transformants were passaged for approximately 50 generations in MRS medium at 37° C. After growth, aliquots were plated for single colonies on MRS containing 1 μg/ml of erythromycin. The erythromycin-resistant colonies were screened by PCR amplification with primers ldhD Seq F1 (SEQ ID NO:115) and D check R (SEQ ID NO:116) to distinguish between wildtype and clones carrying the sco event. To obtain clones with a double crossover, the sco strains were passaged for approximately 30 generations in MRS medium with 20 mM D, L-lactate (Sigma, St. Louis, Mo.) at 37° C. and then plated for single colonies on MRS with lactate. Colonies were picked and patched onto MRS with lactate and MRS with lactate containing 1 μg/ml of erythromycin to find colonies sensitive to erythromycin. Sensitive colonies were screened by PCR amplification using primer D check R (SEQ ID NO:116) and D check F3 (SEQ ID NO:1117). Wildtype colonies gave a 3.2 kbp product and deletion clones, called PN0512ΔldhD, gave a 2.3 kbp PCR product.

ΔldhDΔldhL1

A deletion of the ldhL1 gene was made in the PN0512ΔldhD strain background in order to make a double ΔldhL1ΔldhD deletion strain. The knockout cassette to delete the ldhL1 gene was amplified from PN0512 genomic DNA. The ldhL1 left homologous arm was amplified using primers oBP31 (SEQ ID NO:118) containing a BglII restriction site and oBP32 (SEQ ID NO:119) containing an XhoI restriction site. The ldhL1 right homologous arm was amplified using primers oBP33 (SEQ ID NO:120) containing an XhoI restriction site and oBP34 (SEQ ID NO:121) containing an XmaI restriction site. The ldhL1 left homologous arm was cloned into the BglII/XhoI sites and the ldhL1 right homologous arm was cloned into the XhoI/XmaI sites of pFP996pyrFΔerm, a derivative of pFP996. pFP996pyrFΔerm contains the pyrF sequence (SEQ ID NO:122) encoding orotidine-5′-phosphate decarboxylase from Lactobacillus plantarum PN0512 in place of the erythromycin coding region in pFP996. The plasmid-borne pyrF gene, in conjunction with the chemical 5-fluoroorotic acid in a ΔpyrF strain, can be used as an effective counter-selection method in order to isolate the second homologous crossover. The XmaI fragment containing the ldhL1 homologous arms was isolated following XmaI digestion and cloned into the XmaI restriction site of pFP996, yielding a 900 bp left homologous region and a 1200 bp right homologous region resulting in vector pFP996-ldhL1-arms.

PN0512ΔldhD was transformed with pFP996-ldhL1-arms and grown at 30° C. in Lactobacilli MRS medium with lactate (20 mM) and erythromycin (1 μg/ml) for approximately 10 generations. Transformants were then grown under non-selective conditions at 37° C. for about 50 generations by serial inoculations in MRS+lactate before cultures were plated on MRS containing lactate and erythromycin (1 μg/ml). Isolates were screened by colony PCR for a single crossover using chromosomal specific primer oBP49 (SEQ ID NO:123) and plasmid specific primer oBP42 (SEQ ID NO:124). Single crossover integrants were grown at 37° C. for approximately 40 generations by serial inoculations under non-selective conditions in MRS with lactate before cultures were plated on MRS medium with lactate. Isolates were patched to MRS with lactate plates, grown at 37° C., and then patched onto MRS plates with lactate and erythromycin (1 μg/ml). Erythromycin sensitive isolates were screened by colony PCR for the presence of a wild-type or deletion second crossover using chromosomal specific primers oBP49 (SEQ ID NO:123) and oBP56 (SEQ ID NO:125). A wild-type sequence yielded a 3505 bp product and a deletion sequence yielded a 2545 bp product. The deletions were confirmed by sequencing the PCR product and absence of plasmid was tested by colony PCR with primers oBP42 (SEQ ID NO:124) and oBP57 (SEQ ID NO:126).

The Lactobacillus plantarum PN0512 double ldhDldhL1 deletion strain was designated PNP0001. The ΔldhD deletion included 83 bp upstream of where the ldhD start codon was through amino acid 279 of 332. The ΔldhL1 deletion included the fMet through the final amino acid.

The chromosomal integration of a single copy of the L. lactis ilvD coding region expressed from the ldhL1 promoter was constructed by the same two-step homologous recombination procedure to yield an unmarked integration as described above using the pFP996 shuttle vector except that the second crossover event yielded the wild type sequence or the intended integration rather than the deletion. Two segments of DNA containing sequences upstream and downstream of the intended integration site were cloned into the plasmid to provide the regions of homology for two genetic crossovers.

Two DNA segments (homologous arms) were designed to provide regions of homology for the two genetic cross-overs such that integration would place the ilvD coding region downstream of the ldhL1 promoter in strain PN0512ΔldhDΔldhL1. The left and right homologous arms cloned into the plasmid were each approximately 1200 base pairs. The left homologous arm was amplified from L. plantarum PN0512 genomic DNA with primers oBP31 (SEQ ID NO:118), containing a BglII restriction site, and oBP32 (SEQ ID NO119), containing an XhoI restriction site using Phusion High-Fidelity PCR Master Mix. The right homologous arm was amplified from L. plantarum PN0512 genomic DNA with primers oBP33 (SEQ ID NO:120), containing an XhoI restriction site and oBP34 (SEQ ID NO:121), containing an XmaI restriction site using Phusion High-Fidelity PCR Master Mix. The left homologous arm was digested with BglII and XhoI and the right homologous arm was digested with XhoI and XmaI. The two homologous arms were ligated with T4 DNA Ligase into the corresponding restriction sites of pFP996, after digestion with the appropriate restriction enzymes, to generate the vector pFP996-ldhL1 arms.

A DNA fragment containing the ilvD coding region from Lactococcus lactis (SEQ ID NO:94) and a ribosome binding sequence (RBS; SEQ ID NO:127) was amplified from pDM20-ilvD(L. lactis) (SEQ ID NO:128). Construction of pDM20-ilvD(L. lactis) was described in U.S. Patent Application No. 61/100,809, which is herein incorporated by reference. This plasmid is pDM20 containing the ilvD coding region derived by PCR from L. lactis subsp lactis NCDO2118 (NCIMB 702118) [Godon et al., J. Bacteriol. (1992) 174:6580-6589] and a ribosome binding sequence (SEQ ID NO:1127) added in the 5″ PCR primer. pDM20 is modified pDM1 (SEQ ID NO:129) which contains a minimal pLF1 replicon (˜0.7 Kbp) and pemK-peml toxin-antitoxin(TA) from Lactobacillus plantarum ATCC14917 plasmid pLF1, a P15A replicon from pACYC184, chloramphenicol resistance marker for selection in both E. coli and L. plantarum, and P30 synthetic promoter [Rud et al., Microbiology (2006) 152:1011-1019]. Vector pDM1 was modified by deleting nucleotides 3281-3646 spanning the lacZ region which were replaced with a multi cloning site. Primers oBP120 (SEQ ID NO:1130), containing an XhoI site, and oBP182 (SEQ ID NO:131), containing DrdI, PstI, HindIII, and BamHI sites, were used to amplify the P30 promoter from pDM1 with Phusion High-Fidelity PCR Master Mix. The resulting PCR product and pDM1 vector were digested with XhoI and DrdI, which drops out lacZ and P30. The PCR product and the large fragment of the pDM1 digestion were ligated to yield vector pDM20 in which the P30 promoter was reinserted, bounded by XhoI and DrdI restriction sites.

The DNA fragment containing the ilvD coding region and RBS (SEQ ID NO:132) was obtained by PCR using pDM20-ilvD(L. lactis) as the template with primers oBP246 (SEQ ID NO:133), containing an XhoI restriction site, and oBP237 (SEQ ID NO:134), containing an XhoI restriction site, using Phusion High-Fidelity PCR Master Mix. The resulting PCR product and pFP996-ldhL1arms were ligated with T4 DNA Ligase after digestion with XhoI. Clones were screened by PCR for the insert in the same orientation as the ldhL1 promoter in the left homologous arm using vector specific primer oBP57 (SEQ ID NO:126) and ilvD-specific primer oBP237 (SEQ ID NO:134). A clone that had the correctly oriented insert was named pFP996-ldhL1arms-ilvDLI.

Integration of the L. lactis ilvD coding region was obtained by transforming L. plantarum PN0512ΔldhDΔldhL1 with pFP996-ldhL1arms-ilvDLI. 5 ml of Lactobacilli MRS medium (Accumedia, Neogen Corporation, Lansing, Mich.) containing 0.5% glycine (Sigma-Aldrich, St. Louis, Mo.) was inoculated with PN0512ΔldhDΔldhL1 and grown overnight at 30° C. 100 ml MRS medium with 0.5% glycine was inoculated with overnight culture to an OD600 of 0.1 and grown to an OD600 of 0.7 at 30° C. Cells were harvested at 3700×g for 8 min at 4° C., washed with 100 ml cold 1 mM MgCl₂ (Sigma-Aldrich, St. Louis, Mo.), centrifuged at 3700×g for 8 min at 4° C., washed with 100 ml cold 30% PEG-1000 (Sigma-Aldrich, St. Louis, Mo.), recentrifuged at 3700×g for 20 min at 4° C., then resuspended in 1 ml cold 30% PEG-1000. 60 μl of cells were mixed with ˜100 ng of plasmid DNA in a cold 1 mm gap electroporation cuvette and electroporated in a BioRad Gene Pulser (Hercules, Calif.) at 1.7 kV, 25 μF, and 400Ω. Cells were resuspended in 1 ml MRS medium containing 500 mM sucrose (Sigma-Aldrich, St. Louis, Mo.) and 100 mM MgCl₂, incubated at 30° C. for 2 hrs, and then plated on MRS medium plates containing 2 μg/ml of erythromycin (Sigma-Aldrich, St. Louis, Mo.).

Transformants were screened by PCR using ilvD specific primers oBP237 (SEQ ID NO:134) and oBP246 (SEQ ID NO:133). Transformants were grown at 30° C. in Lactobacilli MRS medium with erythromycin (1 μg/ml) for approximately 8 generations and then at 37° C. for approximately 40 generations by serial inoculations in Lactobacilli MRS medium. The cultures were plated on Lactobacilli MRS medium with erythromycin (0.5 μg/ml). The isolates were screened by colony PCR for a single crossover with chromosomal specific primer oBP49 (SEQ ID NO:123) and plasmid specific primer oBP42 (SEQ ID NO:124).

Single crossover integrants were grown at 37° C. for approximately 43 generations by serial inoculations in Lactobacilli MRS medium. The cultures were plated on MRS medium. Colonies were patched to MRS plates and grown at 37° C. The isolates were then patched onto MRS medium with erythromycin (0.5 μg/ml). Erythromycin sensitive isolates were screened by (colony) PCR for the presence of a wild-type or integration second crossover using chromosomal specific primers oBP49 (SEQ ID NO:123) and oBP56 (SEQ ID NO:125). A wild-type sequence yielded a 2600 bp product and an integration sequence yielded a 4300 bp product. The integration was confirmed by sequencing the PCR product and an identified integration strain was designated PN0512ΔldhDΔldhL1::ilvDLI⁺.

### Example 2

### Construction of a suf Operon Promoter Integration Vector and PN0512ΔldhDΔldhL1::ilvDLI⁺suf::P5P4⁺ Integration Strain

This Example describes integration of two promoters into the chromosome of L. plantarum PN0512ΔldhDΔldhL1::ilvDLI⁺. The promoters were integrated upstream of the suf operon, whose gene products are responsible for Fe—S cluster assembly. The promoter integration results in a strain with increased expression of the endogenous Fe—S cluster machinery.

The suf operon chromosomal promoter integration was constructed by a two-step homologous recombination procedure to yield an unmarked integration using the shuttle vector pFP996 (SEQ ID NO:110) as described above.

The suf operon promoter integration vector was constructed in three steps. In the first step, a right homologous arm fragment containing the 5′ portion of the suf operon (sufC and part of sufD) was cloned into pFP996. In the second step, the synthetic promoters P5 and P4 [Rud et al., Microbiology (2006) 152:1011] were cloned into the pFP996-right arm clone upstream of the right arm. In the final step, a left homologous arm fragment containing the native suf promoter and sequences upstream into the feoBA operon was cloned into the pFP996-P5P4-right arm clone upstream of the P5P4 promoters.

The right homologous arm DNA fragment (SEQ ID NO:135) was PCR amplified from L. plantarum PN0512 genomic DNA with primers AA199 (SEQ ID NO:136), containing an XmaI restriction site, and AA200 (SEQ ID NO:137), containing a KpnI restriction site, using Phusion High-Fidelity PCR Master Mix. The right homologous arm PCR fragment and pFP996 were ligated with T4 DNA Ligase after digestion with XmaI and KpnI to generate pFP996-sufCD. A DNA fragment containing promoters P5 and P4 was generated by performing PCR with two partially complementary primer sequences. Primer AA203 (SEQ ID NO:138), containing an XhoI site, the P5 promoter sequence, and part of the P4 promoter sequence, was combined with primer AA204 (SEQ ID NO:139), containing an XmaI site and the P4 promoter sequence, and PCR was performed with Phusion High-Fidelity PCR Master Mix. The resulting PCR product was then amplified with primers AA206 (SEQ ID NO:140) and AA207 (SEQ ID NO:141) with Phusion High-Fidelity PCR Master Mix. The P5P4 PCR product and pFP996-sufCD were ligated after digestion with XhoI and XmaI to generate pFP996-P5P4-sufCD. The left homologous arm DNA fragment (SEQ ID NO:142) was amplified from L. plantarum PN0512 genomic DNA with primers AA201 (SEQ ID NO:143), containing an EcoRI restriction site, and AA202 (SEQ ID NO:144), containing an XhoI restriction site, using Phusion High-Fidelity PCR Master Mix. The left homologous arm and pFP996-P5P4-sufCD were ligated with T4 DNA Ligase after digestion with EcoRI and XhoI to generate pFP996-feoBA-P5P4-sufCD. The vector was confirmed by sequencing. The vector had a five base pair deletion (TTGTT), encompassing part of the −35 hexamer in the upstream P5 promoter.

Integration of the synthetic promoters (P5P4) upstream of the suf operon was obtained by transforming L. plantarum PN0512ΔldhDΔldhL1::ilvDLI⁺ with pFP996-feoBA-P5P4-sufCD as described above. Transformants were grown at 30° C. in Lactobacilli MRS medium with erythromycin (2 μg/ml) for approximately 20 generations. The cultures were plated on Lactobacilli MRS medium with erythromycin (0.5 μg/ml). Isolates were screened by colony PCR for a single crossover with chromosomal specific primer AA209 (SEQ ID NO:145) and plasmid specific primer AA210 (SEQ ID NO:146). Single crossover integrants were grown at 37° C. for approximately 30 generations by serial inoculations in Lactobacilli MRS medium. The cultures were plated on MRS medium. Isolates were screened for erythromycin sensitivity. Isolates were screened by (colony) PCR for the presence of a wild-type or integration second crossover using P5 specific primer AA211 (SEQ ID NO:147) and chromosomal specific primer oBP126 (SEQ ID NO:148). An identified integration strain was designated PN0512ΔldhDΔldhL1::ilvDLI⁺suf::P5P4⁺.

### Example 3

### Construction of the Tn5-Transposon Vector (pTN6) and its Use for Integration of PgroE-kivD(o)-sadB(o) Cassette

Tn5 is a bacterial transposon which has been well characterized in E. coli (Johnson & Reznikoff, Nature (1983) 304:280-282). A Tn5-mediated transposition system for lactic acid bacteria (LAB) was described in U.S. Provisional Patent Application No. 61/246,717, incorporated herein by reference. In this Example, use of a Tn5-transposon vector as a delivery system for random gene integration into the chromosome of LAB was developed. The developed Tn5-transposon vector (pTN6) (SEQ ID NO:149) is an E. coli-L. plantarum shuttle vector. Plasmid pTN6 contains a transposase gene (tnp), transposase recognition nucleotide sequences Tn5IE (19 base pairs inside end) and Tn5OE (19 base pairs outside end), two antibiotic resistance markers; one for resistance to chloramphenicol and the other for resistance to erythromycin, P15A replication origin for E. coli, pE194 replication origin for L. plantarum which is temperature sensitive (Horinouchi and Weisblum J. Bacteriol. (1982) 150:804-814), and two loxP nucleotide sequences (34 base pairs). The chloramphenicol resistance gene is flanked by loxP sites for later excision by Cre recombinase. Multiple cloning sites (MSC) that contain restriction sites for BamHI, NotI, ScaI, and SpeI are located between the loxP and Tn5OE sites. The chloramphenicol resistance gene, two loxP sites, and MCS are flanked by Tn5IE and Tn5OE.

To construct the Tn5-transposon vector pTN6, first the 1,048 bp Tn5IE-loxP-cm-loxP cassette containing Tn5IE, loxP, chloramphenicol resistant gene (cm), and loxP was synthesized by Genscript Corp (Piscataway, N.J.) (SEQ ID NO:150). The Tn5IE-loxP-cm-Pspac-loxP cassette was cloned in the pUC57 vector (Genscript Corp, Piscataway, N.J.), producing plasmid pUC57-Tn5IE-loxP-cm-loxP. The chloramphenicol resistance gene is expressed under the control of the spac promoter (Yansura & Henner, (1984) Proc Natl Acad Sci USA. 81:439-443) for selection in both E. coli and L. plantarum. Plasmid pUC57-Tn5IE-loxP-cm-loxP was digested with NsiI and SacI, and the 1,044 bp Tn5IE-loxP-cm-loxP fragment was gel-purified. Plasmid pFP996 (SEQ ID NO:110) was digested with NsiI and SacI, and the 4,417 bp pFP996 fragment containing the pBR322 and pE194 replication origins was gel-purified. The Tn5IE-loxP-cm-loxP fragment was ligated with the 4,417 bp pFP996 fragment to generate pTnCm.

Second, the pBR322 replication origin on pTnCm was replaced by the P15A replication origin. Plasmid pTnCm was digested with AatlI and SalI, and the 2,524 bp pTnCm fragment containing the pE194 replication origin and Tn5IE-loxP-cm-loxP cassette was gel-purified. The 913 bp p15A replication origin was PCR-amplified from pACYC184 [Chang and Cohen, J. Bacteriol. (1978)134:1141-1156] with primers T-P15A(SalITn5OE) (SEQ ID NO:151) that contains a SalI restriction site and 19 bp Tn5OE nucleotide sequence, and B-P15A(AatlI) (SEQ ID NO:152) that contains an AatlI restriction site by using Phusion High-Fidelity PCR Master Mix (New England Biolabs, Ipswich, Mass.). The P15A fragment, after digestion with SalI and AatlI restriction enzymes, was ligated with the 2,524 bp pTnCm fragment to generate pTN5.

Third, the erythromycin resistance gene (erm) was cloned into the HindIII site on pTN5. The 1,132 bp erythromycin resistant gene (erm) DNA fragment was generated from vector pFP996 (SEQ ID NO:110) by PCR amplification with primers T-erm(HindIII) (SEQ ID NO:153) containing an NsiI restriction site and B-erm(HindIII) (SEQ ID NO:154) containing an NsiI restriction site by using Phusion High-Fidelity PCR Master Mix, and cloned into the HindIII restriction site on pTN5, producing pTN5-erm.

Finally, a tnp gene sequence encoding transposase was fused to the npr (neutral protease from Bacillus amyloliquefaciens) promoter [Nagarajan et al., J. Bacteriol (1984) 159:811-819] by SOE (splicing by overlap extension) PCR, and cloned into the NsiI site on pTN5-erm. A DNA fragment containing the Pnpr promoter (SEQ ID NO:155) was PCR-amplified from pBE83 [Nagarajan et al., Appl Environ Microbiol (1993) 59:3894-3898] with primer set T-Pnpr(NsiI) (SEQ ID NO:156) containing an NsiI restriction site and B-Pnpr(tnp) (SEQ ID NO:157) containing a 17 bp overlapping sequence by using Phusion High-Fidelity PCR Master Mix. A tnp coding region (SEQ ID NO:108) was PCR-amplified from pUTmTn5-(Sharpe et al., Appl Environ Microbiol (2007) 73:1721-1728) with primer set T-tnp(Pnpr) (SEQ ID NO:1158) containing a 21 bp overlapping sequence and B-tnp(NsiI) (SEQ ID NO:159) containing an NsiI restriction site by using Phusion High-Fidelity PCR Master Mix. The PCR products of the two reactions were mixed and amplified using outer primers (T-Pnpr(NsiI) and B-tnp(NsiI)), resulting in the production of a Pnpr-tnp fusion DNA fragment (SEQ ID NO:160). Plasmid pTN5-erm was digested with NsiI and treated with Calf Intestinal Phosphatase (New England Biolabs, MA) to prevent self-ligation. The digested pTN5-erm vector was ligated with the Pnpr-tnp fragment digested with NsiI. The ligation mixture was transformed into E. coli Top10 cells (Invitrogen Corp, Carlsbad, Calif.) by electroporation. Transformants were selected on LB plates containing 25 μg/mL chloramphenicol at 37° C. Transformants then were screened by colony PCR with outer primers of the Pnpr-tnp cassette, and confirmed by DNA sequencing with primers pTnCm(711) (SEQ ID NO:161), pTnCm(1422) (SEQ ID NO: 162), and pTnCm(3025) (SEQ ID NO:163). The resulting plasmid was named pTN6.

This Tn5-transposon vector pTN6 was used as a random gene delivery system for integration of a PgroE-kivD(o)-sadB(o) cassette into the chromosome of the PN0512ΔldhDΔldhL1::ilvDLI⁺suf::P5P4⁺ strain. A DNA fragment containing a PgroE promoter (Yuan and Wong, J. Bacteriol (1995) 177:5427-5433) (SEQ ID NO:164) was PCR-amplified from genomic DNA of Bacillus subtilis with primer set T-groE (SalIKpnI) (SEQ ID NO:165) containing SalI and KpnI restriction sites and B-groE (BamHI) (SEQ ID NO:166) containing a BamHI restriction site by using Phusion High-Fidelity PCR Master Mix. The resulting 154 bp PgroE promoter fragment, after digesting with SalI and BamHI restriction enzymes, was cloned into SalI and BamHI sites of plasmid pTN6, generating pTN6-PgroE. The coding region of the kivD gene encoding the branched-chain ketol acid decarboxylase from Lactococcus lactis was codon optimized for expression in L. plantarum. The optimized coding region sequence called kivD(o) (SEQ ID NO:90) with a RBS was synthesized by Genscript Corp (Piscataway, N.J.). The kivD(o) coding region together with a RBS (SEQ ID NO:167) was cloned in the pUC57 vector, producing plasmid pUC57-kivD(o). Plasmid pUC57-kivD(o) was digested with BamHI and NotI, and the 1,647 bp RBS-kivD(o) fragment was gel-purified. The RBS-kivD(o) fragment was cloned into BamHI and NotI restriction sites on pTN6-PgroE, producing pTN6-PgroE-kivD(o). The correct clone was confirmed by colony PCR with primers T-groE(SalIKpnI) and kivD(o)R (SEQ ID NO:165 and 168), producing a 1,822 bp fragment of the expected size. Then, the sadB gene coding region for branched-chain alcohol dehydrogenase from Achromobacter xylosoxidans, that was described in U.S. patent application Ser. No. 12/430,356, was cloned downstream of the kivD(o) coding region of pTN6-PgroE-kivD(o). The A. xylosoxidans sadB coding region was codon optimized for expression in L. plantarum. The new coding region called sadB(o) (SEQ ID NO:105) with a RBS was synthesized by Genscript Corp (Piscataway, N.J.), and cloned in the pUC57 vector, producing plasmid pUC57-sadB(o). A 1,089 bp DNA fragment (SEQ ID NO:169) containing the RBS and sadB(o) coding region was PCR-amplified from pUC57-sadB(o) with primer set T-sadB(o)(NotI) (SEQ ID NO:170) containing a NotI restriction site and B-sadB(o)(NotI) (SEQ ID NO:171) containing a NotI restriction site by using Phusion High-Fidelity PCR Master Mix. The RBS-sadB(o) gene fragment, after digesting with NotI, was cloned into NotI restriction site of pTN6-PgroE-kivD(o), producing pTN6-PgroE-kivD(o)-sadB(o). The correct clone was confirmed by DNA sequencing with kivD(o)1529 (SEQ ID NO:172) and B-spac(cm) (SEQ ID NO:173) primers. In this construction sadB(o) and kivD(o) coding regions are expressed in an operon from PgroE promoter.

The resulting plasmid pTN6-PgroE-kivD(o)-sadB(o) was transformed into PN0512ΔldhDΔldhL1::ilvDLI⁺suf::P5P4⁺ by electroporation as described in General Methods. Transformants were selected on Lactobacilli MRS medium supplemented with 7.5 μg/ml chloramphenicol. The chloramphenicol resistant colonies were grown in Lactobacilli MRS medium with 7.5 μg/ml chloramphenicol at the permissive temperature of 30° C. for approximately 10 generations. The culture was inoculated at 1/100 dilution in fresh MRS medium and grown at 37° C. for approximately 20 generations by serial inoculation in Lactobacilli MRS medium. The cultures were plated on Lactobacilli MRS with 7.5 μg/ml chloramphenicol. The isolates were screened by re-streaking colonies on Lactobacilli MRS plates containing 1.5 μg/ml erythromycin for erythromycin sensitive colonies that were presumed to contain a chromosomally integrated PgroE-kivD(o)-sadB(o) cassette along with the transposon. The transposon-mediated integrants were confirmed by colony PCR with the kivD(o) sequence specific primer KivD(o)1529 and sadB(o) sequence specific primer B-sadB(o)(NotI), to produce the expected sized PCR product (1,220 bp).

To excise the chloramphenicol resistance marker that is flanked by loxP sites from the chromosome, a helper plasmid pFP352 (SEQ ID NO:174) expressing a Cre recombinase was transformed into the transposon-mediated integrant, according to the protocol as described in General Methods, and grown on Lactobacillus MRS plate containing 1.5 μg/ml erythromycin at 30° C. The cre recombinase excises the chloramphenicol marker from the chromosome by a recombiniation event between the loxP sites. The erythromycin resistant transformants were inoculated in MRS medium and grown at 37° C. for approximately 10 generations. The cultures were plated on Lactobacilli MRS without antibiotic and grown at 30° C. The isolates were screened for both erythromycin and chloramphenicol sensitive colonies by testing growth of colonies on Lactobacilli MRS plates containing 1.5 μg/ml erythromycin and Lactobacilli MRS plates containing chloramphenicol (7.5 μg/ml), separately, to verify loss of pFP352 and the chloramphenicol marker removal. Finally, the integrant was confirmed by genomic DNA sequencing with primer B-groE(BamHI). Genomic DNA was prepared using MasterPure DNA Purification® kit (Enpicentre, Inc., Madison, Wis.). The DNA sequencing result indicated that the PgroE-kivD(o)-sadB(o) cassette was Inserted within the coding region of the glgB gene encoding glycogen branching enzyme that catalyzes the transfer of a segment of a 1,4-alpha-D-glucan chain to a primary hydroxy group in a similar glucan chain. The resulting integrant was named PN0512ΔldhDΔldhL1::ilvD(LI) suf::P5P4⁺ glgB::Tn5-PgroE-kivD(o)-sadB(o).

### Example 4

### Construction of the pDM5-PldhL1-ilvC(L. lactis) Vector

The purpose of this example is to describe cloning of the ilvC coding region (SEQ ID NO:67) for ketol-acid reductoisomerase from Lactococcus lactis subsp lactis NCDO2118 (NCIMB 702118) [Godon et al., J. Bacteriol. (1992) 174:6580-6589] into the pDM5 vector.

Plasmid pDM5 (SEQ ID NO:175) was constructed by replacing the P30 promoter of pDM1 with the B. subtilis groE promoter (PgroE) fused to a lacO operator sequence and a lacI repressor gene. Plasmid pDM1 is described in Example 1. Plasmid pHTO1 (Mo Bi Tec, Goettingen, Germany) was digested with SacI, treated with Klenow fragment to make blunt ends, digested with BamHI, and then the 1,548 bp lacI-PgroE/lacO fragment (SEQ ID NO:176) was gel-purified. The lacI-PgroE/lacO fragment was cloned into KpnI (blunt ended by Klenow fragment) and BamHI sites of pDM1 in place of the P30 promoter, generating pDM5.

A DNA fragment, PldhL1-ilvC(L. lactis), containing a ldhL1 (L-lactate dehydrogenase from Lactobacillus plantarum PN0512) promoter (PldhL1) and ilvC coding region from Lactococcus lactis subsp lactis NCDO2118 was generated by SOE (splicing by overlap extension) PCR. The DNA fragment containing a PldhL1 promoter was PCR-amplified from the genomic DNA of Lactobacillus plantarum PN0512 with primer set T-ldhL1(NotI) (SEQ ID NO:177) containing a NotI restriction site and B-ldhLI(CLI) (SEQ ID NO:178) containing a 19 bp overlapping sequence by using Phusion High-Fidelity PCR Master Mix. An ilvC coding region was PCR-amplified from the genomic DNA of Lactococcus lactis subsp lactis NCDO2118 with primer set T-CLI(ldh) (SEQ ID NO:179) containing a 17 bp overlapping sequence and B-CLI(PvuI) (SEQ ID NO:180) containing a PvuI restriction site by using Phusion High-Fidelity PCR Master Mix. The PCR products of the two fragments were mixed and amplified using outer primers T-ldhL1(NotI) and B-CLI(PvuI), resulting in the production of a PldhL1-ilvC(L. lactis) fusion DNA fragment. Plasmid pDM5 was digested with NotI and PvuI restriction enzymes, and ligated with the PldhL1-ilvC(L. lactis) cassette after digesting with NotI and PvuI restriction enzymes. The ligation mixture was transformed into E. coli Top10 cells (Invitrogen Corp, Carlsbad, Calif.) by electroporation. Transformants were selected on LB plates containing 25 μg/mL chloramphenicol at 37° C. Transformants then were screened by colony PCR with outer primers of the PldhL1-ilvC(L. lactis) cassette, and confirmed by DNA sequencing with T-ldhL1(NotI) (SEQ ID NO:177) and pDM(R)new (SEQ ID NO:181). The resulting plasmid was named pDM5-PldhL1-ilvC(L. lactis) (SEQ ID NO:182).

### Example 5

### Construction of the pDM5-PldhL1-ilvC (P. fluorescence 5) Vector

The purpose of this example is to describe cloning of the ilvC coding region for ketol-acid reductoisomerase from Pseudomonas fluorescens PF5 into a expression vector.

The P. fluorescens PF5 ilvC coding region was codon optimized for expression in Lactobacillus plantarum as primary host. This optimized coding region called ilvC(P. fluorescens PF5) or ilvC(Pf5) together with a RBS (SEQ ID NO:183) was synthesized by Genscript Corp (Piscataway, N.J.). The coding region ilvC(P. fluorescens PF5) and RBS fragment was cloned in the pUC57 vector, producing plasmid pUC57-ilvC(P. fluorescens PF5), and then sub-cloned into pFP996-PldhL1 (SEQ ID NO:184) as an XhoI-KpnI fragment to generate pFP996-PldhL1-ilvC(P. fluorescens PF5). pFP996-PldhL1 contains the PldhL1 DNA fragment described in Example 4. The PldhL1-ilvC(P. fluorescens PF5) DNA fragment (SEQ ID NO:185) containing an ldhL1 (L-lactate dehydrogenase from Lactobacillus plantarum PN0512) promoter (PldhL1) and ilvC coding region from Pseudomonas fluorescens PF5 was generated by PCR from pFP996-PldhL1-ilvC(P. fluorescens PF5) with primer set T-ldhL1(NotI) (SEQ ID NO:186) containing a NotI restriction site and B-CPf(o)(EcoRV) (SEQ ID NO:187) containing a EcoRV restriction site. The 1,297 bp PldhL1-ilvC(P. fluorescens PF5) fragment was digested with NotI and EcoRV, and ligated into NotI and PvuII sites of pDM1-ilvD(L. lactis) creating pDM1-ilvD(L. lactis)-PldhL1-ilvC(P. fluorescens PF5). The construction of pDM1-ilvD(L. lactis) was described in Example 1 of U.S. Provisional Patent Application No. 61/100,810 as follows.

The Lactococcus lactis ilvD coding region (SEQ ID NO:94) was PCR-amplified from Lactococcus lactis subsp lactis NCDO2118 genomic DNA with primers 3T-ilvDLI(BamHI) (SEQ ID NO:188) and 5B-ilvDLI(NotI) (SEQ ID NO:189). L. lactis subsp lactis NCDO2118 genomic DNA was prepared with a Puregene Gentra Kit (QIAGEN; Valencia, Calif.). The 1.7 Kbp L. lactis ilvD PCR product (ilvDLI) was digested with NotI and treated with the Klenow fragment of DNA polymerase to make blunt ends. The resulting L. lactis ilvD coding region fragment was digested with BamHI and gel-purified using a QIAGEN gel extraction kit (QIAGEN). Plasmid pDM1 was digested with ApaLI, treated with the Klenow fragment of DNA polymerase to make blunt ends, and then digested with BamHI. The gel purified L. lactis ilvD coding region fragment was ligated into the BamHI and ApaLI(blunt) sites of the plasmid pDM1. The ligation mixture was transformed into E. coli Top10 cells (Invitrogen; Carlsbad, Calif.). Transformants were plated for selection on LB chloramphenicol plates. Positive clones were screened by SalI digestion, giving one fragment with an expected size of 5.3 Kbp. The positive clones were further confirmed by DNA sequencing. The correct clone was named pDM1-ilvD(L. lactis), which has the L. lactis ilvD coding region expressed from P30.

The P30 promoter of pDM1-ilvD(L. lactis)-PldhL1-ilvC(P. fluorescens PF5) was replaced with the B. subtilis groE promoter (PgroE) fused to a lacO operator sequence and a tact repressor gene. Plasmid pHTO1 (Mo Bi Tec, Goettingen, Germany) was digested with SacI, treated with Klenow fragment to make blunt ends, digested with BamHI, and then the 1,548 bp lacI-PgroE/lacO fragment (SEQ ID NO:176) was gel-purified. The lacI-PgroE/lacO DNA fragment was cloned into KpnI (blunt end by Klenow fragment) and BamHI sites of pDM1-ilvD(L. lactis)-PldhL1-ilvC(P. fluorescens PF5) in place of the P30 promoter, producing pDM5-ilvD(L. lactis)-PldhL1-ilvC(P. fluorescens PF5).

To remove the ilvD(L. lactis) fragment pDM5-ilvD(L. lactis)-PldhL1-ilvC(P. fluorescens PF5) was digested with NotI and BamHI, and treated with CIP to make blunt ends. The 6,207 bp DNA fragment containing a pDM5 backbone, ldhL1 promoter (PldhL1) and P. fluorescens PF5 ilvC coding region was gel-purified and self-ligated, producing pDM5-PldhL1-ilvC(P. fluorescens PF5).

### Example 6

### Construction of the pDM5-PldhL1-ilvC(S. mutans), pDM5-PldhL1-ilvC(S. thermophilus), and pDM5-PldhL1-ilvC(L. mesenteroides) Vectors

The purpose of this example is to describe the cloning of the Streptococcus mutans UA159 ilvC coding region for ketol-acid reductoisomerase (coding SEQ ID NO:27; protein SEQ ID NO:28), the Streptococcus thermophilus LMD-9 ilvC coding region for ketol-acid reductoisomerase ((coding SEQ ID NO:55; protein SEQ ID NO:56), and the Leuconostoc mesenteroides subsp. mesenteroides ATCC8293 ilvC coding region for ketol-acid reductoisomerase (coding SEQ ID NO:39; protein SEQ ID NO:40) into expression vectors.

To clone the S. mutans, S. thermophilus, and L. mesenteroides ilvC coding regions under the control of the same promoters as the L. lactis and P. fluorescens PF5 ilvC genes in Examples 4 and 5, vector pDM5-PldhL1-MCS was first constructed. The ldhL1 promoter from L. plantarum PN0512 genomic DNA was amplified with primers AA234 (SEQ ID NO:190), containing a NotI restriction site, and AA179 (SEQ ID NO:191), containing multiple restriction sites for a multi cloning site (MCS) and a DrdI restriction site, using Phusion High-Fidelity PCR Master Mix. The resulting DNA fragment was digested with NotI and DrdI. Vector pDM5-PldhL1-ilvC(P. fluorescens Pf5) was digested with NotI and DrdI and the larger fragment (4712 bp) was gel purified to remove the PldhL1-ilvC(P. fluorescens Pf5) sequence. The gel purified fragment was ligated with the digested PldhL1-MCS PCR to create vector pDM5-PldhL1-MCS with unique restriction sites KasI, PacI, AvrII, SacI, and PmeI. The vector was confirmed by PCR and sequencing.

The Streptococcus mutans UA159 ilvC coding region (SEQ ID NO:27) from S. mutans UA159 genomic DNA was amplified with primers AA235 (SEQ ID NO:192), containing an AvrII restriction site and a ribosome binding sequence, and AA236 (SEQ ID NO:193), containing a SacI restriction site, using Phusion High-Fidelity PCR Master Mix. The Streptococcus thermophilus LMD-9 (ATCC BAA-491) ilvC coding region (SEQ ID NO:55) was amplified by colony PCR with primers AA237 (SEQ ID NO:194), containing an AvrII restriction site and a ribosome binding sequence, and AA238 (SEQ ID NO::195), containing a SacI restriction site, using Phusion High-Fidelity PCR Master Mix. The Leuconostoc mesenteroides subsp. mesenteroides ATCC 8293 ilvC coding region (SEQ ID NO:39) from L. mesenteroides subsp. mesenteroides ATCC 8293 genomic DNA was amplified with primers AA239 (SEQ ID NO:196), containing an AvrII restriction site and ribosome binding sequence, and AA240 (SEQ ID NO::197), containing a SacI restriction site, using Phusion High-Fidelity PCR Master Mix. The three ilvC genes were cut with AvrII and SacI and ligated individually into the corresponding restriction sites of pDM5-PldhL1-MCS after digestion with AvrII and SacI to create vectors pDM5-PldhL1-ilvC(S. mutans), pDM5-PldhL1-ilvC(S. thermophilus), and pDM5-PldhL1-ilvC(L. mesenteroides). The vectors were confirmed by PCR and sequencing.

### Example 7

### Production of Isobutanol Using PN0512ΔldhDΔldhL1::ilvD(LI) suf::P5P4⁺ glgB::Tn5-PgroE-kivD(o)-sadB(o) Containing Vector pDM5-PldhL1-ilvC(L. lactis), pDM5-PldhL1-ilvC(S. mutans), pDM5-PldhL1-ilvC(S. thermophilus), pDM5-PldhL1-ilvC(L. mesenteroides), or pDM5-PldhL1-ilvC(P. fluorescens Pf5)

The purpose of this example is to demonstrate the increased production of isobutanol in PN0512ΔldhDΔldhL1::ilvD(LI) suf::P5P4⁺ glgB::Tn5-PgroE-kivD(o)-sadB(o) containing vector pDM5-PldhL1-ilvC(L. lactis), pDM5-PldhL1-ilvC(S. mutans), pDM5-PldhL1-ilvC(S. thermophilus), or pDM5-PldhL1-ilvC(L. mesenteroides), compared to PN0512ΔldhDΔldhL1::ilvD(LI) suf::P5P4⁺ glgB::Tn5-PgroE-kivD(o)-sadB(o) containing vector pDM5-PldhL1-ilvC(P. fluorescens Pf5).

To construct the recombinant Lactobacillus plantarum expressing the genes of the isobutanol biosynthetic pathway, competent cells of PN0512ΔldhDΔldhL1::ilvD(LI) suf::P5P4⁺ glgB::Tn5-PgroE-kivD(o)-sadB(o) were prepared as described in General Methods and transformed with plasmid pDM5-PldhL1-ilvC(P. fluorescens Pf5), pDM5-PldhL1-ilvC(L. lactis), pDM5-PldhL1-ilvC(S. mutans), pDM5-PldhL1-ilvC(S. thermophilus), or pDM5-PldhL1-ilvC(L. mesenteroides), yielding PN0512ΔldhDΔldhL1::ilvD(LI) suf::P5P4⁺ glgB::Tn5-PgroE-kivD(o)-sadB(o)/pDM5-PldhL1-ilvC(P. fluorescens Pf5), PN0512ΔldhDΔldhL1::ilvD(LI) suf::P5P4⁺ glgB::Tn5-PgroE-kivD(o)-sadB(o)/pDM5-PldhL1-ilvC(L. lactis), PN0512ΔldhDΔldhL1::ilvD(LI) suf::P5P4⁺ glgB::Tn5-PgroE-kivD(o)-sadB(o)/pDM5-PldhL1-ilvC(S. mutans), PN0512ΔldhDΔldhL1::ilvD(LI) suf::P5P4⁺ glgB::Tn5-PgroE-kivD(o)-sadB(o)/pDM5-PldhL1-ilvC(S. thermophilus), and PN0512ΔldhDΔldhL1::ilvD(LI) suf::P5P4⁺ glgB::Tn5-PgroE-kivD(o)-sadB(o)/pDM5-PldhL1-ilvC(L. mesenteroides), respectively. The first enzyme for the isobutanol pathway, acetolactate synthase, was provided by native expression from the endogenous gene.

The five strains of PN0512ΔldhDΔldhL1::ilvD(LI) suf::P5P4⁺ glgB::Tn5-PgroE-kivD(o)-sadB(o) containing vector pDM5-PldhL1-ilvC(P. fluorescens Pf5), pDM5-PldhL1-ilvC(L. lactis), pDM5-PldhL1-ilvC(S. mutans), pDM5-PldhL1-ilvC(S. thermophilus), or pDM5-PldhL1-ilvC(L. mesenteroides) were inoculated in Lactobacilli MRS medium containing 10 μg/ml chloramphenicol in culture tubes and grown aerobically at 30° C. overnight. Overnight cultures were used to inoculate 20 ml MRS medium containing 100 mM 3-Morpholinopropanesulfonic acid (MOPS) pH7, 10 μg/ml chloramphenicol, 40 μM ferric citrate, and 0.5 mM cysteine in 120 ml serum bottles to an initial OD600 of 0.3. Cultures were grown with shaking (100 RPM) anaerobically at 37° C. for 72 hours. Samples of the cultures were centrifuged at 3700×g for 10 minutes at 4° C. and the supernatants filtered through a 0.2 μm filter (Pall Life Sciences, Ann Arbor, Mich.). The filtered supernatants were analyzed by GC with column HP-Innowax Polyethylene Glycol (19091N-113, Agilent Technologies, Santa Clara, Calif.) and flame ionization detection. Results in Table 3 show the production of isobutanol for the five strains. The amount of isobutanol produced by PN0512ΔldhDΔldhL1::ilvD(LI) suf::P5P4⁺ glgB::Tn5-PgroE-kivD(o)-sadB(o) containing vector pDM5-PldhL1-ilvC(L. lactis) was 4.9 mM, which is approximately 5-fold higher than the isobutanol level (1.0 mM) produced by PN0512ΔldhDΔldhL1::ilvD(LI) suf::P5P4⁺ glgB::Tn5-PgroE-kivD(o)-sadB(o) containing vector pDM5-PldhL1-ilvC(P. fluorescens Pf5). Each of the other KARIs had at least 3-fold higher isobutanol production than Pf5.

TABLE 3
Production of isobutanol by the recombinant
Lactobacillus plantarum strains.

| | Isobutanol |
|---|---|
| Strain | (mM) |
| PN0512ΔldhDΔldhL1::ilvD(LI) | 1.0 |
| suf::P5P4⁺ glgB::Tn5-PgroE-kivD(o)- | |
| sadB(o)/pDM5-PldhL1-ilvC(P. fluorescens Pf5) | |
| PN0512ΔldhDΔldhL1::ilvD(LI) | 4.9 |
| suf::P5P4⁺ glgB::Tn5-PgroE-kivD(o)- | |
| sadB(o)/pDM5-PldhL1-ilvC(L. lactis) | |
| PN0512ΔldhDΔldhL1::ilvD(LI) | 3.8 |
| suf::P5P4⁺ glgB::Tn5-PgroE-kivD(o)- | |
| sadB(o)/pDM5-PldhL1-ilvC(S. mutans) | |
| PN0512ΔldhDΔldhL1::ilvD(LI) | 3.2 |
| suf::P5P4⁺ glgB::Tn5-PgroE-kivD(o)- | |
| sadB(o)/pDM5-PldhL1-ilvC(S. thermophilus) | |
| PN0512ΔldhDΔldhL1::ilvD(LI) | 3.6 |
| suf::P5P4⁺ glgB::Tn5-PgroE-kivD(o)- | |
| sadB(o)/pDM5-PldhL1-ilvC(L. mesenteroides) | |

### Example 8

### Expression of Different KARI Enzymes for Isobutanol Production in Yeast Vector Construction

A two plasmid system was used to engineer the isobutanol pathway in yeast. The first plasmid for expression of ketol-acid reductoisomerase (KARI) and acetolactate synthase (ALS) was designated as pYZ090 (SEQ ID NO:198). pYZ090 was constructed to contain a chimeric gene having the coding region of the alsS gene from Bacillus subtilis (nt position 457-2172) expressed from the yeast CUP1 promoter (nt 2-449) and followed by the CYC1 terminator (nt 2181-2430) for expression of ALS, and a chimeric gene having the coding region of the ilvC gene from Lactococcus lactis (nt 3634-4656) expressed from the yeast ILV5 promoter (2433-3626) and followed by the ILV5 terminator (nt 4682-5304) for expression of KARI. The L. lactis KARI coding region in this vector was a DNA fragment obtained by PCR using primer set LLKARI-PmeI (SEQ ID N0,199) and LLKARI-SfiI SEQ ID NO; 200) and pLH475-IlvC(LI) as the template. To construct pLH475-IlvC(LI), the ilvC-LI coding region was amplified with primer set IlvC(Lactis)-F and IlvC(Lactis)-R (SEQ ID NOs:201 and 202) using as template pDM5-PldhL1-ilvC(L. lactis), which was described above in Example 4. The PCR product was digested with AvrII and SfiI and cloned into corresponding sites of a pLH475-based vector creating the construct pLH475-IlvC (LI) (SEQ ID NO:203), also called pLH475-IlvC (L. lactis).

Plasmid pYZ091 (SEQ ID NO:204) is the same as pYZ090 except that the L. lactis ilvC coding region is replaced with the Pseudomonas fluorescens ilvC coding region (ilvC(Pf-5). The IlvC(Pf-5) coding region was amplified with primer set pILVCy-PmeII (SEQ ID NO:205) and pilvCy-SfiI (SEQ ID NO:206) using pLH532 as template DNA. pLH532 (SEQ ID NO:207) is a pHR81 vector (ATCC #87541) in which the ILV5 coding region (nt 8118-9167) is located between the FBA promoter (nt 7454-8110) and CYC1 terminator (nt 9176-9425), and the IlvC coding region from P. fluorescence Pf-5 (nt 10192-11208) is located between the ILV5 promoter (nt 11200-12390) and the ILV5 terminator (nt 9434-10191). This gene is the reverse complement of the sequence of pLH532 in SEQ ID NO:207. The Pf-5 coding region had been codon optimized for expression in S. cerevisiae.

Plasmid pYZ058 (SEQ ID NO:208) is the same as pYZ090 except that the L. lactis ilvC coding region is replaced with the Saccharomyces cerevisiae ILV5 coding region (ILV5(Sc)). The S. cerevisiae ILV5 coding region without the mitochondrial signal peptide was amplified with primer set pilv5-PmeI (SEQ ID NO:209) and pilv5-SfiI (SEQ ID NO:210) using pLH532 as the template DNA.

A second plasmid with the remaining isobutanol pathway genes was designated as pYZ067 (SEQ ID NO:211). This vector was was constructed to contain the following chimeric genes: 1) the coding region of the ilvD gene from S. mutans UA159 with a C-terminal Lumio tag (nt position 2260-3996) expressed from the yeast FBA1 promoter (nt 1161-2250) followed by the FBA1 terminator (nt 4005-4317) for expression of dihydroxy acid dehydratase (DHAD), 2) the coding region for horse liver ADH (nt 4680-5807) expressed from the yeast GPM1 promoter (nt 5819-6575) followed by the ADH1 terminator (nt 4356-4671) for expression of alcohol dehydrogenase, and 3) the coding region of the KivD gene from Lactococcus lactis (nt 7175-8821) expressed from the yeast TDH3 promoter (nt 8830-9493) followed by the TDH3 terminator (nt 6582-7161) for expression of ketoisovalerate decarboxylase.

### Isobutanol Production Host Strain

Strain NYLA84 with the genotype BY4700 pdc6::P_{GPM1}-sadB-ADH1t pdc1::P_{PDC1}-ilvD-FBA1t Δhis3 Δhxk2 pdc5::kanMX4, described in U.S. Provisional Patent Application No. 61/246,709, filed on Sep. 29, 2009, was used for isobutanol production. This strain was constructed by insertion-inactivation of endogenous PDC1, PDC5, and PDC6 genes of S. cerevisiae. PDC1, PDC5, and PDC6 genes encode the three major isozymes of pyruvate decarboxylase.

Construction of pdc6::P_{GPM1}-sadB Integration Cassette and PDC6 Deletion:

A pdc6::P_{GPM1}-sadB-ADH1t-URA3r integration cassette was made by joining the GPM-sadB-ADHt segment (SEQ ID NO:156) from pRS425::GPM-sadB (described above) to the URA3r gene from pUC19-URA3r. pUC19-URA3r (SEQ ID NO:212) contains the URA3 marker from pRS426 (ATCC #77107) flanked by 75 bp homologous repeat sequences to allow homologous recombination in vivo and removal of the URA3 marker. The two DNA segments were joined by SOE PCR (as described by Horton et al. (1989) Gene 77:61-68) using as template pRS425::GPM-sadB and pUC19-URA3r plasmid DNAs, with Phusion DNA polymerase (New England Biolabs Inc., Beverly, Mass.; catalog no. F-540S) and primers 114117-11A through 114117-11D (SEQ ID NOs:213, 214, 215 and 216), and 114117-13A and 114117-13B (SEQ ID NOs:217 and 218).

The outer primers for the SOE PCR (114117-13A and 114117-13B) contained 5′ and 3′ ˜50 bp regions homologous to regions upstream and downstream of the PDC6 promoter and terminator, respectively. The completed cassette PCR fragment was transformed into BY4700 (ATCC #200866) and transformants were maintained on synthetic complete media lacking uracil and supplemented with 2% glucose at 30° C. using standard genetic techniques (Methods in Yeast Genetics, 2005, Cold Spring Harbor Laboratory Press, Cold Spring Harbor, N.Y., pp. 201-202). Transformants were screened by PCR using primers 112590-34G and 112590-34H (SEQ ID NOs:219 and 220), and 112590-34F and 112590-49E (SEQ ID NOs:221 and 222) to verify integration at the PDC6 locus with deletion of the PDC6 coding region. The URA3r marker was recycled by plating on synthetic complete media supplemented with 2% glucose and 5-FOA at 30° C. following standard protocols. Marker removal was confirmed by patching colonies from the 5-FOA plates onto SD −URA media to verify the absence of growth. The resulting identified strain has the genotype: BY4700 pdc6::P_{GPM1}-sadB-ADH1t.

Construction of pdc1::P_{PDC1}-ilvD Integration Cassette and PDC1 Deletion:

A pdc1::P_{PDC1}-ilvD-FBA1t-URA3r integration cassette was made by joining the ilvD-FBA1t segment (SEQ ID NO:223) from pLH468 (described above) to the URA3r gene from pUC19-URA3r by SOE PCR (as described by Horton et al. (1989) Gene 77:61-68) using as template pLH468 and pUC19-URA3r plasmid DNAs, with Phusion DNA polymerase (New England Biolabs Inc., Beverly, Mass.; catalog no. F-540S) and primers 114117-27A through 114117-27D (SEQ ID NOs:224, 225, 226 and 227).

The outer primers for the SOE PCR (114117-27A and 114117-27D) contained 5′ and 3′ ˜50 bp regions homologous to regions downstream of the PDC1 promoter and downstream of the PDC1 coding sequence. The completed cassette PCR fragment was transformed into BY4700 pdc6::P_{GPM1}-sadB-ADH1t and transformants were maintained on synthetic complete media lacking uracil and supplemented with 2% glucose at 30° C. using standard genetic techniques (Methods in Yeast Genetics, 2005, Cold Spring Harbor Laboratory Press, Cold Spring Harbor, N.Y., pp. 201-202). Transformants were screened by PCR using primers 114117-36D and 135 (SEQ ID NOs 228 and 229), and primers 112590-49E and 112590-30F (SEQ ID NOs:222 and 230) to verify integration at the PDC1 locus with deletion of the PDC1 coding sequence. The URA3r marker was recycled by plating on synthetic complete media supplemented with 2% glucose and 5-FOA at 30° C. following standard protocols. Marker removal was confirmed by patching colonies from the 5-FOA plates onto SD −URA media to verify the absence of growth. The resulting identified strain “NYLA67” has the genotype: BY4700 pdc6::P_{GPM1}-sadB-ADH1t pdc1::_P_{PDC1}-ilvD-FBA1t.

### HIS3 Deletion

To delete the endogenous HIS3 coding region, a his3::URA3r2 cassette was PCR-amplified from URA3r2 template DNA (SEQ ID NO:231). URA3r2 contains the URA3 marker from pRS426 (ATCC #77107) flanked by 500 bp homologous repeat sequences to allow homologous recombination in vivo and removal of the URA3 marker. PCR was done using Phusion DNA polymerase and primers 114117-45A and 114117-45B (SEQ ID NOs:232 and 233) which generated a ˜2.3 kb PCR product. The HIS3 portion of each primer was derived from the 5′ region upstream of the HIS3 promoter and 3′ region downstream of the coding region such that integration of the URA3r2 marker results in replacement of the HIS3 coding region. The PCR product was transformed into NYLA67 using standard genetic techniques (Methods in Yeast Genetics, 2005, Cold Spring Harbor Laboratory Press, Cold Spring Harbor, N.Y., pp. 201-202) and transformants were selected on synthetic complete media lacking uracil and supplemented with 2% glucose at 30° C. Transformants were screened to verify correct integration by replica plating of transformants onto synthetic complete media lacking histidine and supplemented with 2% glucose at 30° C. The URA3r marker was recycled by plating on synthetic complete media supplemented with 2% glucose and 5-FOA at 30° C. following standard protocols. Marker removal was confirmed by patching colonies from the 5-FOA plates onto SD −URA media to verify the absence of growth. The resulting identified strain, called NYLA73, has the genotype: BY4700 pdc6::P_{GPM1}-sadB-ADH1t pdc1::P_{PDC1}-ilvD-FBA1t Δhis3.

Construction of pdc5::kanMX Integration Cassette and PDC5 Deletion:

A pdc5::kanMX4 cassette was PCR-amplified from strain YLR134W chromosomal DNA (ATCC No. 4034091) using Phusion DNA polymerase and primers PDC5::KanMXF and PDC5::KanMXR (SEQ ID NOs:234 and 235) which generated a ˜2.2 kb PCR product. The PDC5 portion of each primer was derived from the 5′ region upstream of the PDC5 promoter and 3′ region downstream of the coding region such that integration of the kanMX4 marker results in replacement of the PDC5 coding region. The PCR product was transformed into NYLA73 using standard genetic techniques (Methods in Yeast Genetics, 2005, Cold Spring Harbor Laboratory Press, Cold Spring Harbor, N.Y., pp. 201-202) and transformants were selected on YP media supplemented with 1% ethanol and geneticin (200 μg/ml) at 30° C. Transformants were screened by PCR to verify correct integration at the PDC locus with replacement of the PDC5 coding region using primers PDC5kofor and N175 (SEQ ID NOs:236 and 237). The identified correct transformants have the genotype: BY4700 pdc6::P_{GPM1}-sadB-ADH1t pdc1::P_{PDC1}-ilvD-FBA1t Δhis3 pdc5::kanMX4. The strain was named NYLA74.

### Deletion of HXK2 (Hexokinase II):

A hxk2::URA3r cassette was PCR-amplified from URA3r2 template (described above) using Phusion DNA polymerase and primers 384 and 385 (SEQ ID NOs:238 and 239) which generated a ˜2.3 kb PCR product. The HXK2 portion of each primer was derived from the 5′ region upstream of the HXK2 promoter and 3′ region downstream of the coding region such that integration of the URA3r2 marker results in replacement of the HXK2 coding region. The PCR product was transformed into NYLA73 using standard genetic techniques (Methods in Yeast Genetics, 2005, Cold Spring Harbor Laboratory Press, Cold Spring Harbor, N.Y., pp. 201-202) and transformants were selected on synthetic complete media lacking uracil and supplemented with 2% glucose at 30° C. Transformants were screened by PCR to verify correct integration at the HXK2 locus with replacement of the HXK2 coding region using primers N869 and N871 (SEQ ID NOs:240 and 241). The URA3r2 marker was recycled by plating on synthetic complete media supplemented with 2% glucose and 5-FOA at 30° C. following standard protocols. Marker removal was confirmed by patching colonies from the 5-FOA plates onto SD −URA media to verify the absence of growth, and by PCR to verify correct marker removal using primers N946 and N947 (SEQ ID NOs:242 and 243). The resulting identified strain named NYLA83 has the genotype: BY4700 pdc6::P_{GPM1}-sadB-ADH1t pdc1::P_{PDC1}-ilvD-FBA1t Δhis3 Δhxk2.

Construction of pdc5::kanMX Integration Cassette and PDC5 Deletion

A pdc5::kanMX4 cassette was PCR-amplified as described above. The PCR fragment was transformed into NYLA83, and transformants were selected and screened as described above. The identified correct transformants named NYLA84 have the genotype: BY4700 pdc6::P_{GPM1}-sadB-ADH1t pdc1::P_{PDC1}-ilvD-FBA1t Δhis3 Δhxk2 pdc5::kanMX4.

### Isobutanol Production

Plasmids pYZ067 along with either pYZ090, pYZ091 or pYZ058 were transformed into yeast strain NYLA84 using LiAc/PEG method and transformants were selected on yeast agar culture plates that contained yeast drop-out medium (without histidine and uracil) supplemented with 2% glucose and 0.1% ethanol (SEG). After 5-6 days at 30° C., individual colonies were patched on similar agar plates with 0.1% ethanol and 2% glucose (SEG plate) as carbon sources and cultured at 30° C. for 2-3 days before the following shake flask test.

The patches from individual colonies were inoculated first into 3 ml of SEG (2% glucose, 0.1% EtOH) medium and grown overnight (20 hr) at 30 C in a rotary drum at 150 rpm. The overnight cultures were inoculated into 20 ml SEG medium to an OD600 of about 0.3 in 125 ml flasks with tightly secured caps. The cultures were grown at 30° C. with shaking at 150 rpm. Samples were taken at various time points for analysis by HPLC as in General Methods.

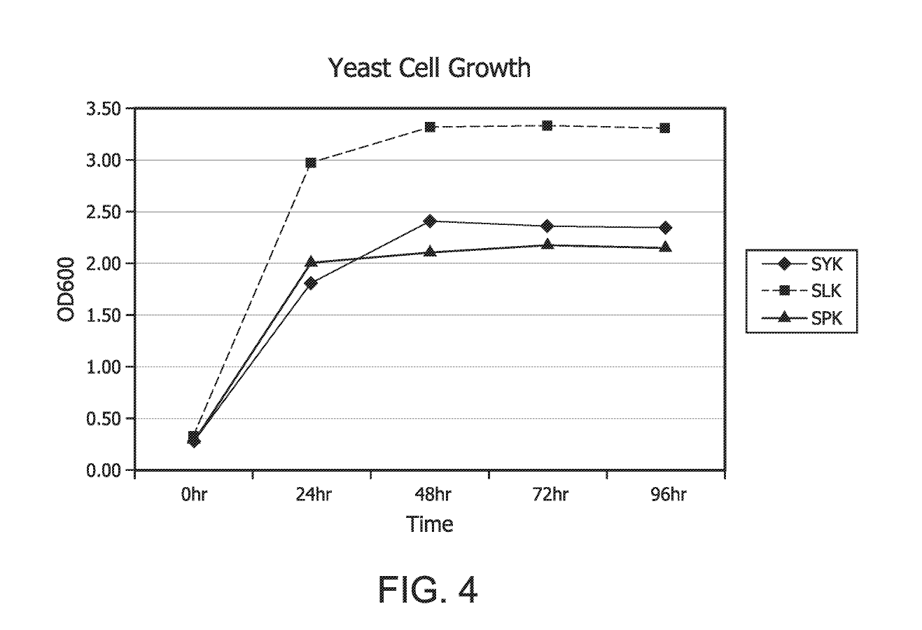

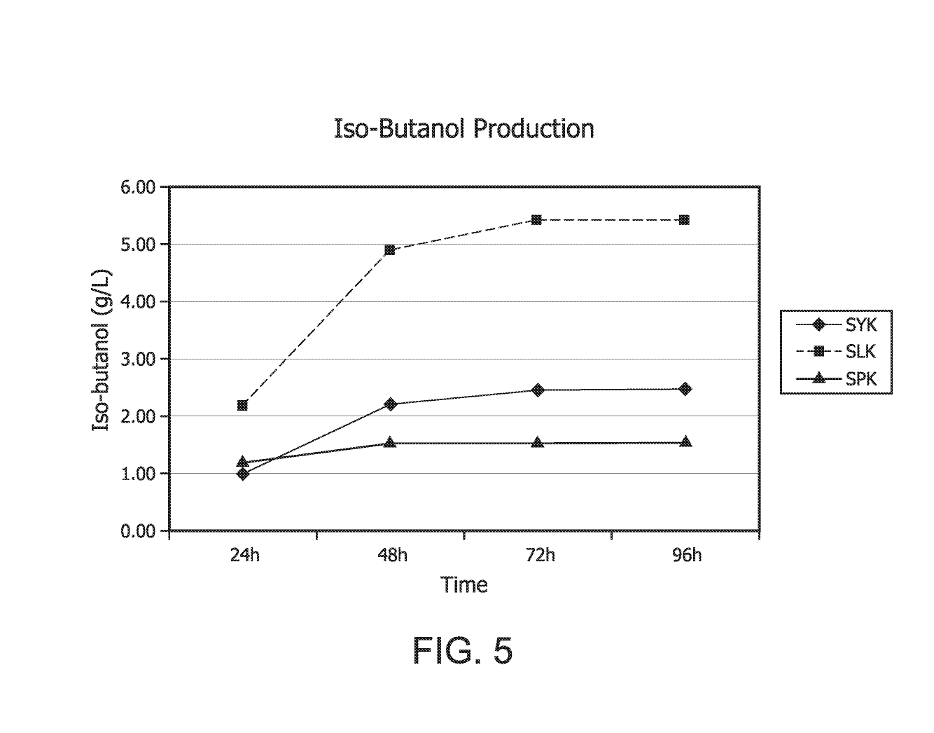

The results as shown in FIGS. 4 and 5 indicate that the yeast strain containing the IlvC coding region from L. lactis grew faster and produced more isobutanol than yeast strains containing ilvC from Pseudomonas fluorescens and ILV5 from Saccharomyces cerevisiae.

1-18. (canceled)

19. A recombinant yeast cell comprising at least one nucleic acid molecule encoding a polypeptide having ketol-acid reductoisomerase (KARI) activity wherein the polypeptide having KARI activity has an amino acid sequence that is at least 90% identical to the amino acid sequence of SEQ ID NO: 28, 36, 38, 40, 56, 60, or 68 and wherein the yeast cell comprises:
(i) at least one inactivated pyruvate decarboxylase (PDC) gene,
(ii) a modification to reduce glycerol-3-phosphate dehydrogenase activity, and
(iii) a heterologous polynucleotide encoding a polypeptide with phosphoketolase activity.

20. The recombinant yeast cell of claim 19, wherein the inactivated pyruvate decarboxylase (PDC) gene is PDC1, PDC5, or PDC6.

21. The recombinant yeast cell of claim 19, wherein the recombinant yeast cell further comprises at least one deletion, mutation, or substitution in an endogenous gene encoding a polypeptide affecting Fe—S cluster biosynthesis.

22. The recombinant yeast cell of claim 19, wherein the yeast cell expresses an engineered isobutanol biosynthetic pathway.

23. The recombinant yeast cell of claim 22, wherein the engineered isobutanol biosynthetic pathway comprises the following substrate to product conversions:
(i) pyruvate to acetolactate catalyzed by acetolactate synthase,
(ii) 2,3-dihydroxyisovalerate to a-ketoisovalerate catalyzed by dihydroxy-acid dehydratase, and
(iii) α-ketoisovalerate to isobutyraldehyde catalyzed by branched-chain a-keto acid decarboxylase.

24. The recombinant yeast cell of claim 23, wherein the acetolactate synthase has an EC number 1.1.1.86.

25. The recombinant yeast cell of claim 23, wherein the dihydroxy-acid dehydratase has an EC number 4.2.1.9.

26. The recombinant yeast cell of claim 23, wherein the branched-chain a-keto acid decarboxylase has an EC number 4.1.1.72.

27. The recombinant yeast cell of claim 23, wherein the acetolactate synthase has an amino acid sequence selected from the group consisting of SEQ ID NO: 70, 73, 75, 77, 79, 81, 83, 85, and 87.

28. The recombinant yeast cell of claim 23, wherein the dihydroxy-acid dehydratase has an amino acid sequence selected from the group consisting of SEQ ID NO: 95, 97, and 99.

29. The recombinant yeast cell of claim 23, wherein the branched-chain a-keto acid decarboxylase has an amino acid sequence selected from the group consisting of SEQ ID NO: 101 and 102.

30. The recombinant yeast cell of claim 19, the yeast cell is selected from the group consisting of Saccharomyces, Schizosaccharomyces, Hansenula, Candida, Kluyveromyces, Yarrowia, Issatchenkia, and Pichia.

31. A method for the production of isobutanol comprising:
a) providing a recombinant yeast cell comprising an isobutanol biosynthetic pathway comprising at least one nucleic acid molecule encoding a polypeptide having ketol-acid reductoisomerase activity, wherein the polypeptide having KARI activity has an amino acid sequence that is at least 90% identical to the amino acid sequence of SEQ ID NO: 28, 36, 38, 40, 56, 60, or 68;
b) growing the recombinant yeast cell of step (a) under conditions wherein isobutanol is produced.

32. The method of claim 31, wherein the isobutanol is recovered.

33. The method of claim 32, wherein the isobutanol is recovered by distillation, azeotropic distillation, liquid-liquid extraction, adsorption, gas stripping, membrane evaporation, or pervaporation.


SEQUENCE LISTING (245 sequences)

SEQ ID NO: 1 - DNA, 1002 bp, Staphylococcus capitis
atgacaaaagtttattatgatcaatcagttgaaaaagatgctttacaaggtaaaaaaattgcagtaattggttacggttcacaaggacatgcacacgctcaaaatttaaaagacaatggttatgatgtagtcgttggtattcgacctggtcattctttcgatagagctaaagaagatggatttgatgtttatccagtgaatgaggctactaaacaagcagatgtagtaatgattttattacctgatgaaatccaaggtaatgtatataaaaatgaaattgaacctaacttagaagcaggcaatgcattagcttttgcgcacggatttaacattcactttggagttattcaaccccctgctgatgtagatgtattcttagtagcacctaaaggtcctggtcatttagtaagacgtacctttgtagaaggtactgctgtaccagcattattcggtgtacaacaagatgattcaggtcatgcacgtgatatcgcactaagctatgccaaaggtattggtgcaacacgtgctggtgttattgaaactacattcaaagaagaaactgttactgatttattcggtgaacaagctgtactttgcgggggtatccataaattaattcaaagtggttttgaaactttagtagaagctgggtatcaaaaagagttagcatacttcgaagtattacatgaaatgaaattaatcgttgatttaatgtacgaaggcggtatggaaaacgttcgctactctatttcaaatacagctgaatttggtgattatgtatctggaccacgtgttatcactccagaagttaaaaacaacatgaaaactgtattagaagatattcaaaacggtaactttgctaatcgttttgttaaagataatgaaaatggctttaaagaattttaccaattacgtgaacaacaacatggtcatgaaatcgaagcggttggtcgtgaattaagaaaaatgatgccatttattaaagctaaaagtattcaaaaa

SEQ ID NO: 2 - protein, 334 aa, Staphylococcus capitis
MTKVYYDQSVEKDALQGKKIAVIGYGSQGHAHAQNLKDNGYDVVVGIRPGHSFDRAKEDGFDVYPVNEATKQADVVMILLPDEIQGNVYKNEIEPNLEAGNALAFAHGFNIHFGVIQPPADVDVFLVAPKGPGHLVRRTFVEGTAVPALFGVQQDDSGHARDIALSYAKGIGATRAGVIETTFKEETVTDLFGEQAVLCGGIHKLIQSGFETLVEAGYQKELAYFEVLHEMKLIVDLMYEGGMENVRYSISNTAEFGDYVSGPRVITPEVKNNMKTVLEDIQNGNFANRFVKDNENGFKEFYQLREQQHGHEIEAVGRELRKMMPFIKAKSIQK

SEQ ID NO: 3 - DNA, 1002 bp, Staphylococcus epidermidis
atgacaaaagtttattatgatggatctgtaactaaagacgcattacaaggtaaaaaaattgcagtaatcggatatggctcacaaggacatgcccacgcgcaaaacttaaaagatagtggctatgatgtcatcatcggtatccgtcctggacactcatacgataaagctaaaaaagatggtttcgatgtgtatccagtaagtgaagctacaaaacaagcagatgtcgttatggttttattaccagatgaaatccaaggtaacgtatacaaaaatgaaatcgcacctaacttagaagctggtaatgccctagctttcgcccacggttttaacattcactttggtgttattcaacctcctgctgatgtagatgtatttttagtagctcctaaaggaccaggtcaccttgtaagacgtacattcgttgaaggcagtgcagtaccttcattatttggtgttcaacaagatgctactggaaatgcacgtgatatcgctttaagctatgccaaaggtattggtgctacacgtgctggtgttattgaaacaacatttaaagaagaaactgaaactgatttattcggtgagcaagctgtactttgtggtggtatccataaattaattcaaagtggttttgaaactttagtagaagctggttatcaaaaagaattagcttacttcgaagtattacatgaaatgaaattaatcgttgatttaatgtatgaaggcggtatggaaaacgttcgctattctatttcaaatactgccgaatttggtgattatgtatctggaccacgtgtcatcactccagaagttaaaaacaacatgaaaactgtattagaagatatccaaaacggtaactttgctaatcgttttgttaaagataatgaaaatggctttaaagaattctatcaattacgtgaacaacaacatggtcatgaaattgaagcggtaggtcgtgagcttcgtgaaatgatgccatttattaaatctaaaagcattgaaaaa

SEQ ID NO: 4 - protein, 334 aa, Staphylococcus epidermidis
MTKVYYDGSVTKDALQGKKIAVIGYGSQGHAHAQNLKDSGYDVIIGIRPGHSYDKAKKDGFDVYPVSEATKQADVVMVLLPDEIQGNVYKNEIAPNLEAGNALAFAHGFNIHFGVIQPPADVDVFLVAPKGPGHLVRRTFVEGSAVPSLFGVQQDATGNARDIALSYAKGIGATRAGVIETTFKEETETDLFGEQAVLCGGIHKLIQSGFETLVEAGYQKELAYFEVLHEMKLIVDLMYEGGMENVRYSISNTAEFGDYVSGPRVITPEVKNNMKTVLEDIQNGNFANRFVKDNENGFKEFYQLREQQHGHEIEAVGRELREMMPFIKSKSIEK

SEQ ID NO: 5 - DNA, 1002 bp, Staphylococcus aureus
atgacaacagtttattatgatcaagacgtaaaaacggacgctttacaaggcaaaaaaattgcagtagtaggttatggatcacaaggccacgcgcatgcacaaaacttaaaagacaatggatatgatgtagtcatcggtattcgcccaggtcgttcttttgacaaggctaaagaagatggatttgatgtgttccctgttgcagaagcagttaagcaagctgatgtaattatggtgctattacctgatgaaattcaaggtgatgtatacaaaaacgaaattgaaccaaatttagaaaaacataatgtgcttgcatttgctcatggctttaacattcattttggtgttattcaaccaccagctgatgttgatgtatttttagtagctcctaaaggaccgggtcatttagttagacgtacatttgttgaaggttctgctgtaccatcactatttggtattcaacaagacgcttcaggtcaagcacgtaatattgctttaagttatgcaaaaggtattggtgcaactcgtgcaggtgttattgaaacaacatttaaagaagaaactgagacagatttatttggtgaacaagcagtactttgcggtggtgtatcgaaattaattcaaagtggctttgaaacattagtagaagcgggttatcaaccagaattagcttattttgaagtattacatgaaatgaaattaatcgttgatttgatgtatgaaggcggtatggaaaatgtacgttactcaatttcaaatactgctgaatttggtgactatgtttcaggaccacgtgttatcacaccagatgttaaagaaaatatgaaagctgtattaactgatatccaaaatggtaacttcagtaatcgctttatcgaagacaataaaaatggattcaaagaattttataaattacgcgaagaacaacatggtcatcaaattgaaaaagttggtcgtgaattacgcgaaatgatgccttttattaaatctaaaagcattgaaaaa

SEQ ID NO: 6 - protein, 334 aa, Staphylococcus aureus
MTTVYYDQDVKTDALQGKKIAVVGYGSQGHAHAQNLKDNGYDVVIGIRPGRSFDKAKEDGFDVFPVAEAVKQADVIMVLLPDEIQGDVYKNEIEPNLEKHNVLAFAHGFNIHFGVIQPPADVDVFLVAPKGPGHLVRRTFVEGSAVPSLFGIQQDASGQARNIALSYAKGIGATRAGVIETTFKEETETDLFGEQAVLCGGVSKLIQSGFETLVEAGYQPELAYFEVLHEMKLIVDLMYEGGMENVRYSISNTAEFGDYVSGPRVITPDVKENMKAVLTDIQNGNFSNRFIEDNKNGFKEFYKLREEQHGHQIEKVGRELREMMPFIKSKSIEK

SEQ ID NO: 7 - DNA, 1002 bp, Staphylococcus warneri
atgacaacagtttattatgatgaatcagtaaaaacagacgctttagaaggtaaaaaaattgctgtgattggatatggttcacaaggccatgctcacgcacaaaacttaaaagataacggatatgatgttgttatcggtatccgtccaggacgctcttttgataaagctaaggaaaatggtttcgatgtatatcctgtggcagaagcaacgaaacaagctgacgttatcatggttttaattccagatgaaatccaaggtgatgtatataaaaatgaaattgaacctaatttagaagctggtaacgcacttgcatttgctcatggtttcaatattcactttggtgttattcaaccccctgcagatgtcgatgttttcttagttgcaccaaaaggaccaggtcacttagttagacgtacatttgaagaaggtagtgcagtgccatctttatttggtgttcaacaagatgctacaggtcatgcaagagatattgcattaagttatgcaaaaggtatcggatctacaagagctggtgtaattgaaacaacatttaaagaagaaacagaaacggatttatttggtgaacaagctgtactatgtggcggtatccataaattaattcaaagtggatttgaaacattagttgaagctggttaccaaccagaattagcctattttgaagtattacatgaaatgaaattaatcgttgatttaatgtatgaaggcggtatggataatgtccgttattcaatttctaatactgcagagtatggtgactatgtatctggacgtcgtatcatcactccagatgtcaaagacaatatgaaagctgtattaacagatattcaaaacggtaaatttgctaatagttttgttgaagataataaaaatggatttaaagaattctataaaatgcgtgaagaacaagctggtcatccaattgaaaaagtgggccgtaaattacgtgatatgatgccatttattaaatcaaaaagcattgaaaag

SEQ ID NO: 8 - protein, 334 aa, Staphylococcus warneri
MTTVYYDESVKTDALEGKKIAVIGYGSQGHAHAQNLKDNGYDVVIGIRPGRSFDKAKENGFDVYPVAEATKQADVIMVLIPDEIQGDVYKNEIEPNLEAGNALAFAHGFNIHFGVIQPPADVDVFLVAPKGPGHLVRRTFEEGSAVPSLFGVQQDATGHARDIALSYAKGIGSTRAGVIETTFKEETETDLFGEQAVLCGGIHKLIQSGFETLVEAGYQPELAYFEVLHEMKLIVDLMYEGGMDNVRYSISNTAEYGDYVSGRRIITPDVKDNMKAVLTDIQNGKFANSFVEDNKNGFKEFYKMREEQAGHPIEKVGRKLRDMMPFIKSKSIEK

SEQ ID NO: 9 - DNA, 1002 bp, Staphylococcus epidermidis
atgacaaaagtatattacgatgaaacagtaactcaagatgcattacaaggtaaaaaaattgctgtcattggttatggctcacaaggacatgcacatgcacaaaatttaaaggacaatggttatgatgtagtcattggtctacgtccaggacgatcatttaataaagctaaagaagatggatttgatgtttatacggtaagtgatgctacacaacaagctgatgtagtgatggtactattgcctgatgaaattcaaggtaaagtatataacgaggaaattaaaccacatttagaaaaaggaaatgctttagcattcgcacacggttttaatatccacttcagtgttatcgaaccacctagtgatgtcgatgtctttttagtagcacctaaaggaccaggtcatttagttagacgtacatttgttgaaggaagtgccgtaccagcattatttggtgttcaacaagatgctacaggccaagcaagaaacattgctttaagctacgcaaaaggcattggtgctactcgtgccggggtcattgaaacgacatttaaagaagaaactgaaacagatttattcggtgaacaagctgttctttgtggaggagtttccaaattaattcagagtggtttcgaaacacttgtagaagcaggttaccaacctgaattagcttattttgaagtcttacacgaaatgaaattaattgttgatttaatgtatgaaggcggaatggaaaacgttcgttattctatctctaacactgctgaatttggcgactatgtttctggaccaagagtaattacacctgatgttaaagaaaatatgaaaaaagtacttgaagatattcaaaatggtaactttagccgtagatttgttgaagataacaaaaatggctttaaagaattctatcaattacgtgaagatcaacatggtcatcaaattgaacaagttggacgtgaattaagagaaatgatgccattcattaaatctaaaagtattgaaaaa

SEQ ID NO: 10 - protein, 334 aa, Staphylococcus epidermidis
MTKVYYDETVTQDALQGKKIAVIGYGSQGHAHAQNLKDNGYDVVIGLRPGRSFNKAKEDGFDVYTVSDATQQADVVMVLLPDEIQGKVYNEEIKPHLEKGNALAFAHGFNIHFSVIEPPSDVDVFLVAPKGPGHLVRRTFVEGSAVPALFGVQQDATGQARNIALSYAKGIGATRAGVIETTFKEETETDLFGEQAVLCGGVSKLIQSGFETLVEAGYQPELAYFEVLHEMKLIVDLMYEGGMENVRYSISNTAEFGDYVSGPRVITPDVKENMKKVLEDIQNGNFSRRFVEDNKNGFKEFYQLREDQHGHQIEQVGRELREMMPFIKSKSIEK

SEQ ID NO: 11 - DNA, 1002 bp, Staphylococcus saprophyticus
atgacaacagtttattatgaccaatcagttacgaaagacgctttacaaggtaaaaaagtagcaatcataggctatggttctcaaggacacgcacatgctcaaaatttaaaagacaacggttatgatgtcattatcggaatcagacctggatgttcttttgacaaagctaaagatgatggcttcgaagtatatcctgtagatgaagcagcaaaacaagctgatgtcatcatggtgttattaccagatgaaattcaaggccaagtatataaagaagaaattgaaccaaacttagaagcaaacaatgcattagtattcgcgcatggttttaatattcatttcggtgttattcaaccaccagaaaacgtagatgtattcttagtagcgcctaaaggacctggacatttagtacgtcgtacatttgctgaaggaagcgcagtccctgccctattcgcagttgaacaagatccgagtggtgaagctagagatttagcattaagctatgctaaaggtatcggtgcaacacgtgcaggtgtattagaaacatcatttaaagaagaaacagaaacagatttattcggtgaacaagcagtgctttgtggtggtacgactaaattagtacaatctggtttcgaaacgttagtagaagcaggttaccaaccagaaattgcatactttgaagtattgcatgaaatgaaattgattgttgatttaatgtatgaaggcggtatggaaaatatgcgctattcaatttcaaatacagctgaatttggtgactatgtttctggaccacgtattatcacaccggatgttaaagataatatgaaagctgtattagatgatattcaaaaaggaaacttcagtgatcgattcattaaagataatcaaaataattttgaagaattccataaattaagagaagaacaacatggtcatcaaatcgaagcggttggtagagaactccgtgatatgatgccattcatcaaatctaagagcattgaaaaa

SEQ ID NO: 12 - protein, 334 aa, Staphylococcus saprophyticus
MTTVYYDQSVTKDALQGKKVAIIGYGSQGHAHAQNLKDNGYDVIIGIRPGCSFDKAKDDGFEVYPVDEAAKQADVIMVLLPDEIQGQVYKEEIEPNLEANNALVFAHGFNIHFGVIQPPENVDVFLVAPKGPGHLVRRTFAEGSAVPALFAVEQDPSGEARDLALSYAKGIGATRAGVLETSFKEETETDLFGEQAVLCGGTTKLVQSGFETLVEAGYQPEIAYFEVLHEMKLIVDLMYEGGMENMRYSISNTAEFGDYVSGPRIITPDVKDNMKAVLDDIQKGNFSDRFIKDNQNNFEEFHKLREEQHGHQIEAVGRELRDMMPFIKSKSIEK

SEQ ID NO: 13 - DNA, 1002 bp, Staphylococcus carnosus
atgacaacagtttattatgacgaaacagtaaaagaagatgctttaaaaggtaaaaaaattgcagtagttggttatggttctcaaggccatgctcatgcacaaaacttaaaagacaatggttatgacgttgtaatcggtatccgcccaggtaattctttcgataaagctaaaaaagacggctttgatgtatatccagttgatgaagcagtgaaacaagctgatgtagtaatggtattattacctgatgaaatccaaggtaaagtatatgaagaagaaatcgcaccaaacttagaagctggcaacgctttagcttttgcacatggcttcaacatccactttgatgttatccaaccacctgaagatgttgatgtattcttagttgcccctaaaggaccaggacatttagtaagacgtacatttactgaaggaagctcagtacctgccctattcggtgtagaacaagatgcttctggcaatgctttcaacttagctttaagttatgcaaaaggtatcggagcaacaaaagctggggttatcgaaactacatttaaagaagaaactgaaactgatttatttggtgaacaagctgtactttgcggcggtgtaacaagattaatccaaagtggatttgaaacattagttgaagctggttaccaaccagaattagcttattttgaagtattacatgaaatgaaattaatcgttgatttaatgtatgaaggcggaatggaaacaatgcgttattctatctcaaatacagcagaatacggcgattatgtttctggtccacgtgtcatcactgatgatgttaaacaaaacatgaaagctgtcttagaagatatccaaaacggtaaattcgcaaacagtttcattgaagataataaaaatggtttcaaagagttctacaaattgcgtgaagaagcacaagatcatcaaattcaagaagttggtaaagaacttcgtgaaatgatgccttttgttgaaaataaaactattgaaaaa

SEQ ID NO: 14 - protein, 334 aa, Staphylococcus carnosus
MTTVYYDETVKEDALKGKKIAVVGYGSQGHAHAQNLKDNGYDVVIGIRPGNSFDKAKKDGFDVYPVDEAVKQADVVMVLLPDEIQGKVYEEEIAPNLEAGNALAFAHGFNIHFDVIQPPEDVDVFLVAPKGPGHLVRRTFTEGSSVPALFGVEQDASGNAFNLALSYAKGIGATKAGVIETTFKEETETDLFGEQAVLCGGVTRLIQSGFETLVEAGYQPELAYFEVLHEMKLIVDLMYEGGMETMRYSISNTAEYGDYVSGPRVITDDVKQNMKAVLEDIQNGKFANSFIEDNKNGFKEFYKLREEAQDHQIQEVGKELREMMPFVENKTIEK

SEQ ID NO: 15 - DNA, 993 bp, Listeria monocytogenes
atgacaaaagtttattatgaagatgcagtaaaaaacaacgcactagaaggtaaaacagtagcagtaatcgggtacggttcgcaaggtcacgcacattctcaaaatctacgtgacaatggcaataacgttattatcggcattcgcgaaggaaaatctgccgaatctgctagaaacgatggctttgatgtttattctgttagcgaagccgctgataaagctgatgtcatcatgattcttttgccagatgaaacacaaggcgaaacatacgaaaatgaaattaaacctaacctaaaagctggtaattcacttgttttcgctcacggttttaacattcattttgacgtaattaatcctccaagcgatgtagatgtttttctagtagctccaaaaggtcctggtcacttagttcgccgcacatttgttgaaggtggcgctgttccttccctattcgctatctatcaagatgccactggaaacgcacgcgacacagccctttcctatgcaaaaggtattggcgcaactcgtgctggcgttatcgaaaccactttcaaagaagaaaccgaaaccgatctatttggcgaacaagcagttctttgtgggggtgcgactcaccttatccaagctggtttcgaaacacttgtagaggctggctaccaaccagaacttgcttattttgaagtattacatgaaatgaaactaattgttgatttgatgtatgaaggtggtatggaaaaaatgcgccactcgatctccaatacagcagaatatggtgattatgtttccggtcctcgagttgttacagccgatacgaaaaaagcaatgaaagaagtacttaccgacattcaaaatggtaactttgctaaatctttcatcaatgacaacaaaaatggttttaaagaattccatagaatgcgtaaagaacaacaaggtcatcaaatcgaaaaagttggtgcagaacttcgcgaaatgatgccatttgtcaaaccacaacat

SEQ ID NO: 16 - protein, 331 aa, Listeria monocytogenes
MTKVYYEDAVKNNALEGKTVAVIGYGSQGHAHSQNLRDNGNNVIIGIREGKSAESARNDGFDVYSVSEAADKADVIMILLPDETQGETYENEIKPNLKAGNSLVFAHGFNIHFDVINPPSDVDVFLVAPKGPGHLVRRTFVEGGAVPSLFAIYQDATGNARDTALSYAKGIGATRAGVIETTFKEETETDLFGEQAVLCGGATHLIQAGFETLVEAGYQPELAYFEVLHEMKLIVDLMYEGGMEKMRHSISNTAEYGDYVSGPRVVTADTKKAMKEVLTDIQNGNFAKSFINDNKNGFKEFHRMRKEQQGHQIEKVGAELREMMPFVKPQH

SEQ ID NO: 17 - DNA, 993 bp, Listeria grayi
atgacaaaagtatattatgaagatgctgtaaaagaaaatgcactaacggggaagaaagttgccgttatcggttatggatcacaaggacatgcgcattcgcaaaatctgcgtgacaatggcaatgacgttatcatcggtatccgcgaaggacgttctaaagaaagtgcgaaaaacgacggcttcgatgtttactccgttgccgaagcgagtaaacaagcagatgtcatcatgatccttctaccagacgaaatccaaggagatacatacgaaaaagaaatcagcccaaacttggaagcaggaaatgccattgttttcgcccatggtttcaacatccattttgatgtgatcaaacctcctacagatgtagatgttttcctagttgcaccaaaaggccccggacatctagtaagacgtacctttgtagaaggcggtgccgtcccttccctcttcgccgtccaccaagatgccactggcaaagcgcaggacctcgccctttcttatgccaaaggaatcggagctacacgtgccggcgttatcgaaacaactttcaaagaagaaacagaaacggatctattcggtgaacaagctgtcctatgcggcggtgccactcaccttatccaagcaggctttgaaacacttgtagaagcaggctatcaaccggaactcgcttacttcgaagtactgcatgagatgaaactgatcgtagacctgatgtacgaaggcggaatggaaaaaatgcgctattcgatctccaatacagcagaatatggcgactatgtatccggtccgcgcatcgtgaccagcgatacgaagaaagcgatgaaagaagtattgaccgatatccaaaatggtaattttgccaaatcgtttatcgatgacaataaaaacggcttcaaagaatttcaccgaatgcgcaaagaacagcaaggtcatcaaatcgaagcagtcggcaaaaatctccgcgaaatgatgccgttcgtaaatccaagaaaa

SEQ ID NO: 18 - protein, 331 aa, Listeria grayi
MTKVYYEDAVKENALTGKKVAVIGYGSQGHAHSQNLRDNGNDVIIGIREGRSKESAKNDGFDVYSVAEASKQADVIMILLPDEIQGDTYEKEISPNLEAGNAIVFAHGFNIHFDVIKPPTDVDVFLVAPKGPGHLVRRTFVEGGAVPSLFAVHQDATGKAQDLALSYAKGIGATRAGVIETTFKEETETDLFGEQAVLCGGATHLIQAGFETLVEAGYQPELAYFEVLHEMKLIVDLMYEGGMEKMRYSISNTAEYGDYVSGPRIVTSDTKKAMKEVLTDIQNGNFAKSFIDDNKNGFKEFHRMRKEQQGHQIEAVGKNLREMMPFVNPRK

SEQ ID NO: 19 - DNA, 987 bp, Enterococcus casseliflavus
atggtaaaagtatattacaacgaggaagttacagataatttaggagacaagaagattgcgatcgtcggctacggctctcaagggcacgctcatgcgcaaaatttacgggacacaggtcatgaagtctttatcggcattcgtgaaggaaaatcagcggaagccgcaagaaacgatggatttgaggttttcccagtagctgaagccgttcaaaaagcggatgtagtcatgatcttggcacccgatgagatccaaggatcgttgtataaaaatgagatcgaaccgaatctttctgcgggcaatgcattggcatttgctcatggtttcaatattcattttgatgtgatcgcgccaccaaaagaggtggatgtttttctagttgcgccaaaaggaccaggtcatttggtgcgtcgaacctttaccgaagggtttgcggtaccagccttgtttgcagtttatcaagatgcgactggtaacgcacaagcaacggctttatcctatgcaaaaggcattggtgccacacgggtcggcgttttggaaacgacctttaaagaagagaccgaaaccgatcttttcggtgagcaagccgtactttgtggcggtttgaccagtatgatcgaagcgggctttgaaacattggtagaagcggggtaccaaccggaattggcttattttgaagtttgtcatgaattgaaactgatcgttgatttgatttatgaaggaggcttcaaaaaaatgcgccattcgatctcgaatacggctgagtatggtgactacgtttctggtgctcgggtgatcactgcacaagccaaagaaaacatgaaagccgtattgacagacatccaaaacggccaatttgcgaaaggattcattgaagacaatcaaaacggcttccctgaattccataaaatgcgtgaggaaaacggcaatcatcaaattgaaaaagtcggcagcgagttacggaaaatgatgccgtttgttgcgaaagat

SEQ ID NO: 20 - protein, 329 aa, Enterococcus casseliflavus
MVKVYYNEEVTDNLGDKKIAIVGYGSQGHAHAQNLRDTGHEVFIGIREGKSAEAARNDGFEVFPVAEAVQKADVVMILAPDEIQGSLYKNEIEPNLSAGNALAFAHGFNIHFDVIAPPKEVDVFLVAPKGPGHLVRRTFTEGFAVPALFAVYQDATGNAQATALSYAKGIGATRVGVLETTFKEETETDLFGEQAVLCGGLTSMIEAGFETLVEAGYQPELAYFEVCHELKLIVDLIYEGGFKKMRHSISNTAEYGDYVSGARVITAQAKENMKAVLTDIQNGQFAKGFIEDNQNGFPEFHKMREENGNHQIEKVGSELRKMMPFVAKD

SEQ ID NO: 21 - DNA, 984 bp, Enterococcus gallinarum
atggtaaaagtatattataacgaagtgacagacaatttaggagataagactattgcaatcattggttacggttctcaaggacatgcccatgcacagaatttacgggatacaggacacaacgtcgtgatcgggattcgtgcaggaaaatcagctgagaacgcaaagaatgatggatttaccgtgcttccagtttctgaagctgtacaacaagcaaatgttgtgatgatcttagctcctgatgagatccaaggagaactttacgagaaggaaattgcgcctaatctttctgcaggggatgcattagcctttgctcatggctttaatattcattttgaggtcatcacgcctccaaaagaggtggatgtcttcctggtggcacctaaaggaccaggccatttggtgcgccggacctttagcgaaggatttgctgtccctgcgttgtttgctgtttatcaagatgctactggcaatgcacaagagacagccttgtcctatgccaaaggaatcggggctacaagagtcggtgtactagaaacaacgttcaaggaagagacggaaacagacttgtttggggaacaagccgtattatgtggcggactgacaagtatgatcgagactgggtttgaaacgctggtagaagccggctatcagccagaactagcctattttgaagtttgtcacgaaatgaaactgattgttgacttgatttatgaaggaggattcgccaagatgcgtgattcaatctccaatacagcagaatatggggattatgtctctggttcacgaatcattaccgaacaaacaaaagccaatatgaagaatgtcttgaaagatattcaaaacggcacgtttgccaaaggttttattgcggataatcaagctggttttccggaattcaaaaaaatgcgggaacaaaacgggaatcatccaatcgaaaaagttgggtcagaattaagaaaaatgatgccttttgttacgaaagat

SEQ ID NO: 22 - protein, 328 aa, Enterococcus gallinarum
MVKVYYNEVTDNLGDKTIAIIGYGSQGHAHAQNLRDTGHNVVIGIRAGKSAENAKNDGFTVLPVSEAVQQANVVMILAPDEIQGELYEKEIAPNLSAGDALAFAHGFNIHFEVITPPKEVDVFLVAPKGPGHLVRRTFSEGFAVPALFAVYQDATGNAQETALSYAKGIGATRVGVLETTFKEETETDLFGEQAVLCGGLTSMIETGFETLVEAGYQPELAYFEVCHEMKLIVDLIYEGGFAKMRDSISNTAEYGDYVSGSRIITEQTKANMKNVLKDIQNGTFAKGFIADNQAGFPEFKKMREQNGNHPIEKVGSELRKMMPFVTKD

SEQ ID NO: 23 - DNA, 990 bp, Macrococcus caseolyticus
atggctaaagtatattatgataacgatgtaaataaggaatacttaaaaaataagaaaattgcagtattagggtacggctctcaaggacacgcgcatgcattgaatcttaaggacagtggctatgacgtagtcatcggagtacgtgaagggcagtccagaacaaaggcagaagaagatggatttcaagcatatgacgtcagtgaagctgtggaacgtgcggatgttaccgtcgtccttatgccggacgaagtacagcagcgtgtgttcaacgaggaaatcgtacctcatctcaaaccacaatcggctctagttttcgctcatggatttaatgtgcatttcggtgcaataaaagctcctgacgatgttgatgtcttcttagtcgcaccaaaaggaccaggacatcttgttcgacgcgaatatgttaagggcagtgctgtccctgcgttatttgcagtcgatcaggatgtaactggtgatgcaaaggcccttgcactgaactatgcacacggcatcggtgcaacgcgcgccggcgtcatcgaaacgaccttcaaggaagaaacagagacagacctttttggtgagcaagcggtactctgcggcggtatcacaaagctcatccactacggttttgaaacactgacggaagcaggttatcagccagaactcgcctatttcgaagtactgcatgaaatgaagctgattgtggaccttatgtacgagggcggtatggagaagatgagacattccatctcaaataccgctgagtttggagattatgtctctggtgcgcgcgttattacccccgaagttaaggaaaatatgaaacaggtattagctgatatccagtctggcacattcagcaggcagtttatcgaagacaataataatggatttgagtcttttaaatctatgagaagtacccaggcaggacatcctatagagaaagtcggtgcagacctaagaatgatgatgccgtttatcaataataac

SEQ ID NO: 24 - protein, 330 aa, Macrococcus caseolyticus
MAKVYYDNDVNKEYLKNKKIAVLGYGSQGHAHALNLKDSGYDVVIGVREGQSRTKAEEDGFQAYDVSEAVERADVTVVLMPDEVQQRVFNEEIVPHLKPQSALVFAHGFNVHFGAIKAPDDVDVFLVAPKGPGHLVRREYVKGSAVPALFAVDQDVTGDAKALALNYAHGIGATRAGVIETTFKEETETDLFGEQAVLCGGITKLIHYGFETLTEAGYQPELAYFEVLHEMKLIVDLMYEGGMEKMRHSISNTAEFGDYVSGARVITPEVKENMKQVLADIQSGTFSRQFIEDNNNGFESFKSMRSTQAGHPIEKVGADLRMMMPFINNN

SEQ ID NO: 25 - DNA, 1020 bp, Streptococcus vestibularis
atggcagttcaaatggaatatgaaaaagacgtaaaagtagcagcacttgacggtaaaaaaatcgccgttatcggttacggttcacaaggtcatgcacatgcacaaaacttgcgtgattcaggtcacgatgtgatcatcggtgttcgtccaggtaaatcatttgataaagctaaagaagatggttttgatacttacacagtagcagaagcaactaaattggctgatatcattatggttttggcaccagatgaaattcaaaaagatatctataaagatgaaattgagccaaacttgagtgcaggcaaagctcttggttttgcacacggatttaacatccacttcgaattcatcaaggttcctaaagatgttgatgtctttatggttgctcctaaaggaccaggtcacttggtacgccgtacatatacagaaggctttggtgttccagcactttacgcagtataccaagatgctacaggcaatgctaaagacatcgctatggactgggctaaaggtattggttctgcacgtgttggattacttgtaacaacatttaaagaagaaactgaagaagatttgtttggtgagcaagcggttcttatgggtgggttgacacatcttattgaagctggttttgaagtcttgactgaagcaggctatgcaccacaattggcttactttgaagtgcttcacgaaatgaaattgatcgttgacttgatctacgaaggtggattcaagaaaatgcgtcaatcatgttcaaatactgcagaatttggtgactttgtaactggtccacgtgtcatcggtccagaagtcaaagaaaacatgaaagctgcccttgcggatatccaatcaggtaaatttgcacgtgaattcgttgaagaccatgatgctggcttcccacgtttgaaagcttaccgtaaagaagctgaagagcttgaaattgaaaagattggtgctgaattgcgtaaagcaatgccattcgttggtcaaaatgacgatgacgcattcaaaatctataac

SEQ ID NO: 26 - protein, 340 aa, Streptococcus vestibularis
MAVQMEYEKDVKVAALDGKKIAVIGYGSQGHAHAQNLRDSGHDVIIGVRPGKSFDKAKEDGFDTYTVAEATKLADIIMVLAPDEIQKDIYKDEIEPNLSAGKALGFAHGFNIHFEFIKVPKDVDVFMVAPKGPGHLVRRTYTEGFGVPALYAVYQDATGNAKDIAMDWAKGIGSARVGLLVTTFKEETEEDLFGEQAVLMGGLTHLIEAGFEVLTEAGYAPQLAYFEVLHEMKLIVDLIYEGGFKKMRQSCSNTAEFGDFVTGPRVIGPEVKENMKAALADIQSGKFAREFVEDHDAGFPRLKAYRKEAEELEIEKIGAELRKAMPFVGQNDDDAFKIYN

SEQ ID NO: 27 - DNA, 1020 bp, Streptococcus mutans
atggcagtagaaatgttatatgaagcagatgtaaaagtagcagcacttgatggtaagaagattgctgttattggttatggatcacaaggacatgctcatgcccaaaacttgagagattcaggacacgatgttatcatcggtgtgcgtcatgggaaatcgtttgacaaggctaaagaagatggttttgatacctatgaagtgggtgaagctactaaattggctgatattatcatggttttagcaccggatgaaattcaaaaagatatctacaaagatgaaattgctcctaatttaagtgctggtaaggctcttggttttgctcatggttttaatatccatttcggttatattaaggcaccagaagatgtagatgttttcatggttgcccctaaaggaccgggccatcttgtacgtcgtacttacacagaaggatttggagtaccttctttatatgctgtttatcaaaatccaactggtaatgctgaaaatattgctttagattgggctaagggtattggttctgcgcgtgtaggtctccttgtaaccactttcaaagaagaaacggaagaagacctttttggtgaacaagcggttcttatgggtggtttaactcaccttattgaagcaggttttgaagtgcttactgaagcagggtatgctccacaattggcttattttgaagtgcttcatgaaatgaaattgattgtagaccttatctatgaaggtggtttcaagaaaatgcgtcaatcatgttcaaataccgctgaatttggtgactttgtgacaggcccacgtgttatcggtccagaagtaaaagaaaacatgaaggctgctcttgctgatattcaatcaggtaaatttgcccgtgaattcgtggaagatcatgatgctggcttcccacgtttgaaagctttccgtaaagaagctgaaggactcgaaatcgaaaaaattggtgcagaacttcgtaaagcaatgccatttgttaatcaaaacgatgacgatgctttcaaaatttataac

SEQ ID NO: 28 - protein, 340 aa, Streptococcus mutans
MAVEMLYEADVKVAALDGKKIAVIGYGSQGHAHAQNLRDSGHDVIIGVRHGKSFDKAKEDGFDTYEVGEATKLADIIMVLAPDEIQKDIYKDEIAPNLSAGKALGFAHGFNIHFGYIKAPEDVDVFMVAPKGPGHLVRRTYTEGFGVPSLYAVYQNPTGNAENIALDWAKGIGSARVGLLVTTFKEETEEDLFGEQAVLMGGLTHLIEAGFEVLTEAGYAPQLAYFEVLHEMKLIVDLIYEGGFKKMRQSCSNTAEFGDFVTGPRVIGPEVKENMKAALADIQSGKFAREFVEDHDAGFPRLKAFRKEAEGLEIEKIGAELRKAMPFVNQNDDDAFKIYN

SEQ ID NO: 29 - DNA, 1020 bp, Streptococcus gordonii
atggcagttcaaatggaatacgaaaaagatgtaaaagtagcagcgcttgacggtaagaaaattgccgtaatcggctatggttcacaaggtcatgctcatgcacaaaatctgcgtgatacaggccatgatgtgattatcggtgttcgccctggtaagtcattcgataaggctaaagaagatggctttgatacttacacagtagcagaggcagctaagttggctgatgtaatcatgattttggcgccagatgaaattcaacaagaactctacgaagcagaaatcgctccaaacttggaagctggtaatgcagttggatttgcccatggtttcaatatccattttgaatttatcaaagttcctgctgatgtagatgtctttatgtgtgcccctaaaggtcctggtcacttggttcgtcgtacttttgaagaaggatttggtgttccggctctttatgcagtctaccaagatgcaacaggaaatgcgaaaaacattgctatggactggtgtaaaggtgttggtgcagctcgtgttggtttgcttgaaacaacttacaaagaagaaacagaagaagatctctttggtgaacaagctgtcctttgtggtggtttgactgcccttatcgaagcaggttttgaagtcttgacagaagcaggttacgctcctgaattggcttactttgaagttcttcatgaaatgaaattgatcgttgacttgatctacgagggtggattcaagaaaatgcgccaatcaatttcaaatactgctgaatatggtgattacgtatcaggcccacgtgtaattactgagcaagtcaaagaaaacatgaaggctgttttggcagacattcaaaatggtaaatttgcaaatgactttgtaaatgactacaaggctggccgtccaaaactcactgcttaccgtgaacaagcagccaaccttgaaatcgaaaaagtaggtgctgaattgcgtaaagcaatgccatttgttggtaaaaatgacgacgacgcatttaaaatctacaac

SEQ ID NO: 30 - protein, 340 aa, Streptococcus gordonii
MAVQMEYEKDVKVAALDGKKIAVIGYGSQGHAHAQNLRDTGHDVIIGVRPGKSFDKAKEDGFDTYTVAEAAKLADVIMILAPDEIQQELYEAEIAPNLEAGNAVGFAHGFNIHFEFIKVPADVDVFMCAPKGPGHLVRRTFEEGFGVPALYAVYQDATGNAKNIAMDWCKGVGAARVGLLETTYKEETEEDLFGEQAVLCGGLTALIEAGFEVLTEAGYAPELAYFEVLHEMKLIVDLIYEGGFKKMRQSISNTAEYGDYVSGPRVITEQVKENMKAVLADIQNGKFANDFVNDYKAGRPKLTAYREQAANLEIEKVGAELRKAMPFVGKNDDDAFKIYN

SEQ ID NO: 31 - DNA, 1020 bp, Streptococcus suis
atgacagtaacaatgcaatatgaaaaagatgtaacagtggcagcacttgacggtaaacgtatcgccgttatcggttatggttcacaaggtcatgcccatgctcaaaacttgcgtgatacaggacacgatgtcatcatcggtgtgcgtgcaggtaagtcatttgacaaggcaaaagaagacggttttgaaacttttgaagtagcagaggcagcaaaacaagctgatgtcattatgattttggctccagacgaaattcaagcagatttgtatgagaacgaagttgctccaaacttggaagctggaaatgcgcttggttttgctcatggtttcaacgttcactttgaatttatcaaggtaccagcagatgtggatgtcttcatgtgtgcaccaaaaggtccaggtcacttggtccgccgtacctttgaagaaggttttggagtaccagccctctatgcggtctaccaagatgcaactggcaatgcaaaacatattgcgatggactgggcaaaaggtgttggttcagcccgtgttggtcttttggaaacaactttcaaagaagagacagaagaagatttgtttggtgagcaagctgttctctgcggtggtttgacagcccttatgcaggcaggttttgaagtcttgacagaagctggttatgcaccagaattggcttatttcgaagttctccacgagatgaaactcatcgttgaccttgtctacgaaggtggctttaagaaaatgcgccaatctatttcaaacactgctgaatttggtgactatgtatcaggtccacgcgtgattacagaccaagtcaaagaaaacatgaaggcagtccttgcagatattcaatctggtaaatttgcaaatgactttgtaaatgactataaagcaggccgtccacgtatggaagcttatcgtaaagaagcagagaatcttgaaatcgaaaaagtaggtgcagaactccgcaaagcaatgccatttgtcggacgcaatgatgacgacgcattcaaaatctataat

SEQ ID NO: 32 - protein, 340 aa, Streptococcus suis
MTVTMQYEKDVTVAALDGKRIAVIGYGSQGHAHAQNLRDTGHDVIIGVRAGKSFDKAKEDGFETFEVAEAAKQADVIMILAPDEIQADLYENEVAPNLEAGNALGFAHGFNVHFEFIKVPADVDVFMCAPKGPGHLVRRTFEEGFGVPALYAVYQDATGNAKHIAMDWAKGVGSARVGLLETTFKEETEEDLFGEQAVLCGGLTALMQAGFEVLTEAGYAPELAYFEVLHEMKLIVDLVYEGGFKKMRQSISNTAEFGDYVSGPRVITDQVKENMKAVLADIQSGKFANDFVNDYKAGRPRMEAYRKEAENLEIEKVGAELRKAMPFVGRNDDDAFKIYN

SEQ ID NO: 33 - DNA, 1020 bp, Streptococcus infantarius
atggcagtaacaatggaatacgaaaaagacgtaaaagtagcagctcttgatggtaaaaaaattgccgttattggttatggatcacaaggtcatgctcatgctcaaaacttgcgtgactcaggtcacgatgttatcattggggttcgccatggtaaatcattcgataaagcaaaagaagatggatttgatacttatgaagtagcagaagcaacaaaacttgctgatgttatcatggtattggctcctgatgaaatccaagctaaactttatgctgaagaaatcgctccaaaccttgaagctggtaacgctcttggatttgcacatggttttaatatccgttttggatacattaaagctccagaaacagtagatgtcttcatgtgtgctcctaaaggaccaggtcaccttgttcgtcgtacttacacagaaggatttggtgtaccagcactttacgctgtttaccaagatgctactggtaatgctaaagacatcgcaatggattggtctaaaggtatcggtgctgcacgtgttggacttcttgaaacaacatttaaagaagaaactgaagaagacctctttggtgaacaagcagtactttgtggtggtttaactgctcttatcgaagctggttttgaagttcttactgaagctggctatgctccagaattggcttactttgaagttcttcatgaaatgaaacttatcgttgaccttatctacgaaggtggattcaagaaaatgcgtcaatcaatttcaaatacagctgaatttggtgactacgtatctggaccacgtgttatcactaaagatgttaaagaaaatatgaaagctgttcttgctgatatccaatcaggtaaattcgctgaagattttgttaacgactaccaagcaggtcgtccaaaacttgaagcataccgtaaagaagctgcagctcttgaaattgaaaaagtgggtgctgaacttcgtaaagcaatgccttttgttaaccaaaacgatgacgatgcattcaaaatttataac

SEQ ID NO: 34 - protein, 340 aa, Streptococcus infantarius
MAVTMEYEKDVKVAALDGKKIAVIGYGSQGHAHAQNLRDSGHDVIIGVRHGKSFDKAKEDGFDTYEVAEATKLADVIMVLAPDEIQAKLYAEEIAPNLEAGNALGFAHGFNIRFGYIKAPETVDVFMCAPKGPGHLVRRTYTEGFGVPALYAVYQDATGNAKDIAMDWSKGIGAARVGLLETTFKEETEEDLFGEQAVLCGGLTALIEAGFEVLTEAGYAPELAYFEVLHEMKLIVDLIYEGGFKKMRQSISNTAEFGDYVSGPRVITKDVKENMKAVLADIQSGKFAEDFVNDYQAGRPKLEAYRKEAAALEIEKVGAELRKAMPFVNQNDDDAFKIYN

SEQ ID NO: 35 - DNA, 1020 bp, Lactococcus lactis
atggcagttacaatgtattatgaagaagatgtagaagtagccgcactcgcgggtaagaaaatcgcagtgattggatatggctcacaaggacacgctcatgcacaaaacttgcgtgattctggtcatgatgtgattattggtgtccgtcaggggaaatcttttgataaagcaaaagaagatggttttgaaacatttgaagtaggagaagcagtagctaaagctgacgtcattatggttctggcacctgatgaacttcaacaatctatttatgaagaggacataaaaccaaatttgaaagcaggttcagcacttggttttgcccatggtttcaatattcattttggctatattgaagttccagaagatgttgatgtcttcatggttgcgccaaaagcgccgggacatctcgttcggcggacttttaccgaaggtttcggaacgccagctttgttcgtttcgcatcaaaatgccactggtcatgcgcgtgaaattgccatggactgggccaaaggaattggctgtgcccgtgtcggtatcattgaaacaactttcaaagaagaaacagaagaagatttgtttggcgaacaggccgtgctttgtggcggtttgacagcacttgttgaagctggttttgaaacactgacagaagctggatatgctggcgaattggcttactttgaagtgctgcatgaaatgaaattgattgttgaccttatgtacgaaggtggtttcactaaaatgcgtcagtcaatctcaaacactgccgaatttggtgattatgtgactggaccacgcattattactgacgaagttaaaaagaatatgaaactcgtgttggctgacattcaatcaggaaaatttgcgcaagatttcgttgatgatttcaaagctggacgtccaaaattaactgcttatcgtgaagcagctaaaaatctggaaattgaaaaaatcggtgcagaactacgtaaagcaatgccatttacacaatctggtgatgacgacgcctttaaaatttatcaa

SEQ ID NO: 36 - protein, 340 aa, Lactococcus lactis
MAVTMYYEEDVEVAALAGKKIAVIGYGSQGHAHAQNLRDSGHDVIIGVRQGKSFDKAKEDGFETFEVGEAVAKADVIMVLAPDELQQSIYEEDIKPNLKAGSALGFAHGFNIHFGYIEVPEDVDVFMVAPKAPGHLVRRTFTEGFGTPALFVSHQNATGHAREIAMDWAKGIGCARVGIIETTFKEETEEDLFGEQAVLCGGLTALVEAGFETLTEAGYAGELAYFEVLHEMKLIVDLMYEGGFTKMRQSISNTAEFGDYVTGPRIITDEVKKNMKLVLADIQSGKFAQDFVDDFKAGRPKLTAYREAAKNLEIEKIGAELRKAMPFTQSGDDDAFKIYQ

SEQ ID NO: 37 - DNA, 1032 bp, Lactococcus lactis
atggcagttacaatgtattatgaagatgatgtagaagtatcagcacttgctggaaagcaaattgcagtaatcggttatggttcacaaggacatgctcacgcacagaatttgcgtgattctggtcacaacgttatcattggtgtgcgccacggaaaatcttttgataaagcaaaagaagatggctttgaaacatttgaagtaggagaagcagtagctaaagctgatgttattatggttttggcaccagatgaacttcaacaatccatttatgaagaggacatcaaaccaaacttgaaagcaggttcagcacttggttttgctcacggatttaatatccattttggctatattaaagtaccagaagacgttgacgtctttatggttgcgcctaaggctccaggtcaccttgtccgtcggacttatactgaaggttttggtacaccagctttgtttgtttcacaccaaaatgcaagtggtcatgcgcgtgaaatcgcaatggattgggccaaaggaattggttgtgctcgagtgggaattattgaaacaacttttaaagaagaaacagaagaagatttgtttggagaacaagctgttctatgtggaggtttgacagcacttgttgaagccggttttgaaacactgacagaagctggatacgctggcgaattggcttactttgaagttttgcacgaaatgaaattgattgttgacctcatgtatgaaggtggttttactaaaatgcgtcaatccatctcaaatactgctgagtttggcgattatgtgactggtccacggattattactgacgaagttaaaaagaatatgaagcttgttttggctgatattcaatctggaaaatttgctcaagatttcgttgatgacttcaaagcggggcgtccaaaattaatagcctatcgcgaagctgcaaaaaatcttgaaattgaaaaaattggggcagagcacgtcaagcaatgccattcacacaatctggtgatgacgatgcctttaaaatctatcagtaatttctcttat

SEQ ID NO: 38 - protein, 344 aa, Lactococcus lactis
MAVTMYYEDDVEVSALAGKQIAVIGYGSQGHAHAQNLRDSGHNVIIGVRHGKSFDKAKEDGFETFEVGEAVAKADVIMVLAPDELQQSIYEEDIKPNLKAGSALGFAHGFNIHFGYIKVPEDVDVFMVAPKAPGHLVRRTYTEGFGTPALFVSHQNASGHAREIAMDWAKGIGCARVGIIETTFKEETEEDLFGEQAVLCGGLTALVEAGFETLTEAGYAGELAYFEVLHEMKLIVDLMYEGGFTKMRQSISNTAEFGDYVTGPRIITDEVKKNMKLVLADIQSGKFAQDFVDDFKAGRPKLIAYREAAKNLEIEKIGAEHVKQCHSHNLVMTMPLKSISNFSY

SEQ ID NO: 39 - DNA, 1041 bp, Leuconcstoc mesenteroides
atgactacaaaaatgttttatgataaagatattgatacaacaccattggaaaacaaaaagattgcagttattggctatggtgcacaagggcatgctcaagcgaataatctacgcgactcaggatttgatgtcatcatgggattgcgcccaggaaaatcttttgatagtgctaaaaaagatgggttcgaagtttactcagctgctgaagcaacggcacaagcggatgttgttatgatggaaacccctgacgaattacaagcggcagtttgggagaaagaagttgagcctaaccttaaggcaggttcttaccttggattttctcatgggttcaacattgtttatggtttgattaagcccaatgctgatattaatgtcatgatcattgcgccaaagggtccaggaaacattgaacgtcgccaattcgttgaagggggtgggattccttctttgtatggtgttcatcaagatcctacgggtgatacagctgaagtggccaaggcctatgcgaaaggtattggttcaggtcgcgcaggtatcttggaaacgacttttgaagaagaaacaacagaagacttgtttggtgaacaagctgtactttgtggtggcttaacacaattgattgaggcaggatttaacacattggtggaagcaggttactcacctgaattggcttatttcgaaacatctcatgaaatgaagatgattgttgatttgatctttgaaggtggtttcgagaagatgagacacgattgctcaaatacttgtgaatatggtgaaatgttgaacggaccacgtatcatcactgaagaatcaaagcaaggaatgcgtgatgttttgaaggatatccaagatggtacttatgctaagaagtggttggcagaatacaattctggtttgaaggatttggaaaagatgcggacagagtataagagcggtttgtacgagcaaacaggtaagaaggttcgtgctatgatgccatggatttcagatgcagataagtactcgacagcagcagatactgagcaattctcagcagcaaag

SEQ ID NO: 40 - protein, 347 aa, Leuconostoc mesenteroides
MTTKMFYDKDIDTTPLENKKIAVIGYGAQGHAQANNLRDSGFDVIMGLRPGKSFDSAKKDGFEVYSAAEATAQADVVMMETPDELQAAVWEKEVEPNLKAGSYLGFSHGFNIVYGLIKPNADINVMIIAPKGPGNIERRQFVEGGGIPSLYGVHQDPTGDTAEVAKAYAKGIGSGRAGILETTFEEETTEDLFGEQAVLCGGLTQLIEAGFNTLVEAGYSPELAYFETSHEMKMIVDLIFEGGFEKMRHDCSNTCEYGEMLNGPRIITEESKQGMRDVLKDIQDGTYAKKWLAEYNSGLKDLEKMRTEYKSGLYEQTGKKVRAMMPWISDADKYSTAADTEQFSAAK

SEQ ID NO: 41 - DNA, 1044 bp, Lactobacillus buchneri
atgagtgtagaaatgttgtatgacaaggatgttaccaccaattatcttcaaggaaagaaaattgcttttatcggttatggatcacaaggtcatgcccaagctaacaacttaagagattcaggttatgacgttatcgttggtgttcgcccaggacaatcatttgaaaatgccaaagttgatggatttgatgtatacacaccagccgaagcagctcgtcgtgcagactggattcaaatgttaacgcctgatgaagttatgggcgacgtttataaaaatgaaattgcacctaacctagaggaaggcaatgtattaggcttttcacatggcttcaacattcattataaagaaatcgtaccaccagcaaacgttgatgttgttatgatggcacctaaaggcccaggtaatctttgtcgtcgtacatacgttgaaggctccggtgttccagcattatatggctatttccaagactattcaggtcatgccgaagatttatccaaggaattcgccaaaggaaatggtgcagcacgtgccggactattgaagacaaccttcaaagaagaaactgaagaagatttgtttggtgagcaaaacgtccttatgggtggtgttaccgcgcttatcgaaaccggttatgaagtcttaaccgaagcaggatactcaccacagttggcatacttcgaagtggatcacgaaatgaaattgatctgtgaccttatctacgaaggtgggttcaacaagatgtatgctgactgctcgaacacttctgaatatggttcatatgtagttggtccaaaggtcgttggtaaggaatccaagcaagctatgaaagacgctttgaagcggattcaagacggttcatttgctaaagaattcatggatgactaccgtaacggcttcaagaagctttacaagatgcgtgaacgttcagctaactcacttctttcacgtgttggtgctgatcttcgtgatcacatgtcatttgttggtgaagccgacaagtacagtacacctactgaagaaaaagcagaagcagaagctgaaaag

SEQ ID NO: 42 - protein, 348 aa, Lactobacillus buchneri
MSVEMLYDKDVTTNYLQGKKIAFIGYGSQGHAQANNLRDSGYDVIVGVRPGQSFENAKVDGFDVYTPAEAARRADWIQMLTPDEVMGDVYKNEIAPNLEEGNVLGFSHGFNIHYKEIVPPANVDVVMMAPKGPGNLCRRTYVEGSGVPALYGYFQDYSGHAEDLSKEFAKGNGAARAGLLKTTFKEETEEDLFGEQNVLMGGVTALIETGYEVLTEAGYSPQLAYFEVDHEMKLICDLIYEGGFNKMYADCSNTSEYGSYVVGPKVVGKESKQAMKDALKRIQDGSFAKEFMDDYRNGFKKLYKMRERSANSLLSRVGADLRDHMSFVGEADKYSTPTEEKAEAEAEK

SEQ ID NO: 43 - DNA, 1002 bp, Staphylococcus haemolyticus
atgactaaagtttattacgatcaatcagttgagaaagatgcattacaaggtaaaaaaattgcaattatcggttacggttcacaaggacacgcgcatgcacaaaaccttaaagacaacggttatgacgttattgttggtattcgccctggtcattcttttgataaagctaaagaagatggcttcgatgtttatccagttgcagaagctgttaaacaagcagatgtcatcatggtgttattgccagatgaaatccaaggaaatgtctataaaaatgaaattgaacctaacttagaagctggaaatgccctagcatttgcgcatggttttaatatccactttgaagttatcaaaccacctaaagatgtggatgtcttcttagtagctcctaaaggacctggtcatttagtaagacgtacctttgttgaaggaactgccgtaccagcactatttggtgtacaacaagatgctactggtcaagcacgcgatatttcattaagttatgcgaaaggtatcggagctacgcgtgcaggtgtaattgaaacaacatttaaagaagaaactgaaacagatttattcggtgagcaagcagtactttgtggtggtattcataaattaatccaaagtggctttgaaactttagttgaagcgggctatcaaaaagaattagcatacttcgaagtattacatgaaatgaagttaattgttgatttaatgtatgaaggcggtatggagaacgttcgttactcaatttctaacacagcagagtatggtgactatgtttcaggacctagagtcatcactcctgatgttaaagacaatatgaaagcagtattaaaagatattcaaaatggtaattttgcaaatagctttgttaaagacaatgaaaatggctttaaagaattctataaattacgtgaacaacaacacggtcatgaaatcgaagcagttggtcgcgaattaagaaaaatgatgccattcattaaatctaaaagtattcaaaaa

SEQ ID NO: 44 - protein, 334 aa, Staphylococcus haemolyticus
MTKVYYDQSVEKDALQGKKIAIIGYGSQGHAHAQNLKDNGYDVIVGIRPGHSFDKAKEDGFDVYPVAEAVKQADVIMVLLPDEIQGNVYKNEIEPNLEAGNALAFAHGFNIHFEVIKPPKDVDVFLVAPKGPGHLVRRTFVEGTAVPALFGVQQDATGQARDISLSYAKGIGATRAGVIETTFKEETETDLFGEQAVLCGGIHKLIQSGFETLVEAGYQKELAYFEVLHEMKLIVDLMYEGGMENVRYSISNTAEYGDYVSGPRVITPDVKDNMKAVLKDIQNGNFANSFVKDNENGFKEFYKLREQQHGHEIEAVGRELRKMMPFIKSKSIQK

SEQ ID NO: 45 - DNA, 1002 bp, Staphylococcus epidermidis
atgacaaaagtatattacgacgaaacagtaactcaggatgcattacaaggtaaaaaaattgctgtcattggttatggctcacaaggacatgcacatgcacaaaatttaaaggacaatggttatgatgtagtcattggcctgcgtccaggacgatcatttaataaagctaaagaagatggatttgatgtttatacggtaagtgaagcaacacaacaagcagatgtagtgatggtactattgcctgatgaaattcaaggtgaagtatataacaaggaaattaaaccatatttagaaaaaggaaatgctttagcattcgcacacggttttaatatccatttcagtgttatcgaaccacctagtgatgtcgatgtctttttagtagcacctaaaggacctggtcatttagttagacgtacatttgttgaaggaagtgccgtaccagcattatttggtgttcaacaagatgctacaggccaagctagaaacattgctttaagctacgcaaaaggcattggtgctactcgtgccggggtcattgaaacgacatttaaagaagaaactgaaacagatttattcggtgaacaagctgtactttgtggaggagtttccaaattaattcagagtggattcgaaacacttgtggaagcaggttaccaacctgaattagcttattttgaagtcttacacgaaatgaaattaattgttgatttaatgtatgaaggcggaatggaaaatgtccgttattctatctctaacactgctgaatttggcgactatgtttctggaccaagagtaattacacctaatgttaaagaaaatatgaaaaaagtacttgaagatattcaaaatggtaactttagccgtagatttgttgaagataacaaaaatggctttaaagaattctatcaattacgtgaagatcaacatggtcatcaaattgaacaagttggacgtgaattaagagaaatgatgccattcattaaatctaaaagtattgaaaaa

SEQ ID NO: 46 - protein, 334 aa, Staphylococcus epidermidis
MTKVYYDETVTQDALQGKKIAVIGYGSQGHAHAQNLKDNGYDVVIGLRPGRSFNKAKEDGFDVYTVSEATQQADVVMVLLPDEIQGEVYNKEIKPYLEKGNALAFAHGFNIHFSVIEPPSDVDVFLVAPKGPGHLVRRTFVEGSAVPALFGVQQDATGQARNIALSYAKGIGATRAGVIETTFKEETETDLFGEQAVLCGGVSKLIQSGFETLVEAGYQPELAYFEVLHEMKLIVDLMYEGGMENVRYSISNTAEFGDYVSGPRVITPNVKENMKKVLEDIQNGNFSRRFVEDNKNGFKEFYQLREDQHGHQIEQVGRELREMMPFIKSKSIEK

SEQ ID NO: 47 - DNA, 1021 bp, Streptococcus pneumoniae
atgacagttcaaatggaatatgaaaaagatgttaaagtagcagcacttgacggtaaaaaaatcgccgttatcggttatggttcacaagggcatgcgcatgctcaaaacttgcgtgattcaggtcgtgacgttattatcggtgtacgtccaggtaaatcttttgataaagcaaaagaagatggatttgatacttacacagtaacagaagctactaagttggctgatgttatcatgatcttggcgccagacgaaattcaacaagaattgtacgaagcagaaatcgctccaaacttggaagctggaaacgcagttggatttgcccatggtttcaacatccactttgaatttatcaaagttcctgcggatgtagatgtcttcatgtgtgctcctaaaggaccaggacacttggtacgtcgtacttacgaagaaggatttggtgttccagctctttatgcagtataccaagatgcaacaggaaatgctaaaaacattgctatggactggtgtaaaggtgttggagcggctcgtgtaggtcttcttgaaacaacttacaaagaagaaactgaagaagatttgtttggtgaacaagctgtactttgtggtggtttgactgcccttatcgaagcaggtttcgaagtcttgacagaagcaggttacgctccagaattggcttactttgaagttcttcacgaaatgaaattgatcgttgacttgatctacgaaggtggattcaagaaaatgcgtcaatctatttcaaacactgctgaatacggtgactatgtatcaggtccacgtgtaatcactgaacaagttaaagaaaatatgaaggctgtcttggcagacatccaaaatggtaaatttgcaaatgactttgtaaatgactataaagctggacgtccaaaattgactgcttaccgtgaacaagcagctaaccttgaaattgaaaaagttggtgcagaattgcgtaaagcaatgccattcgttggtaaaaacgatgatgatgcattcaaaatctataact

SEQ ID NO: 48 - protein, 340 aa, Streptococcus pneumoniae
MTVQMEYEKDVKVAALDGKKIAVIGYGSQGHAHAQNLRDSGRDVIIGVRPGKSFDKAKEDGFDTYTVTEATKLADVIMILAPDEIQQELYEAEIAPNLEAGNAVGFAHGFNIHFEFIKVPADVDVFMCAPKGPGHLVRRTYEEGFGVPALYAVYQDATGNAKNIAMDWCKGVGAARVGLLETTYKEETEEDLFGEQAVLCGGLTALIEAGFEVLTEAGYAPELAYFEVLHEMKLIVDLIYEGGFKKMRQSISNTAEYGDYVSGPRVITEQVKENMKAVLADIQNGKFANDFVNDYKAGRPKLTAYREQAANLEIEKVGAELRKAMPFVGKNDDDAFKIYN

SEQ ID NO: 49 - DNA, 1008 bp, Streptococcus pneumoniae
atggaatatgaaaaagatgttaaagtagcagcacttgacggtaaaaaaatcgccgttatcggttatggttcacaagggcatgcgcatgctcaaaacttgcgtgattcaggtcgtgacgttattatcggtgtacgtccaggtaaatcttttgataaagcaaaagaagatggatttgatacttacacagtagcagaagctactaagttggctgatgttatcatgatcttggcgccagacgaaattcaacaagaattgtacgaagcagaaatcgctccaaacttggaagctggaaacgcagttggatttgcccatggtttcaacatccactttgaatttatcaaagttcctgcggatgtagatgtcttcatgtgtgctcctaaaggaccaggacacttggtacgtcgtacttacgaagaaggatttggtgttccagctctttatgcagtataccaagatgcaacaggaaatgctaaaaacattgctatggactggtgtaaaggtgttggagcggctcgtgtaggtcttcttgaaacaacttacaaagaagaaactgaagaagatttgtttggtgaacaagctgtactttgtggtggtttgactgcccttatcgaagcaggtttcgaagtcttgacagaagcaggttacgctccagaattggcttactttgaagttcttcacgaaatgaaattgatcgttgacttgatctacgaaggtggattcaagaaaatgcgtcaatctatttcaaacactgctgaatacggtgactatgtatcaggtccacgtgtaatcactgaacaagttaaagaaaatatgaaggctgtcttggcagacatccaaaatggtaaatttgcaaatgactttgtaaatgactataaagctggacgtccaaaattgactgcttaccgtgaacaagcagctaaccttgaaattgaaaaagttggtgcagaattgcgtaaagcaatgccattcgttggtaaaaacgacgatgatgcattcaaaatctataac

SEQ ID NO: 50 - protein, 336 aa, Streptococcus pneumoniae
MEYEKDVKVAALDGKKIAVIGYGSQGHAHAQNLRDSGRDVIIGVRPGKSFDKAKEDGFDTYTVAEATKLADVIMILAPDEIQQELYEAEIAPNLEAGNAVGFAHGFNIHFEFIKVPADVDVFMCAPKGPGHLVRRTYEEGFGVPALYAVYQDATGNAKNIAMDWCKGVGAARVGLLETTYKEETEEDLFGEQAVLCGGLTALIEAGFEVLTEAGYAPELAYFEVLHEMKLIVDLIYEGGFKKMRQSISNTAEYGDYVSGPRVITEQVKENMKAVLADIQNGKFANDFVNDYKAGRPKLTAYREQAANLEIEKVGAELRKAMPFVGKNDDDAFKIYN

SEQ ID NO: 51 - DNA, 1020 bp, Streptococcus sanguinis
atggcagtaacaatggaatacgaaaaagatgttaaagtagcagcgcttgacggtaaaaaaatcgccgttatcggttatggttcacaaggacatgcgcatgcacaaaacttgcgtgatacaggccacgatgtgattatcggtgttcgtcctggtaagtcatttgacaaggctaaagaagatggctttgatacttatacagtagcagaagcagctaaattagctgatgtcatcatgattttggctccagatgaaatccaacaagatctctatgaagcagaaatcgctccaaacttggaagctggaaacgcagttgggtttgctcatggtttcaacatccattttgagtttatcaaagttcctgccgatgtagatgtctttatgtgtgctcctaaaggccctggtcacttggttcgccgtactttcgaagaaggttttggtgtaccagctctgtatgcagtctaccaagacgctactggaaatgccaaagacatcgcaatggactggtgtaaaggtgttggttcagctcgtgttggacttcttgaaacaacttataaagaagaaacagaagaagatctctttggtgaacaagctgtgctttgtggtggcttgactgccctgattgaagcaggatttgaagttctgactgaagcaggttatgcgccagaattggcttactttgaagtgctgcacgaaatgaaactgattgtagacttgatctatgaaggtggcttcaagaagatgcgccaatctatctcaaatacagctgaatatggtgactatgtatctggtccgcgcgtcattactgagcaagtcaaagaaaacatgaaggcagtcttggctgatatccaaaacggtaaatttgcgaacgacttcgttgatgattacaaggctggtcgtccaaaactcactgcttaccgcgagcaagctgctaatctggagattgaaaaggtcggtgcagaattgcgtaaagcaatgccattcgttggtaaaaacgacgacgatgctttcaagatttataat

SEQ ID NO: 52 - protein, 340 aa, Streptococcus sanguinis
MAVTMEYEKDVKVAALDGKKIAVIGYGSQGHAHAQNLRDTGHDVIIGVRPGKSFDKAKEDGFDTYTVAEAAKLADVIMILAPDEIQQDLYEAEIAPNLEAGNAVGFAHGFNIHFEFIKVPADVDVFMCAPKGPGHLVRRTFEEGFGVPALYAVYQDATGNAKDIAMDWCKGVGSARVGLLETTYKEETEEDLFGEQAVLCGGLTALIEAGFEVLTEAGYAPELAYFEVLHEMKLIVDLIYEGGFKKMRQSISNTAEYGDYVSGPRVITEQVKENMKAVLADIQNGKFANDFVDDYKAGRPKLTAYREQAANLEIEKVGAELRKAMPFVGKNDDDAFKIYN

SEQ ID NO: 53 - DNA, 1020 bp, Streptococcus salivarius
atggcagttcaaatggaatatgaaaaagacgtaaaagtagcagcacttgacggtaaaaaaatcgccgttatcggttacggttcacaaggtcatgcacatgcacaaaacttgcgtgatacaggtcacgatgtgattatcggtgttcgtccaggtaaatcatttgataaagctaaagaagatggttttgacacttacacagtagcagaagcaactaaattggctgatgttatcatgattttggctccagacgaaatccaacaagaactttacgaagcagaaattgctccaaaccttgaagctggtaatgctgttggttttgcacacggatttaacatccacttcgaattcatcaaagttcctaaagatgttgatgtcttcatgtgtgctcctaaaggaccaggtcacttggtacgtcgtacatttgaagaaggatttggtgttcctgcactttatgcagtataccaagatgctacaggtaacgctaaagatatcgctatggactggtgtaaaggtgttggtgcagcacgtgttggtcttcttgaaacaacatacaaagaagaaactgaagaagatttgttcggtgaacaagcagttctttgtggtggtttgactgcccttatcgaagctggttttgaagtcttgactgaagctggatatgctccagaattggcttactttgaagtgcttcacgaaatgaaattgatcgttgacttgatttacgaaggtggattcaagaaaatgcgtcaatcaatttctaacactgctgaattcggtgactacgtatcaggtccacgtgttatcactgaacaagtgaaagaaaacatgaaagcagttcttgctgacatccaaaacggtaaattcgctaacgacttcgttaacgactacaaagctggacgtccaaaacttactgcttaccgtgaagaagctgctaaccttgaaatcgaaaaagttggtgctgaattgcgtaaagcaatgccattcgttggtcaaaacgacgatgacgcattcaaaatctataac

SEQ ID NO: 54 - protein, 340 aa, Streptococcus salivarius
MAVQMEYEKDVKVAALDGKKIAVIGYGSQGHAHAQNLRDTGHDVIIGVRPGKSFDKAKEDGFDTYTVAEATKLADVIMILAPDEIQQELYEAEIAPNLEAGNAVGFAHGFNIHFEFIKVPKDVDVFMCAPKGPGHLVRRTFEEGFGVPALYAVYQDATGNAKDIAMDWCKGVGAARVGLLETTYKEETEEDLFGEQAVLCGGLTALIEAGFEVLTEAGYAPELAYFEVLHEMKLIVDLIYEGGFKKMRQSISNTAEFGDYVSGPRVITEQVKENMKAVLADIQNGKFANDFVNDYKAGRPKLTAYREEAANLEIEKVGAELRKAMPFVGQNDDDAFKIYN

SEQ ID NO: 55 - DNA, 1020 bp, Streptococcus thermophilus
atggcagttcaaatggaatatgaaaaagacgtaaaagtaccagcacttgatggtaaaaaaatcgccgttatcggttacggttcacaaggtcacgctcactcacaaaacttgcgtgatacaggtcacgatgttatcatcggtgttcgtccaggtaaatcatttgataaagctaaagaagatggttttgatacttacacagtagcagaagcaactaaattggctgatgttatcatgattttggctccagacgaaatccaacaagaactttacgaagcagaaatcgctccaaatcttgaagctggtaacgctgttggttttgctcacggtttcaacatccactttgaatttattaaagtaccagcagatgttgatgtatttatgtgtgcacctaaaggaccaggtcacttggtacgtcgtacatttgaagaaggttttggtgtacctgccctttacgcagtataccaagatgccacaggtaacgctaaagacatcgctatggactggtgtaaaggtattggtgcagcacgtgtaggtcttcttgaaacaacatacaaagaagaaactgaagaagatttgtttggtgaacaagcagttctttgtggtggtttgactgcccttatcgaaactggttttgaagtcttgactgaagctggatatgcaccagaattggcttactttgaagtacttcacgaaatgaaattaatcgttgacttgatttacgaaggtggattcaagaaaatgcgtcaatcaatttctaacactgctgaattcggtgactatgtatcaggtccacgtgttatcactgaacaagttaaagaaaacatgaaagcagttcttgctgatatccaaaatggtaaattcgctaatgacttcgttaacgactacaaagctggacgtccaaaacttactgcttaccgtgaagaagctgctaaccttgaaatcgaaaaagttggtgctgaattgcgtaaagcaatgccattcgttggtcaaaacgacgatgatgcattcaaaatctataac

SEQ ID NO: 56 - protein, 340 aa, Streptococcus thermophilus
MAVQMEYEKDVKVPALDGKKIAVIGYGSQGHAHSQNLRDTGHDVIIGVRPGKSFDKAKEDGFDTYTVAEATKLADVIMILAPDEIQQELYEAEIAPNLEAGNAVGFAHGFNIHFEFIKVPADVDVFMCAPKGPGHLVRRTFEEGFGVPALYAVYQDATGNAKDIAMDWCKGIGAARVGLLETTYKEETEEDLFGEQAVLCGGLTALIETGFEVLTEAGYAPELAYFEVLHEMKLIVDLIYEGGFKKMRQSISNTAEFGDYVSGPRVITEQVKENMKAVLADIQNGKFANDFVNDYKAGRPKLTAYREEAANLEIEKVGAELRKAMPFVGQNDDDAFKIYN

SEQ ID NO: 57 - DNA, 975 bp, Streptococcus pneumoniae
ttgacggtaaaaaaatacgccgttatcggttatggttcacaagggcatgcgcatgctcaaaacttgcgtgattcaggtcgtgacgttattataggtgtacgtccaggtaaatcttttgataaagcaaaagaagatggatttgatacttacacagtagcagaagctactaagttggctgatgttatcatgatcttggcgccagacgaaattcaacaagaattgtacgaagcagaaatcgctccaaacttggaagctggaaacgcagttggatttgcccatggtttcaacatccactttgaatttatcaaagttcctgcggatgtagatgtcttcatgtgtgctcctaaaggaccaggacacttggtacgtcgtacttacgaagaaggatttggtgttccagctctttatgcagtataccaagatgcaacaggaaatgctaaaaacattgctatggactggtgtaaaggtgttggagcggctcgtgtaggtcttcttgaaacaacttacaaagaagaaactgaagaagatttgtttggtgaacaagctgtactttgtggtggtttgactgcccttatcgaagcaggtttcgaagtcttgacagaagcaggttacgctccagaattggcttactttgaagttcttcacgaaatgaaattgatcgttgacttgatctacgaaggtggattcaagaaaatgcgtcaatctatttcaaacactgctgaatacggtgactatgtatcaggtccacgtgtaatcactgaacaagttaaagaaaatatgaaggctgtcttggcagacatccaaaatggtaaatttgcaaatgactttgtaaatgactataaagctggacgtccaaaattgactgcttaccgtgaacaagcagctaaccttgaaattgaaaaagttggtgcagaattgcgtaaagcaatgccattcgttggtaaaaacgacgatgatgcattcaaaatctataac

SEQ ID NO: 58 - protein, 325 aa, Streptococcus pneumoniae
MTVKKYAVIGYGSQGHAHAQNLRDSGRDVIIGVRPGKSFDKAKEDGFDTYTVAEATKLADVIMILAPDEIQQELYEAEIAPNLEAGNAVGFAHGFNIHFEFIKVPADVDVFMCAPKGPGHLVRRTYEEGFGVPALYAVYQDATGNAKNIAMDWCKGVGAARVGLLETTYKEETEEDLFGEQAVLCGGLTALIEAGFEVLTEAGYAPELAYFEVLHEMKLIVDLIYEGGFKKMRQSISNTAEYGDYVSGPRVITEQVKENMKAVLADIQNGKFANDFVNDYKAGRPKLTAYREQAANLEIEKVGAELRKAMPFVGKNDDDAFKIYN

SEQ ID NO: 59 - DNA, 1020 bp, Lactococcus lactis
atggcagttacaatgtattatgaagatgatgtagaagtatcagcacttgctggaaagcaaattgcagtaatcggttatggttcacaaggacatgctcacgcacagaatttgcgtgattctggtcacaacgttatcattggtgtgcgccacggaaaatcttttgataaagcaaaagaagatggctttgaaacatttgaagtaggagaagcggtagctaaagctgatgttattatggttttggcgccagatgaacttcaacaatccatttatgaagaggacatcaaaccaaacttgaaagcaggttcagcacttggttttgctcacggatttaatatccattttggctatattaaagtaccagaagacgttgacgtctttatggttgcacctaaggctccaggtcaccttgtccgtcggacttatactgaaggttttggtacaccagctttgtttgtttcacaccaaaatgcaagtggtcatgcgcgtgaaatcgcaatggattgggccaaaggaattggttgtgctcgagtgggaattattgaaacaacctttaaagaagaaacagaagaagatttgtttggagaacaagctgttctatgtggaggtttgacagcacttgttgaagccggttttgaaacactgacagaagctggatacgctggcgaattggcttactttgaagttttgcacgaaatgaaattgattgttgacctcatgtatgaaggtggttttactaaaatgcgtcaatccatctcaaatactgctgagtttggcgattatgtgactggtccaaggattattactgacgcagttaaaaagaatatgaagcttgttttggctgatattcaatctggaaaatttgctcaagatttcgttgatgacttcaaagcggggcgtccaaaattaacagcctatcgcgaagctgctaaaaatcttgaaattgaaaaaattggggcagaattacgtaaagcaatgccattcacacaatctggtgatgacgatgcctttaaaatctatcag

SEQ ID NO: 60 - protein, 340 aa, Lactococcus lactis
MAVTMYYEDDVEVSALAGKQIAVIGYGSQGHAHAQNLRDSGHNVIIGVRHGKSFDKAKEDGFETFEVGEAVAKADVIMVLAPDELQQSIYEEDIKPNLKAGSALGFAHGFNIHFGYIKVPEDVDVFMVAPKAPGHLVRRTYTEGFGTPALFVSHQNASGHAREIAMDWAKGIGCARVGIIETTFKEETEEDLFGEQAVLCGGLTALVEAGFETLTEAGYAGELAYFEVLHEMKLIVDLMYEGGFTKMRQSISNTAEFGDYVTGPRIITDAVKKNMKLVLADIQSGKFAQDFVDDFKAGRPKLTAYREAAKNLEIEKIGAELRKAMPFTQSGDDDAFKIYQ

SEQ ID NO: 61 - DNA, 1041 bp, Leuconostoc mesenteroides
atgactacaaaaatgttttatgacaaagatattgatacaaaaccattggaaaacaaaaagattgcagttattggctatggtgcacaagggcatgctcaagcgaataatcttcgcgactcaggatttgatgtcatcatgggattgcgcccaggaaaatattttgatagtgctaaaaaagatgggttcgaagtttactcagctgctgaagcaacggcacaagcggattttgttatgatggaaacccctgacgaattacaagcggcagtttgggagaaagaagttgagcctaaccttaaggcaggttcttacattggattttctcatgggttcaacattgtttatggtttgattaagcccaatgctgatattaatgtcatgatcatcgcgccaaagggtccaggaaacattgaacgtcgccaattcgttgaaggggttgggattccttctttgtatggtgttcatcaagatcctacgggtgatacagctgaagtggccaaggcctatgcgaaaggtattggttcaggtcgcgcaggtatcttggaaacgacttttgaagaagaaacaacagaaaacttgtttggtgaacaggctgtactttgtggtggcttaacacaattgattgaggcaggatttaacacattggtggaagcaggttactcacctgaattggcttatttcgaaacatctcatgaaatgaagatgattgttgatttgatctttgaaggtggtttcgagaagatgagacatgattgctcaaatacttgtgaatatggtgaaatgttgaacggaccacgtatcatcactgaagaatcaaagcaaggaatgcgtgatgttttgaaggatatccaagatggtacttatgctaagaagtggttggcagaatacaattctggtttgaaggatttggaaaagatgcggacagagtataagagcggtttgtacgagcaaacaggtaagaaggttcgtgctatgatgccatggatttcagatgcagataagtactcgacagcagcagatactgagcaattctcagcagcaaag

SEQ ID NO: 62 - protein, 347 aa, Leuconostoc mesenteroides
MTTKMFYDKDIDTKPLENKKIAVIGYGAQGHAQANNLRDSGFDVIMGLRPGKYFDSAKKDGFEVYSAAEATAQADFVMMETPDELQAAVWEKEVEPNLKAGSYIGFSHGFNIVYGLIKPNADINVMIIAPKGPGNIERRQFVEGVGIPSLYGVHQDPTGDTAEVAKAYAKGIGSGRAGILETTFEEETTENLFGEQAVLCGGLTQLIEAGFNTLVEAGYSPELAYFETSHEMKMIVDLIFEGGFEKMRHDCSNTCEYGEMLNGPRIITEESKQGMRDVLKDIQDGTYAKKWLAEYNSGLKDLEKMRTEYKSGLYEQTGKKVRAMMPWISDADKYSTAADTEQFSAAK

SEQ ID NO: 63 - DNA, 1041 bp, Leuconostoc mesenteroides
atgactacaaaaatgttttatgacaaagatattgatacaaaaccattggaaaacaaaaagattgcagttattggctatggtgcacaagggcatgctcaagcgaataatcttcgcgtctcaggatttgatgtcatcatgggattgcgcccaggaaaatcttttgatagtgttaaaaaagatgggttcgaagtttactcagctgctgaagcaacggcacaagcggatgttgttatgatggaaacccctgacgaattacaagcggcagtttgggagaaagaagttgagcctaaccttaaggcaggttctgaccttggattttctcatgggttcaacattgtttatggtttgattaagcccaatgctgatattaatgtcatgatcattgcgccaaagggtccaggaaacattgaacgtcgccaattcgttgaagggggtgggattccttctttgtatggtgttcatcaagatcctacgggtgatacagctgaagtggccaaggcctatgcgaaaggtattggttcaggttgcgcaggtatcttggaaacgacttttgaagaagaaacaacagaagacttgtttggtgaacaagctgtactttgtggtggcttaacacaattgattgaggcaggatttaacacattggtggaagcaggttactcacctgaattggcttatttcgaaacatctcatgaaatgaagatgattgttgatttgatctttgaaggtggtttcgagaagatgagacatgattgctcaaatacttgtgaatatggtgaaatgttgaacggaccacgtatcatcactgaagaatcaaagcaaggaatgcgtgatgttttgaaggatatccaagatggtacttatgctaagaagtggttggcagaatacaattctggtttgaaggatttggaaaagatgcggacagagtataagagcggtttgtacgagcaaacaggtaagaaggttcgtgctatgatgccatggatttcagatgcagataagtactcgacagcagcagatactgagcaattctcagcagcaaag

SEQ ID NO: 64 - protein, 347 aa, Leuconostoc mesenteroides
MTTKMFYDKDIDTKPLENKKIAVIGYGAQGHAQANNLRVSGFDVIMGLRPGKSFDSVKKDGFEVYSAAEATAQADVVMMETPDELQAAVWEKEVEPNLKAGSDLGFSHGFNIVYGLIKPNADINVMIIAPKGPGNIERRQFVEGGGIPSLYGVHQDPTGDTAEVAKAYAKGIGSGCAGILETTFEEETTEDLFGEQAVLCGGLTQLIEAGFNTLVEAGYSPELAYFETSHEMKMIVDLIFEGGFEKMRHDCSNTCEYGEMLNGPRIITEESKQGMRDVLKDIQDGTYAKKWLAEYNSGLKDLEKMRTEYKSGLYEQTGKKVRAMMPWISDADKYSTAADTEQFSAAK

SEQ ID NO: 65 - DNA, 1038 bp, Lactococcus brevis
atgagtgtagaaatgttgtatgacaaggatgttaccaccaattatcttcaaggaaagaaaattgcttttatcggttatggatctcaaggccatgcccaagctaacaacttaagagattcaggttatgacgttatcgttggtgttcgcccaggacaatcatttgaaaatgccaaaatcgatggattcgatgtttacacaccagccgaagcagctcgtcgtgcagactggattcaaatgttaacgcctgatgaagttatgggtgatgtttataaaaatgaaattgcacctaaccttgaggaaggcaatgtattaggcttttcacatggcttcaacattcattataaagaaatcgtaccaccagcaaacgttgatgttgttatgatggcacctaaaggcccaggtaatctttgtcgtcgtacatacgttgaaggctccggtgttccagcattatatggttatttccaagactattcaggccatgccgaagatttatccaaggaattcgccaaaggaaatggtgcagcacgtgccggactattgaagacaaccttcaaagaagaaactgaagaagatttgtttggtgagcaaaacgtccttatgggtggtgttaccgcgctcattgaaaccggttatgaagtcttaaccgaagcaggatactcaccacagttggcatactttgaagtagatcatgaaatgaaattgatctgtgaccttatctacgaaggtgggttcaacaagatgtatgctgactgctcgaacacttctgaatatggttcatatgtagttggtccaaaggtcgttggtaaggaatccaagcaagctatgaaagacgctttgaagcggattcaagacggttcatttgctaaagaattcatggatgactaccgtaacggcttcaagaagctttacaagatgcgtgaacgttcagctaactcacttctttcacgtgttggtgctgatcttcgtgatcacatgtcatttgttggtgaagccgacaagtacagtacacctactgaagaaaaagcagaagctgaaaag

SEQ ID NO: 66 - protein, 346 aa, Lactococcus brevis
MSVEMLYDKDVTTNYLQGKKIAFIGYGSQGHAQANNLRDSGYDVIVGVRPGQSFENAKIDGFDVYTPAEAARRADWIQMLTPDEVMGDVYKNEIAPNLEEGNVLGFSHGFNIHYKEIVPPANVDVVMMAPKGPGNLCRRTYVEGSGVPALYGYFQDYSGHAEDLSKEFAKGNGAARAGLLKTTFKEETEEDLFGEQNVLMGGVTALIETGYEVLTEAGYSPQLAYFEVDHEMKLICDLIYEGGFNKMYADCSNTSEYGSYVVGPKVVGKESKQAMKDALKRIQDGSFAKEFMDDYRNGFKKLYKMRERSANSLLSRVGADLRDHMSFVGEADKYSTPTEEKAEAEK

SEQ ID NO: 67 - DNA, 1020 bp, Lactococcus lactis
atggcagttacaatgtattatgaagatgatgtagaagtatcagcacttgctggaaagcaaattgcagtaatcggttatggttcacaaggacatgctcacgcacagaatttgcgtgattctggtcacaacgttatcattggtgtgcgccacggaaaatcttttgataaagcaaaagaagatggctttgaaacatttgaagtaggagaagcagtagctaaagctgatgttattatggttttggcaccagatgaacttcaacaatccatttatgaagaggacatcaaaccaaacttgaaagcaggttcagcacttggttttgctcacggatttaatatccattttggctatattaaagtaccagaagacgttgacgtctttatggttgcgcctaaggctccaggtcaccttgtccgtcggacttatactgaaggttttggtacaccagctttgtttgtttcacaccaaaatgcaagtggtcatgcgcgtgaaatcgcaatggattgggccaaaggaattggttgtgctcgagtgggaattattgaaacaacttttaaagaagaaacagaagaagatttgtttggagaacaagctgttctatgtggaggtttgacagcacttgttgaagccggttttgaaacactgacagaagctggatacgctggcgaattggcttactttgaagttttgcacgaaatgaaattgattgttgacctcatgtatgaaggtggttttactaaaatgcgtcaatccatctcaaatactgctgagtttggcgattatgtgactggtccacggattattactgacgaagttaaaaagaatatgaagcttgttttggctgatattcaatctggaaaatttgctcaagatttcgttgatgacttcaaagcggggcgtccaaaattaatagcctatcgcgaagctgcaaaaaatcttgaaattgaaaaaattggggcagagctacgtcaagcaatgccattcacacaatctggtgatgacgatgcctttaaaatctatcag

SEQ ID NO: 68 - protein, 340 aa, Lactococcus lactis
MAVTMYYEDDVEVSALAGKQIAVIGYGSQGHAHAQNLRDSGHNVIIGVRHGKSFDKAKEDGFETFEVGEAVAKADVIMVLAPDELQQSIYEEDIKPNLKAGSALGFAHGFNIHFGYIKVPEDVDVFMVAPKAPGHLVRRTYTEGFGTPALFVSHQNASGHAREIAMDWAKGIGCARVGIIETTFKEETEEDLFGEQAVLCGGLTALVEAGFETLTEAGYAGELAYFEVLHEMKLIVDLMYEGGFTKMRQSISNTAEFGDYVTGPRIITDEVKKNMKLVLADIQSGKFAQDFVDDFKAGRPKLIAYREAAKNLEIEKIGAELRQAMPFTQSGDDDAFKIYQ

SEQ ID NO: 69 - DNA, 1716 bp, Bacillus subtilis
atgttgacaaaagcaacaaaagaacaaaaatcccttgtgaaaaacagaggggcggagcttgttgttgattgcttagtggagcaaggtgtcacacatgtatttggcattccaggtgcaaaaattgatgcggtatttgacgctttacaagataaaggacctgaaattatcgttgcccggcacgaacaaaacgcagcattcatggcccaagcagtcggccgtttaactggaaaaccgggagtcgtgttagtcacatcaggaccgggtgcctctaacttggcaacaggcctgctgacagcgaacactgaaggagaccctgtcgttgcgcttgctggaaacgtgatccgtgcagatcgtttaaaacggacacatcaatctttggataatgcggcgctattccagccgattacaaaatacagtgtagaagttcaagatgtaaaaaatataccggaagctgttacaaatgcatttaggatagcgtcagcagggcaggctggggccgcttttgtgagctttccgcaagatgttgtgaatgaagtcacaaatacgaaaaacgtgcgtgctgttgcagcgccaaaactcggtcctgcagcagatgatgcaatcagtgcggccatagcaaaaatccaaacagcaaaacttcctgtcgttttggtcggcatgaaaggcggaagaccggaagcaattaaagcggttcgcaagcttttgaaaaaggttcagcttccatttgttgaaacatatcaagctgccggtaccctttctagagatttagaggatcaatattttggccgtatcggtttgttccgcaaccagcctggcgatttactgctagagcaggcagatgttgttctgacgatcggctatgacccgattgaatatgatccgaaattctggaatatcaatggagaccggacaattatccatttagacgagattatcgctgacattgatcatgcttaccagcctgatcttgaattgatcggtgacattccgtccacgatcaatcatatcgaacacgatgctgtgaaagtggaatttgcagagcgtgagcagaaaatcctttctgatttaaaacaatatatgcatgaaggtgagcaggtgcctgcagattggaaatcagacagagcgcaccctcttgaaatcgttaaagagttgcgtaatgcagtcgatgatcatgttacagtaacttgcgatatcggttcgcacgccatttggatgtcacgttatttccgcagctacgagccgttaacattaatgatcagtaacggtatgcaaacactcggcgttgcgcttccttgggcaatcggcgcttcattggtgaaaccgggagaaaaagtggtttctgtctctggtgacggcggtttcttattctcagcaatggaattagagacagcagttcgactaaaagcaccaattgtacacattgtatggaacgacagcacatatgacatggttgcattccagcaattgaaaaaatataaccgtacatctgcggtcgatttcggaaatatcgatatcgtgaaatatgcggaaagcttcggagcaactggcttgcgcgtagaatcaccagaccagctggcagatgttctgcgtcaaggcatgaacgctgaaggtcctgtcatcatcgatgtcccggttgactacagtgataacattaatttagcaagtgacaagcttccgaaagaattcggggaactcatgaaaacgaaagctctctag

SEQ ID NO: 70 - protein, 571 aa, Bacillus subtilis
MLTKATKEQKSLVKNRGAELVVDCLVEQGVTHVFGIPGAKIDAVFDALQDKGPEIIVARHEQNAAFMAQAVGRLTGKPGVVLVTSGPGASNLATGLLTANTEGDPVVALAGNVIRADRLKRTHQSLDNAALFQPITKYSVEVQDVKNIPEAVTNAFRIASAGQAGAAFVSFPQDVVNEVTNTKNVRAVAAPKLGPAADDAISAAIAKIQTAKLPVVLVGMKGGRPEAIKAVRKLLKKVQLPFVETYQAAGTLSRDLEDQYFGRIGLFRNQPGDLLLEQADVVLTIGYDPIEYDPKFWNINGDRTIIHLDEIIADIDHAYQPDLELIGDIPSTINHIEHDAVKVEFAEREQKILSDLKQYMHEGEQVPADWKSDRAHPLEIVKELRNAVDDHVTVTCDIGSHAIWMSRYFRSYEPLTLMISNGMQTLGVALPWAIGASLVKPGEKVVSVSGDGGFLFSAMELETAVRLKAPIVHIVWNDSTYDMVAFQQLKKYNRTSAVDFGNIDIVKYAESFGATGLRVESPDQLADVLRQGMNAEGPVIIDVPVDYSDNINLASDKLPKEFGELMKTKAL

SEQ ID NO: 71 - DNA, 1713 bp, artificial sequence
  codon optimized coding region for expression in Lactobacillus plantarum
atgttgaccaaggctaccaaagaacaaaagagtttagtcaaaaaccgtggtgctgaattagtcgtggattgtttggttgaacaaggtgtgacgcatgtttttggtattccaggagctaaaattgatgccgtttttgatgcgttacaagataagggtccagaaattattgtggcacgtcatgaacaaaatgcagcgtttatggctcaagcagttggtcggttgactggcaaaccaggtgtggttttagtgacgtcaggtccaggtgcgagtaatttagcgactggcttgttaacggcgaatactgaaggtgatccagtcgttgctttggcaggcaatgtcattcgtgccgatcgtttaaagcggacccatcagagtttggataatgcagccttgtttcaaccgattacgaaatattcagttgaagtccaagatgtcaagaatattccagaagcggttacgaatgcgtttcgtattgcatcagctggccaagcaggcgcagcgtttgtgagttttccacaagatgtcgtgaatgaagttactaacaccaagaatgtccgtgcagtcgcagctccaaagttaggtccagcagctgacgatgccattagtgcagctattgccaaaattcagactgcaaaattgccggttgtgttagttggcatgaaaggtggtcgtccagaagccattaaagcggttcgtaagttattgaaaaaggttcaattaccatttgttgaaacgtatcaagctgcaggtacgttaagtcgtgacttagaagatcaatattttggtcggattggtttgtttcgtaatcaaccaggtgatttgttattagaacaagctgatgtggttttaactattggctatgatccgattgaatatgatccaaagttttggaatattaatggtgatcgtaccatcattcatttggatgaaatcattgctgatattgatcacgcttatcaaccggatttggaattaattggtgacattccaagtacgattaatcacattgaacatgatgctgtgaaggttgagtttgcggaacgggaacagaaaattttatcagatttgaagcaatatatgcatgaaggtgaacaagtgccagcagattggaagtcagatcgggcccatccattagaaattgttaaagaattacggaatgcagtggacgatcatgtgaccgtgacttgtgatattggtagtcatgctatttggatgagtcgttactttcggtcatatgaaccgttaactttaatgatttcaaacggtatgcaaactttaggtgttgccttgccatgggccattggtgcgtcattggtcaaaccaggtgaaaaggtcgtgtcagtcagtggagatggtggcttcttattcagtgctatggaattagaaaccgctgtgcggttgaaggcaccgattgtgcatattgtgtggaacgatagtacttatgatatggtcgcatttcaacagttgaagaaatataatcgtacctcagcagtggattttggtaatatcgatattgtcaagtatgccgaaagttttggtgccaccggtttgcgtgtcgaatcaccagatcaattagctgatgtcttgcgtcaaggtatgaatgcggaaggcccagttattattgatgtgccagttgattacagtgataacattaatttagctagtgataagttgccgaaagaatttggtgaattaatgaagacgaaagcgtta

SEQ ID NO: 72 - DNA, 1680 bp, K. pneumoniae
atggacaaacagtatccggtacgccagtgggcgcacggcgccgatctcgtcgtcagtcagctggaagctcagggagtacgccaggtgttcggcatccccggcgccaaaatcgacaaggtctttgattcactgctggattcctccattcgcattattccggtacgccacgaagccaacgccgcatttatggccgccgccgtcggacgcattaccggcaaagcgggcgtggcgctggtcacctccggtccgggctgttccaacctgatcaccggcatggccaccgcgaacagcgaaggcgacccggtggtggccctgggcggcgcggtaaaacgcgccgataaagcgaagcaggtccaccagagtatggatacggtggcgatgttcagcccggtcaccaaatacgccatcgaggtgacggcgccggatgcgctggcggaagtggtctccaacgccttccgcgccgccgagcagggccggccgggcagcgcgttcgttagcctgccgcaggatgtggtcgatggcccggtcagcggcaaagtgctgccggccagcggggccccgcagatgggcgccgcgccggatgatgccatcgaccaggtggcgaagcttatcgcccaggcgaagaacccgatcttcctgctcggcctgatggccagccagccggaaaacagcaaggcgctgcgccgtttgctggagaccagccatattccagtcaccagcacctatcaggccgccggagcggtgaatcaggataacttctctcgcttcgccggccgggttgggctgtttaacaaccaggccggggaccgtctgctgcagctcgccgacctggtgatctgcatcggctacagcccggtggaatacgaaccggcgatgtggaacagcggcaacgcgacgctggtgcacatcgacgtgctgcccgcctatgaagagcgcaactacaccccggatgtcgagctggtgggcgatatcgccggcactctcaacaagctggcgcaaaatatcgatcatcggctggtgctctccccgcaggcggcggagatcctccgcgaccgccagcaccagcgcgagctgctggaccgccgcggcgcgcagctcaaccagtttgccctgcatcccctgcgcatcgttcgcgccatgcaggatatcgtcaacagcgacgtcacgttgaccgtggacatgggcagcttccatatctggattgcccgctacctgtacacgttccgcgcccgtcaggtgatgatctccaacggccagcagaccatgggcgtcgccctgccctgggctatcggcgcctggctggtcaatcctgagcgcaaagtggtctccgtctccggcgacggcggcttcctgcagtcgagcatggagctggagaccgccgtccgcctgaaagccaacgtgctgcatcttatctgggtcgataacggctacaacatggtcgctatccaggaagagaaaaaatatcagcgcctgtccggcgtcgagtttgggccgatggattttaaagcctatgccgaatccttcggcgcgaaagggtttgccgtggaaagcgccgaggcgctggagccgaccctgcgcgcggcgatggacgtcgacggcccggcggtagtggccatcccggtggattatcgcgataacccgctgctgatgggccagctgcatctgagtcagattctgtaa

SEQ ID NO: 73 - protein, 559 aa, K. pneumoniae
MDKQYPVRQWAHGADLVVSQLEAQGVRQVFGIPGAKIDKVFDSLLDSSIRIIPVRHEANAAFMAAAVGRITGKAGVALVTSGPGCSNLITGMATANSEGDPVVALGGAVKRADKAKQVHQSMDTVAMFSPVTKYAIEVTAPDALAEVVSNAFRAAEQGRPGSAFVSLPQDVVDGPVSGKVLPASGAPQMGAAPDDAIDQVAKLIAQAKNPIFLLGLMASQPENSKALRRLLETSHIPVTSTYQAAGAVNQDNFSRFAGRVGLFNNQAGDRLLQLADLVICIGYSPVEYEPAMWNSGNATLVHIDVLPAYEERNYTPDVELVGDIAGTLNKLAQNIDHRLVLSPQAAEILRDRQHQRELLDRRGAQLNQFALHPLRIVRAMQDIVNSDVTLTVDMGSFHIWIARYLYTFRARQVMISNGQQTMGVALPWAIGAWLVNPERKVVSVSGDGGFLQSSMELETAVRLKANVLHLIWVDNGYNMVAIQEEKKYQRLSGVEFGPMDFKAYAESFGAKGFAVESAEALEPTLRAAMDVDGPAVVAIPVDYRDNPLLMGQLHLSQIL

SEQ ID NO: 74 - DNA, 1665 bp, Lactococcus lactis
atgtctgagaaacaatttggggcgaacttggttgtcgatagtttgattaaccataaagtgaagtatgtatttgggattccaggagcaaaaattgaccgggtttttgatttattagaaaatgaagaaggccctcaaatggtcgtgactcgtcatgagcaaggagctgctttcatggctcaagctgtcggtcgtttaactggcgaacctggtgtagtagttgttacgagtgggcctggtgtatcaaaccttgcgactccgcttttgaccgcgacatcagaaggtgatgctattttggctatcggtggacaagttaaacgaagtgaccgtcttaaacgtgcgcaccaatcaatggataatgctggaatgatgcaatcagcaacaaaatattcagcagaagttcttgaccctaatacactttctgaatcaattgccaacgcttatcgtattgcaaaatcaggacatccaggtgcaactttcttatcaatcccccaagatgtaacggatgccgaagtatcaatcaaagccattcaaccactttcagaccctaaaatggggaatgcctctattgatgacattaattatttagcacaagcaattaaaaatgctgtattgccagtaattttggttggagctggtgcttcagatgctaaagtcgcttcatccttgcgtaatctattgactcatgttaatattcctgtcgttgaaacattccaaggtgcaggggttatttcacatgatttagaacatactttttatggacgtatcggtcttttccgcaatcaaccaggcgatatgcttctgaaacgttctgaccttgttattgctgttggttatgacccaattgaatatgaagctcgtaactggaatgcagaaattgatagtcgaattatcgttattgataatgccattgctgaaattgatacttactaccaaccagagcgtgaattaattggtgatatcgcagcaacattggataatcttttaccagctgttcgtggctacaaaattccaaaaggaacaaaagattatctcgatggccttcatgaagttgctgagcaacacgaatttgatactgaaaatactgaagaaggtagaatgcaccctcttgatttggtcagcactttccaagaaatcgtcaaggatgatgaaacagtaaccgttgacgtaggttcactctacatttggatggcacgtcatttcaaatcatacgaaccacgtcatctcctcttctcaaacggaatgcaaacactcggagttgcacttccttgggcaattacagccgcattgttgcgcccaggtaaaaaagtttattcacactctggtgatggaggcttccttttcacagggcaagaattggaaacagctgtacgtttgaatcttccaatcgttcaaattatctggaatgacggccattatgatatggttaaattccaagaagaaatgaaatatggtcgttcagcagccgttgattttggctatgttgattacgtaaaatatgctgaagcaatgagagcaaaaggttaccgtgcacacagcaaagaagaacttgctgaaattctcaaatcaatcccagatactactggaccggtggtaattgacgttcctttggactattctgataacattaaattagcagaaaaattattgcctgaagagttttattga

SEQ ID NO: 75 - protein, 554 aa, Lactococcus lactis
MSEKQFGANLVVDSLINHKVKYVFGIPGAKIDRVFDLLENEEGPQMVVTRHEQGAAFMAQAVGRLTGEPGVVVVTSGPGVSNLATPLLTATSEGDAILAIGGQVKRSDRLKRAHQSMDNAGMMQSATKYSAEVLDPNTLSESIANAYRIAKSGHPGATFLSIPQDVTDAEVSIKAIQPLSDPKMGNASIDDINYLAQAIKNAVLPVILVGAGASDAKVASSLRNLLTHVNIPVVETFQGAGVISHDLEHTFYGRIGLFRNQPGDMLLKRSDLVIAVGYDPIEYEARNWNAEIDSRIIVIDNAIAEIDTYYQPERELIGDIAATLDNLLPAVRGYKIPKGTKDYLDGLHEVAEQHEFDTENTEEGRMHPLDLVSTFQEIVKDDETVTVDVGSLYIWMARHFKSYEPRHLLFSNGMQTLGVALPWAITAALLRPGKKVYSHSGDGGFLFTGQELETAVRLNLPIVQIIWNDGHYDMVKFQEEMKYGRSAAVDFGYVDYVKYAEAMRAKGYRAHSKEELAEILKSIPDTTGPVVIDVPLDYSDNIKLAEKLLPEEFY

SEQ ID NO: 76 - DNA, 1665 bp, Staphylococcus aureus
  CDS (1)..(1665)
atgactgataaaaagtacactgcagccgatatggttattgatactttgaaaaataatggggtagaatatgtttttggtattccgggtgcaaagatagactatctatttaatgctttaattgatgatggtcctgaacttattgtcactcgtcatgaacaaaatgctgcaatgatggcacaaggtattggaagattaacaggtaaaccgggtgtagtacttgttacaagtggccctggtgtaagtaatttaacgactggactattaacagctacatctgaaggggatcctgtattagcgttaggtggccaagtgaaacgtaatgatttattacgattaacgcatcaaagtattgataatgctgcgctattaaaatattcatcaaaatacagtgaagaagtacaagatcctgaatcattatcagaagttatgacaaatgcaattcgaattgctacttcaggaaaaaatggcgcaagttttattagtattccgcaagacgttatttcttcaccagttgaatctaaagctatatcactttgccaaaaaccaaatttaggagtaccgagtgaacaagatattaatgatgtcattgaagcgattaaaaatgcatcatttcctgttttattagctggtatgagaagttcaagtgcagaagaaacaaatgccattcgcaaattagttgagcgcacgaatttaccagttgtagaaacattccaaggtgcaggtgtaattagtcgtgaattagaaaatcatttcttcggtcgtgtgggcttattccgcaatcaagttggtgatgaattattacgtaaaagtgatttagttgttacaatcggttatgatccaattgaatacgaagctagtaactggaataaagaattagaaacacaaattatcaatattgacgaagttcaagctgaaattactaattatatgcaaccgaaaaaagagttgattggtaatattgctaaaacgattgaaatgatttctgaaaaagtggatgagccatttataaatcaacaacatttagacgaattagaacaattaagaacacatattgatgaagaaactggtattaaagcgacgcatgaagaaggaattctacatccagtggaaattattgaatctatgcaaaaggtattaactgatgatactactgtaacagttgatgttggaagtcactatatttggatggcacgtaatttcagaagttacaatccaagacatttattatttagcaatggtatgcaaacgcttggtgtagcattaccgtgggcaatttcagctgcacttgtgcgccctaatacgcaagttgtgtccgttgctggcgatggtggctttttattttcatcacaagatttagaaacggccgtacgtaaaaatttaaatatcatccagcttatttggaatgatggaaaatataacatggttgaattccaagaagaaatgaaatataaacgttcgtcaggtgtagacttcggtcctgtagattttgtaaaatatgcagaatcatttggcgcgaaaggtttacgagttactaatcaagaagaattagaagcggcaattaaagagggctatgaaacagatggtccagtattaattgatatacctgtaaattacaaagataatatcaaactttcaacaaatatgttacctgacgtatttaactaa

SEQ ID NO: 77 - protein, 554 aa, Staphylococcus aureus
MTDKKYTAADMVIDTLKNNGVEYVFGIPGAKIDYLFNALIDDGPELIVTRHEQNAAMMAQGIGRLTGKPGVVLVTSGPGVSNLTTGLLTATSEGDPVLALGGQVKRNDLLRLTHQSIDNAALLKYSSKYSEEVQDPESLSEVMTNAIRIATSGKNGASFISIPQDVISSPVESKAISLCQKPNLGVPSEQDINDVIEAIKNASFPVLLAGMRSSSAEETNAIRKLVERTNLPVVETFQGAGVISRELENHFFGRVGLFRNQVGDELLRKSDLVVTIGYDPIEYEASNWNKELETQIINIDEVQAEITNYMQPKKELIGNIAKTIEMISEKVDEPFINQQHLDELEQLRTHIDEETGIKATHEEGILHPVEIIESMQKVLTDDTTVTVDVGSHYIWMARNFRSYNPRHLLFSNGMQTLGVALPWAISAALVRPNTQVVSVAGDGGFLFSSQDLETAVRKNLNIIQLIWNDGKYNMVEFQEEMKYKRSSGVDFGPVDFVKYAESFGAKGLRVTNQEELEAAIKEGYETDGPVLIDIPVNYKDNIKLSTNMLPDVFN

SEQ ID NO: 78 - DNA, 1698 bp, Listeria monocytogenes
  CDS (1)..(1698)
atggcgaaactagaaaaagaccaagaaaaagtaataacacaagggaaatcaggagcggatttagttgtagacagcttaattaatcaaggtgttacgcatgtattcgggattccgggagcgaaaattgataaagtttttgatgtgatggaagaacgtggaccagaattaattgtcagtcgtcatgaacaaaatgcggcgtttatggctgctgctatcggtcgtctaaccgggaaacctggtgttgtacttgtaactagtggacctggcgcatcgaatcttgcaacagggcttgtaaccgcaactgcagaaggagatccagtcgttgcgattgctggtaacgtaacaaggcaagaccgcttaaaaagaacccaccaatcaatggataatgcagcacttttccgtccgattacaaaatacagcgaagaagtagttcacgccgaaagtattccagaagcaatcactaacgcttttcgctcggcaacagaaccaaaccaaggcgctgcttttgtcagtttgccacaagatatcgtgaacgaaccaaacgtaccagtaaaagcgattcgcccacttgctaaaccagaaaatggtcctgcttccaaagaacaagttgcaaaacttgttacacgtttgaaaaaagcgaaattaccggtattgctattgggtatgcgagcatctagtccagaagtaactggtgcaattcgtcgcttactccaaaaaacaagtatcccagtagtagaaactttccaagcagctggcgtcatttcacgcgacttagaagataacttctttggacgtgttggtctgttccgcaaccaaccaggggatattttgttaaataaagctgatttagttattacagtgggttatgatccaattgaatacgatccaaaagcttggaatgcctctggtgatagaacgattgtccatttagacgacattcgcgctgatattgatcattattaccaaccagtgacagagctagtcggaaacatcgcgcttactttagaccgagtgaatgcgaaattcagcggtttagaattagcggaaaaagaacttgaaacattaaaagaacttcatgctcaattagaagagcgagatgttccgccagaaagtgatgaaactaaccgagtacatccattgtcggtcattcaaacactacgttcggcaattgatgacaacgtaactgtgacagtcgacgttggttcacattatatttggatggcacgtcatttccgctcctatgaaccacgccgtctgcttttcagtaacggtatgcaaacgcttggtgttgcgcttccttggggaattgctgcaacacttgtacatccgggtgaaaaagtggtttcgatttctggtgacggtggtttcttattttccgcgatggaattagaaacagctgtccgcttgcgtgcgccacttgtacacctagtatggaatgacggaagctatgacatggttgctttccaacaaaaaatgaaatacggcaaagaagcagctgttcgttttggcgatgttgatatcgtaaaatttgcagaaagtttcggagcaaaaggtcttcgcgtaacaaatccagcagaactttctgatgtgttaaaagaagcgcttgaaacagaaggacccgtcgttgtagatattccaattgattaccgtgataacatcaaacttggcgaaactttactacctgaccaattttattaa

SEQ ID NO: 79 - protein, 565 aa, Listeria monocytogenes
MAKLEKDQEKVITQGKSGADLVVDSLINQGVTHVFGIPGAKIDKVFDVMEERGPELIVSRHEQNAAFMAAAIGRLTGKPGVVLVTSGPGASNLATGLVTATAEGDPVVAIAGNVTRQDRLKRTHQSMDNAALFRPITKYSEEVVHAESIPEAITNAFRSATEPNQGAAFVSLPQDIVNEPNVPVKAIRPLAKPENGPASKEQVAKLVTRLKKAKLPVLLLGMRASSPEVTGAIRRLLQKTSIPVVETFQAAGVISRDLEDNFFGRVGLFRNQPGDILLNKADLVITVGYDPIEYDPKAWNASGDRTIVHLDDIRADIDHYYQPVTELVGNIALTLDRVNAKFSGLELAEKELETLKELHAQLEERDVPPESDETNRVHPLSVIQTLRSAIDDNVTVTVDVGSHYIWMARHFRSYEPRRLLFSNGMQTLGVALPWGIAATLVHPGEKVVSISGDGGFLFSAMELETAVRLRAPLVHLVWNDGSYDMVAFQQKMKYGKEAAVRFGDVDIVKFAESFGAKGLRVTNPAELSDVLKEALETEGPVVVDIPIDYRDNIKLGETLLPDQFY

SEQ ID NO: 80 - DNA, 1680 bp, Streptococcus mutans
  CDS (1)..(1680)
atgaccgaaataaataaggaaggctatggggctgacctgattgtagacagcctcattaatcatgatgtcaactatgtttttggaatccctggtgcaaaaattgatcgtgtctttgataccttagaagataaggggccagaacttattgtagcacgccatgagcaaaatgctgcttttatggctcaaggaattggccgtattactggtgagcctggtgttgtgattacaaccagcggtcccggtgtttccaatctggtgactggtcttgttactgcgacagctgagggagatcctgtccttgctattggtggtcaggttaaacgtgctgatttgctcaaacgggctcaccagtcaatgaataatgttgctatgctcgatcccattaccaaatattcagcagaaattcaggatcccgcaacactttcagaaaatattgctaatgcctatcgtttggctaaagcaggaaagccgggagctagtttcttatctattcctcaagatataactgatagtcctgttactgtcaaggcgattaagcccttgacagatcctaaactaggttcagcgtcagttgctgatattaattatttggcacaggccataaaaaatgcggtccttcctgtcttacttttaggaaatggtgcgtcaacggctgcagttacagcttctattcgccgtttgttaggagctgtcaagctgccagtcgttgaaactttccaaggagctggtattgtttcaagagatttagaagaggacactttttttggtcgtgtggggctttttcgtaatcagcccggagatatgttgctgaagcgttctgacttagttatcgctattggctatgatcctattgaatatgaagcgcgcaattggaatgctgaaatttcggctcgcattatcgttattgatgttgctccagctgaaattgatacttatttccaacctgaacgtgaattaattggtgatatagctgaaacacttgatttactcctacctgctattagtggctactcacttccaaaaggttctcttgactatctcaaaggccttcgtgataatgtagtagaagatgtcaaatttgataagacagtcaaatccggtctggttcatccgcttgatgtgattgatgtccttcaaaagcaaacgactgatgatatgacagtaacggttgatgttggcagccattatatttggatggctcgttattttaaaagctatgaagcacggcacttacttttctcaaatggtatgcaaaccttaggtgttgctttgccttgggcaatttcggcagctcttgtacggccaaatgagaagattatttctatttcaggtgatggtggtttcctcttttctggccaagaattggaaacagctgttcgtttacatttaccaattgttcatatcatttggaatgatggtaaatataatatggttgaattccaagaagaaatgaaatacggccgttcagcaggtgttgattttggtcctgttgattttgtcaagtatgctgatagtttcggtgctaaaggttaccgtgctgatagtaaagaaaagtttgatcaagttcttcaaacagcactcaaggaagctgcaaatggcccagttctcattgatgttccaatggactataaagataatgtaaaattgggtgaaactattttgcctgatgaattctactaa

SEQ ID NO: 81 - protein, 559 aa, Streptococcus mutans
MTEINKEGYGADLIVDSLINHDVNYVFGIPGAKIDRVFDTLEDKGPELIVARHEQNAAFMAQGIGRITGEPGVVITTSGPGVSNLVTGLVTATAEGDPVLAIGGQVKRADLLKRAHQSMNNVAMLDPITKYSAEIQDPATLSENIANAYRLAKAGKPGASFLSIPQDITDSPVTVKAIKPLTDPKLGSASVADINYLAQAIKNAVLPVLLLGNGASTAAVTASIRRLLGAVKLPVVETFQGAGIVSRDLEEDTFFGRVGLFRNQPGDMLLKRSDLVIAIGYDPIEYEARNWNAEISARIIVIDVAPAEIDTYFQPERELIGDIAETLDLLLPAISGYSLPKGSLDYLKGLRDNVVEDVKFDKTVKSGLVHPLDVIDVLQKQTTDDMTVTVDVGSHYIWMARYFKSYEARHLLFSNGMQTLGVALPWAISAALVRPNEKIISISGDGGFLFSGQELETAVRLHLPIVHIIWNDGKYNMVEFQEEMKYGRSAGVDFGPVDFVKYADSFGAKGYRADSKEKFDQVLQTALKEAANGPVLIDVPMDYKDNVKLGETILPDEFY

SEQ ID NO: 82 - DNA, 1683 bp, Streptococcus thermophilus
  CDS (1)..(1683)
gtgttcatgtcagaagaaaagcaattgtatggtgcagatttagtggttgatagtttgatcaaccatgatgttgagtatgtctttgggattccaggcgcaaaaatcgatagggtttttgataccttggaagataagggacctgaattgattgttgcccgtcatgagcaaaatgctgcttttatggctcaaggtgttggacgtattactgggaaaccaggtgtagtattggtaacatctggtccaggtgtctccaatttggctactggtttggtaacagcgacggatgaaggagaccctgttcttgctattggtggtcaggttaagcgtgcagatctcttgaaacgtgcccaccaatcaatgaataacgttgctatgcttgagccaattaccaaatatgctgctgaagtacatgatgctaacaccctttctgaaacggttgctaatgcctatcgtcacgctaagtcagggaaaccaggtgcaagcttcatttcaattcctcaagacgtgacggatgctccggtcagtgttaaggctattaagcctatgacagatccaaaacttggttcagcatctgtttctgatattaactatctagcacaagccattaaaaatgcagtgttgccagtctttcttttggggaatggtgcctcatcagaagccgtaacttactctattcgccaaattttgaagcatgttaaattgccagttgttgaaactttccaaggtgccggtatcgtgtcacgtgaccttgaagaagatactttctttggtcgtgtaggtcttttccgtaaccaacccggagacatgttgcttaaaaaatccgacttagttattgccattggttatgatccaatcgaatatgaagcacgtaactggaatgctgaaatttcagcacgtatcatcgttattgatgtcgagccggccgaggtggacacttacttccaaccggaacgtgaattgattggtaatgtagaagcgagcttagacttgcttttgcccgctattcaaggttataaattgcctgaaggtgcggttgaatatcttaaaggtttgaaaaacaatgttgttgaggatgttaagtttgaccgtcagcctgatgaaggtacggtgcatccgctagatttcatcgaaaatttgcaagaacacacagatgatgatatgactgttacgtttgatgttggtagtcactatatttggatggcacgttatctcaaatcgtatgaaccacgtcatttgcttttctcaaatgggatgcaaacgataggtattgctattacatgggctatctctgcagcattggttcgtcctaagacaaaagtgatttctgtatctggtgatggtggtttcctcttctcagcacaagaattggaaacagcagttcgtttgaaattgccaattgtccatattatctggaacgatggtcattacaatatggtggaattccaggaagaaatgaagtacggtcgttcatctggggttgactttggtcctgtagattttgtaaaatatgctgagagctttggagccaaaggttatcgtgcaacaagtaaagcagcgtttgctagcttgcttcaagaggctttgactcaggctgtagatggaccagtccttattgatgttccaattgactataaagataacattaaactcggcgaaactattttgccagatgaattttactaa

SEQ ID NO: 83 - protein, 560 aa, Streptococcus thermophilus
VFMSEEKQLYGADLVVDSLINHDVEYVFGIPGAKIDRVFDTLEDKGPELIVARHEQNAAFMAQGVGRITGKPGVVLVTSGPGVSNLATGLVTATDEGDPVLAIGGQVKRADLLKRAHQSMNNVAMLEPITKYAAEVHDANTLSETVANAYRHAKSGKPGASFISIPQDVTDAPVSVKAIKPMTDPKLGSASVSDINYLAQAIKNAVLPVFLLGNGASSEAVTYSIRQILKHVKLPVVETFQGAGIVSRDLEEDTFFGRVGLFRNQPGDMLLKKSDLVIAIGYDPIEYEARNWNAEISARIIVIDVEPAEVDTYFQPERELIGNVEASLDLLLPAIQGYKLPEGAVEYLKGLKNNVVEDVKFDRQPDEGTVHPLDFIENLQEHTDDDMTVTFDVGSHYIWMARYLKSYEPRHLLFSNGMQTIGIAITWAISAALVRPKTKVISVSGDGGFLFSAQELETAVRLKLPIVHIIWNDGHYNMVEFQEEMKYGRSSGVDFGPVDFVKYAESFGAKGYRATSKAAFASLLQEALTQAVDGPVLIDVPIDYKDNIKLGETILPDEFY

SEQ ID NO: 84 - DNA, 1665 bp, Vibrio angustum
  CDS (1)..(1665)
atgtcggataaaaccgtctctggtgctgaactggttgttgaaactttaaatgcacataacgttccacacatttttggtattcctggagcaaaggtggatgctgttttcgatgctgtttgtgataacggaccagaaatcattatttgtcatcatgaacaaaatgcagcgtttatggcagcagcaactgggcgtttaacgggtaaagcaggcatttgtttagcaacctctggaccaggcgcatcaaaccttgtcacaggcgttgcaacagcgaatagtgaaggtgatcctgtggttgcacttgcaggtgctgtacctctttctatgtattctcacaatactcatcaatccatggatacccgttcactgtttactcctatcaccaagttttcagcagaagtgatggatagcagctcggtatctgatgttgtacataaagcttttcgtattgcagagcaacctacccaaggtgctagctttgttagtctaccgcaagatattctaactaaccgtattccttaccagccagtacaacagcctaatccaattttgttcggtggtgcacacccacaagctattcgtcaggctgctgatcgcattaatgctgcaaaaaatccggtgttattactgggcatggatgcaagccagccttttgttgctgatgctattcgccaactactcaaacaaacaccaattgccgttgtgaatacgtttgccgcagctggggttatttctcatgatttatacaactgctttttaggtcgtgttggcttatttaaaaatcaacccggtgatattgcattaaacagtgcagatttaatcattaccattggctacagcccaattgaatacgatccgattctttggaataaagatgcaaacacaccaattattcatattggttatcaacaagcagatttagaaattagctataaccctgtttgtgaagttgtgggtgacttagcggtgtctgtcacgtctattgcttctgaattagataagcgagaatcattagaaaataaccaacaaatccaattattacgccacgatttacaacatattatgcagatgggggtaaataaaacctcaacaaacggcgttcacccgcttcgttttgttcatgagttacgtcgctttgttagtgacgacaccactgtatgttgtgatgtaggctctatttatatttggatggcacgttacttccacagctttgaacctcgtcgtttattgttcagcaatggccaacaaacattgggcgtagctttaccttgggcaattgcagcttcccttcttcaccctaatgaaaaagtaatttccatgtctggtgatggtggcttcctattctcatcaatggaattagccacggccgttcgccataaatgtaatatcgttcactttgtttggacagatcacagttatgacatggttaagatccaacagcttaaaaagtatggtcgagagagtgccgtcagctttataggtcctgatattgttaagtacgcagaaagcttcggcgcacatggtttagcgatcaatactgccgatgatattgagcctgttatgcgaaaagctatgagcttaagtggcccagtattggtcaacgtcaatgttgattatagcgataacagtcgcctacttgatcaacttcatccatgccaacaagattaa

SEQ ID NO: 85 - protein, 554 aa, Vibrio angustum
MSDKTVSGAELVVETLNAHNVPHIFGIPGAKVDAVFDAVCDNGPEIIICHHEQNAAFMAAATGRLTGKAGICLATSGPGASNLVTGVATANSEGDPVVALAGAVPLSMYSHNTHQSMDTRSLFTPITKFSAEVMDSSSVSDVVHKAFRIAEQPTQGASFVSLPQDILTNRIPYQPVQQPNPILFGGAHPQAIRQAADRINAAKNPVLLLGMDASQPFVADAIRQLLKQTPIAVVNTFAAAGVISHDLYNCFLGRVGLFKNQPGDIALNSADLIITIGYSPIEYDPILWNKDANTPIIHIGYQQADLEISYNPVCEVVGDLAVSVTSIASELDKRESLENNQQIQLLRHDLQHIMQMGVNKTSTNGVHPLRFVHELRRFVSDDTTVCCDVGSIYIWMARYFHSFEPRRLLFSNGQQTLGVALPWAIAASLLHPNEKVISMSGDGGFLFSSMELATAVRHKCNIVHFVWTDHSYDMVKIQQLKKYGRESAVSFIGPDIVKYAESFGAHGLAINTADDIEPVMRKAMSLSGPVLVNVNVDYSDNSRLLDQLHPCQQD

SEQ ID NO: 86 - DNA, 1689 bp, Bacillus cereus
ttgagtacaggtgtaaaagcaaacgacgtgaagacaaaaacaaaaggagcagatcttgttgttgattgtttaattaaacaaggtgttacacatgttttcggtattccaggagcaaagattgactctgtatttgatgtactgcaagaaagaggaccagagttaattgtttgtcgtcatgaacaaaatgcagcatttatggcagctgctattggtagattaacaggaaaaccgggcgtatgtcttgtaacttcaggaccagggacatcaaatttagcgacaggtcttgttactgcgaatgcggagagtgatcccgttgttgctttagctggtgcagttccgcgtacggatcgattaaaacgtacacatcaatctatggataatgctgcactattcgaaccaatcacaaaatatagcgtagaagtagagcatcctgataatgtgccagaagcattatcaaatgcattccgaagtgcgacttctacaaatccaggagcaactttagtaagtttgccgcaagacgttatgactgcggaaacgactgtagagtctatcggtgcgctttctaagccacagcttggaatcgctcccacacatgatattacatatgtagtagataaaataaaagcagcgaaattaccagttattttactcggtatgagagcgagcacaaatgaagtgacgaaagccgttcgtaaattaattgcggatacagaacttcctgtcgttgaaacatatcaagcggctggtgccatttcacgtgagttagaagatcatttcttcggccgtgttggactattccgtaaccaaccaggtgatattttactagaagaggcagatcttgttatttctatcggttatgatccaattgagtatgatccaaagttctggaataaacttggagacagaacgattattcatcttgatgaccatcaagcagatatagatcatgattaccaaccagagcgtgaattaattggtgatattgccttaacagtaaatagcatcgcagaaaagttaccgaaacttgtgttaagtacgaaatcagaagcagtgttagaacgattacgcgcgaaattatcagaacaagcagaagttccaaatcgtccttcagaaggtgttacacatccgcttcaagtgattcgtacacttcgttctttaattagtgacgacacaaccgttacatgtgacatcggttcccattctatttggatggcgagatgtttccgttcttatgaaccacgtagattattatttagtaacggtatgcagacgttaggtgttgcacttccttgggcaattgctgctactttagtagaaccaggtaaaaaagtagtttccgtatcaggtgacggtggtttcttattctcagcgatggagttagaaacggcggtacgtttaaattctccaatcgtccatcttgtttggagagacggcacatatgatatggttgcattccaacaaatgatgaaatacggcagaacatcagctacagagtttggtgatgttgatcttgttaaatatgcggaaagtttcggggcgttaggtcttcgtgttaacacgcctgatgaattagaaggggtattgaaagaagcactagcagcagacggccctgtcattattgatattccaattgactatcgtgacaacattaaattaagcgaaaaattattaccaaaccaattaaactaa

SEQ ID NO: 87 - protein, 562 aa, Bacillus cereus
MSTGVKANDVKTKTKGADLVVDCLIKQGVTHVFGIPGAKIDSVFDVLQERGPELIVCRHEQNAAFMAAAIGRLTGKPGVCLVTSGPGTSNLATGLVTANAESDPVVALAGAVPRTDRLKRTHQSMDNAALFEPITKYSVEVEHPDNVPEALSNAFRSATSTNPGATLVSLPQDVMTAETTVESIGALSKPQLGIAPTHDITYVVDKIKAAKLPVILLGMRASTNEVTKAVRKLIADTELPVVETYQAAGAISRELEDHFFGRVGLFRNQPGDILLEEADLVISIGYDPIEYDPKFWNKLGDRTIIHLDDHQADIDHDYQPERELIGDIALTVNSIAEKLPKLVLSTKSEAVLERLRAKLSEQAEVPNRPSEGVTHPLQVIRTLRSLISDDTTVTCDIGSHSIWMARCFRSYEPRRLLFSNGMQTLGVALPWAIAATLVEPGKKVVSVSGDGGFLFSAMELETAVRLNSPIVHLVWRDGTYDMVAFQQMMKYGRTSATEFGDVDLVKYAESFGALGLRVNTPDELEGVLKEALAADGPVIIDIPIDYRDNIKLSEKLLPNQLN

SEQ ID NO: 88 - DNA, 1014 bp, Pseudomonas fluorescens
atgaaagttttctacgataaagactgcgacctgtcgatcatccaaggtaagaaagttgccatcatcggctacggttcccagggccacgctcaagcatgcaacctgaaggattccggcgtagacgtgactgttggcctgcgtaaaggctcggctaccgttgccaaggctgaagcccacggcttgaaagtgaccgacgttgctgcagccgttgccggtgccgacttggtcatgatcctgaccccggacgagttccagtcccagctgtacaagaacgaaatcgagccgaacatcaagaagggcgccactctggccttctcccacggcttcgcgatccactacaaccaggttgtgcctcgtgccgacctcgacgtgatcatgatcgcgccgaaggctccaggccacaccgtacgttccgagttcgtcaagggcggtggtattcctgacctgatcgcgatctaccaggacgcttccggcaacgccaagaacgttgccctgtcctacgccgcaggcgtgggcggcggccgtaccggcatcatcgaaaccaccttcaaggacgagactgaaaccgacctgttcggtgagcaggctgttctgtgtggcggtaccgtcgagctggtcaaagccggtttcgaaaccctggttgaagctggctacgctccagaaatggcctacttcgagtgcctgcacgaactgaagctgatcgttgacctcatgtacgaaggcggtatcgccaacatgaactactcgatctccaacaacgctgaatacggcgagtacgtgactggtccagaagtcatcaacgccgaatcccgtcaggccatgcgcaatgctctgaagcgcatccaggacggcgaatacgcgaagatgttcatcagcgaaggcgctaccggctacccatcgatgaccgccaagcgtcgtaacaacgctgctcacggtatcgaaatcatcggcgagcaactgcgctcgatgatgccttggatcggtgccaacaaaatcgtcgacaaagccaagaac

SEQ ID NO: 89 - protein, 338 aa, Pseudomonas fluorescens
MKVFYDKDCDLSIIQGKKVAIIGYGSQGHAQACNLKDSGVDVTVGLRKGSATVAKAEAHGLKVTDVAAAVAGADLVMILTPDEFQSQLYKNEIEPNIKKGATLAFSHGFAIHYNQVVPRADLDVIMIAPKAPGHTVRSEFVKGGGIPDLIAIYQDASGNAKNVALSYAAGVGGGRTGIIETTFKDETETDLFGEQAVLCGGTVELVKAGFETLVEAGYAPEMAYFECLHELKLIVDLMYEGGIANMNYSISNNAEYGEYVTGPEVINAESRQAMRNALKRIQDGEYAKMFISEGATGYPSMTAKRRNNAAHGIEIIGEQLRSMMPWIGANKIVDKAKN

SEQ ID NO: 90 - DNA, 1014 bp, artificial sequence
  PF5 -ilvC coding seq optimized for expression in Lactobacillus plantarum
atgaaagtcttttatgataaggattgtgatttgtcaattattcaaggtaagaaggtcgctattattggttatggttcacaaggtcacgcccaagcctgtaatttgaaggatagtggtgttgatgttacggttggtttacggaagggtagtgctacggttgcgaaagccgaagcgcatggtttgaaggttacggatgttgcagctgccgtcgccggtgcggatttggttatgattttaacgccagatgaatttcaaagtcaattatataaaaatgaaattgaaccaaatattaaaaaaggtgctacgttagcttttagtcatggttttgcgattcattataatcaagttgtcccacgggctgatttagatgtcattatgattgcaccaaaagctccaggtcatacggttcgtagtgaatttgtcaagggtggtggtattccagatttgattgcaatttatcaagatgcaagtggtaatgccaaaaatgtcgctttaagttatgcggccggtgttggtggtggtcggacgggtattattgaaacgacgtttaaagatgaaacggaaacggatttgtttggtgaacaagccgtcttatgtggtggtacggttgaattagttaaggcgggttttgaaacgttggttgaagctggttatgcaccagaaatggcgtattttgaatgtttacatgaattaaaattaattgttgatttgatgtatgaaggtggtattgcaaatatgaattatagtattagtaataatgcagaatatggtgaatatgttacgggtccagaagttattaatgcagaaagtcggcaagcaatgcgtaatgccttaaaacggattcaagatggtgaatatgccaaaatgtttatttcagaaggtgcgacgggttatccaagtatgacggctaagcggcggaataatgccgcccatggtattgaaattattggtgaacaattacggagtatgatgccatggattggtgctaataaaattgttgataaggcaaagaat

SEQ ID NO: 91 - DNA, 1014 bp, artificial sequence
  PF5 ilvC codon optimized for expression in Saccharomyces cerevisiae
atgaaggtgttttacgataaagactgcgatctgagcatcatccagggaaagaaggttgctattataggatatggttcccaaggacacgcacaagcctgtaacttgaaagattctggggtcgacgtgacagtaggtctgagaaaaggtagtgctaccgttgcaaaggctgaagcacatggcttgaaagtcacagatgttgcagcggctgttgccggcgctgatttagtcatgattttaacgccagatgaatttcaatcgcaattgtacaaaaatgaaatagaaccaaacattaagaagggcgctaccttggccttcagtcatggatttgccattcattacaatcaagtagtccccagggcagatttggacgttattatgattgcacctaaggctccggggcatactgttaggagcgaatttgttaagggtggtggtattccagatttgatcgctatataccaagacgcaagcggaaacgctaagaatgtagctttaagctacgcagcaggagttggtggcgggagaacgggtataatagaaaccacttttaaagacgagactgagacagatttatttggagaacaagcggttctgtgcggaggaactgttgaattggttaaagcaggctttgagacgcttgtcgaagcagggtacgctcccgaaatggcatacttcgaatgtctacatgaattgaagttgatagtagacttaatgtatgaaggtggtatagctaatatgaactattccatttcaaataatgcagaatatggtgagtatgtcaccggacctgaagtcattaacgcagaatcaagacaagccatgagaaatgccttgaaacgtatccaggacggtgaatacgctaagatgttcataagtgaaggcgctacgggttacccgagtatgactgctaaaagaagaaacaatgcagcacatggtatcgaaattattggtgaacagttaaggtctatgatgccctggatcggtgctaataagatcgtagacaaggcgaaaaat

SEQ ID NO: 92 - DNA, 1047 bp, Saccharomyces cerevisiae
atgttgaagcaaatcaacttcggtggtactgttgaaaccgtctacgaaagagctgactggccaagagaaaagttgttggactacttcaagaacgacacttttgctttgatcggttacggttcccaaggttacggtcaaggtttgaacttgagagacaacggtttgaacgttatcattggtgtccgtaaagatggtgcttcttggaaggctgccatcgaagacggttgggttccaggcaagaacttgttcactgttgaagatgctatcaagagaggtagttacgttatgaacttgttgtccgatgccgctcaatcagaaacctggcctgctatcaagccattgttgaccaagggtaagactttgtacttctcccacggtttctccccagtcttcaaggacttgactcacgttgaaccaccaaaggacttagatgttatcttggttgctccaaagggttccggtagaactgtcagatctttgttcaaggaaggtcgtggtattaactcttcttacgccgtctggaacgatgtcaccggtaaggctcacgaaaaggcccaagctttggccgttgccattggttccggttacgtttaccaaaccactttcgaaagagaagtcaactctgacttgtacggtgaaagaggttgtttaatgggtggtatccacggtatgttcttggctcaatacgacgtcttgagagaaaacggtcactccccatctgaagctttcaacgaaaccgtcgaagaagctacccaatctctatacccattgatcggtaagtacggtatggattacatgtacgatgcttgttccaccaccgccagaagaggtgctttggactggtacccaatcttcaagaatgctttgaagcctgttttccaagacttgtacgaatctaccaagaacggtaccgaaaccaagagatctttggaattcaactctcaacctgactacagagaaaagctagaaaaggaattagacaccatcagaaacatggaaatctggaaggttggtaaggaagtcagaaagttgagaccagaaaaccaa

SEQ ID NO: 93 - protein, 349 aa, Saccharomyces cerevisiae
MLKQINFGGTVETVYERADWPREKLLDYFKNDTFALIGYGSQGYGQGLNLRDNGLNVIIGVRKDGASWKAAIEDGWVPGKNLFTVEDAIKRGSYVMNLLSDAAQSETWPAIKPLLTKGKTLYFSHGFSPVFKDLTHVEPPKDLDVILVAPKGSGRTVRSLFKEGRGINSSYAVWNDVTGKAHEKAQALAVAIGSGYVYQTTFEREVNSDLYGERGCLMGGIHGMFLAQYDVLRENGHSPSEAFNETVEEATQSLYPLIGKYGMDYMYDACSTTARRGALDWYPIFKNALKPVFQDLYESTKNGTETKRSLEFNSQPDYREKLEKELDTIRNMEIWKVGKEVRKLRPENQ

SEQ ID NO: 94 - DNA, 1713 bp, Lactococcus lactis
atggaattcaaatataacggaaaagttgaatcaatagagctcaataagtattcaaaaacattgacacaagacccaacacagccagcgacccaagccatgcactatggcattggttttaaagatgaggacttcaaaaaagctcaggtcggaatcgtcagcatggattgggacggaaatccctgtaacatgcacttgggcacactcggtagtaaaatcaaaaattctgtcaatcaaactgacggactgattgggcttcaatttcacacgattggggtttctgacggaattgccaacggaaagcttggcatgagatattccttggtcagtcgtgaagttattgctgacagtattgaaaccaacgctggcgccgaatattacgatgcaattgtcgctgttcccggttgtgacaaaaatatgccaggctcaatcattggcatggctcggctcaatcgtccgtcaattatggtttatggtggaacgattgaacatggcgaatacaaaggcgaaaagttaaatattgtttcggcttttgaagcgcttggacaaaaaatcactggaaatatttccgaggaagattatcacggcgtcatttgtaatgccattccgggacagggtgcttgtgggggcatgtatacagcaaatacactggcttcggcaattgaaactttgggaatgagtttgccttattcggcttcaaatccagcggtcagtcaagaaaaagaagacgaatgtgatgaaattggtctggcaatcaaaaatttgctagaaaaagacatcaaaccaagcgatatcatgaccaaggaagcttttgaaaatgccataacgatcgtcatggttctcggtggttcaactaatgctgtgcttcatatcattgccatggctaatgccatcggtgtcgaaattacgcaagatgattttcaacgtatttccgatgtcacgcctgtgcttggcgacttcaagccaagtggcaagtacatgatggaagatttgcacaaaattggtggcgtgcctgctgttttgaaatatttgctcaaagagggcaagcttcatggcgactgtttgacagttactggtaaaactctagctgaaaatgttgaaacagcactggatttggactttgacagccaagacattattcgaccacttgaaaatcctatcaaagcaacaggtcatttgcaaattctctatggcaatcttgctgaaggtggttctgtggcaaaaatttctggaaaagaaggggaatttttcaaaggaacagctcgtgtctttgatggcgaacaacattttattgacggaattgagtcaggtcgtttgcacgcaggagatgtcgctgtcattcgtaatatcggtccagtcggaggcccaggaatgccagaaatgttaaaaccgacctcagccttgattggagcaggtcttggaaaatcttgtgctttgattactgacggacgtttctctggtggcacacatggctttgtggttggccacattgtccccgaagcggttgagggtggattgattggcttggtcgaagatgatgatattatcgagattgatgcggtcaataacagcattagtttgaaagttgctgacgatgagattgctagacgacgtgccaattatcaaaaacctgctcctaaagcaacgcgtggggttcttgctaaatttgctaaactcacgcgcccagccagtgaaggctgtgtgactgatttatag

SEQ ID NO: 95 - protein, 570 aa, Lactococcus lactis
MEFKYNGKVESIELNKYSKTLTQDPTQPATQAMHYGIGFKDEDFKKAQVGIVSMDWDGNPCNMHLGTLGSKIKNSVNQTDGLIGLQFHTIGVSDGIANGKLGMRYSLVSREVIADSIETNAGAEYYDAIVAVPGCDKNMPGSIIGMARLNRPSIMVYGGTIEHGEYKGEKLNIVSAFEALGQKITGNISEEDYHGVICNAIPGQGACGGMYTANTLASAIETLGMSLPYSASNPAVSQEKEDECDEIGLAIKNLLEKDIKPSDIMTKEAFENAITIVMVLGGSTNAVLHIIAMANAIGVEITQDDFQRISDVTPVLGDFKPSGKYMMEDLHKIGGVPAVLKYLLKEGKLHGDCLTVTGKTLAENVETALDLDFDSQDIIRPLENPIKATGHLQILYGNLAEGGSVAKISGKEGEFFKGTARVFDGEQHFIDGIESGRLHAGDVAVIRNIGPVGGPGMPEMLKPTSALIGAGLGKSCALITDGRFSGGTHGFVVGHIVPEAVEGGLIGLVEDDDIIEIDAVNNSISLKVADDEIARRRANYQKPAPKATRGVLAKFAKLTRPASEGCVTDL

SEQ ID NO: 96 - DNA, 1713 bp, Streptococcus mutans
atgactgacaaaaaaactcttaaagacttaagaaatcgtagttctgtttacgattcaatggttaaatcacctaatcgtgctatgttgcgtgcaactggtatgcaagatgaagactttgaaaaacctatcgtcggtgtcatttcaacttgggctgaaaacacaccttgtaatatccacttacatgactttggtaaactagccaaagtcggtgttaaggaagctggtgcttggccagttcagttcggaacaatcacggtttctgatggaatcgccatgggaacccaaggaatgcgtttctccttgacatctcgtgatattattgcagattctattgaagcagccatgggaggtcataatgcggatgcttttgtagccattggcggttgtgataaaaacatgcccggttctgttatcgctatggctaacatggatatcccagccatttttgcttacggcggaacaattgcacctggtaatttagacggcaaagatatcgatttagtctctgtctttgaaggtgtcggccattggaaccacggcgatatgaccaaagaagaagttaaagctttggaatgtaatgcttgtcccggtcctggaggctgcggtggtatgtatactgctaacacaatggcgacagctattgaagttttgggacttagccttccgggttcatcttctcacccggctgaatccgcagaaaagaaagcagatattgaagaagctggtcgcgctgttgtcaaaatgctcgaaatgggcttaaaaccttctgacattttaacgcgtgaagcttttgaagatgctattactgtaactatggctctgggaggttcaaccaactcaacccttcacctcttagctattgcccatgctgctaatgtggaattgacacttgatgatttcaatactttccaagaaaaagttcctcatttggctgatttgaaaccttctggtcaatatgtattccaagacctttacaaggtcggaggggtaccagcagttatgaaatatctccttaaaaatggcttccttcatggtgaccgtatcacttgtactggcaaaacagtcgctgaaaatttgaaggcttttgatgatttaacacctggtcaaaaggttattatgccgcttgaaaatcctaaacgtgaagatggtccgctcattattctccatggtaacttggctccagacggtgccgttgccaaagtttctggtgtaaaagtgcgtcgtcatgtcggtcctgctaaggtctttaattctgaagaagaagccattgaagctgtcttgaatgatgatattgttgatggtgatgttgttgtcgtacgttttgtaggaccaaagggcggtcctggtatgcctgaaatgctttccctttcatcaatgattgttggtaaagggcaaggtgaaaaagttgcccttctgacagatggccgcttctcaggtggtacttatggtcttgtcgtgggtcatatcgctcctgaagcacaagatggcggtccaatcgcctacctgcaaacaggagacatagtcactattgaccaagacactaaggaattacactttgatatctccgatgaagagttaaaacatcgtcaagagaccattgaattgccaccgctctattcacgcggtatccttggtaaatatgctcacatcgtttcgtctgcttctaggggagccgtaacagacttttggaagcctgaagaaactggcaaaaaa

SEQ ID NO: 97 - protein, 571 aa, Streptococcus mutans
MTDKKTLKDLRNRSSVYDSMVKSPNRAMLRATGMQDEDFEKPIVGVISTWAENTPCNIHLHDFGKLAKVGVKEAGAWPVQFGTITVSDGIAMGTQGMRFSLTSRDIIADSIEAAMGGHNADAFVAIGGCDKNMPGSVIAMANMDIPAIFAYGGTIAPGNLDGKDIDLVSVFEGVGHWNHGDMTKEEVKALECNACPGPGGCGGMYTANTMATAIEVLGLSLPGSSSHPAESAEKKADIEEAGRAVVKMLEMGLKPSDILTREAFEDAITVTMALGGSTNSTLHLLAIAHAANVELTLDDFNTFQEKVPHLADLKPSGQYVFQDLYKVGGVPAVMKYLLKNGFLHGDRITCTGKTVAENLKAFDDLTPGQKVIMPLENPKREDGPLIILHGNLAPDGAVAKVSGVKVRRHVGPAKVFNSEEEAIEAVLNDDIVDGDVVVVRFVGPKGGPGMPEMLSLSSMIVGKGQGEKVALLTDGRFSGGTYGLVVGHIAPEAQDGGPIAYLQTGDIVTIDQDTKELHFDISDEELKHRQETIELPPLYSRGILGKYAHIVSSASRGAVTDFWKPEETGKK

SEQ ID NO: 98 - DNA, 1758 bp, Saccharomyces cerevisiae
atgggcttgttaacgaaagttgctacatctagacaattctctacaacgagatgcgttgcaaagaagctcaacaagtactcgtatatcatcactgaacctaagggccaaggtgcgtcccaggccatgctttatgccaccggtttcaagaaggaagatttcaagaagcctcaagtcggggttggttcctgttggtggtccggtaacccatgtaacatgcatctattggacttgaataacagatgttctcaatccattgaaaaagcgggtttgaaagctatgcagttcaacaccatcggtgtttcagacggtatctctatgggtactaaaggtatgagatactcgttacaaagtagagaaatcattgcagactcctttgaaaccatcatgatggcacaacactacgatgctaacatcgccatcccatcatgtgacaaaaacatgcccggtgtcatgatggccatgggtagacataacagaccttccatcatggtatatggtggtactatcttgcccggtcatccaacatgtggttcttcgaagatctctaaaaacatcgatatcgtctctgcgttccaatcctacggtgaatatatttccaagcaattcactgaagaagaaagagaagatgttgtggaacatgcatgcccaggtcctggttcttgtggtggtatgtatactgccaacacaatggcttctgccgctgaagtgctaggtttgaccattccaaactcctcttccttcccagccgtttccaaggagaagttagctgagtgtgacaacattggtgaatacatcaagaagacaatggaattgggtattttacctcgtgatatcctcacaaaagaggcttttgaaaacgccattacttatgtcgttgcaaccggtgggtccactaatgctgttttgcatttggtggctgttgctcactctgcgggtgtcaagttgtcaccagatgatttccaaagaatcagtgatactacaccattgatcggtgacttcaaaccttctggtaaatacgtcatggccgatttgattaacgttggtggtacccaatctgtgattaagtatctatatgaaaacaacatgttgcacggtaacacaatgactgttaccggtgacactttggcagaacgtgcaaagaaagcaccaagcctacctgaaggacaagagattattaagccactctcccacccaatcaaggccaacggtcacttgcaaattctgtacggttcattggcaccaggtggagctgtgggtaaaattaccggtaaggaaggtacttacttcaagggtagagcacgtgtgttcgaagaggaaggtgcctttattgaagccttggaaagaggtgaaatcaagaagggtgaaaaaaccgttgttgttatcagatatgaaggtccaagaggtgcaccaggtatgcctgaaatgctaaagccttcctctgctctgatgggttacggtttgggtaaagatgttgcattgttgactgatggtagattctctggtggttctcacgggttcttaatcggccacattgttcccgaagccgctgaaggtggtcctatcgggttggtcagagacggcgatgagattatcattgatgctgataataacaagattgacctattagtctctgataaggaaatggctcaacgtaaacaaagttgggttgcacctccacctcgttacacaagaggtactctatccaagtatgctaagttggtttccaacgcttccaacggttgtgttttagatgcttga

SEQ ID NO: 99 - protein, 585 aa, Saccharomyces cerevisiae
MGLLTKVATSRQFSTTRCVAKKLNKYSYIITEPKGQGASQAMLYATGFKKEDFKKPQVGVGSCWWSGNPCNMHLLDLNNRCSQSIEKAGLKAMQFNTIGVSDGISMGTKGMRYSLQSREIIADSFETIMMAQHYDANIAIPSCDKNMPGVMMAMGRHNRPSIMVYGGTILPGHPTCGSSKISKNIDIVSAFQSYGEYISKQFTEEEREDVVEHACPGPGSCGGMYTANTMASAAEVLGLTIPNSSSFPAVSKEKLAECDNIGEYIKKTMELGILPRDILTKEAFENAITYVVATGGSTNAVLHLVAVAHSAGVKLSPDDFQRISDTTPLIGDFKPSGKYVMADLINVGGTQSVIKYLYENNMLHGNTMTVTGDTLAERAKKAPSLPEGQEIIKPLSHPIKANGHLQILYGSLAPGGAVGKITGKEGTYFKGRARVFEEEGAFIEALERGEIKKGEKTVVVIRYEGPRGAPGMPEMLKPSSALMGYGLGKDVALLTDGRFSGGSHGFLIGHIVPEAAEGGPIGLVRDGDEIIIDADNNKIDLLVSDKEMAQRKQSWVAPPPRYTRGTLSKYAKLVSNASNGCVLDA

SEQ ID NO: 100 - DNA, 1647 bp, Lactococcus lactis
atgtatacagtaggagattacctattagaccgattacacgagttaggaattgaagaaatttttggagtccctggagactataacttacaatttttagatcaaattatttcccacaaggatatgaaatgggtcggaaatgctaatgaattaaatgcttcatatatggctgatggctatgctcgtactaaaaaagctgccgcatttcttacaacctttggagtaggtgaattgagtgcagttaatggattagcaggaagttacgccgaaaatttaccagtagtagaaatagtgggatcacctacatcaaaagttcaaaatgaaggaaaatttgttcatcatacgctggctgacggtgattttaaacactttatgaaaatgcacgaacctgttacagcagctcgaactttactgacagcagaaaatgcaaccgttgaaattgaccgagtactttctgcactattaaaagaaagaaaacctgtctatatcaacttaccagttgatgttgctgctgcaaaagcagagaaaccctcactccctttgaaaaaggaaaactcaacttcaaatacaagtgaccaagaaattttgaacaaaattcaagaaagcttgaaaaatgccaaaaaaccaatcgtgattacaggacatgaaataattagttttggcttagaaaaaacagtcactcaatttatttcaaagacaaaactacctattacgacattaaactttggtaaaagttcagttgatgaagccctcccttcatttttaggaatctataatggtacactctcagagcctaatcttaaagaattcgtggaatcagccgacttcatcttgatgcttggagttaaactcacagactcttcaacaggagccttcactcatcatttaaatgaaaataaaatgatttcactgaatatagatgaaggaaaaatatttaacgaaagaatccaaaattttgattttgaatccctcatctcctctctcttagacctaagcgaaatagaatacaaaggaaaatatatcgataaaaagcaagaagactttgttccatcaaatgcgcttttatcacaagaccgcctatggcaagcagttgaaaacctaactcaaagcaatgaaacaatcgttgctgaacaagggacatcattctttggcgcttcatcaattttcttaaaatcaaagagtcattttattggtcaacccttatggggatcaattggatatacattcccagcagcattaggaagccaaattgcagataaagaaagcagacaccttttatttattggtgatggttcacttcaacttacagtgcaagaattaggattagcaatcagagaaaaaattaatccaatttgctttattatcaataatgatggttatacagtcgaaagagaaattcatggaccaaatcaaagctacaatgatattccaatgtggaattactcaaaattaccagaatcgtttggagcaacagaagatcgagtagtctcaaaaatcgttagaactgaaaatgaatttgtgtctgtcatgaaagaagctcaagcagatccaaatagaatgtactggattgagttaattttggcaaaagaaggtgcaccaaaagtactgaaaaaaatgggcaaactatttgctgaacaaaataaatcataa

SEQ ID NO: 101 - protein, 548 aa, Lactococcus lactis
MYTVGDYLLDRLHELGIEEIFGVPGDYNLQFLDQIISHKDMKWVGNANELNASYMADGYARTKKAAAFLTTFGVGELSAVNGLAGSYAENLPVVEIVGSPTSKVQNEGKFVHHTLADGDFKHFMKMHEPVTAARTLLTAENATVEIDRVLSALLKERKPVYINLPVDVAAAKAEKPSLPLKKENSTSNTSDQEILNKIQESLKNAKKPIVITGHEIISFGLEKTVTQFISKTKLPITTLNFGKSSVDEALPSFLGIYNGTLSEPNLKEFVESADFILMLGVKLTDSSTGAFTHHLNENKMISLNIDEGKIFNERIQNFDFESLISSLLDLSEIEYKGKYIDKKQEDFVPSNALLSQDRLWQAVENLTQSNETIVAEQGTSFFGASSIFLKSKSHFIGQPLWGSIGYTFPAALGSQIADKESRHLLFIGDGSLQLTVQELGLAIREKINPICFIINNDGYTVEREIHGPNQSYNDIPMWNYSKLPESFGATEDRVVSKIVRTENEFVSVMKEAQADPNRMYWIELILAKEGAPKVLKKMGKLFAEQNKS

SEQ ID NO: 102 - DNA, 1644 bp, artificial sequence
  codon optimized coding region for expression in L. plantarum
atgtataccgttggtgactatttgttggatcggttacacgaattaggcatcgaagaaatcttcggtgttccaggcgattacaatttacaatttttggaccaaatcatcagtcacaaagacatgaaatgggtcggtaacgcaaatgaattaaatgcgagttacatggctgacggttatgctcggactaagaaagctgcagcctttttgacgacttttggtgttggtgaattatcagccgttaatggtttggccggtagttacgccgaaaatttaccggtcgttgaaattgttggcagtccaacgagtaaggttcaaaacgaaggtaaatttgtccatcataccttggcagatggtgattttaaacactttatgaagatgcatgaaccggtcactgctgcacggactttattaactgcggaaaatgccaccgtcgaaattgaccgcgttttatcagccttgttaaaagaacgcaaaccggtttacattaatttaccggtcgatgtggcagcggccaaagctgaaaaaccgagtttgccgttaaagaaggaaaatagtacctcaaatacctcagatcaagaaatcttgaacaagatccaagaatcattaaaaaatgctaaaaaaccaatcgttattaccggtcatgaaattattagttttggtttggaaaaaacggtgacgcaatttatttcaaagacgaaattaccaattacgaccttgaattttggtaaatcaagtgttgacgaagcgttgccaagtttcttgggtatctataatggtactttaagtgaaccgaatttaaaagaatttgtggaaagtgcagattttattttaatgttgggtgtcaagttaactgattcatcaactggcgcgttcacgcatcatttaaatgaaaataaaatgatttcattgaatattgatgaaggcaaaatcttcaacgaacggattcaaaattttgattttgaatcattgattagtagtttgttagatttatcagaaatcgaatacaaaggcaagtatattgataagaagcaggaagattttgttccgagtaatgcattgttaagtcaagatcgcttatggcaggcggttgaaaacttaactcaatcaaacgaaaccattgttgctgaacaaggtacttcattcttcggcgcatcaagtatctttttaaaatcaaaaagtcatttcatcggtcaaccattatggggtagtattggttacaccttcccagcggcgttaggtagtcaaattgctgacaaagaatcacgacacttattattcattggtgacggtagtttacaattgacggtccaggaattaggtttggccattcgcgaaaagatcaacccaatttgtttcattatcaataatgacggttatactgttgaacgggaaattcacggtccgaaccaaagttacaatgatattccaatgtggaactactcaaaattgccggaaagttttggcgccaccgaagatcgggtcgttagtaaaattgtgcggaccgaaaatgaattcgtgtcagtgatgaaggaagcacaagccgatccgaaccggatgtattggattgaattaatcttggctaaggaaggtgccccgaaggttttaaagaagatgggcaagttatttgcagaacaaaataaaagt

SEQ ID NO: 103 - DNA, 1047 bp, Achromobacter xylosoxidans
atgaaagctctggtttatcacggtgaccacaagatctcgcttgaagacaagcccaagcccacccttcaaaagcccacggatgtagtagtacgggttttgaagaccacgatctgcggcacggatctcggcatctacaaaggcaagaatccagaggtcgccgacgggcgcatcctgggccatgaaggggtaggcgtcatcgaggaagtgggcgagagtgtcacgcagttcaagaaaggcgacaaggtcctgatttcctgcgtcacttcttgcggctcgtgcgactactgcaagaagcagctttactcccattgccgcgacggcgggtggatcctgggttacatgatcgatggcgtgcaggccgaatacgtccgcatcccgcatgccgacaacagcctctacaagatcccccagacaattgacgacgaaatcgccgtcctgctgagcgacatcctgcccaccggccacgaaatcggcgtccagtatgggaatgtccagccgggcgatgcggtggctattgtcggcgcgggccccgtcggcatgtccgtactgttgaccgcccagttctactccccctcgaccatcatcgtgatcgacatggacgagaatcgcctccagctcgccaaggagctcggggcaacgcacaccatcaactccggcacggagaacgttgtcgaagccgtgcataggattgcggcagagggagtcgatgttgcgatcgaggcggtgggcataccggcgacttgggacatctgccaggagatcgtcaagcccggcgcgcacatcgccaacgtcggcgtgcatggcgtcaaggttgacttcgagattcagaagctctggatcaagaacctgacgatcaccacgggactggtgaacacgaacacgacgcccatgctgatgaaggtcgcctcgaccgacaagcttccgttgaagaagatgattacccatcgcttcgagctggccgagatcgagcacgcctatcaggtattcctcaatggcgccaaggagaaggcgatgaagatcatcctctcgaacgcaggcgctgcctga

SEQ ID NO: 104 - protein, 348 aa, Achromobacter xylosoxidans
MKALVYHGDHKISLEDKPKPTLQKPTDVVVRVLKTTICGTDLGIYKGKNPEVADGRILGHEGVGVIEEVGESVTQFKKGDKVLISCVTSCGSCDYCKKQLYSHCRDGGWILGYMIDGVQAEYVRIPHADNSLYKIPQTIDDEIAVLLSDILPTGHEIGVQYGNVQPGDAVAIVGAGPVGMSVLLTAQFYSPSTIIVIDMDENRLQLAKELGATHTINSGTENVVEAVHRIAAEGVDVAIEAVGIPATWDICQEIVKPGAHIANVGVHGVKVDFEIQKLWIKNLTITTGLVNTNTTPMLMKVASTDKLPLKKMITHRFELAEIEHAYQVFLNGAKEKAMKIILSNAGAA

SEQ ID NO: 105 - DNA, 1047 bp, artificial sequence
  codon optimized coding region of sadB for expression in L. plantarum
atgaaagctttggtttaccatggtgaccacaaaattagtttagaagataagccaaaaccaactttgcagaagccaactgatgtcgtggtccgtgtcttaaagacgaccatttgtggcacggacttgggtatctataagggcaagaatccagaagttgccgacggtcgtatcttaggtcatgaaggcgtcggtgttatcgaagaagttggtgaaagtgttacccaattcaagaagggtgacaaagttttaatcagttgtgttacgagttgtggttcatgtgattactgtaagaaacaattgtacagtcattgtcgtgacggtggttggatcttgggttacatgattgatggtgtccaagctgaatacgtccgtattccacacgcagataattcattgtataaaattccacaaactattgacgacgaaattgctgttttgttatcagatatcttaccaacgggccatgaaattggtgtccaatatggcaacgtccaaccaggcgatgccgttgcaattgttggcgccggtccagtgggcatgagtgttttattaacggctcaattctattcaccatcaactatcattgtcatcgacatggatgaaaatcgtttgcaattggctaaggaattgggcgctacgcacactatcaattcaggtacggaaaatgttgttgaagcagtccatcgtattgcagctgaaggcgtcgatgtggcaattgaagccgtgggcattccagcaacgtgggatatttgtcaagaaattgttaagccaggcgcccatatcgccaatgtgggtgtgcatggtgtgaaggttgattttgaaattcaaaaattgtggattaaaaatttgacgattactactggtttggttaacacgaatacgactccaatgttaatgaaggtcgcatcaaccgataaattaccattgaagaagatgattactcaccgttttgaattagcagaaattgaacatgcctatcaagttttcttaaacggcgccaaggaaaaagcaatgaaaattattttatcaaacgccggtgcagcttaa

SEQ ID NO: 106 - DNA, 1125 bp, artificial sequence
  horse ADH coding region codon optimized for S. cerevisiae expression
atgtcaacagccggtaaagttattaagtgtaaagcggcagttttgtgggaagagaaaaagccgtttagcatagaagaagtagaagtagcgccaccaaaagcacacgaggttagaatcaagatggttgccaccggaatctgtagatccgacgaccatgtggtgagtggcactctagttactcctttgccagtaatcgcgggacacgaggctgccggaatcgttgaatccataggtgaaggtgttaccactgttcgtcctggtgataaagtgatcccactgttcactcctcaatgtggtaagtgtagagtctgcaaacatcctgagggtaatttctgccttaaaaatgatttgtctatgcctagaggtactatgcaggatggtacaagcagatttacatgcagagggaaacctatacaccatttccttggtacttctacattttcccaatacacagtggtggacgagatatctgtcgctaaaatcgatgcagcttcaccactggaaaaagtttgcttgatagggtgcggattttccaccggttacggttccgcagttaaagttgcaaaggttacacagggttcgacttgtgcagtattcggtttaggaggagtaggactaagcgttattatggggtgtaaagctgcaggcgcagcgaggattataggtgtagacatcaataaggacaaatttgcaaaagctaaggaggtcggggctactgaatgtgttaaccctcaagattataagaaaccaatacaagaagtccttactgaaatgtcaaacggtggagttgatttctcttttgaagttataggccgtcttgatactatggtaactgcgttgtcctgctgtcaagaggcatatggagtcagtgtgatcgtaggtgttcctcctgattcacaaaatttgtcgatgaatcctatgctgttgctaagcggtcgtacatggaagggagctatatttggcggttttaagagcaaggatagtgttccaaaacttgttgccgactttatggcgaagaagtttgctcttgatcctttaattacacatgtattgccattcgagaaaatcaatgaagggtttgatttgttaagaagtggtgaatctattcgtacaattttaactttt

SEQ ID NO: 107 - protein, 375 aa, Equus caballus
MSTAGKVIKCKAAVLWEEKKPFSIEEVEVAPPKAHEVRIKMVATGICRSDDHVVSGTLVTPLPVIAGHEAAGIVESIGEGVTTVRPGDKVIPLFTPQCGKCRVCKHPEGNFCLKNDLSMPRGTMQDGTSRFTCRGKPIHHFLGTSTFSQYTVVDEISVAKIDAASPLEKVCLIGCGFSTGYGSAVKVAKVTQGSTCAVFGLGGVGLSVIMGCKAAGAARIIGVDINKDKFAKAKEVGATECVNPQDYKKPIQEVLTEMSNGGVDFSFEVIGRLDTMVTALSCCQEAYGVSVIVGVPPDSQNLSMNPMLLLSGRTWKGAIFGGFKSKDSVPKLVADFMAKKFALDPLITHVLPFEKINEGFDLLRSGESIRTILTF

SEQ ID NO: 108 - DNA, 1440 bp, Escherichia coli
atggtaacgttcatgataacttctgctcttcatcgtgcggccgactgggctaaatctgtgttctcttcggcggcgctgggtgatcctcgccgtactgcccgcttggttaacgtcgccgcccaattggcaaaatattctggtaaatcaataaccatctcatcagagggtagtgaagccatgcaggaaggcgcttaccgatttatccgcaatcccaacgtttctgccgaggcgatcagaaaggctggcgccatgcaaacagtcaagttggctcaggagtttcccgaactgctggccattgaggacaccacctctttgagttatcgccaccaggtcgccgaagagcttggcaagctgggctctattcaggataaatcccgcggatggtgggttcactccgttctcttgctcgaggccaccacattccgcaccgtaggattactgcatcaggagtggtggatgcgcccggatgaccctgccgatgcggatgaaaaggagagtggcaaatggctggcagccgccgcaactagccggttacgcatgggcagcatgatgagcaacgtgattgcggtctgtgaccgcgaagccgatattcatgcttatctgcaggacaaactggcgcataacgagcgcttcgtggtgcgctccaagcacccacgcaaggacgtagagtctgggttgtatctgtacgaccatctgaagaaccaaccggagttgggtggctatcagatcagcattccgcaaaagggcgtggtggataaacgcggtaaacgtaaaaatcgaccagcccgcaaggcgagcttgagcctgcgcagtgggcgcatcacgctaaaacaggggaatatcacgctcaacgcggtgctggccgaggagattaacccgcccaagggtgagaccccgttgaaatggttgttgctgaccagcgaaccggtcgagtcgctagcccaagccttgcgcgtcatcgacatttatacccatcgctggcggatcgaggagttccataaggcatggaaaaccggagcaggagccgagaggcaacgcatggaggagccggataatctggagcggatggtctcgatcctctcgtttgttgcggtcaggctgttacagctcagagaaagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctgggaaggttgggaagccctgcaaagtaaactggatggctttcttgccgccaaggatctgatggcgcaggggatcaagatc

SEQ ID NO: 109 - protein, 480 aa, Escherichia coli
MVTFMITSALHRAADWAKSVFSSAALGDPRRTARLVNVAAQLAKYSGKSITISSEGSEAMQEGAYRFIRNPNVSAEAIRKAGAMQTVKLAQEFPELLAIEDTTSLSYRHQVAEELGKLGSIQDKSRGWWVHSVLLLEATTFRTVGLLHQEWWMRPDDPADADEKESGKWLAAAATSRLRMGSMMSNVIAVCDREADIHAYLQDKLAHNERFVVRSKHPRKDVESGLYLYDHLKNQPELGGYQISIPQKGVVDKRGKRKNRPARKASLSLRSGRITLKQGNITLNAVLAEEINPPKGETPLKWLLLTSEPVESLAQALRVIDIYTHRWRIEEFHKAWKTGAGAERQRMEEPDNLERMVSILSFVAVRLLQLRESFTLPQALRAQGLLKEAEHVESQSAETVLTPDECQLLGYLDKGKRKRKEKAGSLQWAYMAIARLGGFMDSKRTGIASWGALWEGWEALQSKLDGFLAAKDLMAQGIKI

SEQ ID NO: 110 - DNA, 5323 bp, artificial sequence
  vector
gaattcagatctctcgagcccgggatcgatggtacctcgcgaaagcttggatgttgtacaggataatgtccagaaggtcgatagaaagcgtgagaaacagcgtacagacgatttagagatgtagaggtacttttatgccgagaaaactttttgcgtgtgacagtccttaaaatatacttagagcgtaagcgaaagtagtagcgacagctattaactttcggttgcaaagctctaggatttttaatggacgcagcgcatcacacgcaaaaaggaaattggaataaatgcgaaatttgagatgttaattaaagacctttttgaggtctttttttcttagatttttggggttatttaggggagaaaacataggggggtactacgacctcccccctaggtgtccattgtccattgtccaaacaaataaataaatattgggtttttaatgttaaaaggttgttttttatgttaaagtgaaaaaaacagatgttgggaggtacagtgatagttgtagatagaaaagaagagaaaaaagttgctgttactttaagacttacaacagaagaaaatgagatattaaatagaatcaaagaaaaatataatattagcaaatcagatgcaaccggtattctaataaaaaaatatgcaaaggaggaatacggtgcattttaaacaaaaaaagatagacagcactggcatgctgcctatctatgactaaattttgttaagtgtattagcaccgttattatatcatgagcgaaaatgtaataaaagaaactgaaaacaagaaaaattcaagaggacgtaattggacatttgttttatatccagaatcagcaaaagccgagtggttagagtatttaaaagagttacacattcaatttgtagtgtctccattacatgatagggatactgatacagaaggtaggatgaaaaaagagcattatcatattctagtgatgtatgagggtaataaatcttatgaacagataaaaataattaacagaagaattgaatgcgactattccgcagattgcaggaagtgtgaaaggtcttgtgagatatatgcttcacatggacgatcctaataaatttaaatatcaaaaagaagatatgatagtttatggcggtgtagatgttgatgaattattaaagaaaacaacaacagatagatataaattaattaaagaaatgattgagtttattgatgaacaaggaatcgtagaatttaagagtttaatggattatgcaatgaagtttaaatttgatgattggttcccgcttttatgtgataactcggcgtatgttattcaagaatatataaaatcaaatcggtataaatctgaccgatagattttgaatttaggtgtcacaagacactcttttttcgcaccagcgaaaactggtttaagccgactgcgcaaaagacataatcgattcacaaaaaataggcacacgaaaaacaagttaagggatgcagtttatgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttttgtttatctttgtgtgatattcttgatttttttctacttaatctgataagtgagctattcactttaggtttaggatgaaaatattctcttggaaccatacttaatatagaaatatcaacttctgccattaaaagtaatgccaatgagcgttttgtatttaataatcttttagcaaacccgtattccacgattaaataaatctcattagctatactatcaaaaacaattttgcgtattatatccgtacttatgttataaggtatattaccatatattttataggattggtttttaggaaatttaaactgcaatatatccttgtttaaaacttggaaattatcgtgatcaacaagtttattttctgtagttttgcataatttatggtctatttcaatggcagttacgaaattacacctctttactaattcaagggtaaaatggccttttcctgagccgatttcaaagatattatcatgttcatttaatcttatatttgtcattattttatctatattatgttttgaagtaataaagttttgactgtgttttatatttttctcgttcattataaccctctttaatttggttatatgaattttgcttattaacgattcattataaccacttattttttgtttggttgataatgaactgtgctgattacaaaaatactaaaaatgcccatattttttcctccttataaaattagtataattatagcacgagctctgataaatatgaacatgatgagtgatcgttaaatttatactgcaatcggatgcgattattgaataaaagatatgagagatttatctaatttcttttttcttgtaaaaaaagaaagttcttaaaggttttatagttttggtcgtagagcacacggtttaacgacttaattacgaagtaaataagtctagtgtgttagactttatgaaatctatatacgtttatatatatttattatccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaa

SEQ ID NO: 111 - DNA, 31 bp, artificial sequence
  primer
catgaattcgtgctaagagccagattgtgga

SEQ ID NO: 112 - DNA, 50 bp, artificial sequence
  primer
catgaagaccacgcgtaggccttctagagctaaattttcacatcgtgagc

SEQ ID NO: 113 - DNA, 49 bp, artificial sequence
  primer
atttagctctagaaggcctacgcgtggtcttcatgaacttgttcaaccg

SEQ ID NO: 114 - DNA, 31 bp, artificial sequence
  primer
catctcgagccaagctcagtcacgcatttaa

SEQ ID NO: 115 - DNA, 22 bp, artificial sequence
  primer
aagcacaacgggaagcgaacat

SEQ ID NO: 116 - DNA, 23 bp, artificial sequence
  primer
atacaactatgacgctggaagcg

SEQ ID NO: 117 - DNA, 23 bp, artificial sequence
  primer
gtaggttttcccgtccttgatag

SEQ ID NO: 118 - DNA, 35 bp, artificial sequence
  primer
tataagatcttgactctggtgaacttgtcgcaacc

SEQ ID NO: 119 - DNA, 34 bp, artificial sequence
  primer
atatctcgagaataagtcatcctctcgtagtgaa

SEQ ID NO: 120 - DNA, 34 bp, artificial sequence
  primer
tatactcgagtaatcatttcatacgattaaatgt

SEQ ID NO: 121 - DNA, 32 bp, artificial sequence
  primer
atatcccggggtgagcgggtaaagtccttgcc

SEQ ID NO: 122 - DNA, 710 bp, Lactobacillus plantarum
atgaagcgaccaattatcattgcgttagattttcccaccgccgaacgggccttagcttttttagaccaatttccggctgatttacatgtcactgtcaaaatcggcatggagttattttatgcagcgggaccgagtattgtgacggacgtgcaagctcgcggccatgcggttttcttagatttgaaactacatgatattcccaataccgtcgaatccgcaatgcgggtgatcgggcggttaggggtaacctatacgacggttcatgctgcgggtgggcacgtgatgctttcagccgccaaacgaggattggtcgcgggtgcaatggccgctggagtcactgcccccaagttattagcgattacgcagttaacttcgactaatcaagctattttgaatcaggaccagcaaatcatgggaacggttcgggcgagtgtcgtgcattatgccaaactagcacgggcgagtgactgtgatggcgtcatttgttccgcccaagaagttcaggcgattcatacggccgtcggtgctgattttctcggaattacgccgggaattcggccagcgtcggcgcagtcagatgaccagcaacgggtgatgacaccggctgccgctgctaaggctgggagcaacggtctcgtcatcgggcggccaattacgcaggctgcagaaccagttcaagcttaccgagatattatgacagaatggagtaa

SEQ ID NO: 123 - DNA, 21 bp, artificial sequence
  primer
cgggcacctgcaaccgaggtc

SEQ ID NO: 124 - DNA, 22 bp, artificial sequence
  primer
ctgtttctcacgctttctatcg

SEQ ID NO: 125 - DNA, 22 bp, artificial sequence
  primer
gattttctttatcaacttcgac

SEQ ID NO: 126 - DNA, 21 bp, artificial sequence
  primer
ttggaaaacgttcttcggggc

SEQ ID NO: 127 - DNA, 20 bp, artificial sequence
  ribosome binding site
atataggaggaatttttgta

SEQ ID NO: 128 - DNA, 5124 bp, artificial sequence
  constructed plasmid
gacgaaagggccttataacttacaaataacccctcgaaaacattgaaagaataacccccaagatctatattatagatcttgggggttatttgttttaatattaaagaaatgacttcttctatttgtcatcaatactaaacaataatttgtacaaagtgattatttcttctagttcttcacgcgatacatgatcgacaatagtttcatcagtgacatgtcttgcccgtaaatctaaggctatggtttgatctaataatacttttccatatactgtttgactactagttagtcgatgatacattggaaaattacgcttggtactgctaattggagccacaatcgtcatgttacttgtctgacagactagatcattgcttagcgcaatggctggtcgcttattcatctgttcatgaccacggcttggattaaagttaacataaaatatatcaccttggcttaccattgaagttcattaccttctgactttccccaatcaagctcgtgatcccttttcccgtcatctttccaatccttaaatagttcgtgaatattggttgggttcttttttattggtgttaaaacaattgatccattttcaatggttattgtcatatcttggttatcatctaatttcagttgtttaataatttggctaggaattctagcagctttcgagtttccccactttgctaagcgtgtttgttctttaataagttccatattttcccctcctaaattattattacaagtcaagtatatcccatgtagatacacaatgcaaatattcttactggagaaataacaccttaagtctagcaccacccgcacgcatagcggtgcttaaaccatcaagggtcaagcccttaggctctctcaaacagttatcctaatcgtgaataactgcgcttcttttgcagtataaagagagaactctttatcagacaatttaagctcaaccagcccttgcactaactattattagagttggttttagcagcaacccgaataatctgcgttaatagttagcctgtccgtatcatttcctagtcttccagccacgtctttagtcgcgttgatctcgacaaggtttagcatacctatgttgttaactgcaagcggggtcacgaacgacactcacgggaggttttactagctaagaacaggtttccagcctttagttgctttgatggttgctaaccattgaataacaaaaaaacggttgctatcaggtttctgttaagattcccgataacaaccgtttactttaagtatcaatggttgaaaaacttagcctacatgttataatagtaccaagttagatagcttgtattggtagtacttgctatcgaaaatcttatcaggttgtgctgataagtcgtgaatcctaactctgctaagttgagggttcttttttttgcgttcatttattaagttgagtacattataaccgtaatataagattaatacaacctttatcattttaacgtctcaaccagccgaataatccttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataagggcccgcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtctcgaggaccgagcgcagcgagtcagtgagcgaggaagcggaagagcgagggcggagttgttgacagccgaggtaccatgtggtataatcccgagtgtggaattgtgagcggataacaggatccatataggaggaatttttgtaatggaattcaaatataacggaaaagttgaatcagtggaactcaataaatattctaagacattgactcaagatccaacacaaccagcgactcaagcgatgtactacggcattggttttaaagatgaggatttcaaaaaagctcaggtcggaatcgtcagcatggattgggacggaaatccatgtaatatgcacttgggaacacttgggagtaaaatcaaaagttctgtcaaccaaactgacggattgattggacttcaatttcatactattggagtttctgatggaattgctaacggaaagcttggcatgagatattctttggtcagtcgtgaagttattgctgacagcatcgaaaccaacgctggcgcagaatattatgatgccatcgttgccattcccggttgtgataaaaatatgcccgggtcaattatcggaatggctcgcttaaatcgtccgtcaattatggtctatggtggaacgattgaacatggcgaatataaaggtgaaaaattaaatattgtttcggcctttgaagctctggggcaaaaaatcactggaaatatttctgatgaagattatcatggcgttatttgcaatgccattccaggacaaggtgcttgcggaggaatgtacactgccaataccctggctgctgctattgaaactttgggaatgagtttaccttattcctcttccaatccagcagtcagtcaagaaaaacaagaagagtgtgatgacattggtttagccatcaaaaatttattagaaaaagatattaaaccaagtgatatcatgaccaaagaagcttttgaaaatgccataacaattgtcatggtccttggaggctcaaccaatgctgtgcttcatatcattgcaatggcaaatgccattggtgtagaaattacgcaagatgatttccaacgtatttcagatattacccctgttcttggcgatttcaaaccgagcggaaaatatatgatggaagatctgcacaaaattggtggccttcctgctgttttgaaatacctacttaaagaaggaaaacttcacggtgattgtttgaccgtcacaggtaaaactttggctgaaaatgttgaaacagcattagatttggactttgacagtcaagatattatgcgaccactaaaaaatccaattaaagctactggacatttacaaattttgtacggtaatcttgcccaagggggttctgttgcaaaaatttctggtaaagaaggcgaatttttcaaaggaacagctcgtgtttttgacggagaacaacactttatcgatggcattgagtctggccgattgcatgccggtgatgttgcggtcattagaaatattggcccagtcggaggtccgggaatgccagagatgttaaaaccaacctcagcattaattggagcaggacttggaaaatcttgtgccctaattactgacggaagattttctggtggcacacacggctttgttgtgggtcatatcgtccctgaagcagttgaaggtgggttgattggtttagttgaagatgatgatattatcgaaattgatgcggtgaataatagtattagtttaaaagtttctaatgaagaaattgctaaacgacgtgccaattatcaaaaaccaacccctaaagcaacgcgtggtgttcttgcaaaatttgccaaacttacgcgccccgctagtgaaggttgcgttacagatttactgcaggacgggcttgtctgctcccggcatccgcttacagacaagctgtgaccgtctccgggagctgcatgtgtcagaggttttcaccgtcatcaccgaaacgcgcga

SEQ ID NO: 129 - DNA, 3748 bp, artificial sequence
  vector
gacgaaagggccttataacttacaaataacccctcgaaaacattgaaagaataacccccaagatctatattatagatcttgggggttatttgttttaatattaaagaaatgacttcttctatttgtcatcaatactaaacaataatttgtacaaagtgattatttcttctagttcttcacgcgatacatgatcgacaatagtttcatcagtgacatgtcttgcccgtaaatctaaggctatggtttgatctaataatacttttccatatactgtttgactactagttagtcgatgatacattggaaaattacgcttggtactgctaattggagccacaatcgtcatgttacttgtctgacagactagatcattgcttagcgcaatggctggtcgcttattcatctgttcatgaccacggcttggattaaagttaacataaaatatatcaccttggcttaccattgaagttcattaccttctgactttccccaatcaagctcgtgatcccttttcccgtcatctttccaatccttaaatagttcgtgaatattggttgggttcttttttattggtgttaaaacaattgatccattttcaatggttattgtcatatcttggttatcatctaatttcagttgtttaataatttggctaggaattctagcagctttcgagtttccccactttgctaagcgtgtttgttctttaataagttccatattttcccctcctaaattattattacaagtcaagtatatcccatgtagatacacaatgcaaatattcttactggagaaataacaccttaagtctagcaccacccgcacgcatagcggtgcttaaaccatcaagggtcaagcccttaggctctctcaaacagttatcctaatcgtgaataactgcgcttcttttgcagtataaagagagaactctttatcagacaatttaagctcaaccagcccttgcactaactattattagagttggttttagcagcaacccgaataatctgcgttaatagttagcctgtccgtatcatttcctagtcttccagccacgtctttagtcgcgttgatctcgacaaggtttagcatacctatgttgttaactgcaagcggggtcacgaacgacactcacgggaggttttactagctaagaacaggtttccagcctttagttgctttgatggttgctaaccattgaataacaaaaaaacggttgctatcaggtttctgttaagattcccgataacaaccgtttactttaagtatcaatggttgaaaaacttagcctacatgttataatagtaccaagttagatagcttgtattggtagtacttgctatcgaaaatcttatcaggttgtgctgataagtcgtgaatcctaactctgctaagttgagggttcttttttttgcgttcatttattaagttgagtacattataaccgtaatataagattaatacaacctttatcattttaacgtctcaaccagccgaataatccttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataagggcccgcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtctcgaggaccgagcgcagcgagtcagtgagcgaggaagcggaagagcgagggcggagttgttgacagccgaggtaccatgtggtataatcccgagtgtggaattgtgagcggataacaatttcacacaggaaacagctatgaccatgattacgccaagcttggctgcaggtcgacggatccccgggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 130 - DNA, 22 bp, artificial sequence
  primer
ttgtctcgaggaccgagcgcag

SEQ ID NO: 131 - DNA, 60 bp, artificial sequence
  primer
tatagacaagcccgtcctgcagttaagcttttggatcctgttatccgctcacaattccac

SEQ ID NO: 132 - DNA, 1730 bp, artificial sequence
  PCR fragment with ribosome binding site and ilvD coding region from L. lactis
atataggaggaatttttgtaatggaattcaaatataacggaaaagttgaatcagtggaactcaataaatattctaagacattgactcaagatccaacacaaccagcgactcaagcgatgtactacggcattggttttaaagatgaggatttcaaaaaagctcaggtcggaatcgtcagcatggattgggacggaaatccatgtaatatgcacttgggaacacttgggagtaaaatcaaaagttctgtcaaccaaactgacggattgattggacttcaatttcatactattggagtttctgatggaattgctaacggaaagcttggcatgagatattctttggtcagtcgtgaagttattgctgacagcatcgaaaccaacgctggcgcagaatattatgatgccatcgttgccattcccggttgtgataaaaatatgcccgggtcaattatcggaatggctcgcttaaatcgtccgtcaattatggtctatggtggaacgattgaacatggcgaatataaaggtgaaaaattaaatattgtttcggcctttgaagctctggggcaaaaaatcactggaaatatttctgatgaagattatcatggcgttatttgcaatgccattccaggacaaggtgcttgcggaggaatgtacactgccaataccctggctgctgctattgaaactttgggaatgagtttaccttattcctcttccaatccagcagtcagtcaagaaaaacaagaagagtgtgatgacattggtttagccatcaaaaatttattagaaaaagatattaaaccaagtgatatcatgaccaaagaagcttttgaaaatgccataacaattgtcatggtccttggaggctcaaccaatgctgtgcttcatatcattgcaatggcaaatgccattggtgtagaaattacgcaagatgatttccaacgtatttcagatattacccctgttcttggcgatttcaaaccgagcggaaaatatatgatggaagatctgcacaaaattggtggccttcctgctgttttgaaatacctacttaaagaaggaaaacttcacggtgattgtttgaccgtcacaggtaaaactttggctgaaaatgttgaaacagcattagatttggactttgacagtcaagatattatgcgaccactaaaaaatccaattaaagctactggacatttacaaattttgtacggtaatcttgcccaagggggttctgttgcaaaaatttctggtaaagaaggcgaatttttcaaaggaacagctcgtgtttttgacggagaacaacactttatcgatggcattgagtctggccgattgcatgccggtgatgttgcggtcattagaaatattggcccagtcggaggtccgggaatgccagagatgttaaaaccaacctcagcattaattggagcaggacttggaaaatcttgtgccctaattactgacggaagattttctggtggcacacacggctttgttgtgggtcatatcgtccctgaagcagttgaaggtgggttgattggtttagttgaagatgatgatattatcgaaattgatgcggtgaataatagtattagtttaaaagtttctaatgaagaaattgctaaacgacgtgccaattatcaaaaaccaacccctaaagcaacgcgtggtgttcttgcaaaatttgccaaacttacgcgccccgctagtgaaggttgcgttacagattta

SEQ ID NO: 133 - DNA, 33 bp, artificial sequence
  primer
tattctcgagatataggaggaatttttgtaatg

SEQ ID NO: 134 - DNA, 31 bp, artificial sequence
  primer
atatctcgagctataaatctgtaacgcaacc

SEQ ID NO: 135 - DNA, 1014 bp, artificial sequence
  PCR fragment restriction sites added to a 5′ portion of suf operon of L. plantarum
atggcaaccttggaagttaaagatttacacgttgaagtgacggatgatgagcaaaaaaagtctcgtgagattttaaaaggcgtcaatttatctatgaaaactggtgaaattcatgccattatgggaccaaatgggactggtaagtccactttatcacaaactattatgggccaaccggcttatcacgttactcagggtgatatcttgttgaacggcgaaagtatcgtaaacatgccagttgatgaacgtgcacgtaagggactcttcctcggcatgcagtatccagctgaaattcaaggggtcaccaacgctgaatttttacgggcagcaatgaacgcacgccgaccagccgatgatcaaatctcagtgatggcctttcttaaagaactcgacaagaacttggcactacttaatatgagcgaatccatgacggaacgttacctaaacgaaggtttctccggtggtgaaaagaagcgtaacgaaattttgcaattattgatgatcaagccatcattcgccttattggacgaaattgattccgggcttgatatcgatgcgttacaagtggtttctaagggtgttaattcgatgcggggcgataatttcggctcattgattatcacgcattatcaacggctgttaaactacattgtgcccgatgtcgttcacgtgatgatgggtggtcgaatcgtgaagactggtaacgccgacttagcaaagacccttgaaaaagaaggttatgctggtttacgtgacgatttgaacattgatgtcaaacttgttgacgacgaagattagggggtggcagtaatggaagcaactgctgattatgaaactatcaaaacaacgctagctgcggctgctaacgaacatggtgaaccgcactggctcgttgaacgccgtttagcggctttagatgcgatgcaaggcctagcggttcccaaagctgatcgctttagtattcgcgactggccactgacgcccaccgaccaaccactaaagttcagtcgttcg

SEQ ID NO: 136 - DNA, 57 bp, artificial sequence
  primer
tagctagcccgggatatataggaggaatttttgtaatggcaaccttggaagttaaag

SEQ ID NO: 137 - DNA, 39 bp, artificial sequence
  primer
attatgtcaggtacccgaacgactgaactttagtggttg

SEQ ID NO: 138 - DNA, 93 bp, artificial sequence
  primer
tgtaggactcgagagatctcggccgggagttgttgacacacaaaaccagacatggtattataatctataagcgagatctgctcggggagttgt

SEQ ID NO: 139 - DNA, 74 bp, artificial sequence
  primer
tagctagcccggggcgctataccgagattatatcatgccttgctgcgcgtgtcaacaactccccgagcagatct

SEQ ID NO: 140 - DNA, 21 bp, artificial sequence
  primer
tgtaggactcgagagatctcg

SEQ ID NO: 141 - DNA, 16 bp, artificial sequence
  primer
tagctagcccggggcg

SEQ ID NO: 142 - DNA, 1331 bp, artificial sequence
  PCR product; restriction sites and sufP
cgaagacgggtagccctaagaacggattggtgacccacttgtcgattttagcagtcatctcgacgtggctggtgcttgccaagttttgactagcgctggctaaggtttgctcaataaagtctagtcgggtttggaaaatctgctcgtcgaatttttgagcgtcgtaataagcggcttgactagtgagtggtgtcaatcccatgtgtttcgcgtaggctcgaattggcttattttgattaataaattgaatcgtcagccaccgtgcgaagtctgctgtcaaatcaaattttttgatgagctgttggctagcctgacgaatggcttgctcaatcatgaaaggatagttgagtttgagcggtgtcgcggccggatggggacctgccaataagtcttcgcgtagctgatgcagcccttctttattgcgggcgttagtcgctttgatctgacagccaagttgttcagctagcgtgtcaaaatcataataatggcccgtccgtttgaggtcatcaatcatgttgagtgcgataatcaccggtgcgccaaattccaacacttcgatcgacagtagtaagttacgcttgagctgactcgcgttggtcacgttcagaattagatcagggtggttatgtagcagatagttggtgacgactgcttcatctttggtgattggattaagtgaatagactccgggtaaatcgaccatttcaacttcagagtgccgaatccggcccatcttcttctcaacggtcacgccggtccagttaccgacgtacgcgtatttatccgtcagttcattgaatagcgtggtcttaccagtattgggatttccgagcaatgcaaccgtcgtcatcttaatggcctccaatcaacgtttgaaatacggtgtaccgaatcccaatccgttgttgatcaatctgaacaattactggtccgtgaaacggatagtagcggacaacggccagcggactgccgacatgtagccctaagctgtgcaatcgttgaacggtctgattatccaaacctgtgaattgttgaatatgtagttgtgatgtattagtaacggattgacttagcataaggctcagtcctttctgaatattccaatagtgaataattcatcaaaaatattatacctacatcatagcatgaaagggcttttattaatatggggaaaagccttattttcttttgaaatgaaaacggttatactctaagtatagaatacaaaaaggccgatgctacgctatttttgttgaagccgttgactaatcattatatacggaaggaataacggcttgggtaataaattcaattgttggaggatgatttaa

SEQ ID NO: 143 - DNA, 34 bp, artificial sequence
  primer
tgctgattgaattccgaagacgggtagccctaag

SEQ ID NO: 144 - DNA, 37 bp, artificial sequence
  primer
tgtaggactcgagttaaatcatcctccaacaattgaa

SEQ ID NO: 145 - DNA, 22 bp, artificial sequence
  primer
gcatcaagcggtccgtaactag

SEQ ID NO: 146 - DNA, 25 bp, artificial sequence
  primer
gcgctataccgagattatatcatgc

SEQ ID NO: 147 - DNA, 28 bp, artificial sequence
  primer
ccagacatggtattataatctataagcg

SEQ ID NO: 148 - DNA, 22 bp, artificial sequence
  primer
tggtttatcaatcccgcgactc

SEQ ID NO: 149 - DNA, 6249 bp, artificial sequence
  constructed plasmid
cgaaagcttgtctaacacactagacttatttacttcgtaattaagtcgttaaaccgtgtgctctacgaccaaaactataaaacctttaagaactttctttttttacaagaaaaaagaaattagataaatctctcatatcttttattcaataatcgcatccgattgcagtataaatttaacgatcactcatcatgttcatatttatcagagctcgtgctataattatactaattttataaggaggaaaaaatatgggcatttttagtatttttgtaatcagcacagttcattatcaaccaaacaaaaaataagtggttataatgaatcgttaataagcaaaattcatataaccaaattaaagagggttataatgaacgagaaaaatataaaacacagtcaaaactttattacttcaaaacataatatagataaaataatgacaaatataagattaaatgaacatgataatatctttgaaatcggctcaggaaaaggccattttacccttgaattagtaaagaggtgtaatttcgtaactgccattgaaatagaccataaattatgcaaaactacagaaaataaacttgttgatcacgataatttccaagttttaaacaaggatatattgcagtttaaatttcctaaaaaccaatcctataaaatatatggtaatataccttataacataagtacggatataatacgcaaaattgtttttgatagtatagctaatgagatttatttaatcgtggaatacgggtttgctaaaagattattaaatacaaaacgctcattggcattacttttaatggcagaagttgatatttctatattaagtatggttccaagagaatattttcatcctaaacctaaagtgaatagctcacttatcagattaagtagaaaaaaatcaagaatatcacacaaagataaacaaaagtataattatttcgttatgaaatgggttaacaaagaatacaagaaaatatttacaaaaaatcaatttaacaattccttaaaacatgcaggaattgacgatttaaacaatattagctttgaacaattcttatctcttttcaatagctataaattatttaataagtaagttaagggatgcagttcatcgatgaagcttggatgttgtacaggataatgtccagaaggtcgatagaaagcgtgagaaacagcgtacagacgatttagagatgtagaggtacttttatgccgagaaaactttttgcgtgtgacagtccttaaaatatacttagagcgtaagcgaaagtagtagcgacagctattaactttcggttgcaaagctctaggatttttaatggacgcagcgcatcacacgcaaaaaggaaattggaataaatgcgaaatttgagatgttaattaaagacctttttgaggtctttttttcttagatttttggggttatttaggggagaaaacataggggggtactacgacctcccccctaggtgtccattgtccattgtccaaacaaataaataaatattgggtttttaatgttaaaaggttgttttttatgttaaagtgaaaaaaacagatgttgggaggtacagtgatagttgtagatagaaaagaagagaaaaaagttgctgttactttaagacttacaacagaagaaaatgagatattaaatagaatcaaagaaaaatataatattagcaaatcagatgcaaccggtattctaataaaaaaatatgcaaaggaggaatacggtgcattttaaacaaaaaaagatagacagcactggcatgctgcctatctatgactaaattttgttaagtgtattagcaccgttattatatcatgagcgaaaatgtaataaaagaaactgaaaacaagaaaaattcaagaggacgtaattggacatttgttttatatccagaatcagcaaaagccgagtggttagagtatttaaaagagttacacattcaatttgtagtgtctccattacatgatagggatactgatacagaaggtaggatgaaaaaagagcattatcatattctagtgatgtatgagggtaataaatcttatgaacagataaaaataattaacagaagaattgaatgcgactattccgcagattgcaggaagtgtgaaaggtcttgtgagatatatgcttcacatggacgatcctaataaatttaaatatcaaaaagaagatatgatagtttatggcggtgtagatgttgatgaattattaaagaaaacaacaacagatagatataaattaattaaagaaatgattgagtttattgatgaacaaggaatcgtagaatttaagagtttaatggattatgcaatgaagtttaaatttgatgattggttcccgcttttatgtgataactcggcgtatgttattcaagaatatataaaatcaaatcggtataaatctgaccgatagattttgaatttaggtgtcacaagacactcttttttcgcaccagcgaaaactggtttaagccgactgcgcaaaagacataatcgattcacaaaaaataggcacacgaaaaacaagttaagggatgcagtttatgcattcagatcttgatcccctgcgccatcagatccttggcggcaagaaagccatccagtttactttgcagggcttcccaaccttcccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctttctctgagctgtaacagcctgaccgcaacaaacgagaggatcgagaccatccgctccagattatccggctcctccatgcgttgcctctcggctcctgctccggttttccatgccttatggaactcctcgatccgccagcgatgggtataaatgtcgatgacgcgcaaggcttgggctagcgactcgaccggttcgctggtcagcaacaaccatttcaacggggtctcacccttgggcgggttaatctcctcggccagcaccgcgttgagcgtgatattcccctgttttagcgtgatgcgcccactgcgcaggctcaagctcgccttgcgggctggtcgatttttacgtttaccgcgtttatccaccacgcccttttgcggaatgctgatctgatagccacccaactccggttggttcttcagatggtcgtacagatacaacccagactctacgtccttgcgtgggtgcttggagcgcaccacgaagcgctcgttatgcgccagtttgtcctgcagataagcatgaatatcggcttcgcggtcacagaccgcaatcacgttgctcatcatgctgcccatgcgtaaccggctagttgcggcggctgccagccatttgccactctccttttcatccgcatcggcagggtcatccgggcgcatccaccactcctgatgcagtaatcctacggtgcggaatgtggtggcctcgagcaagagaacggagtgaacccaccatccgcgggatttatcctgaatagagcccagcttgccaagctcttcggcgacctggtggcgataactcaaagaggtggtgtcctcaatggccagcagttcgggaaactcctgagccaacttgactgtttgcatggcgccagcctttctgatcgcctcggcagaaacgttgggattgcggataaatcggtaagcgccttcctgcatggcttcactaccctctgatgagatggttattgatttaccagaatattttgccaattgggcggcgacgttaaccaagcgggcagtacggcgaggatcacccagcgccgccgaagagaacacagatttagcccagtcggccgcacgatgaagagcagaagttatcatgaacgttaccataataaatccccctttttgaaaataatgaagacttatattgttataataaaccagcaatctcgcattctgcaatataaaactagactccgcggctgaattgatagaattgtcccatgcgggctgtcggcgggcggtgtcaggggataagccgagagacacgtgttggattagacatgagtcgaatgacgcgattttttctggaaaaaatgacaaatgaagacgggaaaaatgataggggaaaatgttagatcatgcatctgtctcttgatcagatctcacaccgtgaacgcgttgcttaagtgtttaaacgataacttcgtataatgtatgctatacgaagttattctagattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaataattgtgagcgctcacaattccacacattatgccacaccttgtagataaagtcaacaactttttgcaaaatttttcaggaattttagcagaggttgttctggatgtagaacaaaacatctttccgctcttgtgctgttaggatatctttcttggaagctaggtaggcaagggctacctctagaataacttcgtataatgtatgctatacgaagttattaggtccctcgaagaggttcactagtactggccattgcggccgcataggatccatttgtcgactacttgtgtataagagtcaggcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtg

SEQ ID NO: 150 - DNA, 979 bp, artificial sequence
  synthesized fragment Tn5IE-loxP-cm-loxP cassette containing Tn5IE, loxP, chloramphenicol resistant gene (cm), and loxP
ctgtctcttgatcagatctcacaccgtgaacgcgttgcttaagtgtttaaacgataacttcgtataatgtatgctatacgaagttattctagattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaataattgtgagcgctcacaattccacacattatgccacaccttgtagataaagtcaacaactttttgcaaaatttttcaggaattttagcagaggttgttctggatgtagaacaaaacatctttccgctcttgtgctgttaggatatctttcttggaagctaggtaggcaagggctacctctagaataacttcgtataatgtatgctatacgaagttat

SEQ ID NO: 151 - DNA, 57 bp, artificial sequence
  primer
cgatatttgtcgactacttgtgtataagagtcaggcgctagcggagtgtatactggc

SEQ ID NO: 152 - DNA, 42 bp, artificial sequence
  primer
ctttatacgacgtcacaacttatatcgtatggggctgacttc

SEQ ID NO: 153 - DNA, 41 bp, artificial sequence
  primer
ttatactaagcttgtctaacacactagacttatttacttcg

SEQ ID NO: 154 - DNA, 42 bp, artificial sequence
  primer
ctctagtaagcttcatcgatgaactgcatcccttaacttact

SEQ ID NO: 155 - DNA, 251 bp, artificial sequence
  pPnpr promoter
gatctaacattttcccctatcatttttcccgtcttcatttgtcattttttccagaaaaaatcgcgtcattcgactcatgtctaatccaacacgtgtctctcggcttatcccctgacaccgcccgccgacagcccgcatgggacaattctatcaattcagccgcggagtctagttttatattgcagaatgcgagattgctggtttattataacaatataagtcttcattattttcaaaaagggggatttatt

SEQ ID NO: 156 - DNA, 47 bp, artificial sequence
  primer
ggattacgatgcatgatctaacattttcccctatcatttttcccgtc

SEQ ID NO: 157 - DNA, 49 bp, artificial sequence
  primer
atcatgaacgttaccataataaatccccctttttgaaaataatgaaaac

SEQ ID NO: 158 - DNA, 49 bp, artificial sequence
  primer
tttcaaaaagggggatttattatggtaacgttcatgataacttctgctc

SEQ ID NO: 159 - DNA, 46 bp, artificial sequence
  primer
actcatcttagatgcatcagatcttgatcccctgcgccatcagatc

SEQ ID NO: 160 - DNA, 1694 bp, artificial sequence
  PCR fragment with Pnrp and thp coding region
gatctaacattttcccctatcatttttcccgtcttcatttgtcattttttccagaaaaaatcgcgtcattcgactcatgtctaatccaacacgtgtctctcggcttatcccctgacaccgcccgccgacagcccgcatgggacaattctatcaattcagccgcggagtctagttttatattgcagaatgcgagattgctggtttattataacaatataagtcttcattattttcaaaaagggggatttattatggtaacgttcatgataacttctgctcttcatcgtgcggccgactgggctaaatctgtgttctcttcggcggcgctgggtgatcctcgccgtactgcccgcttggttaacgtcgccgcccaattggcaaaatattctggtaaatcaataaccatctcatcagagggtagtgaagccatgcaggaaggcgcttaccgatttatccgcaatcccaacgtttctgccgaggcgatcagaaaggctggcgccatgcaaacagtcaagttggctcaggagtttcccgaactgctggccattgaggacaccacctctttgagttatcgccaccaggtcgccgaagagcttggcaagctgggctctattcaggataaatcccgcggatggtgggttcactccgttctcttgctcgaggccaccacattccgcaccgtaggattactgcatcaggagtggtggatgcgcccggatgaccctgccgatgcggatgaaaaggagagtggcaaatggctggcagccgccgcaactagccggttacgcatgggcagcatgatgagcaacgtgattgcggtctgtgaccgcgaagccgatattcatgcttatctgcaggacaaactggcgcataacgagcgcttcgtggtgcgctccaagcacccacgcaaggacgtagagtctgggttgtatctgtacgaccatctgaagaaccaaccggagttgggtggctatcagatcagcattccgcaaaagggcgtggtggataaacgcggtaaacgtaaaaatcgaccagcccgcaaggcgagcttgagcctgcgcagtgggcgcatcacgctaaaacaggggaatatcacgctcaacgcggtgctggccgaggagattaacccgcccaagggtgagaccccgttgaaatggttgttgctgaccagcgaaccggtcgagtcgctagcccaagccttgcgcgtcatcgacatttatacccatcgctggcggatcgaggagttccataaggcatggaaaaccggagcaggagccgagaggcaacgcatggaggagccggataatctggagcggatggtctcgatcctctcgtttgttgcggtcaggctgttacagctcagagaaagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctgggaaggttgggaagccctgcaaagtaaactggatggctttcttgccgccaaggatctgatggcgcaggggatcaagatctga

SEQ ID NO: 161 - DNA, 29 bp, artificial sequence
  primer
ccaccacgcccttttgcggaatgctgatc

SEQ ID NO: 162 - DNA, 29 bp, artificial sequence
  primer
cggccgcacgatgaagagcagaagttatc

SEQ ID NO: 163 - DNA, 29 bp, artificial sequence
  primer
ctgaccgatagattttgaatttaggtgtc

SEQ ID NO: 164 - DNA, 114 bp, artificial sequence
  promoter fragment PgroE
aatgatgtaagcgtgaaaaattttttatcttatcacttgaaattggaagggagattctttattataagaattgtggaattgtgagcggataacaattcccaattaaaggaggaa

SEQ ID NO: 165 - DNA, 42 bp, artificial sequence
  primer
acattgtcgacggtaccgctaacggaaaagggagcggaaaag

SEQ ID NO: 166 - DNA, 27 bp, artificial sequence
  primer
gacctccccggatccttcctcctttaa

SEQ ID NO: 167 - DNA, 1681 bp, artificial sequence
  PCR fragment with ribosome binding site and kivD coding region optimized for L. plantarum
ggatccgtttaaacataaaatatggaggaatgcgatgtataccgttggtgactatttgttggatcggttacacgaattaggcatcgaagaaatcttcggtgttccaggcgattacaatttacaatttttggaccaaatcatcagtcacaaagacatgaaatgggtcggtaacgcaaatgaattaaatgcgagttacatggctgacggttatgctcggactaagaaagctgcagcctttttgacgacttttggtgttggtgaattatcagccgttaatggtttggccggtagttacgccgaaaatttaccggtcgttgaaattgttggcagtccaacgagtaaggttcaaaacgaaggtaaatttgtccatcataccttggcagatggtgattttaaacactttatgaagatgcatgaaccggtcactgctgcacggactttattaactgcggaaaatgccaccgtcgaaattgaccgcgttttatcagccttgttaaaagaacgcaaaccggtttacattaatttaccggtcgatgtggcagcggccaaagctgaaaaaccgagtttgccgttaaagaaggaaaatagtacctcaaatacctcagatcaagaaatcttgaacaagatccaagaatcattaaaaaatgctaaaaaaccaatcgttattaccggtcatgaaattattagttttggtttggaaaaaacggtgacgcaatttatttcaaagacgaaattaccaattacgaccttgaattttggtaaatcaagtgttgacgaagcgttgccaagtttcttgggtatctataatggtactttaagtgaaccgaatttaaaagaatttgtggaaagtgcagattttattttaatgttgggtgtcaagttaactgattcatcaactggcgcgttcacgcatcatttaaatgaaaataaaatgatttcattgaatattgatgaaggcaaaatcttcaacgaacggattcaaaattttgattttgaatcattgattagtagtttgttagatttatcagaaatcgaatacaaaggcaagtatattgataagaagcaggaagattttgttccgagtaatgcattgttaagtcaagatcgcttatggcaggcggttgaaaacttaactcaatcaaacgaaaccattgttgctgaacaaggtacttcattcttcggcgcatcaagtatctttttaaaatcaaaaagtcatttcatcggtcaaccattatggggtagtattggttacaccttcccagcggcgttaggtagtcaaattgctgacaaagaatcacgacacttattattcattggtgacggtagtttacaattgacggtccaggaattaggtttggccattcgcgaaaagatcaacccaatttgtttcattatcaataatgacggttatactgttgaacgggaaattcacggtccgaaccaaagttacaatgatattccaatgtggaactactcaaaattgccggaaagttttggcgccaccgaagatcgggtcgttagtaaaattgtgcggaccgaaaatgaattcgtgtcagtgatgaaggaagcacaagccgatccgaaccggatgtattggattgaattaatcttggctaaggaaggtgccccgaaggttttaaagaagatgggcaagttatttgcagaacaaaataaaagttaa

SEQ ID NO: 168 - DNA, 29 bp, artificial sequence
  primer
gacacccaacattaaaataaaatctgcac

SEQ ID NO: 169 - DNA, 1067 bp, artificial sequence
  PCR fragment with ribosome binding site and optimized sadB coding region
atataggaggaatttttgtaatgaaagctttggtttaccatggtgaccacaaaattagtttagaagataagccaaaaccaactttgcagaagccaactgatgtcgtggtccgtgtcttaaagacgaccatttgtggcacggacttgggtatctataagggcaagaatccagaagttgccgacggtcgtatcttaggtcatgaaggcgtcggtgttatcgaagaagttggtgaaagtgttacccaattcaagaagggtgacaaagttttaatcagttgtgttacgagttgtggttcatgtgattactgtaagaaacaattgtacagtcattgtcgtgacggtggttggatcttgggttacatgattgatggtgtccaagctgaatacgtccgtattccacacgcagataattcattgtataaaattccacaaactattgacgacgaaattgctgttttgttatcagatatcttaccaacgggccatgaaattggtgtccaatatggcaacgtccaaccaggcgatgccgttgcaattgttggcgccggtccagtgggcatgagtgttttattaacggctcaattctattcaccatcaactatcattgtcatcgacatggatgaaaatcgtttgcaattggctaaggaattgggcgctacgcacactatcaattcaggtacggaaaatgttgttgaagcagtccatcgtattgcagctgaaggcgtcgatgtggcaattgaagccgtgggcattccagcaacgtgggatatttgtcaagaaattgttaagccaggcgcccatatcgccaatgtgggtgtgcatggtgtgaaggttgattttgaaattcaaaaattgtggattaaaaatttgacgattactactggtttggttaacacgaatacgactccaatgttaatgaaggtcgcatcaaccgataaattaccattgaagaagatgattactcaccgttttgaattagcagaaattgaacatgcctatcaagttttcttaaacggcgccaaggaaaaagcaatgaaaattattttatcaaacgccggtgcagcttaa

SEQ ID NO: 170 - DNA, 66 bp, artificial sequence
  primer
acttgatatcgcggccgcatataggaggaatttttgtaatgaaagctttggtttaccatggtgacc

SEQ ID NO: 171 - DNA, 49 bp, artificial sequence
  primer
gttatatgactagcggccgcgagctcttaagctgcaccggcgtttgata

SEQ ID NO: 172 - DNA, 28 bp, artificial sequence
  primer
ggaagcacaagccgatccgaaccggatg

SEQ ID NO: 173 - DNA, 48 bp, artificial sequence
  primer
tcatttgatatgcctcctaaataattgtgagcgctcacaattccacac

SEQ ID NO: 174 - DNA, 6639 bp, artificial sequence
  constructed plasmid
gaattcagatctaattatagcaatcatttacgcgttaatggctaatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagttacccccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacgaacctggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcctgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacaagtttcctctccctctcattttcgtaggaattgttatccgctcacaattccttatacaaattatattttacatatcagtaaaataataacaacccccctttattcctattttttacacagcggacagtctggacagcataaaaaataccctgtctgatgacagacaaggtatttttatggtcttcttcttttctcaaacaatcgatccacttcttcagccaaatcatcagtcatcaaaggctcaatgttttcagccagtcttttcgtatgtgcgggtacctcgcgaaagcttggatgttgtacaggataatgtccagaaggtcgatagaaagcgtgagaaacagcgtacagacgatttagagatgtagaggtacttttatgccgagaaaactttttgcgtgtgacagtccttaaaatatacttagagcgtaagcgaaagtagtagcgacagctattaactttcggttgcaaagctctaggatttttaatggacgcagcgcatcacacgcaaaaaggaaattggaataaatgcgaaatttgagatgttaattaaagacctttttgaggtctttttttcttagatttttggggttatttaggggagaaaacataggggggtactacgacctcccccctaggtgtccattgtccattgtccaaacaaataaataaatattgggtttttaatgttaaaaggttgttttttatgttaaagtgaaaaaaacagatgttgggaggtacagtgatagttgtagatagaaaagaagagaaaaaagttgctgttactttaagacttacaacagaagaaaatgagatattaaatagaatcaaagaaaaatataatattagcaaatcagatgcaaccggtattctaataaaaaaatatgcaaaggaggaatacggtgcattttaaacaaaaaaagatagacagcactggcatgctgcctatctatgactaaattttgttaagtgtattagcaccgttattatatcatgagcgaaaatgtaataaaagaaactgaaaacaagaaaaattcaagaggacgtaattggacatttgttttatatccagaatcagcaaaagccgagtggttagagtatttaaaagagttacacattcaatttgtagtgtctccattacatgatagggatactgatacagaaggtaggatgaaaaaagagcattatcatattctagtgatgtatgagggtaataaatcttatgaacagataaaaataattaacagaagaattgaatgcgactattccgcagattgcaggaagtgtgaaaggtcttgtgagatatatgcttcacatggacgatcctaataaatttaaatatcaaaaagaagatatgatagtttatggcggtgtagatgttgatgaattattaaagaaaacaacaacagatagatataaattaattaaagaaatgattgagtttattgatgaacaaggaatcgtagaatttaagagtttaatggattatgcaatgaagtttaaatttgatgattggttcccgcttttatgtgataactcggcgtatgttattcaagaatatataaaatcaaatcggtataaatctgaccgatagattttgaatttaggtgtcacaagacactcttttttcgcaccagcgaaaactggtttaagccgactgcgcaaaagacataatcgattcacaaaaaataggcacacgaaaaacaagttaagggatgcagtttatgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttttgtttatctttgtgtgatattcttgatttttttctacttaatctgataagtgagctattcactttaggtttaggatgaaaatattctcttggaaccatacttaatatagaaatatcaacttctgccattaaaagtaatgccaatgagcgttttgtatttaataatcttttagcaaacccgtattccacgattaaataaatctcattagctatactatcaaaaacaattttgcgtattatatccgtacttatgttataaggtatattaccatatattttataggattggtttttaggaaatttaaactgcaatatatccttgtttaaaacttggaaattatcgtgatcaacaagtttattttctgtagttttgcataatttatggtctatttcaatggcagttacgaaattacacctctttactaattcaagggtaaaatggccttttcctgagccgatttcaaagatattatcatgttcatttaatcttatatttgtcattattttatctatattatgttttgaagtaataaagttttgactgtgttttatatttttctcgttcattataaccctctttaatttggttatatgaattttgcttattaacgattcattataaccacttattttttgtttggttgataatgaactgtgctgattacaaaaatactaaaaatgcccatattttttcctccttataaaattagtataattatagcacgagctctgataaatatgaacatgatgagtgatcgttaaatttatactgcaatcggatgcgattattgaataaaagatatgagagatttatctaatttcttttttcttgtaaaaaaagaaagttcttaaaggttttatagttttggtcgtagagcacacggtttaacgacttaattacgaagtaaataagtctagtgtgttagactttatgaaatctatatacgtttatatatatttattatccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaa

SEQ ID NO: 175 - DNA, 4895 bp, artificial sequence
  constructed plasmid
gacgaaagggccttataacttacaaataacccctcgaaaacattgaaagaataacccccaagatctatattatagatcttgggggttatttgttttaatattaaagaaatgacttcttctatttgtcatcaatactaaacaataatttgtacaaagtgattatttcttctagttcttcacgcgatacatgatcgacaatagtttcatcagtgacatgtcttgcccgtaaatctaaggctatggtttgatctaataatacttttccatatactgtttgactactagttagtcgatgatacattggaaaattacgcttggtactgctaattggagccacaatcgtcatgttacttgtctgacagactagatcattgcttagcgcaatggctggtcgcttattcatctgttcatgaccacggcttggattaaagttaacataaaatatatcaccttggcttaccattgaagttcattaccttctgactttccccaatcaagctcgtgatcccttttcccgtcatctttccaatccttaaatagttcgtgaatattggttgggttcttttttattggtgttaaaacaattgatccattttcaatggttattgtcatatcttggttatcatctaatttcagttgtttaataatttggctaggaattctagcagctttcgagtttccccactttgctaagcgtgtttgttctttaataagttccatattttcccctcctaaattattattacaagtcaagtatatcccatgtagatacacaatgcaaatattcttactggagaaataacaccttaagtctagcaccacccgcacgcatagcggtgcttaaaccatcaagggtcaagcccttaggctctctcaaacagttatcctaatcgtgaataactgcgcttcttttgcagtataaagagagaactctttatcagacaatttaagctcaaccagcccttgcactaactattattagagttggttttagcagcaacccgaataatctgcgttaatagttagcctgtccgtatcatttcctagtcttccagccacgtctttagtcgcgttgatctcgacaaggtttagcatacctatgttgttaactgcaagcggggtcacgaacgacactcacgggaggttttactagctaagaacaggtttccagcctttagttgctttgatggttgctaaccattgaataacaaaaaaacggttgctatcaggtttctgttaagattcccgataacaaccgtttactttaagtatcaatggttgaaaaacttagcctacatgttataatagtaccaagttagatagcttgtattggtagtacttgctatcgaaaatcttatcaggttgtgctgataagtcgtgaatcctaactctgctaagttgagggttcttttttttgcgttcatttattaagttgagtacattataaccgtaatataagattaatacaacctttatcattttaacgtctcaaccagccgaataatccttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataagggcccgcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtctcgaggaccgagcgcagcgagtcagtgagcgaggaagcggaagagcgagggcggagttgttgacagccgagcaggccttaactcacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttgacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcatcaaaatcgtctccctccgtttgaatatttgattgatcgtaaccagatgaagcactctttccactatccctacagtgttatggcttgaacaatcacgaaacaataattggtacgtacgatctttcagccgactcaaacatcaaatcttacaaatgtagtctttgaaagtattacatatgtaagatttaaatgcaaccgttttttcggaaggaaatgatgacctcgtttccaccggaattagcttggtaccagctattgtaacataatcggtacgggggtgaaaaagctaacggaaaagggagcggaaaagaatgatgtaagcgtgaaaaattttttatcttatcacttgaaattggaagggagattctttattataagaattgtggaattgtgagcggataacaattcccaattaaaggaggaaggatccaaaagcttaactgcaggacgggcttgtctgctcccggcatccgcttacagacaagctgtgaccgtctccgggagctgcatgtgtcagaggttttcaccgtcatcaccgaaacgcgcga

SEQ ID NO: 176 - DNA, 1548 bp, artificial sequence
  PCR fragment with B. subtilis groE promoter (PgroE) fused to a lacO operator sequence and a lacI repressor gene
ccgagcaggccttaactcacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttgacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcatcaaaatcgtctccctccgtttgaatatttgattgatcgtaaccagatgaagcactctttccactatccctacagtgttatggcttgaacaatcacgaaacaataattggtacgtacgatctttcagccgactcaaacatcaaatcttacaaatgtagtctttgaaagtattacatatgtaagatttaaatgcaaccgttttttcggaaggaaatgatgacctcgtttccaccggaattagcttggtaccagctattgtaacataatcggtacgggggtgaaaaagctaacggaaaagggagcggaaaagaatgatgtaagcgtgaaaaattttttatcttatcacttgaaattggaagggagattctttattataagaattgtggaattgtgagcggataacaattcccaattaaaggaggaaggatcc

SEQ ID NO: 177 - DNA, 32 bp, artificial sequence
  primer
gcgttacagatttataggcggccgctaagtcg

SEQ ID NO: 178 - DNA, 49 bp, artificial sequence
  primer
aatacattgtaactgccattacaaaaattcctcctatatatctcgaggc

SEQ ID NO: 179 - DNA, 51 bp, artificial sequence
  primer
taggaggaatttttgtaatggcagttacaatgtattatgaagatgatgtag

SEQ ID NO: 180 - DNA, 47 bp, artificial sequence
  primer
cctacttcagacgatcgttactgatagattttaaaggcatcgtcatc

SEQ ID NO: 181 - DNA, 26 bp, artificial sequence
  primer
tcgcgcgtttcggtgatgacggtgaa

SEQ ID NO: 182 - DNA, 6207 bp, artificial sequence
  constructed plasmid
gacgaaagggccttataacttacaaataacccctcgaaaacattgaaagaataacccccaagatctatattatagatcttgggggttatttgttttaatattaaagaaatgacttcttctatttgtcatcaatactaaacaataatttgtacaaagtgattatttcttctagttcttcacgcgatacatgatcgacaatagtttcatcagtgacatgtcttgcccgtaaatctaaggctatggtttgatctaataatacttttccatatactgtttgactactagttagtcgatgatacattggaaaattacgcttggtactgctaattggagccacaatcgtcatgttacttgtctgacagactagatcattgcttagcgcaatggctggtcgcttattcatctgttcatgaccacggcttggattaaagttaacataaaatatatcaccttggcttaccattgaagttcattaccttctgactttccccaatcaagctcgtgatcccttttcccgtcatctttccaatccttaaatagttcgtgaatattggttgggttcttttttattggtgttaaaacaattgatccattttcaatggttattgtcatatcttggttatcatctaatttcagttgtttaataatttggctaggaattctagcagctttcgagtttccccactttgctaagcgtgtttgttctttaataagttccatattttcccctcctaaattattattacaagtcaagtatatcccatgtagatacacaatgcaaatattcttactggagaaataacaccttaagtctagcaccacccgcacgcatagcggtgcttaaaccatcaagggtcaagcccttaggctctctcaaacagttatcctaatcgtgaataactgcgcttcttttgcagtataaagagagaactctttatcagacaatttaagctcaaccagcccttgcactaactattattagagttggttttagcagcaacccgaataatctgcgttaatagttagcctgtccgtatcatttcctagtcttccagccacgtctttagtcgcgttgatctcgacaaggtttagcatacctatgttgttaactgcaagcggggtcacgaacgacactcacgggaggttttactagctaagaacaggtttccagcctttagttgctttgatggttgctaaccattgaataacaaaaaaacggttgctatcaggtttctgttaagattcccgataacaaccgtttactttaagtatcaatggttgaaaaacttagcctacatgttataatagtaccaagttagatagcttgtattggtagtacttgctatcgaaaatcttatcaggttgtgctgataagtcgtgaatcctaactctgctaagttgagggttcttttttttgcgttcatttattaagttgagtacattataaccgtaatataagattaatacaacctttatcattttaacgtctcaaccagccgaataatccttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataagggcccgcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtctcgaggaccgagcgcagcgagtcagtgagcgaggaagcggaagagcgagggcggagttgttgacagccgagcaggccttaactcacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgttttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcatcaaaatcgtctccctccgtttgaatatttgattgatcgtaaccagatgaagcactctttccactatccctacagtgttatggcttgaacaatcacgaaacaataatcggtacgggggtgaaaaagctaacggaaaagggagcggaaaagaatgatgtaagcgtgaaaaattttttatcttatcacttgaaattggaagggagattctttattataagaattgtggaattgtgagcggataacaattcccaattaaaggaggaaggatcggccgctaagtcgtattggcaccactactcacaccgtgaccgacgcgcccgccagtcaagtgttcaaaagttagcgtttattaagtgcgataagtataccacaaagggcttattgacgcccgccaaagggttttgcggacattgttaataattgtattaaaagcatgctcaatctaacacttattttgcacaaacatggtatactttaaccgtaaaaactaaattttcactacgagaggatgacttattttgtcaagcctcgagatatataggaggaatttttgtaatggcagttacaatgtattatgaagatgatgtagaagtatcagcacttgctggaaagcaaattgcagtaatcggttatggttcacaaggacatgctcacgcacagaatttgcgtgattctggtcacaacgttatcattggtgtgcgccacggaaaatcttttgataaagcaaaagaagatggctttgaaacatttgaagtaggagaagcagtagctaaagctgatgttattatggttttggcaccagatgaacttcaacaatccatttatgaagaggacatcaaaccaaacttgaaagcaggttcagcacttggttttgctcacggatttaatatccattttggctatattaaagtaccagaagacgttgacgtctttatggttgcgcctaaggctccaggtcaccttgtccgtcggacttatactgaaggttttggtacaccagctttgtttgtttcacaccaaaatgcaagtggtcatgcgcgtgaaatcgcaatggattgggccaaaggaattggttgtgctcgagtgggaattattgaaacaacttttaaagaagaaacagaagaagatttgtttggagaacaagctgttctatgtggaggtttgacagcacttgttgaagccggttttgaaacactgacagaagctggatacgctggcgaattggcttactttgaagttttgcacgaaatgaaattgattgttgacctcatgtatgaaggtggttttactaaaatgcgtcaatccatctcaaatactgctgagtttggcgattatgtgactggtccacggattattactgacgaagttaaaaagaatatgaagcttgttttggctgatattcaatctggaaaatttgctcaagatttcgttgatgacttcaaagcggggcgtccaaaattaatagcctatcgcgaagctgcaaaaaatcttgaaattgaaaaaattggggcagagctacgtcaagcaatgccattcacacaatctggtgatgacgatgcctttaaaatctatcagtaacgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaa

SEQ ID NO: 183 - DNA, 1039 bp, artificial sequence
  ribosome binding site and PF5-ilvC coding region optimized for expression in Lactobacillus plantarum
atatataggaggaatttttgtaatgaaagtcttttatgataaggattgtgatttgtcaattattcaaggtaagaaggtcgctattattggttatggttcacaaggtcacgcccaagcctgtaatttgaaggatagtggtgttgatgttacggttggtttacggaagggtagtgctacggttgcgaaagccgaagcgcatggtttgaaggttacggatgttgcagctgccgtcgccggtgcggatttggttatgattttaacgccagatgaatttcaaagtcaattatataaaaatgaaattgaaccaaatattaaaaaaggtgctacgttagcttttagtcatggttttgcgattcattataatcaagttgtcccacgggctgatttagatgtcattatgattgcaccaaaagctccaggtcatacggttcgtagtgaatttgtcaagggtggtggtattccagatttgattgcaatttatcaagatgcaagtggtaatgccaaaaatgtcgctttaagttatgcggccggtgttggtggtggtcggacgggtattattgaaacgacgtttaaagatgaaacggaaacggatttgtttggtgaacaagccgtcttatgtggtggtacggttgaattagttaaggcgggttttgaaacgttggttgaagctggttatgcaccagaaatggcgtattttgaatgtttacatgaattaaaattaattgttgatttgatgtatgaaggtggtattgcaaatatgaattatagtattagtaataatgcagaatatggtgaatatgttacgggtccagaagttattaatgcagaaagtcggcaagcaatgcgtaatgccttaaaacggattcaagatggtgaatatgccaaaatgtttatttcagaaggtgcgacgggttatccaagtatgacggctaagcggcggaataatgccgcccatggtattgaaattattggtgaacaattacggagtatgatgccatggattggtgctaataaaattgttgataaggcaaagaattaa

SEQ ID NO: 184 - DNA, 5581 bp, artificial sequence
  constructed plasmid
gaattcactagtcttaagtaagtcgtattggcaccactactcacaccgtgaccgacgcgcccgccagtcaagtgttcaaaagttagcgtttattaagtgcgataagtataccacaaagggcttattgacgcccgccaaagggttttgcggacattgttaataattgtattaaaagcatgctcaatctaacacttattttgcacaaacatggtatactttaaccgtaaaaactaaattttcactacgagaggatgacttattttgtcaagcctcgagcccgggatcgatggtacctcgcgaaagcttggatgttgtacaggataatgtccagaaggtcgatagaaagcgtgagaaacagcgtacagacgatttagagatgtagaggtacttttatgccgagaaaactttttgcgtgtgacagtccttaaaatatacttagagcgtaagcgaaagtagtagcgacagctattaactttcggttgcaaagctctaggatttttaatggacgcagcgcatcacacgcaaaaaggaaattggaataaatgcgaaatttgagatgttaattaaagacctttttgaggtctttttttcttagatttttggggttatttaggggagaaaacataggggggtactacgacctcccccctaggtgtccattgtccattgtccaaacaaataaataaatattgggtttttaatgttaaaaggttgttttttatgttaaagtgaaaaaaacagatgttgggaggtacagtgatagttgtagatagaaaagaagagaaaaaagttgctgttactttaagacttacaacagaagaaaatgagatattaaatagaatcaaagaaaaatataatattagcaaatcagatgcaaccggtattctaataaaaaaatatgcaaaggaggaatacggtgcattttaaacaaaaaaagatagacagcactggcatgctgcctatctatgactaaattttgttaagtgtattagcaccgttattatatcatgagcgaaaatgtaataaaagaaactgaaaacaagaaaaattcaagaggacgtaattggacatttgttttatatccagaatcagcaaaagccgagtggttagagtatttaaaagagttacacattcaatttgtagtgtctccattacatgatagggatactgatacagaaggtaggatgaaaaaagagcattatcatattctagtgatgtatgagggtaataaatcttatgaacagataaaaataattaacagaagaattgaatgcgactattccgcagattgcaggaagtgtgaaaggtcttgtgagatatatgcttcacatggacgatcctaataaatttaaatatcaaaaagaagatatgatagtttatggcggtgtagatgttgatgaattattaaagaaaacaacaacagatagatataaattaattaaagaaatgattgagtttattgatgaacaaggaatcgtagaatttaagagtttaatggattatgcaatgaagtttaaatttgatgattggttcccgcttttatgtgataactcggcgtatgttattcaagaatatataaaatcaaatcggtataaatctgaccgatagattttgaatttaggtgtcacaagacactcttttttcgcaccagcgaaaactggtttaagccgactgcgcaaaagacataatcgattcacaaaaaataggcacacgaaaaacaagttaagggatgcagtttatgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttttgtttatctttgtgtgatattcttgatttttttctacttaatctgataagtgagctattcactttaggtttaggatgaaaatattctcttggaaccatacttaatatagaaatatcaacttctgccattaaaagtaatgccaatgagcgttttgtatttaataatcttttagcaaacccgtattccacgattaaataaatctcattagctatactatcaaaaacaattttgcgtattatatccgtacttatgttataaggtatattaccatatattttataggattggtttttaggaaatttaaactgcaatatatccttgtttaaaacttggaaattatcgtgatcaacaagtttattttctgtagttttgcataatttatggtctatttcaatggcagttacgaaattacacctctttactaattcaagggtaaaatggccttttcctgagccgatttcaaagatattatcatgttcatttaatcttatatttgtcattattttatctatattatgttttgaagtaataaagttttgactgtgttttatatttttctcgttcattataaccctctttaatttggttatatgaattttgcttattaacgattcattataaccacttattttttgtttggttgataatgaactgtgctgattacaaaaatactaaaaatgcccatattttttcctccttataaaattagtataattatagcacgagctctgataaatatgaacatgatgagtgatcgttaaatttatactgcaatcggatgcgattattgaataaaagatatgagagatttatctaatttcttttttcttgtaaaaaaagaaagttcttaaaggttttatagttttggtcgtagagcacacggtttaacgacttaattacgaagtaaataagtctagtgtgttagactttatgaaatctatatacgtttatatatatttattatccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaa

SEQ ID NO: 185 - DNA, 1297 bp, artificial sequence
  PldhL1-ilvC(P. fluorescens PF5) DNA fragment
taagtcgtattggcaccactactcacaccgtgaccgacgcgcccgccagtcaagtgttcaaaagttagcgtttattaagtgcgataagtataccacaaagggcttattgacgcccgccaaagggttttgcggacattgttaataattgtattaaaagcatgctcaatctaacacttattttgcacaaacatggtatactttaaccgtaaaaactaaattttcactacgagaggatgacttattttgtcaagcctcgagatatataggaggaatttttgtaatgaaagtcttttatgataaggattgtgatttgtcaattattcaaggtaagaaggtcgctattattggttatggttcacaaggtcacgcccaagcctgtaatttgaaggatagtggtgttgatgttacggttggtttacggaagggtagtgctacggttgcgaaagccgaagcgcatggtttgaaggttacggatgttgcagctgccgtcgccggtgcggatttggttatgattttaacgccagatgaatttcaaagtcaattatataaaaatgaaattgaaccaaatattaaaaaaggtgctacgttagcttttagtcatggttttgcgattcattataatcaagttgtcccacgggctgatttagatgtcattatgattgcaccaaaagctccaggtcatacggttcgtagtgaatttgtcaagggtggtggtattccagatttgattgcaatttatcaagatgcaagtggtaatgccaaaaatgtcgctttaagttatgcggccggtgttggtggtggtcggacgggtattattgaaacgacgtttaaagatgaaacggaaacggatttgtttggtgaacaagccgtcttatgtggtggtacggttgaattagttaaggcgggttttgaaacgttggttgaagctggttatgcaccagaaatggcgtattttgaatgtttacatgaattaaaattaattgttgatttgatgtatgaaggtggtattgcaaatatgaattatagtattagtaataatgcagaatatggtgaatatgttacgggtccagaagttattaatgcagaaagtcggcaagcaatgcgtaatgccttaaaacggattcaagatggtgaatatgccaaaatgtttatttcagaaggtgcgacgggttatccaagtatgacggctaagcggcggaataatgccgcccatggtattgaaattattggtgaacaattacggagtatgatgccatggattggtgctaataaaattgttgataaggcaaagaattaa

SEQ ID NO: 186 - DNA, 48 bp, artificial sequence
  primer
aacgaattgcgcggccgctaagtcgtattggcaccactactcacaccg

SEQ ID NO: 187 - DNA, 49 bp, artificial sequence
  primer
catacttgatatcttaattctttgccttatcaacaattttattagcacc

SEQ ID NO: 188 - DNA, 55 bp, artificial sequence
  primer
cagtaacggatccggggaggtcaaaaagataacatatggaattcaaatataacgg

SEQ ID NO: 189 - DNA, 52 bp, artificial sequence
  primer
atttgtggatccgcggccgcctataaatctgtaacgcaaccttcactagcgg

SEQ ID NO: 190 - DNA, 41 bp, artificial sequence
  primer
cttgtaggcgcggccgctaagtcgtattggcaccactactc

SEQ ID NO: 191 - DNA, 95 bp, artificial sequence
  primer
acttgtaggagacgggcttgtcgtttaaacgagctccctaggttaattaaggcgccgcggccgcgcttgacaaaataagtcatcctctcgtagtg

SEQ ID NO: 192 - DNA, 63 bp, artificial sequence
  primer
tgtaggacctaggatatataggaggaatttttgtaatggcagtagaaatgttatatgaagcag

SEQ ID NO: 193 - DNA, 45 bp, artificial sequence
  primer
attatgtcagagctcttagttataaattttgaaagcatcgtcatc

SEQ ID NO: 194 - DNA, 57 bp, artificial sequence
  primer
tgtaggacctaggatatataggaggaatttttgtaatggcagttcaaatggaatatg

SEQ ID NO: 195 - DNA, 43 bp, artificial sequence
  primer
attatgtcagagctcttagttatagattttgaatgcatcatcg

SEQ ID NO: 196 - DNA, 63 bp, artificial sequence
  primer
tgtaggacctaggatatataggaggaatttttgtaatgactacaaaaatgttttatgataaag

SEQ ID NO: 197 - DNA, 39 bp, artificial sequence
  primer
attatgtcagagctcttactttgctgctgagaattgctc

SEQ ID NO: 198 - DNA, 11856 bp, artificial sequence
  constructed plasmid
tcccattaccgacatttgggcgctatacgtgcatatgttcatgtatgtatctgtatttaaaacacttttgtattatttttcctcatatatgtgtataggtttatacggatgatttaattattacttcaccaccctttatttcaggctgatatcttagccttgttactagttagaaaaagacatttttgctgtcagtcactgtcaagagattcttttgctggcatttcttctagaagcaaaaagagcgatgcgtcttttccgctgaaccgttccagcaaaaaagactaccaacgcaatatggattgtcagaatcatataaaagagaagcaaataactccttgtcttgtatcaattgcattataatatcttcttgttagtgcaatatcatatagaagtcatcgaaatagatattaagaaaaacaaactgtacaatcaatcaatcaatcatcgctgaggatgttgacaaaagcaacaaaagaacaaaaatcccttgtgaaaaacagaggggcggagcttgttgttgattgcttagtggagcaaggtgtcacacatgtatttggcattccaggtgcaaaaattgatgcggtatttgacgctttacaagataaaggacctgaaattatcgttgcccggcacgaacaaaacgcagcattcatggcccaagcagtcggccgtttaactggaaaaccgggagtcgtgttagtcacatcaggaccgggtgcctctaacttggcaacaggcctgctgacagcgaacactgaaggagaccctgtcgttgcgcttgctggaaacgtgatccgtgcagatcgtttaaaacggacacatcaatctttggataatgcggcgctattccagccgattacaaaatacagtgtagaagttcaagatgtaaaaaatataccggaagctgttacaaatgcatttaggatagcgtcagcagggcaggctggggccgcttttgtgagctttccgcaagatgttgtgaatgaagtcacaaatacgaaaaacgtgcgtgctgttgcagcgccaaaactcggtcctgcagcagatgatgcaatcagtgcggccatagcaaaaatccaaacagcaaaacttcctgtcgttttggtcggcatgaaaggcggaagaccggaagcaattaaagcggttcgcaagcttttgaaaaaggttcagcttccatttgttgaaacatatcaagctgccggtaccctttctagagatttagaggatcaatattttggccgtatcggtttgttccgcaaccagcctggcgatttactgctagagcaggcagatgttgttctgacgatcggctatgacccgattgaatatgatccgaaattctggaatatcaatggagaccggacaattatccatttagacgagattatcgctgacattgatcatgcttaccagcctgatcttgaattgatcggtgacattccgtccacgatcaatcatatcgaacacgatgctgtgaaagtggaatttgcagagcgtgagcagaaaatcctttctgatttaaaacaatatatgcatgaaggtgagcaggtgcctgcagattggaaatcagacagagcgcaccctcttgaaatcgttaaagagttgcgtaatgcagtcgatgatcatgttacagtaacttgcgatatcggttcgcacgccatttggatgtcacgttatttccgcagctacgagccgttaacattaatgatcagtaacggtatgcaaacactcggcgttgcgcttccttgggcaatcggcgcttcattggtgaaaccgggagaaaaagtggtttctgtctctggtgacggcggtttcttattctcagcaatggaattagagacagcagttcgactaaaagcaccaattgtacacattgtatggaacgacagcacatatgacatggttgcattccagcaattgaaaaaatataaccgtacatctgcggtcgatttcggaaatatcgatatcgtgaaatatgcggaaagcttcggagcaactggcttgcgcgtagaatcaccagaccagctggcagatgttctgcgtcaaggcatgaacgctgaaggtcctgtcatcatcgatgtcccggttgactacagtgataacattaatttagcaagtgacaagcttccgaaagaattcggggaactcatgaaaacgaaagctctctagttaattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcgggcggccgcacctggtaaaacctctagtggagtagtagatgtaatcaatgaagcggaagccaaaagaccagagtagaggcctatagaagaaactgcgataccttttgtgatggctaaacaaacagacatctttttatatgtttttacttctgtatatcgtgaagtagtaagtgataagcgaatttggctaagaacgttgtaagtgaacaagggacctcttttgcctttcaaaaaaggattaaatggagttaatcattgagatttagttttcgttagattctgtatccctaaataactcccttacccgacgggaaggcacaaaagacttgaataatagcaaacggccagtagccaagaccaaataatactagagttaactgatggtcttaaacaggcattacgtggtgaactccaagaccaatatacaaaatatcgataagttattcttgcccaccaatttaaggagcctacatcaggacagtagtaccattcctcagagaagaggtatacataacaagaaaatcgcgtgaacaccttatataacttagcccgttattgagctaaaaaaccttgcaaaatttcctatgaataagaatacttcagacgtgataaaaatttactttctaactcttctcacgctgcccctatctgttcttccgctctaccgtgagaaataaagcatcgagtacggcagttcgctgtcactgaactaaaacaataaggctagttcgaatgatgaacttgcttgctgtcaaacttctgagttgccgctgatgtgacactgtgacaataaattcaaaccggttatagcggtctcctccggtaccggttctgccacctccaatagagctcagtaggagtcagaacctctgcggtggctgtcagtgactcatccgcgtttcgtaagttgtgcgcgtgcacatttcgcccgttcccgctcatcttgcagcaggcggaaattttcatcacgctgtaggacgcaaaaaaaaaataattaatcgtacaagaatcttggaaaaaaaattgaaaaattttgtataaaagggatgacctaacttgactcaatggcttttacacccagtattttccctttccttgtttgttacaattatagaagcaagacaaaaacatatagacaacctattcctaggagttatatttttttaccctaccagcaatataagtaaaaaactgtttaaacagtatggcagttacaatgtattatgaagatgatgtagaagtatcagcacttgctggaaagcaaattgcagtaatcggttatggttcacaaggacatgctcacgcacagaatttgcgtgattctggtcacaacgttatcattggtgtgcgccacggaaaatcttttgataaagcaaaagaagatggctttgaaacatttgaagtaggagaagcagtagctaaagctgatgttattatggttttggcaccagatgaacttcaacaatccatttatgaagaggacatcaaaccaaacttgaaagcaggttcagcacttggttttgctcacggatttaatatccattttggctatattaaagtaccagaagacgttgacgtctttatggttgcgcctaaggctccaggtcaccttgtccgtcggacttatactgaaggttttggtacaccagctttgtttgtttcacaccaaaatgcaagtggtcatgcgcgtgaaatcgcaatggattgggccaaaggaattggttgtgctcgagtgggaattattgaaacaacttttaaagaagaaacagaagaagatttgtttggagaacaagctgttctatgtggaggtttgacagcacttgttgaagccggttttgaaacactgacagaagctggatacgctggcgaattggcttactttgaagttttgcacgaaatgaaattgattgttgacctcatgtatgaaggtggttttactaaaatgcgtcaatccatctcaaatactgctgagtttggcgattatgtgactggtccacggattattactgacgaagttaaaaagaatatgaagcttgttttggctgatattcaatctggaaaatttgctcaagatttcgttgatgacttcaaagcggggcgtccaaaattaatagcctatcgcgaagctgcaaaaaatcttgaaattgaaaaaattggggcagagctacgtcaagcaatgccattcacacaatctggtgatgacgatgcctttaaaatctatcagtaaggccctgcaggccagaggaaaataatatcaagtgctggaaactttttctcttggaatttttgcaacatcaagtcatagtcaattgaattgacccaatttcacatttaagattttttttttttcatccgacatacatctgtacactaggaagccctgtttttctgaagcagcttcaaatatatatattttttacatatttattatgattcaatgaacaatctaattaaatcgaaaacaagaaccgaaacgcgaataaataatttatttagatggtgacaagtgtataagtcctcatcgggacagctacgatttctctttcggttttggctgagctactggttgctgtgacgcagcggcattagcgcggcgttatgagctaccctcgtggcctgaaagatggcgggaataaagcggaactaaaaattactgactgagccatattgaggtcaatttgtcaactcgtcaagtcacgtttggtggacggcccctttccaacgaatcgtatatactaacatgcgcgcgcttcctatatacacatatacatatatatatatatatatatgtgtgcgtgtatgtgtacacctgtatttaatttccttactcgcgggtttttcttttttctcaattcttggcttcctctttctcgagcggaccggatcctccgcggtgccggcagatctatttaaatggcgcgccgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttttctttccaattttttttttttcgtcattataaaaatcattacgaccgagattcccgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagcttgcatgcctgcaggtcgactctagtatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcattttttttttttttttttttttttttttttttttttttttttttttttttttgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggctttaccttcttcaggcaagttcaatgacaatttcaacatcattgcagcagacaagatagtggcgatagggttgaccttattctttggcaaatctggagcggaaccatggcatggttcgtacaaaccaaatgcggtgttcttgtctggcaaagaggccaaggacgcagatggcaacaaacccaaggagcctgggataacggaggcttcatcggagatgatatcaccaaacatgttgctggtgattataataccatttaggtgggttgggttcttaactaggatcatggcggcagaatcaatcaattgatgttgaactttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctaacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcagccttcttggaggcttccagcgcctcatctggaagtggaacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacattacaatggtatatccttgaaatatatataaaaaaaaaaaaaaaaaaaaaaaaaaaaaatgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatatgcatagtaccgagaaactagaggatc

SEQ ID NO: 199 - DNA, 39 bp, artificial sequence
  primer
gcagtttaaacagtatggcagttacaatgtattatgaag

SEQ ID NO: 200 - DNA, 38 bp, artificial sequence
  primer
gataggcctgcagggccttactgatagattttaaaggc

SEQ ID NO: 201 - DNA, 83 bp, artificial sequence
  primer
gagtcctaggagttatatttttttaccctaccagcaatataagtaaaaaataaaaatggcagttacaatgtattatgaagatg

SEQ ID NO: 202 - DNA, 45 bp, artificial sequence
  primer
gataggcctgcagggccttactgatagattttaaaggcatcgtca

SEQ ID NO: 203 - DNA, 16405 bp, artificial sequence
  constructed plasmid
tcccattaccgacatttgggcgctatacgtgcatatgttcatgtatgtatctgtatttaaaacacttttgtattatttttcctcatatatgtgtataggtttatacggatgatttaattattacttcaccaccctttatttcaggctgatatcttagccttgttactagttagaaaaagacatttttgctgtcagtcactgtcaagagattcttttgctggcatttcttctagaagcaaaaagagcgatgcgtcttttccgctgaaccgttccagcaaaaaagactaccaacgcaatatggattgtcagaatcatataaaagagaagcaaataactccttgtcttgtatcaattgcattataatatcttcttgttagtgcaatatcatatagaagtcatcgaaatagatattaagaaaaacaaactgtacaatcaatcaatcaatcatcgctgaggatgttgacaaaagcaacaaaagaacaaaaatcccttgtgaaaaacagaggggcggagcttgttgttgattgcttagtggagcaaggtgtcacacatgtatttggcattccaggtgcaaaaattgatgcggtatttgacgctttacaagataaaggacctgaaattatcgttgcccggcacgaacaaaacgcagcattcatggcccaagcagtcggccgtttaactggaaaaccgggagtcgtgttagtcacatcaggaccgggtgcctctaacttggcaacaggcctgctgacagcgaacactgaaggagaccctgtcgttgcgcttgctggaaacgtgatccgtgcagatcgtttaaaacggacacatcaatctttggataatgcggcgctattccagccgattacaaaatacagtgtagaagttcaagatgtaaaaaatataccggaagctgttacaaatgcatttaggatagcgtcagcagggcaggctggggccgcttttgtgagctttccgcaagatgttgtgaatgaagtcacaaatacgaaaaacgtgcgtgctgttgcagcgccaaaactcggtcctgcagcagatgatgcaatcagtgcggccatagcaaaaatccaaacagcaaaacttcctgtcgttttggtcggcatgaaaggcggaagaccggaagcaattaaagcggttcgcaagcttttgaaaaaggttcagcttccatttgttgaaacatatcaagctgccggtaccctttctagagatttagaggatcaatattttggccgtatcggtttgttccgcaaccagcctggcgatttactgctagagcaggcagatgttgttctgacgatcggctatgacccgattgaatatgatccgaaattctggaatatcaatggagaccggacaattatccatttagacgagattatcgctgacattgatcatgcttaccagcctgatcttgaattgatcggtgacattccgtccacgatcaatcatatcgaacacgatgctgtgaaagtggaatttgcagagcgtgagcagaaaatcctttctgatttaaaacaatatatgcatgaaggtgagcaggtgcctgcagattggaaatcagacagagcgcaccctcttgaaatcgttaaagagttgcgtaatgcagtcgatgatcatgttacagtaacttgcgatatcggttcgcacgccatttggatgtcacgttatttccgcagctacgagccgttaacattaatgatcagtaacggtatgcaaacactcggcgttgcgcttccttgggcaatcggcgcttcattggtgaaaccgggagaaaaagtggtttctgtctctggtgacggcggtttcttattctcagcaatggaattagagacagcagttcgactaaaagcaccaattgtacacattgtatggaacgacagcacatatgacatggttgcattccagcaattgaaaaaatataaccgtacatctgcggtcgatttcggaaatatcgatatcgtgaaatatgcggaaagcttcggagcaactggcttgcgcgtagaatcaccagaccagctggcagatgttctgcgtcaaggcatgaacgctgaaggtcctgtcatcatcgatgtcccggttgactacagtgataacattaatttagcaagtgacaagcttccgaaagaattcggggaactcatgaaaacgaaagctctctagttaattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcgggcggccgctctagaactagtaccacaggtgttgtcctctgaggacataaaatacacaccgagattcatcaactcattgctggagttagcatatctacaattgggtgaaatggggagcgatttgcaggcatttgctcggcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttactcttaattaatcaagcatctaaaacacaaccgttggaagcgttggaaaccaacttagcatacttggatagagtacctcttgtgtaacgaggtggaggtgcaacccaactttgtttacgttgagccatttccttatcagagactaataggtcaatcttgttattatcagcatcaatgataatctcatcgccgtctctgaccaacccgataggaccaccttcagcggcttcgggaacaatgtggccgattaagaacccgtgagaaccaccagagaatctaccatcagtcaacaatgcaacatctttacccaaaccgtaacccatcagagcagaggaaggctttagcatttcaggcatacctggtgcacctcttggaccttcatatctgataacaacaacggttttttcacccttcttgatttcacctctttccaaggcttcaataaaggcaccttcctcttcgaacacacgtgctctacccttgaagtaagtaccttccttaccggtaattttacccacagctccacctggtgccaatgaaccgtacagaatttgcaagtgaccgttggccttgattgggtgggagagtggcttaataatctcttgtccttcaggtaggcttggtgctttctttgcacgttctgccaaagtgtcaccggtaacagtcattgtgttaccgtgcaacatgttgttttcatatagatacttaatcacagattgggtaccaccaacgttaatcaaatcggccatgacgtatttaccagaaggtttgaagtcaccgatcaatggtgtagtatcactgattctttggaaatcatctggtgacaacttgacacccgcagagtgagcaacagccaccaaatgcaaaacagcattagtggacccaccggttgcaacgacataagtaatggcgttttcaaaagcctcttttgtgaggatatcacgaggtaaaatacccaattccattgtcttcttgatgtattcaccaatgttgtcacactcagctaacttctccttggaaacggctgggaaggaagaggagtttggaatggtcaaacctagcacttcagcggcagaagccattgtgttggcagtatacataccaccacaagaaccaggacctgggcatgcatgttccacaacatcttctctttcttcttcagtgaattgcttggaaatatattcaccgtaggattggaacgcagagacgatatcgatgtttttagagatcctgttaaaacctctagtggagtagtagatgtaatcaatgaagcggaagccaaaagaccagagtagaggcctatagaagaaactgcgataccttttgtgatggctaaacaaacagacatctttttatatgtttttacttctgtatatcgtgaagtagtaagtgataagcgaatttggctaagaacgttgtaagtgaacaagggacctcttttgcctttcaaaaaaggattaaatggagttaatcattgagatttagttttcgttagattctgtatccctaaataactcccttacccgacgggaaggcacaaaagacttgaataatagcaaacggccagtagccaagaccaaataatactagagttaactgatggtcttaaacaggcattacgtggtgaactccaagaccaatatacaaaatatcgataagttattcttgcccaccaatttaaggagcctacatcaggacagtagtaccattcctcagagaagaggtatacataacaagaaaatcgcgtgaacaccttatataacttagcccgttattgagctaaaaaaccttgcaaaatttcctatgaataagaatacttcagacgtgataaaaatttactttctaactcttctcacgctgcccctatctgttcttccgctctaccgtgagaaataaagcatcgagtacggcagttcgctgtcactgaactaaaacaataaggctagttcgaatgatgaacttgcttgctgtcaaacttctgagttgccgctgatgtgacactgtgacaataaattcaaaccggttatagcggtctcctccggtaccggttctgccacctccaatagagctcagtaggagtcagaacctctgcggtggctgtcagtgactcatccgcgtttcgtaagttgtgcgcgtgcacatttcgcccgttcccgctcatcttgcagcaggcggaaattttcatcacgctgtaggacgcaaaaaaaaaataattaatcgtacaagaatcttggaaaaaaaattgaaaaattttgtataaaagggatgacctaacttgactcaatggcttttacacccagtattttccctttccttgtttgttacaattatagaagcaagacaaaaacatatagacaacctattcctaggagttatatttttttaccctaccagcaatataagtaaaaaataaaaatggcagttacaatgtattatgaagatgatgtagaagtatcagcacttgctggaaagcaaattgcagtaatcggttatggttcacaaggacatgctcacgcacagaatttgcgtgattctggtcacaacgttatcattggtgtgcgccacggaaaatcttttgataaagcaaaagaagatggctttgaaacatttgaagtaggagaagcagtagctaaagctgatgttattatggttttggcaccagatgaacttcaacaatccatttatgaagaggacatcaaaccaaacttgaaagcaggttcagcacttggttttgctcacggatttaatatccattttggctatattaaagtaccagaagacgttgacgtctttatggttgcgcctaaggctccaggtcaccttgtccgtcggacttatactgaaggttttggtacaccagctttgtttgtttcacaccaaaatgcaagtggtcatgcgcgtgaaatcgcaatggattgggccaaaggaattggttgtgctcgagtgggaattattgaaacaacttttaaagaagaaacagaagaagatttgtttggagaacaagctgttctatgtggaggtttgacagcacttgttgaagccggttttgaaacactgacagaagctggatacgctggcgaattggcttactttgaagttttgcacgaaatgaaattgattgttgacctcatgtatgaaggtggttttactaaaatgcgtcaatccatctcaaatactgctgagtttggcgattatgtgactggtccacggattattactgacgaagttaaaaagaatatgaagcttgttttggctgatattcaatctggaaaatttgctcaagatttcgttgatgacttcaaagcggggcgtccaaaattaatagcctatcgcgaagctgcaaaaaatcttgaaattgaaaaaattggggcagagctacgtcaagcaatgccattcacacaatctggtgatgacgatgcctttaaaatctatcagtaaggccctgcaggccagaggaaaataatatcaagtgctggaaactttttctcttggaatttttgcaacatcaagtcatagtcaattgaattgacccaatttcacatttaagattttttttttttcatccgacatacatctgtacactaggaagccctgtttttctgaagcagcttcaaatatatatattttttacatatttattatgattcaatgaacaatctaattaaatcgaaaacaagaaccgaaacgcgaataaataatttatttagatggtgacaagtgtataagtcctcatcgggacagctacgatttctctttcggttttggctgagctactggttgctgtgacgcagcggcattagcgcggcgttatgagctaccctcgtggcctgaaagatggcgggaataaagcggaactaaaaattactgactgagccatattgaggtcaatttgtcaactcgtcaagtcacgtttggtggacggcccctttccaacgaatcgtatatactaacatgcgcgcgcttcctatatacacatatacatatatatatatatatatatgtgtgcgtgtatgtgtacacctgtatttaatttccttactcgcgggtttttcttttttctcaattcttggcttcctctttctcgagtatataatttttcaggtaaaatttagtacgatagtaaaatacttctcgaactcgtcacatatacgtgtacataatgtctgaaccagctcaaaagaaacaaaaggttgctaacaactctctagagcggccgcccgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgattaattaattattggttttctggtctcaactttctgacttccttaccaaccttccagatttccatgtttctgatggtgtctaattccttttctagcttttctctgtagtcaggttgagagttgaattccaaagatctcttggtttcggtaccgttcttggtagattcgtacaagtcttggaaaacaggcttcaaagcattcttgaagattgggtaccagtccaaagcacctcttctggcggtggtggaacaagcatcgtacatgtaatccataccgtacttaccgatcaatgggtatagagattgggtagcttcttcgacggtttcgttgaaagcttcagatggggagtgaccgttttctctcaagacgtcgtattgagccaagaacataccgtggataccacccattaaacaacctctttcaccgtacaagtcagagttgacttctctttcgaaagtggtttggtaaacgtaaccggaaccaatggcaacggccaaagcttgggccttttcgtgagccttaccggtgacatcgttccagacggcgtaagaagagttaataccacgaccttccttgaacaaagatctgacagttctaccggaaccctttggagcaaccaagataacatctaagtcctttggtggttcaacgtgagtcaagtccttgaagactggggagaaaccgtgggagaagtacaaagtcttacccttggtcaacaatggcttgatagcaggccaggtttctgattgagcggcatcggacaacaagttcataacgtaactacctctcttgatagcatcttcaacagtgaacaagttcttgcctggaacccaaccgtcttcgatggcagccttccaagaagcaccatctttacggacaccaatgataacgttcaaaccgttgtctctcaagttcaaaccttgaccgtaaccttgggaaccgtaaccgatcaaagcaaaagtgtcgttcttgaagtagtccaacaacttttctcttggccagtcagctctttcgtagacggtttcaacagtaccaccgaagttgatttgcttcaacatcctcagctctagatttgaatatgtattacttggttatggttatatatgacaaaagaaaaagaagaacagaagaataacgcaaggaagaacaataactgaaattgatagagaagtattatgtctttgtctttttataataaatcaagtgcagaaatccgttagacaacatgagggataaaatttaacgtgggcgaagaagaaggaaaaaagtttttgtgagggcgtaattgaagcgatctgttgattgtagattttttttttttgaggagtcaaagtcagaagagaacagacaaatggtattaaccatccaatacttttttggagcaacgctaagctcatgcttttccattggttacgtgctcagttgttagatatggaaagagaggatgctcacggcagcgtgactccaattgagcccgaaagagaggatgccacgttttcccgacggctgctagaatggaaaaaggaaaaatagaagaatcccattcctatcattatttacgtaatgacccacacatttttgagattttcaactattacgtattacgataatcctgctgtcattatcattattatctatatcgacgtatgcaacgtatgtgaagccaagtaggcaattatttagtactgtcagtattgttattcatttcagatctatccgcggtggagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttttctttccaattttttttttttcgtcattataaaaatcattacgaccgagattcccgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagcttgcatgcctgcaggtcgactctagtatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcattttttttttttttttttttttttttttttttttttttttttttttttttttgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggctttaccttcttcaggcaagttcaatgacaatttcaacatcattgcagcagacaagatagtggcgatagggttgaccttattctttggcaaatctggagcggaaccatggcatggttcgtacaaaccaaatgcggtgttcttgtctggcaaagaggccaaggacgcagatggcaacaaacccaaggagcctgggataacggaggcttcatcggagatgatatcaccaaacatgttgctggtgattataataccatttaggtgggttgggttcttaactaggatcatggcggcagaatcaatcaattgatgttgaactttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctaacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcagccttcttggaggcttccagcgcctcatctggaagtggaacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacattacaatggtatatccttgaaatatatataaaaaaaaaaaaaaaaaaaaaaaaaaaaaatgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatatgcatagtaccgagaaactagaggatc

SEQ ID NO: 204 - DNA, 11838 bp, artificial sequence
  constructed plasmid
tcccattaccgacatttgggcgctatacgtgcatatgttcatgtatgtatctgtatttaaaacacttttgtattatttttcctcatatatgtgtataggtttatacggatgatttaattattacttcaccaccctttatttcaggctgatatcttagccttgttactagttagaaaaagacatttttgctgtcagtcactgtcaagagattcttttgctggcatttcttctagaagcaaaaagagcgatgcgtcttttccgctgaaccgttccagcaaaaaagactaccaacgcaatatggattgtcagaatcatataaaagagaagcaaataactccttgtcttgtatcaattgcattataatatcttcttgttagtgcaatatcatatagaagtcatcgaaatagatattaagaaaaacaaactgtacaatcaatcaatcaatcatcgctgaggatgttgacaaaagcaacaaaagaacaaaaatcccttgtgaaaaacagaggggcggagcttgttgttgattgcttagtggagcaaggtgtcacacatgtatttggcattccaggtgcaaaaattgatgcggtatttgacgctttacaagataaaggacctgaaattatcgttgcccggcacgaacaaaacgcagcattcatggcccaagcagtcggccgtttaactggaaaaccgggagtcgtgttagtcacatcaggaccgggtgcctctaacttggcaacaggcctgctgacagcgaacactgaaggagaccctgtcgttgcgcttgctggaaacgtgatccgtgcagatcgtttaaaacggacacatcaatctttggataatgcggcgctattccagccgattacaaaatacagtgtagaagttcaagatgtaaaaaatataccggaagctgttacaaatgcatttaggatagcgtcagcagggcaggctggggccgcttttgtgagctttccgcaagatgttgtgaatgaagtcacaaatacgaaaaacgtgcgtgctgttgcagcgccaaaactcggtcctgcagcagatgatgcaatcagtgcggccatagcaaaaatccaaacagcaaaacttcctgtcgttttggtcggcatgaaaggcggaagaccggaagcaattaaagcggttcgcaagcttttgaaaaaggttcagcttccatttgttgaaacatatcaagctgccggtaccctttctagagatttagaggatcaatattttggccgtatcggtttgttccgcaaccagcctggcgatttactgctagagcaggcagatgttgttctgacgatcggctatgacccgattgaatatgatccgaaattctggaatatcaatggagaccggacaattatccatttagacgagattatcgctgacattgatcatgcttaccagcctgatcttgaattgatcggtgacattccgtccacgatcaatcatatcgaacacgatgctgtgaaagtggaatttgcagagcgtgagcagaaaatcctttctgatttaaaacaatatatgcatgaaggtgagcaggtgcctgcagattggaaatcagacagagcgcaccctcttgaaatcgttaaagagttgcgtaatgcagtcgatgatcatgttacagtaacttgcgatatcggttcgcacgccatttggatgtcacgttatttccgcagctacgagccgttaacattaatgatcagtaacggtatgcaaacactcggcgttgcgcttccttgggcaatcggcgcttcattggtgaaaccgggagaaaaagtggtttctgtctctggtgacggcggtttcttattctcagcaatggaattagagacagcagttcgactaaaagcaccaattgtacacattgtatggaacgacagcacatatgacatggttgcattccagcaattgaaaaaatataaccgtacatctgcggtcgatttcggaaatatcgatatcgtgaaatatgcggaaagcttcggagcaactggcttgcgcgtagaatcaccagaccagctggcagatgttctgcgtcaaggcatgaacgctgaaggtcctgtcatcatcgatgtcccggttgactacagtgataacattaatttagcaagtgacaagcttccgaaagaattcggggaactcatgaaaacgaaagctctctagttaattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcgggcggccgcacctggtaaaacctctagtggagtagtagatgtaatcaatgaagcggaagccaaaagaccagagtagaggcctatagaagaaactgcgataccttttgtgatggctaaacaaacagacatctttttatatgtttttacttctgtatatcgtgaagtagtaagtgataagcgaatttggctaagaacgttgtaagtgaacaagggacctcttttgcctttcaaaaaaggattaaatggagttaatcattgagatttagttttcgttagattctgtatccctaaataactcccttacccgacgggaaggcacaaaagacttgaataatagcaaacggccagtagccaagaccaaataatactagagttaactgatggtcttaaacaggcattacgtggtgaactccaagaccaatatacaaaatatcgataagttattcttgcccaccaatttaaggagcctacatcaggacagtagtaccattcctcagagaagaggtatacataacaagaaaatcgcgtgaacaccttatataacttagcccgttattgagctaaaaaaccttgcaaaatttcctatgaataagaatacttcagacgtgataaaaatttactttctaactcttctcacgctgcccctatctgttcttccgctctaccgtgagaaataaagcatcgagtacggcagttcgctgtcactgaactaaaacaataaggctagttcgaatgatgaacttgcttgctgtcaaacttctgagttgccgctgatgtgacactgtgacaataaattcaaaccggttatagcggtctcctccggtaccggttctgccacctccaatagagctcagtaggagtcagaacctctgcggtggctgtcagtgactcatccgcgtttcgtaagttgtgcgcgtgcacatttcgcccgttcccgctcatcttgcagcaggcggaaattttcatcacgctgtaggacgcaaaaaaaaaataattaatcgtacaagaatcttggaaaaaaaattgaaaaattttgtataaaagggatgacctaacttgactcaatggcttttacacccagtattttccctttccttgtttgttacaattatagaagcaagacaaaaacatatagacaacctattcctaggagttatatttttttaccctaccagcaatataagtaaaaaactgtttaaacagtatgaaggtgttttacgataaagactgcgatctgagcatcatccagggaaagaaggttgctattataggatatggttcccaaggacacgcacaagcctgtaacttgaaagattctggggtcgacgtgacagtaggtctgagaaaaggtagtgctaccgttgcaaaggctgaagcacatggcttgaaagtcacagatgttgcagcggctgttgccggcgctgatttagtcatgattttaacgccagatgaatttcaatcgcaattgtacaaaaatgaaatagaaccaaacattaagaagggcgctaccttggccttcagtcatggatttgccattcattacaatcaagtagtccccagggcagatttggacgttattatgattgcacctaaggctccggggcatactgttaggagcgaatttgttaagggtggtggtattccagatttgatcgctatataccaagacgcaagcggaaacgctaagaatgtagctttaagctacgcagcaggagttggtggcgggagaacgggtataatagaaaccacttttaaagacgagactgagacagatttatttggagaacaagcggttctgtgcggaggaactgttgaattggttaaagcaggctttgagacgcttgtcgaagcagggtacgctcccgaaatggcatacttcgaatgtctacatgaattgaagttgatagtagacttaatgtatgaaggtggtatagctaatatgaactattccatttcaaataatgcagaatatggtgagtatgtcaccggacctgaagtcattaacgcagaatcaagacaagccatgagaaatgccttgaaacgtatccaggacggtgaatacgctaagatgttcataagtgaaggcgctacgggttacccgagtatgactgctaaaagaagaaacaatgcagcacatggtatcgaaattattggtgaacagttaaggtctatgatgccctggatcggtgctaataagatcgtagacaaggcgaaaaattaaggccctgcaggcctatcaagtgctggaaactttttctcttggaatttttgcaacatcaagtcatagtcaattgaattgacccaatttcacatttaagattttttttttttcatccgacatacatctgtacactaggaagccctgtttttctgaagcagcttcaaatatatatattttttacatatttattatgattcaatgaacaatctaattaaatcgaaaacaagaaccgaaacgcgaataaataatttatttagatggtgacaagtgtataagtcctcatcgggacagctacgatttctctttcggttttggctgagctactggttgctgtgacgcagcggcattagcgcggcgttatgagctaccctcgtggcctgaaagatggcgggaataaagcggaactaaaaattactgactgagccatattgaggtcaatttgtcaactcgtcaagtcacgtttggtggacggcccctttccaacgaatcgtatatactaacatgcgcgcgcttcctatatacacatatacatatatatatatatatatatgtgtgcgtgtatgtgtacacctgtatttaatttccttactcgcgggtttttcttttttctcaattcttggcttcctctttctcgagcggaccggatcctccgcggtgccggcagatctatttaaatggcgcgccgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttttctttccaattttttttttttcgtcattataaaaatcattacgaccgagattcccgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagcttgcatgcctgcaggtcgactctagtatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcattttttttttttttttttttttttttttttttttttttttttttttttttttgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggctttaccttcttcaggcaagttcaatgacaatttcaacatcattgcagcagacaagatagtggcgatagggttgaccttattctttggcaaatctggagcggaaccatggcatggttcgtacaaaccaaatgcggtgttcttgtctggcaaagaggccaaggacgcagatggcaacaaacccaaggagcctgggataacggaggcttcatcggagatgatatcaccaaacatgttgctggtgattataataccatttaggtgggttgggttcttaactaggatcatggcggcagaatcaatcaattgatgttgaactttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctaacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcagccttcttggaggcttccagcgcctcatctggaagtggaacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacattacaatggtatatccttgaaatatatataaaaaaaaaaaaaaaaaaaaaaaaaaaaaatgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatatgcatagtaccgagaaactagaggatc

SEQ ID NO: 205 - DNA, 36 bp, artificial sequence
  primer
gcagtttaaacagtatgaaggtgttttacgataaag

SEQ ID NO: 206 - DNA, 42 bp, artificial sequence
  primer
gataggcctgcagggccttaatttttcgccttgtctacgatc

SEQ ID NO: 207 - DNA, 16387 bp, artificial sequence
  constructed plasmid
gatcctctagtttctcggtactatgcatatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttaacaaagatatgctattgaagtgcaagatggaaacgcagaaaatgaaccggggatgcgacgtgcaagattacctatgcaatagatgcaatagtttctccaggaaccgaaatacatacattgtcttccgtaaagcgctagactatatattattatacaggttcaaatatactatctgtttcagggaaaactcccaggttcggatgttcaaaattcaatgatgggtaacaagtacgatcgtaaatctgtaaaacagtttgtcggatattaggctgtatctcctcaaagcgtattcgaatatcattgagaagctgcattttttttttttttttttttttttttttttatatatatttcaaggatataccattgtaatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgttccacttccagatgaggcgctggaagcctccaagaaggctgatgccgttttgttaggtgctgtgggtggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgttagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgaattccctacattgaaagttcaacatcaattgattgattctgccgccatgatcctagttaagaacccaacccacctaaatggtattataatcaccagcaacatgtttggtgatatcatctccgatgaagcctccgttatcccaggctccttgggtttgttgccatctgcgtccttggcctctttgccagacaagaacaccgcatttggtttgtacgaaccatgccatggttccgctccagatttgccaaagaataaggtcaaccctatcgccactatcttgtctgctgcaatgatgttgaaattgtcattgaacttgcctgaagaaggtaaagccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacaaaaaaaaaaaaaaaaaaaaaaaaaaaaaaaaaaaaaaaaaaaaaaaaaaaatgcagcgtcacatcggataataatgatggcagccattgtagaagtgccttttgcatttctagtctctttctcggtctagctagttttactacatcgcgaagatagaatcttagatcacactgcctttgctgagctggatcaatagagtaacaaaagagtggtaaggcctcgttaaaggacaaggacctgagcggaagtgtatcgtacagtagacggagtatactagagtcgacctgcaggcatgcaagcttttcaattcatcattttttttttattcttttttttgatttcggtttccttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaatagaccgaaatcggcaaaatcccttataaatcaaaagaatagcccgagatagagttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctaaggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctccaccgcggatagatctgaaatgaataacaatactgacagtactaaataattgcctacttggcttcacatacgttgcatacgtcgatatagataataatgataatgacagcaggattatcgtaatacgtaatagttgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaatctagagctgaggatgttgaagcaaatcaacttcggtggtactgttgaaaccgtctacgaaagagctgactggccaagagaaaagttgttggactacttcaagaacgacacttttgctttgatcggttacggttcccaaggttacggtcaaggtttgaacttgagagacaacggtttgaacgttatcattggtgtccgtaaagatggtgcttcttggaaggctgccatcgaagacggttgggttccaggcaagaacttgttcactgttgaagatgctatcaagagaggtagttacgttatgaacttgttgtccgatgccgctcaatcagaaacctggcctgctatcaagccattgttgaccaagggtaagactttgtacttctcccacggtttctccccagtcttcaaggacttgactcacgttgaaccaccaaaggacttagatgttatcttggttgctccaaagggttccggtagaactgtcagatctttgttcaaggaaggtcgtggtattaactcttcttacgccgtctggaacgatgtcaccggtaaggctcacgaaaaggcccaagctttggccgttgccattggttccggttacgtttaccaaaccactttcgaaagagaagtcaactctgacttgtacggtgaaagaggttgtttaatgggtggtatccacggtatgttcttggctcaatacgacgtcttgagagaaaacggtcactccccatctgaagctttcaacgaaaccgtcgaagaagctacccaatctctatacccattgatcggtaagtacggtatggattacatgtacgatgcttgttccaccaccgccagaagaggtgctttggactggtacccaatcttcaagaatgctttgaagcctgttttccaagacttgtacgaatctaccaagaacggtaccgaaaccaagagatctttggaattcaactctcaacctgactacagagaaaagctagaaaaggaattagacaccatcagaaacatggaaatctggaaggttggtaaggaagtcagaaagttgagaccagaaaaccaataattaattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcgggcggccgctctagagagttgttagcaaccttttgtttcttttgagctggttcagacattatgtacacgtatatgtgacgagttcgagaagtattttactatcgtactaaattttacctgaaaaattatatactcgagaaagaggaagccaagaattgagaaaaaagaaaaacccgcgagtaaggaaattaaatacaggtgtacacatacacgcacacatatatatatatatatatatgtatatgtgtatataggaagcgcgcgcatgttagtatatacgattcgttggaaaggggccgtccaccaaacgtgacttgacgagttgacaaattgacctcaatatggctcagtcagtaatttttagttccgctttattcccgccatctttcaggccacgagggtagctcataacgccgcgctaatgccgctgcgtcacagcaaccagtagctcagccaaaaccgaaagagaaatcgtagctgtcccgatgaggacttatacacttgtcaccatctaaataaattatttattcgcgtttcggttcttgttttcgatttaattagattgttcattgaatcataataaatatgtaaaaaatatatatatttgaagctgcttcagaaaaacagggcttcctagtgtacagatgtatgtcggatgaaaaaaaaaaaatcttaaatgtgaaattgggtcaattcaattgactatgacttgatgttgcaaaaattccaagagaaaaagtttccagcacttgatattattttcctctttaatttttcgccttgtctacgatcttattagcaccgatccagggcatcatagaccttaactgttcaccaataatttcgataccatgtgctgcattgtttcttcttttagcagtcatactcgggtaacccgtagcgccttcacttatgaacatcttagcgtattcaccgtcctggatacgtttcaaggcatttctcatggcttgtcttgattctgcgttaatgacttcaggtccggtgacatactcaccatattctgcattatttgaaatggaatagttcatattagctataccaccttcatacattaagtctactatcaacttcaattcatgtagacattcgaagtatgccatttcgggagcgtaccctgcttcgacaagcgtctcaaagcctgctttaaccaattcaacagttcctccgcacagaaccgcttgttctccaaataaatctgtctcagtctcgtctttaaaagtggtttctattatacccgttctcccgccaccaactcctgctgcgtagcttaaagctacattcttagcgtttccgcttgcgtcttggtatatagcgatcaaatctggaataccaccacccttaacaaattcgctcctaacagtatgccccggagccttaggtgcaatcataataacgtccaaatctgccctggggactacttgattgtaatgaatggcaaatccatgactgaaggccaaggtagcgcccttcttaatgtttggttctatttcatttttgtacaattgcgattgaaattcatctggcgttaaaatcatgactaaatcagcgccggcaacagccgctgcaacatctgtgactttcaagccatgtgcttcagcctttgcaacggtagcactaccttttctcagacctactgtcacgtcgaccccagaatctttcaagttacaggcttgtgcgtgtccttgggaaccatatcctataatagcaaccttctttccctggatgatgctcagatcgcagtctttatcgtaaaacaccttcatgttttattttttacttatattgctggtagggtaaaaaaatataactcctaggaataggttgtctatatgtttttgtcttgcttctataattgtaacaaacaaggaaagggaaaatactgggtgtaaaagccattgagtcaagttaggtcatcccttttatacaaaatttttcaattttttttccaagattcttgtacgattaattattttttttttgcgtcctacagcgtgatgaaaatttccgcctgctgcaagatgagcgggaacgggcgaaatgtgcacgcgcacaacttacgaaacgcggatgagtcactgacagccaccgcagaggttctgactcctactgagctctattggaggtggcagaaccggtaccggaggagaccgctataaccggtttgaatttattgtcacagtgtcacatcagcggcaactcagaagtttgacagcaagcaagttcatcattcgaactagccttattgttttagttcagtgacagcgaactgccgtactcgatgctttatttctcacggtagagcggaagaacagataggggcagcgtgagaagagttagaaagtaaatttttatcacgtctgaagtattcttattcataggaaattttgcaaggttttttagctcaataacgggctaagttatataaggtgttcacgcgattttcttgttatgtatacctcttctctgaggaatggtactactgtcctgatgtaggctccttaaattggtgggcaagaataacttatcgatattttgtatattggtcttggagttcaccacgtaatgcctgtttaagaccatcagttaactctagtattatttggtcttggctactggccgtttgctattattcaagtcttttgtgccttcccgtcgggtaagggagttatttagggatacagaatctaacgaaaactaaatctcaatgattaactccatttaatccttttttgaaaggcaaaagaggtcccttgttcacttacaacgttcttagccaaattcgcttatcacttactacttcacgatatacagaagtaaaaacatataaaaagatgtctgtttgtttagccatcacaaaaggtatcgcagtttcttctataggcctctactctggtcttttggcttccgcttcattgattacatctactactccactagaggttttaacaggatctctaaaaacatcgatatcgtctctgcgttccaatcctacggtgaatatatttccaagcaattcactgaagaagaaagagaagatgttgtggaacatgcatgcccaggtcctggttcttgtggtggtatgtatactgccaacacaatggcttctgccgctgaagtgctaggtttgaccattccaaactcctcttccttcccagccgtttccaaggagaagttagctgagtgtgacaacattggtgaatacatcaagaagacaatggaattgggtattttacctcgtgatatcctcacaaaagaggcttttgaaaacgccattacttatgtcgttgcaaccggtgggtccactaatgctgttttgcatttggtggctgttgctcactctgcgggtgtcaagttgtcaccagatgatttccaaagaatcagtgatactacaccattgatcggtgacttcaaaccttctggtaaatacgtcatggccgatttgattaacgttggtggtacccaatctgtgattaagtatctatatgaaaacaacatgttgcacggtaacacaatgactgttaccggtgacactttggcagaacgtgcaaagaaagcaccaagcctacctgaaggacaagagattattaagccactctcccacccaatcaaggccaacggtcacttgcaaattctgtacggttcattggcaccaggtggagctgtgggtaaaattaccggtaaggaaggtacttacttcaagggtagagcacgtgtgttcgaagaggaaggtgcctttattgaagccttggaaagaggtgaaatcaagaagggtgaaaaaaccgttgttgttatcagatatgaaggtccaagaggtgcaccaggtatgcctgaaatgctaaagccttcctctgctctgatgggttacggtttgggtaaagatgttgcattgttgactgatggtagattctctggtggttctcacgggttcttaatcggccacattgttcccgaagccgctgaaggtggtcctatcgggttggtcagagacggcgatgagattatcattgatgctgataataacaagattgacctattagtctctgataaggaaatggctcaacgtaaacaaagttgggttgcacctccacctcgttacacaagaggtactctatccaagtatgctaagttggtttccaacgcttccaacggttgtgttttagatgcttgattaattaagagtaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacctgtggtactagttctagagcggccgcccgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgattaattaactagagagctttcgttttcatgagttccccgaattctttcggaagcttgtcacttgctaaattaatgttatcactgtagtcaaccgggacatcgatgatgacaggaccttcagcgttcatgccttgacgcagaacatctgccagctggtctggtgattctacgcgcaagccagttgctccgaagctttccgcatatttcacgatatcgatatttccgaaatcgaccgcagatgtacggttatattttttcaattgctggaatgcaaccatgtcatatgtgctgtcgttccatacaatgtgtacaattggtgcttttagtcgaactgctgtctctaattccattgctgagaataagaaaccgccgtcaccagagacagaaaccactttttctcccggtttcaccaatgaagcgccgattgcccaaggaagcgcaacgccgagtgtttgcataccgttactgatcattaatgttaacggctcgtagctgcggaaataacgtgacatccaaatggcgtgcgaaccgatatcgcaagttactgtaacatgatcatcgactgcattacgcaactctttaacgatttcaagagggtgcgctctgtctgatttccaatctgcaggcacctgctcaccttcatgcatatattgttttaaatcagaaaggattttctgctcacgctctgcaaattccactttcacagcatcgtgttcgatatgattgatcgtggacggaatgtcaccgatcaattcaagatcaggctggtaagcatgatcaatgtcagcgataatctcgtctaaatggataattgtccggtctccattgatattccagaatttcggatcatattcaatcgggtcatagccgatcgtcagaacaacatctgcctgctctagcagtaaatcgccaggctggttgcggaacaaaccgatacggccaaaatattgatcctctaaatctctagaaagggtaccggcagcttgatatgtttcaacaaatggaagctgaacctttttcaaaagcttgcgaaccgctttaattgcttccggtcttccgcctttcatgccgaccaaaacgacaggaagttttgctgtttggatttttgctatggccgcactgattgcatcatctgctgcaggaccgagttttggcgctgcaacagcacgcacgtttttcgtatttgtgacttcattcacaacatcttgcggaaagctcacaaaagcggccccagcctgccctgctgacgctatcctaaatgcatttgtaacagcttccggtatattttttacatcttgaacttctacactgtattttgtaatcggctggaatagcgccgcattatccaaagattgatgtgtccgttttaaacgatctgcacggatcacgtttccagcaagcgcaacgacagggtctccttcagtgttcgctgtcagcaggcctgttgccaagttagaggcacccggtcctgatgtgactaacacgactcccggttttccagttaaacggccgactgcttgggccatgaatgctgcgttttgttcgtgccgggcaacgataatttcaggtcctttatcttgtaaagcgtcaaataccgcatcaatttttgcacctggaatgccaaatacatgtgtgacaccttgctccactaagcaatcaacaacaagctccgcccctctgtttttcacaagggatttttgttcttttgttgcttttgtcaacatcctcagcgatgattgattgattgattgtacagtttgtttttcttaatatctatttcgatgacttctatatgatattgcactaacaagaagatattataatgcaattgatacaagacaaggagttatttgcttctcttttatatgattctgacaatccatattgcgttggtagtcttttttgctggaacggttcagcggaaaagacgcatcgctctttttgcttctagaagaaatgccagcaaaagaatctcttgacagtgactgacagcaaaaatgtctttttctaactagtaacaaggctaagatatcagcctgaaataaagggtggtgaagtaataattaaatcatccgtataaacctatacacatatatgaggaaaaataatacaaaagtgttttaaatacagatacatacatgaacatatgcacgtatagcgcccaaatgtcggtaatggga

SEQ ID NO: 208 - DNA, 11871 bp, artificial sequence
  constructed plasmid
tcccattaccgacatttgggcgctatacgtgcatatgttcatgtatgtatctgtatttaaaacacttttgtattatttttcctcatatatgtgtataggtttatacggatgatttaattattacttcaccaccctttatttcaggctgatatcttagccttgttactagttagaaaaagacatttttgctgtcagtcactgtcaagagattcttttgctggcatttcttctagaagcaaaaagagcgatgcgtcttttccgctgaaccgttccagcaaaaaagactaccaacgcaatatggattgtcagaatcatataaaagagaagcaaataactccttgtcttgtatcaattgcattataatatcttcttgttagtgcaatatcatatagaagtcatcgaaatagatattaagaaaaacaaactgtacaatcaatcaatcaatcatcgctgaggatgttgacaaaagcaacaaaagaacaaaaatcccttgtgaaaaacagaggggcggagcttgttgttgattgcttagtggagcaaggtgtcacacatgtatttggcattccaggtgcaaaaattgatgcggtatttgacgctttacaagataaaggacctgaaattatcgttgcccggcacgaacaaaacgcagcattcatggcccaagcagtcggccgtttaactggaaaaccgggagtcgtgttagtcacatcaggaccgggtgcctctaacttggcaacaggcctgctgacagcgaacactgaaggagaccctgtcgttgcgcttgctggaaacgtgatccgtgcagatcgtttaaaacggacacatcaatctttggataatgcggcgctattccagccgattacaaaatacagtgtagaagttcaagatgtaaaaaatataccggaagctgttacaaatgcatttaggatagcgtcagcagggcaggctggggccgcttttgtgagctttccgcaagatgttgtgaatgaagtcacaaatacgaaaaacgtgcgtgctgttgcagcgccaaaactcggtcctgcagcagatgatgcaatcagtgcggccatagcaaaaatccaaacagcaaaacttcctgtcgttttggtcggcatgaaaggcggaagaccggaagcaattaaagcggttcgcaagcttttgaaaaaggttcagcttccatttgttgaaacatatcaagctgccggtaccctttctagagatttagaggatcaatattttggccgtatcggtttgttccgcaaccagcctggcgatttactgctagagcaggcagatgttgttctgacgatcggctatgacccgattgaatatgatccgaaattctggaatatcaatggagaccggacaattatccatttagacgagattatcgctgacattgatcatgcttaccagcctgatcttgaattgatcggtgacattccgtccacgatcaatcatatcgaacacgatgctgtgaaagtggaatttgcagagcgtgagcagaaaatcctttctgatttaaaacaatatatgcatgaaggtgagcaggtgcctgcagattggaaatcagacagagcgcaccctcttgaaatcgttaaagagttgcgtaatgcagtcgatgatcatgttacagtaacttgcgatatcggttcgcacgccatttggatgtcacgttatttccgcagctacgagccgttaacattaatgatcagtaacggtatgcaaacactcggcgttgcgcttccttgggcaatcggcgcttcattggtgaaaccgggagaaaaagtggtttctgtctctggtgacggcggtttcttattctcagcaatggaattagagacagcagttcgactaaaagcaccaattgtacacattgtatggaacgacagcacatatgacatggttgcattccagcaattgaaaaaatataaccgtacatctgcggtcgatttcggaaatatcgatatcgtgaaatatgcggaaagcttcggagcaactggcttgcgcgtagaatcaccagaccagctggcagatgttctgcgtcaaggcatgaacgctgaaggtcctgtcatcatcgatgtcccggttgactacagtgataacattaatttagcaagtgacaagcttccgaaagaattcggggaactcatgaaaacgaaagctctctagttaattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcgggcggccgcacctggtaaaacctctagtggagtagtagatgtaatcaatgaagcggaagccaaaagaccagagtagaggcctatagaagaaactgcgataccttttgtgatggctaaacaaacagacatctttttatatgtttttacttctgtatatcgtgaagtagtaagtgataagcgaatttggctaagaacgttgtaagtgaacaagggacctcttttgcctttcaaaaaaggattaaatggagttaatcattgagatttagttttcgttagattctgtatccctaaataactcccttacccgacgggaaggcacaaaagacttgaataatagcaaacggccagtagccaagaccaaataatactagagttaactgatggtcttaaacaggcattacgtggtgaactccaagaccaatatacaaaatatcgataagttattcttgcccaccaatttaaggagcctacatcaggacagtagtaccattcctcagagaagaggtatacataacaagaaaatcgcgtgaacaccttatataacttagcccgttattgagctaaaaaaccttgcaaaatttcctatgaataagaatacttcagacgtgataaaaatttactttctaactcttctcacgctgcccctatctgttcttccgctctaccgtgagaaataaagcatcgagtacggcagttcgctgtcactgaactaaaacaataaggctagttcgaatgatgaacttgcttgctgtcaaacttctgagttgccgctgatgtgacactgtgacaataaattcaaaccggttatagcggtctcctccggtaccggttctgccacctccaatagagctcagtaggagtcagaacctctgcggtggctgtcagtgactcatccgcgtttcgtaagttgtgcgcgtgcacatttcgcccgttcccgctcatcttgcagcaggcggaaattttcatcacgctgtaggacgcaaaaaaaaaataattaatcgtacaagaatcttggaaaaaaaattgaaaaattttgtataaaagggatgacctaacttgactcaatggcttttacacccagtattttccctttccttgtttgttacaattatagaagcaagacaaaaacatatagacaacctattcctaggagttatatttttttaccctaccagcaatataagtaaaaaactgtttaaacagtatgttgaagcaaatcaacttcggtggtactgttgaaaccgtctacgaaagagctgactggccaagagaaaagttgttggactacttcaagaacgacacttttgctttgatcggttacggttcccaaggttacggtcaaggtttgaacttgagagacaacggtttgaacgttatcattggtgtccgtaaagatggtgcttcttggaaggctgccatcgaagacggttgggttccaggcaagaacttgttcactgttgaagatgctatcaagagaggtagttacgttatgaacttgttgtccgatgccgctcaatcagaaacctggcctgctatcaagccattgttgaccaagggtaagactttgtacttctcccacggtttctccccagtcttcaaggacttgactcacgttgaaccaccaaaggacttagatgttatcttggttgctccaaagggttccggtagaactgtcagatctttgttcaaggaaggtcgtggtattaactcttcttacgccgtctggaacgatgtcaccggtaaggctcacgaaaaggcccaagctttggccgttgccattggttccggttacgtttaccaaaccactttcgaaagagaagtcaactctgacttgtacggtgaaagaggttgtttaatgggtggtatccacggtatgttcttggctcaatacgacgtcttgagagaaaacggtcactccccatctgaagctttcaacgaaaccgtcgaagaagctacccaatctctatacccattgatcggtaagtacggtatggattacatgtacgatgcttgttccaccaccgccagaagaggtgctttggactggtacccaatcttcaagaatgctttgaagcctgttttccaagacttgtacgaatctaccaagaacggtaccgaaaccaagagatctttggaattcaactctcaacctgactacagagaaaagctagaaaaggaattagacaccatcagaaacatggaaatctggaaggttggtaaggaagtcagaaagttgagaccagaaaaccaataaggccctgcaggcctatcaagtgctggaaactttttctcttggaatttttgcaacatcaagtcatagtcaattgaattgacccaatttcacatttaagattttttttttttcatccgacatacatctgtacactaggaagccctgtttttctgaagcagcttcaaatatatatattttttacatatttattatgattcaatgaacaatctaattaaatcgaaaacaagaaccgaaacgcgaataaataatttatttagatggtgacaagtgtataagtcctcatcgggacagctacgatttctctttcggttttggctgagctactggttgctgtgacgcagcggcattagcgcggcgttatgagctaccctcgtggcctgaaagatggcgggaataaagcggaactaaaaattactgactgagccatattgaggtcaatttgtcaactcgtcaagtcacgtttggtggacggcccctttccaacgaatcgtatatactaacatgcgcgcgcttcctatatacacatatacatatatatatatatatatatgtgtgcgtgtatgtgtacacctgtatttaatttccttactcgcgggtttttcttttttctcaattcttggcttcctctttctcgagcggaccggatcctccgcggtgccggcagatctatttaaatggcgcgccgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttttctttccaattttttttttttcgtcattataaaaatcattacgaccgagattcccgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagcttgcatgcctgcaggtcgactctagtatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcattttttttttttttttttttttttttttttttttttttttttttttttttttgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggctttaccttcttcaggcaagttcaatgacaatttcaacatcattgcagcagacaagatagtggcgatagggttgaccttattctttggcaaatctggagcggaaccatggcatggttcgtacaaaccaaatgcggtgttcttgtctggcaaagaggccaaggacgcagatggcaacaaacccaaggagcctgggataacggaggcttcatcggagatgatatcaccaaacatgttgctggtgattataataccatttaggtgggttgggttcttaactaggatcatggcggcagaatcaatcaattgatgttgaactttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctaacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcagccttcttggaggcttccagcgcctcatctggaagtggaacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacattacaatggtatatccttgaaatatatataaaaaaaaaaaaaaaaaaaaaaaaaaaaaatgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatatgcatagtaccgagaaactagaggatc

SEQ ID NO: 209 - DNA, 37 bp, artificial sequence
  primer
gcagtttaaacagtatgttgaagcaaatcaacttcgg

SEQ ID NO: 210 - DNA, 40 bp, artificial sequence
  primer
gataggcctgcagggccattattggttttctggtctcaac

SEQ ID NO: 211 - DNA, 13114 bp, artificial sequence
  constructed plasmid
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttccacctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctaggggccgtgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtggccggcttcacatacgttgcatacgtcgatatagataataatgataatgacagcaggattatcgtaatacgtaatagctgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaacacgtgagtatgactgacaaaaaaactcttaaagacttaagaaatcgtagttctgtttacgattcaatggttaaatcacctaatcgtgctatgttgcgtgcaactggtatgcaagatgaagactttgaaaaacctatcgtcggtgtcatttcaacttgggctgaaaacacaccttgtaatatccacttacatgactttggtaaactagccaaagtcggtgttaaggaagctggtgcttggccagttcagttcggaacaatcacggtttctgatggaatcgccatgggaacccaaggaatgcgtttctccttgacatctcgtgatattattgcagattctattgaagcagccatgggaggtcataatgcggatgcttttgtagccattggcggttgtgataaaaacatgcccggttctgttatcgctatggctaacatggatatcccagccatttttgcttacggcggaacaattgcacctggtaatttagacggcaaagatatcgatttagtctctgtctttgaaggtgtcggccattggaaccacggcgatatgaccaaagaagaagttaaagctttggaatgtaatgcttgtcccggtcctggaggctgcggtggtatgtatactgctaacacaatggcgacagctattgaagttttgggacttagccttccgggttcatcttctcacccggctgaatccgcagaaaagaaagcagatattgaagaagctggtcgcgctgttgtcaaaatgctcgaaatgggcttaaaaccttctgacattttaacgcgtgaagcttttgaagatgctattactgtaactatggctctgggaggttcaaccaactcaacccttcacctcttagctattgcccatgctgctaatgtggaattgacacttgatgatttcaatactttccaagaaaaagttcctcatttggctgatttgaaaccttctggtcaatatgtattccaagacctttacaaggtcggaggggtaccagcagttatgaaatatctccttaaaaatggcttccttcatggtgaccgtatcacttgtactggcaaaacagtcgctgaaaatttgaaggcttttgatgatttaacacctggtcaaaaggttattatgccgcttgaaaatcctaaacgtgaagatggtccgctcattattctccatggtaacttggctccagacggtgccgttgccaaagtttctggtgtaaaagtgcgtcgtcatgtcggtcctgctaaggtctttaattctgaagaagaagccattgaagctgtcttgaatgatgatattgttgatggtgatgttgttgtcgtacgttttgtaggaccaaagggcggtcctggtatgcctgaaatgctttccctttcatcaatgattgttggtaaagggcaaggtgaaaaagttgcccttctgacagatggccgcttctcaggtggtacttatggtcttgtcgtgggtcatatcgctcctgaagcacaagatggcggtccaatcgcctacctgcaaacaggagacatagtcactattgaccaagacactaaggaattacactttgatatctccgatgaagagttaaaacatcgtcaagagaccattgaattgccaccgctctattcacgcggtatccttggtaaatatgctcacatcgtttcgtctgcttctaggggagccgtaacagacttttggaagcctgaagaaactggcaaaaaatgttgtcctggttgctgtggttaagcggccgcgttaattcaaattaattgatatagttttttaatgagtattgaatctgtttagaaataatggaatattatttttatttatttatttatattattggtcggctcttttcttctgaaggtcaatgacaaaatgatatgaaggaaataatgatttctaaaattttacaacgtaagatatttttacaaaagcctagctcatcttttgtcatgcactattttactcacgcttgaaattaacggccagtccactgcggagtcatttcaaagtcatcctaatcgatctatcgtttttgatagctcattttggagttcgcgaggatccactagttctagagcggccgctctagaactagtaccacaggtgttgtcctctgaggacataaaatacacaccgagattcatcaactcattgctggagttagcatatctacaattgggtgaaatggggagcgatttgcaggcatttgctcggcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttactcttaattaatcaaaaagttaaaattgtacgaatagattcaccacttcttaacaaatcaaacccttcattgattttctcgaatggcaatacatgtgtaattaaaggatcaagagcaaacttcttcgccataaagtcggcaacaagttttggaacactatccttgctcttaaaaccgccaaatatagctcccttccatgtacgaccgcttagcaacagcataggattcatcgacaaattttgtgaatcaggaggaacacctacgatcacactgactccatatgcctcttgacagcaggacaacgcagttaccatagtatcaagacggcctataacttcaaaagagaaatcaactccaccgtttgacatttcagtaaggacttcttgtattggtttcttataatcttgagggttaacacattcagtagccccgacctccttagcttttgcaaatttgtccttattgatgtctacacctataatcctcgctgcgcctgcagctttacaccccataataacgcttagtcctactcctcctaaaccgaatactgcacaagtcgaaccctgtgtaacctttgcaactttaactgcggaaccgtaaccggtggaaaatccgcaccctatcaagcaaactttttccagtggtgaagctgcatcgattttagcgacagatatctcgtccaccactgtgtattgggaaaatgtagaagtaccaaggaaatggtgtataggtttccctctgcatgtaaatctgcttgtaccatcctgcatagtacctctaggcatagacaaatcatttttaaggcagaaattaccctcaggatgtttgcagactctacacttaccacattgaggagtgaacagtgggatcactttatcaccaggacgaacagtggtaacaccttcacctatggattcaacgattccggcagcctcgtgtcccgcgattactggcaaaggagtaactagagtgccactcaccacatggtcgtcggatctacagattccggtggcaaccatcttgattctaacctcgtgtgcttttggtggcgctacttctacttcttctatgctaaacggctttttctcttcccacaaaactgccgctttacacttaataactttaccggctgttgacatcctcagctagctattgtaatatgtgtgtttgtttggattattaagaagaataattacaaaaaaaattacaaaggaaggtaattacaacagaattaagaaaggacaagaaggaggaagagaatcagttcattatttcttctttgttatataacaaacccaagtagcgatttggccatacattaaaagttgagaaccaccctccctggcaacagccacaactcgttaccattgttcatcacgatcatgaaactcgccgtcagctgaaatttcacctcagtggatctctctttttattcttcatcgttccactaacctttttccatcagctggcagggaacggaaagtggaatcccatttagcgagcttcctcttttcttcaagaaaagacgaagcttgtgtgtgggtgcgcgcgctagtatctttccacattaagaaatataccataaaggttacttagacatcactatggctatatatatatatatatatatatgtaacttagcaccatcgcgcgtgcatcactgcatgtgttaaccgaaaagtttggcgaacacttcaccgacacggtcatttagatctgtcgtctgcattgcacgtcccttagccttaaatcctaggcgggagcattctcgtgtaattgtgcagcctgcgtagcaactcaacatagcgtagtctacccagtttttcaagggtttatcgttagaagattctcccttttcttcctgctcacaaatcttaaagtcatacattgcacgactaaatgcaagcgacgtcagggaaagatatgagctatacagcggaatttccatatcactcagattttgttatctaattttttccttcccacgtccgcgggaatctgtgtatattactgcatctagatatatgttatcttatcttggcgcgtacatttaattttcaacgtattctataagaaattgcgggagtttttttcatgtagatgatactgactgcacgcaaatataggcatgatttataggcatgatttgatggctgtaccgataggaacgctaagagtaacttcagaatcgttatcctggcggaaaaaattcatttgtaaactttaaaaaaaaaagccaatatccccaaaattattaagagcgcctccattattaactaaaatttcactcagcatccacaatgtatcaggtatctactacagatattacatgtggcgaaaaagacaagaacaatgcaatagcgcatcaagaaaaaacacaaagctttcaatcaatgaatcgaaaatgtcattaaaatagtatataaattgaaactaagtcataaagctataaaaagaaaatttatttaaatgcaagatttaaagtaaattcacggccctgcaggcctcagctcttgttttgttctgcaaataacttacccatctttttcaaaactttaggtgcaccctcctttgctagaataagttctatccaatacatcctatttggatctgcttgagcttctttcatcacggatacgaattcattttctgttctcacaattttggacacaactctgtcttccgttgccccgaaactttctggcagttttgagtaattccacataggaatgtcattataactctggttcggaccatgaatttccctctcaaccgtgtaaccatcgttattaatgataaagcagattgggtttatcttctctctaatggctagtcctaattcttggacagtcagttgcaatgatccatctccgataaacaataaatgtctagattctttatctgcaatttggctgcctagagctgcggggaaagtgtatcctatagatccccacaagggttgaccaataaaatgtgatttcgatttcagaaatatagatgaggcaccgaagaaagaagtgccttgttcagccacgatcgtctcattactttgggtcaaattttcgacagcttgccacagtctatcttgtgacaacagcgcgttagaaggtacaaaatcttcttgctttttatctatgtacttgcctttatattcaatttcggacaagtcaagaagagatgatatcagggattcgaagtcgaaattttggattctttcgttgaaaattttaccttcatcgatattcaaggaaatcattttattttcattaagatggtgagtaaatgcacccgtactagaatcggtaagctttacacccaacataagaataaaatcagcagattccacaaattccttcaagtttggctctgacagagtaccgttgtaaatccccaaaaatgagggcaatgcttcatcaacagatgatttaccaaagttcaaagtagtaataggtaacttagtctttgaaataaactgagtaacagtcttctctaggccgaacgatataatttcatggcctgtgattacaattggtttcttggcattcttcagactttcctgtattttgttcagaatctcttgatcagatgtattcgacgtggaattttccttcttaagaggcaaggatggtttttcagccttagcggcagctacatctacaggtaaattgatgtaaaccggctttctttcctttagtaaggcagacaacactctatcaatttcaacagttgcattctcggctgtcaataaagtcctggcagcagtaaccggttcgtgcatcttcataaagtgcttgaaatcaccatcagccaacgtatggtgaacaaacttaccttcgttctgcactttcgaggtaggagatcccacgatctcaacaacaggcaggttctcagcataggagcccgctaagccattaactgcggataattcgccaacaccaaatgtagtcaagaatgccgcagcctttttcgttcttgcgtacccgtcggccatataggaggcatttaactcattagcatttcccacccatttcatatctttgtgtgaaataatttgatctagaaattgcaaattgtagtcacctggtactccgaatatttcttctatacctaattcgtgtaatctgtccaacagatagtcacctactgtatacatgtttaaactttgtttactagtttatgtgtgtttattcgaaactaagttcttggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcagtattggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtc

SEQ ID NO: 212 - DNA, 4280 bp, artificial sequence
  vector
ggggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccccggctctgagacagtagtaggttagtcatcgctctaccgacgcgcaggaaaagaaagaagcattgcggattacgtattctaatgttcagcccgcggaacgccagcaaatcaccacccatgcgcatgatactgagtcttgtacacgctgggcttccagtgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcttcagcccgcggaacgccagcaaatcaccacccatgcgcatgatactgagtcttgtacacgctgggcttccagtgatgatacaacgagttagccaaggtgagcacggatgtctaaattagaattacgttttaatatctttttttccatatctagggctag

SEQ ID NO: 213 - DNA, 30 bp, artificial sequence
  primer
gcatgcttgcatttagtcgtgcaatgtatg

SEQ ID NO: 214 - DNA, 54 bp, artificial sequence
  primer
gaacattagaatacgtaatccgcaatgcactagtaccacaggtgttgtcctctg

SEQ ID NO: 215 - DNA, 54 bp, artificial sequence
  primer
cagaggacaacacctgtggtactagtgcattgcggattacgtattctaatgttc

SEQ ID NO: 216 - DNA, 28 bp, artificial sequence
  primer
caccttggctaactcgttgtatcatcac

SEQ ID NO: 217 - DNA, 100 bp, artificial sequence
  primer
ttttaagccgaatgagtgacagaaaaagcccacaacttatcaagtgatattgaacaaagggcgaaacttcgcatgcttgcatttagtcgtgcaatgtatg

SEQ ID NO: 218 - DNA, 98 bp, artificial sequence
  primer
cccaattggtaaatattcaacaagagacgcgcagtacgtaacatgcgaattgcgtaattcacggcgataacaccttggctaactcgttgtatcatcac

SEQ ID NO: 219 - DNA, 29 bp, artificial sequence
  primer
caaaagcccatgtcccacaccaaaggatg

SEQ ID NO: 220 - DNA, 26 bp, artificial sequence
  primer
caccatcgcgcgtgcatcactgcatg

SEQ ID NO: 221 - DNA, 28 bp, artificial sequence
  primer
tcggtttttgcaatatgacctgtgggcc

SEQ ID NO: 222 - DNA, 22 bp, artificial sequence
  primer
gagaagatgcggccagcaaaac

SEQ ID NO: 223 - DNA, 2745 bp, artificial sequence
  constructed coding region-terminator segment
atgactgacaaaaaaactcttaaagacttaagaaatcgtagttctgtttacgattcaatggttaaatcacctaatcgtgctatgttgcgtgcaactggtatgcaagatgaagactttgaaaaacctatcgtcggtgtcatttcaacttgggctgaaaacacaccttgtaatatccacttacatgactttggtaaactagccaaagtcggtgttaaggaagctggtgcttggccagttcagttcggaacaatcacggtttctgatggaatcgccatgggaacccaaggaatgcgtttctccttgacatctcgtgatattattgcagattctattgaagcagccatgggaggtcataatgcggatgcttttgtagccattggcggttgtgataaaaacatgcccggttctgttatcgctatggctaacatggatatcccagccatttttgcttacggcggaacaattgcacctggtaatttagacggcaaagatatcgatttagtctctgtctttgaaggtgtcggccattggaaccacggcgatatgaccaaagaagaagttaaagctttggaatgtaatgcttgtcccggtcctggaggctgcggtggtatgtatactgctaacacaatggcgacagctattgaagttttgggacttagccttccgggttcatcttctcacccggctgaatccgcagaaaagaaagcagatattgaagaagctggtcgcgctgttgtcaaaatgctcgaaatgggcttaaaaccttctgacattttaacgcgtgaagcttttgaagatgctattactgtaactatggctctgggaggttcaaccaactcaacccttcacctcttagctattgcccatgctgctaatgtggaattgacacttgatgatttcaatactttccaagaaaaagttcctcatttggctgatttgaaaccttctggtcaatatgtattccaagacctttacaaggtcggaggggtaccagcagttatgaaatatctccttaaaaatggcttccttcatggtgaccgtatcacttgtactggcaaaacagtcgctgaaaatttgaaggcttttgatgatttaacacctggtcaaaaggttattatgccgcttgaaaatcctaaacgtgaagatggtccgctcattattctccatggtaacttggctccagacggtgccgttgccaaagtttctggtgtaaaagtgcgtcgtcatgtcggtcctgctaaggtctttaattctgaagaagaagccattgaagctgtcttgaatgatgatattgttgatggtgatgttgttgtcgtacgttttgtaggaccaaagggcggtcctggtatgcctgaaatgctttccctttcatcaatgattgttggtaaagggcaaggtgaaaaagttgcccttctgacagatggccgcttctcaggtggtacttatggtcttgtcgtgggtcatatcgctcctgaagcacaagatggcggtccaatcgcctacctgcaaacaggagacatagtcactattgaccaagacactaaggaattacactttgatatctccgatgaagagttaaaacatcgtcaagagaccattgaattgccaccgctctattcacgcggtatccttggtaaatatgctcacatcgtttcgtctgcttctaggggagccgtaacagacttttggaagcctgaagaaactggcaaaaaatgttgtcctggttgctgtggttaagcggccgcgttaattcaaattaattgatatagttttttaatgagtattgaatctgtttagaaataatggaatattatttttatttatttatttatattattggtcggctcttttcttctgaaggtcaatgacaaaatgatatgaaggaaataatgatttctaaaattttacaacgtaagatatttttacaaaagcctagctcatcttttgtcatgcactattttactcacgcttgaaattaacggccagtccactgcggagtcatttcaaagtcatcctaatcgatctatcgtttttgatagctcattttggagttcgcgattgtcttctgttattcacaactgttttaatttttatttcattctggaactcttcgagttctttgtaaagtctttcatagtagcttactttatcctccaacatatttaacttcatgtcaatttcggctcttaaattttccacatcatcaagttcaacatcatcttttaacttgaatttattctctagctcttccaaccaagcctcattgctccttgatttactggtgaaaagtgatacactttgcgcgcaatccaggtcaaaactttcctgcaaagaattcaccaatttctcgacatcatagtacaatttgttttgttctcccatcacaatttaatatacctgatggattcttatgaagcgctgggtaatggacgtgtcactctacttcgcctttttccctactccttttagtacggaagacaatgctaataaataagagggtaataataatattattaatcggcaaaaaagattaaacgccaagcgtttaattatcagaaagcaaacgtcgtaccaatccttgaatgcttcccaattgtatattaagagtcatcacagcaacatattcttgttattaaattaattattattgatttttgatattgtataaaaaaaccaaatatgtataaaaaaagtgaataaaaaataccaagtatggagaaatatattagaagtctatacgttaaa

SEQ ID NO: 224 - DNA, 99 bp, artificial sequence
  primer
tcctttctcaattattattttctactcataacctcacgcaaaataacacagtcaaatcaatcaaagtatgactgacaaaaaaactcttaaagacttaag

SEQ ID NO: 225 - DNA, 77 bp, artificial sequence
  primer
gaacattagaatacgtaatccgcaatgcttctttcttttccgtttaacgtatagacttctaatatatttctccatac

SEQ ID NO: 226 - DNA, 45 bp, artificial sequence
  primer
aaacggaaaagaaagaagcattgcggattacgtattctaatgttc

SEQ ID NO: 227 - DNA, 88 bp, artificial sequence
  primer
tatttttcgttacataaaaatgcttataaaactttaactaataattagagattaaatcgccaccttggctaactcgttgtatcatcac

SEQ ID NO: 228 - DNA, 27 bp, artificial sequence
  primer
gacttttggaagcctgaagaaactggc

SEQ ID NO: 229 - DNA, 20 bp, artificial sequence
  primer
cttggcagcaacaggactag

SEQ ID NO: 230 - DNA, 26 bp, artificial sequence
  primer
ccaggccaattcaacagactgtcggc

SEQ ID NO: 231 - DNA, 2347 bp, artificial sequence
  constructed URA3 marker with flanking homologous repeat sequences for HIS gene replacement and marker excision
gcattgcggattacgtattctaatgttcaggtgctggaagaagagctgcttaaccgccgcgcccagggtgaagatccacgctactttaccctgcgtcgtctggatttcggcggctgtcgtctttcgctggcaacgccggttgatgaagcctgggacggtccgctctccttaaacggtaaacgtatcgccacctcttatcctcacctgctcaagcgttatctcgaccagaaaggcatctcttttaaatcctgcttactgaacggttctgttgaagtcgccccgcgtgccggactggcggatgcgatttgcgatctggtttccaccggtgccacgctggaagctaacggcctgcgcgaagtcgaagttatctatcgctcgaaagcctgcctgattcaacgcgatggcgaaatggaagaatccaaacagcaactgatcgacaaactgctgacccgtattcagggtgtgatccaggcgcgcgaatcaaaatacatcatgatgcacgcaccgaccgaacgtctggatgaagtcatggtacctactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatctctagagtgctggaagaagagctgcttaaccgccgcgcccagggtgaagatccacgctactttaccctgcgtcgtctggatttcggcggctgtcgtctttcgctggcaacgccggttgatgaagcctgggacggtccgctctccttaaacggtaaacgtatcgccacctcttatcctcacctgctcaagcgttatctcgaccagaaaggcatctcttttaaatcctgcttactgaacggttctgttgaagtcgccccgcgtgccggactggcggatgcgatttgcgatctggtttccaccggtgccacgctggaagctaacggcctgcgcgaagtcgaagttatctatcgctcgaaagcctgcctgattcaacgcgatggcgaaatggaagaatccaaacagcaactgatcgacaaactgctgacccgtattcagggtgtgatccaggcgcgcgaatcaaaatacatcatgatgcacgcaccgaccgaacgtctggatgaagtcatccagtgatgatacaacgagttagccaaggtg

SEQ ID NO: 232 - DNA, 80 bp, artificial sequence
  primer
cttcgaagaatatactaaaaaatgagcaggcaagataaacgaaggcaaaggcattgcggattacgtattctaatgttcag

SEQ ID NO: 233 - DNA, 80 bp, artificial sequence; identical to SEQ ID NO: 232
  primer

SEQ ID NO: 234 - DNA, 26 bp, artificial sequence
  primer
gacttgaataatgcagcggcgcttgc

SEQ ID NO: 235 - DNA, 30 bp, artificial sequence
  primer
ccaccctcttcaattagctaagatcatagc

SEQ ID NO: 236 - DNA, 25 bp, artificial sequence
  primer
aaaaattgattctcatcgtaaatgc

SEQ ID NO: 237 - DNA, 20 bp, artificial sequence
  primer
ctgcagcgaggagccgtaat

SEQ ID NO: 238 - DNA, 90 bp, artificial sequence
  primer
atggttcatttaggtccaaaaaaaccacaagccagaaagggttccatggccgatgtgccagcattgcggattacgtattctaatgttcag

SEQ ID NO: 239 - DNA, 91 bp, artificial sequence
  primer
ttaagcaccgatgataccaacggacttaccttcagcaattcttttttgggccaaagcagccaccttggctaactcgttgtatcatcactgg

SEQ ID NO: 240 - DNA, 24 bp, artificial sequence
  primer
ctaggatgagtagcagcacgttcc

SEQ ID NO: 241 - DNA, 26 bp, artificial sequence
  primer
ccaattccgtgatgtctctttgttgc

SEQ ID NO: 242 - DNA, 20 bp, artificial sequence
  primer
gtgaacgagttcacaaccgc

SEQ ID NO: 243 - DNA, 22 bp, artificial sequence
  primer
gttcgttccagaattatcacgc

SEQ ID NO: 244 - DNA, 1002 bp, Staphylococcus hominis
atgacaaaagtttattatgatcaatcagtagaaaaagatgcactacaaggtaaaaaaatcgctattatcggttacggttctcaaggccatgcacatgctcaaaaccttaaagataacggttacgacgtaagagttggtattcgtccaggtgattcatttaataaagctaaagaagatggattagatgtttatccagtggctgaagcagttaaacaagcagacgttattatggtattacttcctgatgaaattcaaggtaatgtatataaaaatgaaattgctccaaatttagaagctggtaatgcattagcttttgcacatggatttaatattcattttaacgtcatcgaaccacctaaagatgtagatgtcttcttagtagcacctaaaggtcctggtcatctagtaagacgtacatttgttgaaggttcagctgtgccagcattatttggtgttcaacaagatgctacaggtcatgcacgtgacattgctttaagttatgcaaaaggtattggtgctacacgtgctggtgttattgaaacaacatttaaagaggaaactgaaactgacttattcggtgaacaagctgtactttgtgggggaattcataaactgattcaaagtggtttcgagacattagtggaagctggatatcaaaaagaattagcatattttgaagtactacatgaaatgaaacttatcgttgatttaatgtatgaaggcggtatggaaaatgtacgttattcaatttcaaatactgctgaatttggtgactacgtatctggtccacgtgtcattactccagatgttaaagacaatatgaaagaagtacttaaagatattcaaaatggtaatttcgctaatcgctttgttaaagataacgaaaatggatttaaagaattttatgaattgcgtgaacaacaacacggtcatgaaattgaagcagtgggtcgtgaactaagaaaaatgatgccattcattaaatctaaaagcattcaaaaa

SEQ ID NO: 245 - protein, 334 aa, Staphylococcus hominis
MTKVYYDQSVEKDALQGKKIAIIGYGSQGHAHAQNLKDNGYDVRVGIRPGDSFNKAKEDGLDVYPVAEAVKQADVIMVLLPDEIQGNVYKNEIAPNLEAGNALAFAHGFNIHFNVIEPPKDVDVFLVAPKGPGHLVRRTFVEGSAVPALFGVQQDATGHARDIALSYAKGIGATRAGVIETTFKEETETDLFGEQAVLCGGIHKLIQSGFETLVEAGYQKELAYFEVLHEMKLIVDLMYEGGMENVRYSISNTAEFGDYVSGPRVITPDVKDNMKEVLKDIQNGNFANRFVKDNENGFKEFYELREQQHGHEIEAVGRELRKMMPFIKSKSIQK